(19) United States
(12) Patent Application Publication
(10) Pub. No.: US 2021/0355104 A1
(43) Pub. Date: Nov. 18, 2021

(54) ADENOSINE RECEPTOR BINDING COMPOUNDS

(71) Applicant: Nikang Therapeuticsm Inc., Wilmington, DE (US)

(72) Inventors: Yan LOU, Pleasanton, CA (US); Jiping FU, Danville, CA (US); Yigang HE, Newark, DE (US)

(73) Assignee: Nikang Therapeutics, Inc., Wilmington, DE (US)

(21) Appl. No.: 17/258,376

(22) PCT Filed: Jul. 10, 2019
(86) PCT No.: PCT/US2019/041160

§ 371 Date: Jan. 6, 2021

Related U.S. Application Data
(60) Provisional application No. 62/695,877, filed on Jul. 10, 2018.

(51) Int. Cl. C07D 401/14; C07D 401/04; C07D 405/14; C07D 413/14; C07D 487/04; C07D 471/04; C07D 409/14; C07D 417/14

(52) U.S. Cl. CPC C07D 401/14; C07D 401/04; C07D 405/14; C07D 417/14; C07D 487/04; C07D 471/04; C07D 409/14; C07D 413/14

(57) ABSTRACT

The present invention relates to pharmaceutical compounds and compositions of Formula (I) and methods of treatment using the compounds and compositions, especially for the treatment and/or prevention of a proliferation disorder, such as cancer. Compounds of Formula (I) as further described herein are shown modulators of the adenosine A2A receptor and exhibit antiproliferative activity. Accordingly, these compounds are useful to treat proliferative disorders such as cancer, and other adenosine receptor-related conditions including an inflammatory disease, renal disease, diabetes, vascular disease, lung disease, or an autoimmune disease.

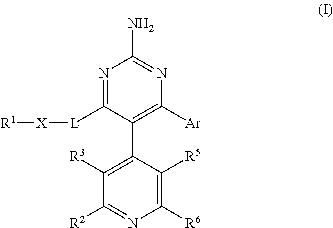

### FIELD OF THE INVENTION

The present application claims priority to U.S. provisional patent application No. 62/695,877, filed on Jul. 10, 2018, the disclosure of which is incorporated by reference in its entirety for all purposes.

The invention provides compounds having pharmaceutical utility based on activity on the Adenosine A_{2A} and/or A_{2B} receptor, and pharmaceutical compositions and methods for treatment of a proliferation disorder such as cancer utilizing these compounds.

### BACKGROUND OF THE INVENTION

G protein-coupled receptors (GPCRs) are transmembrane signaling complexes that are associated with regulation of diverse critical physiologic processes. GPCRs are associated with a variety of disease states, and many of them have been pursued as targets for drug discovery. Indeed, many important marketed drugs act on GPCRs, including aripiprazole (Ability®, an antipsychotic), loratadine (Claritin®, an antihistamine), ranitidine (Zantac®, anti-ulcer medication), and olanzapine (Zyprexa®, for schizophrenia).

Adenosine is an important signaling compound in vivo, and its levels are normally controlled by cellular uptake competing with adenosine deaminase. Local levels of extracellular adenosine triphosphate (ATP) are acutely elevated as a consequence of infection, tissue injury, ischaemia or intervention-induced tumor cell death. Elevated extracellular ATP is recognized by the immune system as a danger signal to initiate multiple pro-inflammatory events, including the recruitment of macrophages and dendritic cells. Successive processing of extracellular ATP by the extracellular ectonucleotidases CD73 and CD39 lowers extracellular ATP levels and can rapidly elevate extracellular adenosine from a low homeostatic level (20-200 nM) to as much as 1 ,000-10,000 nM. These elevated adenosine concentrations engage the immunosuppressive actions of adenosine A_{2A} and A_{2B} receptors on the infiltrating lymphocytes, shielding cells from an excessive inflammatory response and thereby providing a self-limiting mechanism to resolve the immune response. Within the context of a solid tumor, hypoxia has been shown to increase adenosine levels by 10-20-fold compared with normal levels. It has been proposed that adenosine elevation is sufficient to maintain a chronic suppression of the innate immune response, resulting in immune tolerance and, subsequently, uncontrolled malignant growth.

The Adenosine A_{2A} and A_{2B} receptors (A_{2A}R and A_{2B}R) are GPCRs that have been identified as drug discovery targets for inflammation, cardiovascular disease, and Parkinson's disease. A_{2A}R is widely distributed throughout the body, and serves a protective signaling function when localized damage or trauma creates high levels of extracellular adenosine. The tumor microenvironment has been shown to have high levels of adenosine, and many tumors use A_{2A}R to protect themselves from recognition and destruction by the immune system. Mice lacking A_{2A}R have been shown to be better able to resist tumor growth (Waickman, et al., Cancer Immunol Immunother. 2012 June; 61(6): 917-926.) Thus A_{2A}R is of great interest for immunooncology applications, and modulators of A_{2A}R are expected to enhance the ability of the immune system to recognize and attack tumor cells, and may promote effectiveness of other antitumor drugs.

While A_{2A}R and A_{2B}R differ significantly in structure, they share about 59% sequence similarity and are both involved in the adenylyl cyclase pathway. Sun, et al., Frontiers in Chem., vol. 4, August 2016, pp. 1-11. The A_{2B} receptor is notably found in the large intestine and bladder, but is also present in many other tissues and in a variety of immune system cells. A_{2B}R has a lower affinity for adenosine than A_{2A}R does, and its physiological roles differ from those of A_{2A}R. The A_{2B} receptor, in addition to activating adenylate cyclase in a way similar to A_{2A} receptor, can be coupled to distinct intracellular signaling pathways and play physiological roles that differ from those of A_{2A}Rs. It has been reported to play a critical role in some cancers, renal disease, diabetes, vascular diseases, and lung diseases.

Accordingly, A_{2A}R and A_{2B}R ligands have potentially wide utility as pharmaceuticals for use in treating cancer and other disorders. Antagonists of A_{2A}R are of particular interest for use in immunooncology therapy, like checkpoint inhibitors targeting PD-1 and PD-L1 (nivolumab, pembrolizumab, atezolizumab and others) that have been proven useful and approved for use in a variety of antitumor therapies. The present invention provides compounds that block A_{2A}R and/or A_{2B}R signaling, and are useful as pharmaceuticals.

### Disclosure of the Invention

In one aspect, the present disclosure provides for a heterocyclic compound having a structure according to Formula I:

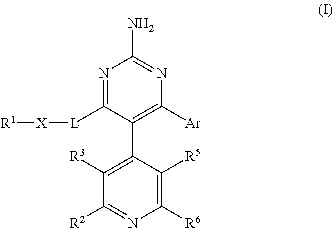

wherein:

L is selected from the group consisting of [X]—(C(R^{a})₂)ₙ—, [X]—(C(R^{a})₂)ₙ—O—, [X]—(C(R^{a})₂)ₙ—NR^{b}—, [X]—(C(R^{a})₂)ₙ—NR^{b}—SO₂—, [X]—(C(R^{a})₂)ₙ—NR^{b}—C(O)—, [X]—(C(R^{a})₂)ₙ—C(O)—, [X]—(C(R^{a})₂)ₙ—C(O)—NR^{b}—, [X]—NR^{b}—(C(R^{a})₂)ₙ—C(O)—, [X]—O—(C(R^{a})₂)ₚ—O—, [X]—NR^{b}—(C(R^{a})₂)ₚ—O—, [X]—(C(R^{a})₂)ₙ—S(O)ₘ— and [X]—(C(R^{a})₂)ₙ—C(O)—;
where [X] indicates which end of L is attached to X in Formula (I); and
each p is independently 1, 2 or 3;
X is absent (i.e., it represents a bond between L and R¹), (CR^{a}₂)ₙ, C(═O), [R¹]—(CR^{a}₂)ₙ—NR^{b}—, [R¹]—(CR^{a}₂)ₙ—O—, [R¹]—O—(CR^{a}₂)ₙ—, [R¹]—NR^{b}—(CR^{a}₂)ₙ—, [R¹]—(CR^{a}₂)ₙ—S(O)ₘ—, [R¹]—S(O)ₘ—(CR^{a}₂)ₙ—, [R¹]—C(O)—NR^{b}—, [R¹]—(CR^{a}₂)ₙ—NR^{b}—C(O)—, [R¹]—NR^{b}—C(O)—NR^{b}—, [R¹]—NR^{b}—C(O)—O—, C₁₋₄ alkyl, C₃-C₈ cycloalkyl, a 3-8 membered heterocyclic ring, phenyl, or a 5-12 membered heteroaryl ring; where [R¹] indicates which end of X is attached to R¹;
   wherein the C₁₋₄ alkyl, C₃-C₈ cycloalkyl, 3-8 membered heterocyclic ring, phenyl, or 5-12 membered heteroaryl ring is optionally substituted with one to three groups selected from C₁-C₃ alkyl, —OH, oxo, COOR¹⁰, —NR⁸R⁹, C(O)NR⁸R⁹, SO₂R¹¹, SO₂NR⁸R⁹, —S(═O)(═NR^{b})R¹¹, NR^{b}C(O)OR¹¹, NR^{b}C(O)NR⁸R⁹, C₁₋₃ alkyl optionally substituted with OH, OMe, Cx or —O—Cx, and C₁₋₃ alkoxy optionally substituted with OH, OMe, Cx, or —O—Cx;
   wherein each Cx is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
   where each Cx is optionally substituted with one or two groups selected from halo, oxo, CN, C₁₋₃ alkyl, C₁₋₃ haloalkyl, C₁₋₃ alkoxy, and OH;
each R^{a} and R^{b} is independently H, —OR^{c}, —COOR^{c}, or C₁-C₃ alkyl optionally substituted with one or two groups selected from halo, oxo, —COOR^{c}, —OR^{c}, and —N(R^{c})₂;
   where each R^{c} is independently H or C₁-C₃ alkyl optionally substituted with one to three groups independently selected from halo, OH, oxo, and methoxy;

R¹ is selected from the group consisting of H, OH, R⁷, OR⁷, —NR⁷R⁸, —NR⁸R⁹, —S(O)ₘR⁷, —(CR^{a}₂)₀₋₂—Cy, (CR^{a}₂)₀₋₂—O—Cy, —O—(CR^{a}₂)₁₋₂—Cy, —C(O)R¹⁰, —C(O)OR¹⁰, —C(O)NR⁸R⁹, —NR^{b}C(O)R¹⁰, —NR^{b}COOR¹¹, —NR^{b}C(O)NR⁸R⁹, —NR^{b}SO₂R¹¹, —NR^{b}SO₂NR⁸R⁹, —SO₂R¹¹, —SO₂NR⁸R⁹, OSO₂R¹¹, —OSO₂NR⁸R⁹, —S(═O)(═NR^{b})R¹¹, —OC(O)NR⁸R⁹, —OC(O)R¹¹, —P(O)(R¹¹)₂, —P(O)(OR¹⁰)₂, —P(O)(OR¹⁰)—R¹¹, —P(O)(NR⁸R⁹)₂, —O—P(O)(OR¹⁰)₂, —O—P(O)(OR¹⁰)—R¹¹, and —P(O)(NR⁸R⁹)—R¹¹;
Cy is a cyclic group selected from phenyl, C₃-C₈ cycloalkyl, a 5-12 membered monocyclic heteroaryl group having up to four heteroatoms selected from N, O and S as ring members, and a 3-8 membered heterocyclic ring comprising one or two heteroateoms selected from N, O and S as ring members, and is optionally fused to a phenyl or a 5-12 membered heteroaryl or a heterocyclic ring having one or two heteroatoms selected from N, O and S as ring members or a C₃-C₈ cycloalkyl ring to form a bicyclic group
   wherein the cyclic or bicyclic group Cy is optionally substituted with up to three groups independently selected from R⁷, —OR⁷, oxo, halo, —OH, C₁-C₃ alkyl, C₁-C₃ haloalkyl, C₃-C₈ cycloalkyl, C₁-C₃ alkoxy, C₃-C₈ cycloalkyl, COOR¹⁰, CN, SO₂R¹¹, C(O)R¹⁰, —NR⁸R⁹, —NR⁷R⁸, —C(O)NR⁸R⁹, NR^{b}COOR¹¹, NR^{b}SO₂R¹¹, and C₁-C₃ alkyl that is substituted with one or two groups selected from OH, OMe, COOR¹⁰, CN, SO₂R¹¹, C(O)R¹⁰, and C(O)NR⁸R⁹;
R⁷ is C₁-C₆ alkyl, C₂-C₆ alkenyl, C₂-C₆ alkynyl, C₁-C₃ haloalkyl, C₃-C₈ cycloalkyl, or 3-8 membered heterocyclic group having one or two heteroatoms selected from N, O and S as ring members,
   wherein the C₁-C₆ alkyl, C₂-C₆ alkenyl, C₂-C₆ alkynyl, C₁-C₃ haloalkyl, C₃-C₈ cycloalkyl, or 3-8 membered heterocyclic group is optionally substituted with one to three groups selected from —OH, OR¹⁰, CN, oxo, COOR¹⁰, C(O)R¹⁰, —NR⁸R⁹, C(O)NR⁸R⁹, SO₂R¹¹, SO₂NR⁸R⁹, —S(═O)(═NR^{b})R¹¹, NR⁸SO₂R¹¹, NR^{b}C(O)OR¹¹, NR^{b}C(O)NR⁸R⁹, OC(O)NR⁸R⁹, Cz, C₁₋₃ alkyl optionally substituted with OH, OMe, Cz, SO₂R¹¹, COOR¹⁰, or —O—Cz, and C₁₋₃ alkoxy optionally substituted with OH, OMe, SO₂R¹¹, COOR¹⁰, Cz, or —O—Cz;
   wherein each Cz is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
      where each Cz is optionally substituted with one or two groups selected from halo, C₁₋₃ alkyl, C₁₋₃ haloalkyl, C₁₋₃ alkoxy, and OH;
R⁸ and R⁹ are independently at each occurrence selected from H, C(O)R¹⁰, C(O)OR¹⁰, haloalkyl, and C₁-C₄ alkyl, C₃-C₈ cycloalkyl or 4-8 membered heterocyclyl having one or two heteroatoms selected from N, O and S as ring members, wherein the C₁-C₄ alkyl, C₃-C₈ cycloalkyl or 4-6 membered heterocyclyl are each optionally substituted with one or two groups independently selected from —OH, Me, —OR¹¹, —NR¹²R¹³, —SO₂R¹¹, COOR¹⁰, C(O)NR¹²R¹³, SO₂NR¹²R¹³, NR^{b}C(O)OR¹¹, and NR^{b}C(O)NR¹²R¹³;
   or R⁸ and R⁹ taken together with N to which both are attached form a 4 to 8 membered heterocyclic ring optionally containing an additional N, O, or S as a ring member and optionally substituted with one or two groups selected from OH, OR¹⁰, oxo, halo, CN, C₁-C₃ alkyl, C₁-C₃ haloalkyl, C₁-C₃ alkoxy, —C(O)R¹⁰, —COOR¹⁰, NR¹²R¹³, C(O)NR¹²R¹³, and —SO₂R¹¹;
R¹⁰ is independently at each occurrence H, C₁-C₄ alkyl optionally substituted with one to three groups selected from halo, —OH, and C₁-C₃ alkoxy;
R¹¹ is independently at each occurrence C₁-C₄ alkyl optionally substituted with one to three groups selected from halo, —OH, and C₁-C₃ alkoxy;
R¹² and R¹³ are independently at each occurrence selected from H, C(O)R¹⁴, C(O)OR¹⁴, haloalkyl, and C₁-C₄ alkyl optionally substituted with —OH or —OR¹⁴;
   where R¹⁴ is independently at each occurrence C₁-C₄ alkyl optionally substituted with one to three groups selected from halo, —OH, and C₁-C₃ alkoxy;
or R¹² and R¹³ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, and C₁-C₃ alkoxy, and C₁-C₄ alkyl substituted with one or two groups selected from —OH, C₁-C₃ alkoxy, CN, SO₂R¹¹, —COOR¹⁰, —NR¹⁵R¹⁶, —NR^{b}C(O)R¹¹, and —CONR¹⁵R¹⁶;

R² and R⁶ are independently selected from H, halo, C₁₋₄ alkoxy, C₁₋₄ haloalkyl, C₁₋₄ haloalkoxy, CN and C₁₋₄ alkyl optionally substituted with one or two groups selected from the group consisting of halo, CN, hydroxy and C₁-C₃ alkoxy;

R³ and R⁵ are independently selected from H, halo, C₁₋₄ alkyl, C₁₋₄ alkoxy, C₁₋₄ haloalkyl, C₁₋₄ haloalkoxy, and CN;

Ar is phenyl or a 5-12 membered heteroaryl ring, and is optionally substituted by 1-3 groups independently selected from halo, hydroxy, C₁₋₄ alkyl, C₁₋₄ alkoxy, C₁₋₄ haloalkyl, C₁₋₄ haloalkoxy, CN, —SO₂R¹¹, —COOR¹⁰, —NR¹⁵R¹⁶, —NR^{b}C(O)R¹⁰, —CONR¹⁵R¹⁶, and C₁-C₄ alkyl substituted with one or two groups selected from —OH, C₁-C₃ alkoxy, CN, SO₂R¹¹, —COOR¹⁰, —NR¹⁵R¹⁶, —NR^{b}C(O)R¹¹, and —CONR¹⁵R¹⁶;
wherein R¹⁵ and R¹⁶ are independently H or C₁₋₄ alkyl;
   or R¹⁵ and R¹⁶ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, C₁-C₃ alkoxy, —C(O)R¹⁰, —COOR¹⁰, and —SO₂R¹¹;

each n is independently an integer selected from 0, 1, 2 and 3; and

each m is independently an integer selected from 0, 1 and 2;

or a pharmaceutically acceptable salt thereof.

The compounds of Formula (I) are further described herein, along with methods to make them and use them as pharmaceuticals.

In another embodiment the invention provides methods to use these compounds for treatment of a proliferative disorder, cancer, an immunologic disease, renal disease, diabetes, vascular disease, or lung disease.

The compound described above can be used for any suitable purpose. In some embodiments, the compound described above can be used in therapy, particularly to treat a proliferative disorder such as cancer, renal disease, diabetes, vascular disease, and lung disease. Particular cancers to be treated with the compounds of the invention are identified herein, and include solid tumors, particularly tumors that cause hypoxia.

In another aspect, the disclosure provides a pharmaceutical composition comprising a compound of Formula (I) as described herein admixed with at least one pharmaceutically acceptable carrier or excipient. Preferably, the compound of Formula (I) or any subgenus or species thereof is admixed with at least two pharmaceutically acceptable excipients.

In yet another aspect, the present disclosure provides a method for treating and/or preventing a proliferation disorder, a cancer, a tumor, an inflammatory disease, renal disease, diabetes, vascular disease, lung disease, or an autoimmune disease, which comprises administering to a subject in need thereof an effective amount of a compound of Formula (I) as described herein, or a pharmaceutical composition containing at least one such compound.

In yet another aspect, the present disclosure provides for a use of a compound of Formula (I) as described herein for the manufacture of a medicament.

In yet another aspect, the present disclosure provides the use of a compound of Formula (I) as described herein in therapy. In some embodiments, the use in therapy is use to treat a proliferative disorder such as cancer, or an inflammatory disease, renal disease, diabetes, vascular disease, lung disease, or an autoimmune disease.

In yet another aspect, the present disclosure provides a combination for treating and/or preventing a proliferation disorder, a cancer, a tumor, an inflammatory disease, lung disease, renal disease, diabetes, or an autoimmune disease in a subject, which combination comprises an effective amount of a compound of Formula (I) as described herein , or a pharmaceutically acceptable salt thereof, and an effective amount of a second prophylactic or therapeutic agent for treating and/or preventing a proliferation disorder, a cancer, a tumor, an inflammatory disease, lung disease, renal disease, diabetes, or an autoimmune disease in a subject.

In yet another aspect, the present disclosure provides a method for treating and/or preventing a proliferation disorder, a cancer, a tumor, a tumor, an inflammatory disease, lung disease, renal disease, diabetes, or an autoimmune disease in a subject, which method comprises administering to a subject in need thereof an effective amount of the combination described above.

In yet another aspect, the present disclosure provides for a method for blocking A_{2A}R signaling, either in vitro or in vivo, which comprises contacting A_{2A}R with a compound of Formula (I) as described herein.

Similarly, in another aspect, the present disclosure provides for a method for blocking A_{2B}R signaling, either in vitro or in vivo, which comprises contacting A_{2B}R with a compound of Formula (I) as described herein.

Other aspects and embodiments of the invention as well as methods to make and use compound of Formula (I) as described herein are described below.

### DETAILED DESCRIPTION

The invention provides compounds of Formula (I), which are useful as pharmaceuticals. Without being bound by theory, it is believed that their pharmaceutical activity arises from their modulation of the adenosine A_{2A} receptor and/or A_{2B} receptor, which activity is demonstrated by data provided herein. The invention is further described and exemplified by the following examples and description.

For purposes of interpreting this specification, the following definitions will apply, and whenever appropriate, terms used in the singular will also include the plural.

Terms used in the specification have the following meanings unless the context clearly indicates otherwise:

As used herein, the term “subject” refers to an animal. In certain aspects, the animal is a mammal. A subject also refers to for example, primates (e.g., humans), cows, sheep, goats, horses, dogs, cats, rabbits, rats, mice, fish, birds and the like. In certain embodiments, the subject is a human A “patient” as used herein refers to a human subject.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder, i.e., slowing or arresting or reducing the development of the disease or at least one of the clinical symptoms thereof. In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

“Optionally substituted” means the group referred to can be substituted at one or more positions by any one or any combination of the radicals suitable for substitution on that group. The number, placement and selection of substituents is understood to encompass only those substitutions that a skilled chemist would expect to be reasonably stable; thus ‘oxo’ would not be a substituent on an aryl or heteroaryl ring, for example, and a single carbon atom would not have three hydroxy or amino substituents. A group can be optionally substituted by a number of substituents up to the number of hydrogen atoms on the unsubstituted version of the group unless otherwise specified; e.g., a methyl group can have up to three substituents.

“Halo” or “halogen”, as used herein, may be fluorine, chlorine, bromine or iodine. In some embodiments, F or Cl is preferred.

“C₁-C₆ alkyl”, or “C1-6 alkyl” as used herein, denotes straight chain or branched alkyl having 1-6 carbon atoms. If a different number of carbon atoms is specified, such as C4 or C3, then the definition is understood to be interpreted accordingly, such as “C1-C4 alkyl” will represent methyl, ethyl, propyl, isopropyl, butyl, isobutyl, sec-butyl and tert-butyl.

“C₁-C₆ alkoxy”, or “C1-6 alkoxy” as used herein, denotes straight chain or branched alkoxy having 1-6 carbon atoms. If a different number of carbon atoms is specified, such as C4 or C3, then the definition is to be amended accordingly, such as “C1-C4 alkoxy” will represent methoxy, ethoxy, propoxy, isopropoxy, butoxy, isobutoxy, sec-butoxy and tert-butoxy.

“C₁-C₄ haloalkyl” or “C1-4 haloalkyl” as used herein, denotes straight chain or branched alkyl having 1-4 carbon atoms wherein at least one hydrogen has been replaced with a halogen. The number of halogen replacements can be from one up to the number of hydrogen atoms on the unsubstituted alkyl group. If a different number of carbon atoms is specified, such as C6 or C3, then the definition is to be amended accordingly. Thus “C₁-C₄ haloalkyl” will represent methyl, ethyl, propyl, isopropyl, butyl, isobutyl, sec-butyl and tert-butyl that have at least one hydrogen substituted with halogen, such as where the halogen is fluorine: typical examples include CF₃CF₂—, (CF₃)₂CH—, CH₃—CF₂—, CF₃CF₂—, CF₃, CF₂H—, CH₂F—, CF₃CF₂CHCF₃ or CF₃CF₂CF₂CF₂—.

“Aryl” as used herein refers to an aromatic carbocyclic group, typically having 6-10 ring atoms; preferably, aryl refers to phenyl or naphthyl, and most commonly phenyl.

“C₃-C₈ cycloalkyl” as used herein refers to a saturated monocyclic hydrocarbon ring of 3 to 8 carbon atoms, or such other number as the term indicates. Examples of such groups include cyclopropyl, cyclobutyl, cyclopentyl and cyclohexyl. If a different number of carbon atoms is specified, such as C₃-C₈, then the definition is to be amended accordingly.

“4- to 8-Membered heterocycle”, “5- to 6-membered heterocyclyl”, “3- to 10-membered heterocyclic”, “3- to 14-membered heterocycle”, “4- to 14-membered heterocyclyl” and “5- to 12-membered heterocyclyl”, refer to 4- to 8-membered, 5- to 6-membered, 3- to 10-membered, 3- to 14-membered, 4- to 14-membered and 5- to 12-membered heterocyclic rings; unless otherwise specified, such rings contain 1 to 7, 1 to 5, or 1 to 3 heteroatoms selected from the group consisting of nitrogen, oxygen and sulphur as ring members, and the rings may be saturated, or partially saturated but not aromatic. The heterocyclic group can be attached at a heteroatom (usually N) or a carbon atom. The term “heterocycle” and variations such as heterocyclic, includes single ring groups, fused ring groups and bridged groups. Examples of such heterocyclyl include, but are not limited to pyrrolidine, piperidine, piperazine, pyrrolidinone, morpholine, tetrahydrofuran, tetrahydrothiophene, tetrahydrothiopyran, tetrahydropyran, 1,4-dioxane, 1,4-oxathiane, 8-aza-bicyclo[3.2.1]octane, 3,8-diazabicyclo[3.2.1]octane, 3-Oxa-8-aza-bicyclo[3.2.1]octane, 8-Oxa-3-aza-bicyclo[3.2.1]octane, 2-Oxa-5-aza-bicyclo[2.2.1]heptane, 2,5-Diaza-bicyclo[2.2.1]heptane, azetidine, ethylenedioxo, oxetane or thiazole.

“Heteroaryl” is a completely unsaturated (aromatic) cyclic group having a heteroatom as part of the aromatic ring, or a ring system comprising at least one such heteroatom-containing aromatic group. The term “heteroaryl” refers to a 5-14 membered monocyclic- or bicyclic- or tricyclic-aromatic ring system, having 1 to 8 heteroatoms selected from N, O and S as ring members. Typically, the heteroaryl is a 5-10 membered ring or ring system (e.g., a 5-6 membered monocyclic group or an 8-10 membered bicyclic group), and is often a 5 or 6 membered ring with one heteroatom selected from N, O and S, or with one to four nitrogen atoms as ring members. Typical heteroaryl groups include furan, isothiazole, thiadiazole, oxadiazole, indazole, indole, quinoline, 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4-triazolyl), 4- or 5-(1,2, 3-triazolyl), tetrazolyl, triazine, pyrimidine, 2-, 3-, or 4-pyridyl, 3- or 4-pyridazinyl, 3-, 4-, or 5-pyrazinyl, 2-pyrazinyl, and 2-, 4-, or 5-pyrimidinyl. 2-Pyridone groups, whether N-substituted or not, are included within the scope of heteroaryl groups for the present disclosure.

The term “hydroxy” or “hydroxyl” refers to the group —OH.

Various embodiments of the invention are described herein. It will be recognized that features specified in each embodiment may be combined with other specified features to provide further embodiments. The following enumerated embodiments are representative of the invention:

1. A compound of Formula (I):

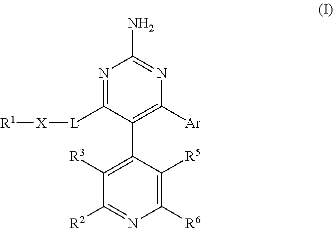

wherein:

L is selected from the group consisting of [X]—(C(R^{a})₂)—, [X]—(C(R^{a})₂)ₙ—O—, [X]—(C(R^{a})₂)ₙ—NR^{b}—, [X]—(C(R^{a})₂)ₙ—NR^{b}—SO₂—, [X]—(C(R^{a})₂)ₙ—NR^{b}—C(O)—, [X]—(C(R^{a})₂)ₙ—C(O)—, [X]—(C(R^{a})₂)ₙ—C(O)—NR^{b}—, [X]—NR^{b}—(C(R^{a})₂)ₙ—C(O)—, [X]—O—(C(R^{a})₂)ₚ—O—, [X]—NR^{b}—(C(R^{a})₂)ₚ—O—, [X]—(C(R^{a})₂)ₙ—S(O)ₘ— and [X]—(C(R^{a})₂)ₙ—C(O)—;
where [X] indicates which end of L is attached to X in Formula (I); and
each p is independently 1, 2 or 3;
X is absent (i.e., it represents a bond between L and R¹), (CR^{a}₂)ₙ, C(═O), [R¹]—(CR^{a}₂)ₙ—NR^{b}—, [R¹]—(CR^{a}₂)ₙ—O—, [R¹]—O—(CR^{a}₂)ₙ—, [R¹]—NR^{b}—(CR^{a}₂)ₙ—, [R¹]—(CR^{a}₂)ₙ—S(O)ₘ—, [R¹]—S(O)ₘ—(CR^{a}₂)ₙ—, [R¹]—C(O)—O—, [R¹]—C(O)—NR^{b}—, [R¹]—(CR^{a}₂)ₙ—NR^{b}—C(O)—, [R¹]—NR^{b}—C(O)—NR^{b}—, [R¹]—NR^{b}—C(O)—O—, C₁₋₄ alkyl, C₃-C₈ cycloalkyl, a 3-8 membered heterocyclic ring, phenyl, or a 5-12 membered heteroaryl ring; where [R¹] indicates which end of X is attached to R¹;
   wherein the C₁₋₄ alkyl, C₃-C₈ cycloalkyl, 3-8 membered heterocyclic ring, phenyl, or 5-12 membered heteroaryl ring is optionally substituted with one to three groups selected from C₁-C₃ alkyl, —OH, oxo, COOR¹⁰, —NR⁸R⁹, C(O)NR⁸R⁹, SO₂R¹¹, SO₂NR⁸R⁹, —S(═O)(═NR^{b})R¹¹, NR^{b}C(O)OR¹¹, NR^{b}C(O)NR⁸R⁹, C₁₋₃ alkyl optionally substituted with OH, OMe, Cx or —O—Cx, and C₁₋₃ alkoxy optionally substituted with OH, OMe, Cx, or —O—Cx;
   wherein each Cx is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
      where each Cx is optionally substituted with one or two groups selected from halo, oxo, CN, C₁₋₃ alkyl, C₁₋₃ haloalkyl, C₁₋₃ alkoxy, and OH;
each R^{a} and R^{b} is independently H, —OR^{c}, —COOR^{c}, or C₁-C₃ alkyl optionally substituted with one or two groups selected from halo, oxo, —COOR^{c}, —OR^{c}, and —N(R^{c})₂;
   where each R^{c} is independently H or C₁-C₃ alkyl optionally substituted with one to three groups independently selected from halo, OH, oxo, and methoxy;

R¹ is selected from the group consisting of H, OH, R⁷, OR⁷, —NR⁷R⁸, —NR⁸R⁹, —S(O)ₘR⁷, —(CR^{a}₂)₀₋₂—Cy, (CR^{a}₂)₀₋₂—O—Cy, —O—(CR^{a}₂)₁₋₂—Cy, —C(O)R¹⁰, —C(O)OR¹⁰, —C(O)NR⁸R⁹, —NR^{b}C(O)R¹⁰, —NR^{b}COOR¹¹, —NR^{b}C(O)NR⁸R⁹, —NR^{b}SO₂R¹¹, —NR^{b}SO₂NR⁸R⁹, —SO₂R¹¹, —SO₂NR⁸R⁹, OSO₂R¹¹, —OSO₂NR⁸R⁹, —S(═O)(═NR^{b})R¹¹, —OC(O)NR⁸R⁹, —OC(O)R¹¹, —P(O)(R¹¹)₂, —P(O)(OR¹⁰)₂, —P(O)(OR¹⁰)—R¹¹, —P(O)(NR⁸R⁹)₂, —O—P(O)(OR¹⁰)₂, —O—P(O)(OR¹⁰)—R¹¹, and —P(O)(NR⁸R⁹)—R¹¹;
Cy is a cyclic group selected from phenyl, C₃-C₈ cycloalkyl, a 5-12 membered monocyclic heteroaryl group having up to four heteroatoms selected from N, O and S as ring members, and a 3-8 membered heterocyclic ring comprising one or two heteroateoms selected from N, O and S as ring members, and is optionally fused to a phenyl or a 5-12 membered heteroaryl or a heterocyclic ring having one or two heteroatoms selected from N, O and S as ring members or a C₃-C₈ cycloalkyl ring to form a bicyclic group
   wherein the cyclic or bicyclic group Cy is optionally substituted with up to three groups independently selected from R⁷, —OR⁷, oxo, halo, —OH, C₁-C₃ alkyl, C₁-C₃ haloalkyl, C₃-C₈ cycloalkyl, C₁-C₃ alkoxy, C₃-C₈ cycloalkyl, COOR¹⁰, CN, SO₂R¹¹, C(O)R¹⁰, —NR⁷R⁸, —C(O)NR⁸R⁹, NR^{b}COOR¹¹, NR^{b}SO₂R¹¹, and C₁-C₃ alkyl that is substituted with one or two groups selected from OH, OMe, COOR¹⁰, CN, SO₂R¹¹, C(O)R¹⁰, and C(O)NR⁸R⁹;
R⁷ is C₁-C₆ alkyl, C₂-C₆ alkenyl, C₂-C₆ alkynyl, C₁-C₃ haloalkyl, C₃-C₈ cycloalkyl, or 3-8 membered heterocyclic group having one or two heteroatoms selected from N, O and S as ring members,
   wherein the C₁-C₆ alkyl, C₂-C₆ alkenyl, C₂-C₆ alkynyl, C₁-C₃ haloalkyl, C₃-C₈ cycloalkyl, or 3-8 membered heterocyclic group is optionally substituted with one to three groups selected from —OH, OR¹⁰, CN, oxo, COOR¹⁰, C(O)R¹⁰, —NR⁸R⁹, C(O)NR⁸R⁹, SO₂R¹¹, SO₂NR⁸R⁹, —S(═O)(═NR^{b})R¹¹, NR⁸SO₂R¹¹, NR^{b}C(O)OR¹¹, NR^{b}C(O)NR⁸R⁹, OC(O)NR⁸R⁹, Cz, C₁₋₃ alkyl optionally substituted with OH, OMe, Cz, SO₂R¹¹, COOR¹⁰, or —O—Cz, and C₁₋₃ alkoxy optionally substituted with OH, OMe, SO₂R¹¹, COOR¹⁰, Cz, or —O—Cz;
   wherein each Cz is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
      where each Cz is optionally substituted with one or two groups selected from halo, C₁₋₃ alkyl, C₁₋₃ haloalkyl, C₁₋₃ alkoxy, and OH;
R⁸ and R⁹ are independently at each occurrence selected from H, C(O)R¹⁰, C(O)OR¹⁰, C₁-C₄ haloalkyl, and C₁-C₄ alkyl, C₃-C₈ cycloalkyl or 4-8 membered heterocyclyl having one or two heteroatoms selected from N, O and S as ring members, wherein the C₁-C₄ alkyl, C₃-C₈ cycloalkyl or 4-6 membered heterocyclyl are each optionally substituted with one or two groups independently selected from —OH, Me, —OR¹¹, —NR¹²R¹³, —SO₂R¹¹, COOR¹⁹, C(O)NR¹²R¹³, SO₂NR¹²R¹³, NRbC(O)OR¹¹, and NR^{b}C(O)NR¹²R¹³;
   or R⁸ and R⁹ taken together with N to which both are attached form a 4 to 8 membered heterocyclic ring optionally containing an additional N, O, or S as a ring member and optionally substituted with one or two groups selected from OH, OR¹⁰, oxo, halo, CN, C₁-C₃ alkyl, C₁-C₃ haloalkyl, C₁-C₃ alkoxy, —C(O)R¹⁹, —COOR¹⁰, NR¹²R¹³, C(O)NR¹²R¹³, and —SO₂R¹¹;
R¹⁰ is independently at each occurrence H, C₁-C₄ alkyl optionally substituted with one to three groups selected from halo, —OH, and C₁-C₃ alkoxy;
R¹¹ is independently at each occurrence C₁-C₄ alkyl optionally substituted with one to three groups selected from halo, —OH, and C₁-C₃ alkoxy;
R¹² and R¹³ are independently at each occurrence selected from H, C(O)R¹⁴, C(O)OR¹⁴, C₁-C₄ haloalkyl, and C₁-C₄ alkyl optionally substituted with —OH or —OR¹⁴;
   where R¹⁴ is independently at each occurrence C₁-C₄ alkyl optionally substituted with one to three groups selected from halo, —OH, and C₁-C₃ alkoxy;
or R¹² and R¹³ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, and C₁-C₃ alkoxy, and C₁-C₄ alkyl substituted with one or two groups selected from —OH, C₁-C₃ alkoxy, CN, SO₂R¹¹, —COOR¹⁰, —NR¹⁵R¹⁶, —NR^{b}C(O)R¹¹, and —CONR¹⁵R¹⁶;

R² and R⁶ are independently selected from H, halo, C₁₋₄ alkoxy, C₁₋₄ haloalkyl, C₁₋₄ haloalkoxy, CN and C₁₋₄ alkyl optionally substituted with one or two groups selected from the group consisting of halo, CN, hydroxy and C₁-C₃ alkoxy;

R³ and R⁵ are independently selected from H, halo, C₁₋₄ alkyl, C₁₋₄ alkoxy, C₁₋₄ haloalkyl, C₁₋₄ haloalkoxy, and CN;

Ar is phenyl or a 5-12 membered heteroaryl ring, and is optionally substituted by 1-3 groups independently selected from halo, hydroxy, C₁₋₄ alkyl, C₁₋₄ alkoxy, C₁₋₄ haloalkyl, C₁₋₄ haloalkoxy, CN, —SO₂R¹¹, —COOR¹⁹, —NR¹⁵R¹⁶, —NR^{b}C(O)R¹⁰, —CONR¹⁵R¹⁶, and C₁-C₄ alkyl substituted with one or two groups selected from —OH, C₁-C₃ alkoxy, CN, SO₂R¹¹, —COOR¹⁹, —NR¹⁵R¹⁶, —NR^{b}C(O)R¹¹, and —CONR¹⁵R¹⁶;
wherein R¹⁵ and R¹⁶ are independently H or C₁₋₄ alkyl;
   or R¹⁵ and R¹⁶ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, C₁-C₃ alkoxy, —C(O)R¹⁰, —COOR¹⁰, and —SO₂R¹¹;
each n is independently an integer selected from 0, 1, 2 and 3; and
each m is independently an integer selected from 0, 1 and 2;
   or a pharmaceutically acceptable salt thereof.

2. The compound of embodiment 1, wherein R² is H, halo, C₁₋₄ alkyl, C₁₋₄ alkoxy, C₁₋₄ haloalkyl, or C₁₋₄ haloalkoxy.

3. The compound of embodiment 1 or 2, wherein R⁶ is halo, C₁₋₄ alkyl, C₁₋₄ alkoxy, C₁₋₄ haloalkyl, or C₁₋₄ haloalkoxy.

4. The compound of any one of the preceding embodiments, wherein R³ is H, halo, C₁₋₄ alkyl, or C₁₋₄ haloalkyl.

5. The compound of any one of the preceding embodiments, wherein R⁵ is is H, halo, C₁₋₄ alkyl, or C₁₋₄ haloalkyl.

6. The compound of any one of the preceding embodiments, wherein L is [X]—(C(R^{a})₂)ₙ—O—.

7. The compound of any one of the preceding embodiments, wherein Ar is phenyl or furanyl and is optionally substituted with one or two groups selected from halo, hydroxy, C₁₋₄ alkyl, C₁₋₄ alkoxy, C₁₋₄ haloalkyl, C₁₋₄ haloalkoxy, and CN.

8. The compound of embodiment 7, wherein Ar is phenyl optionally substituted with one or two groups selected from halo, C₁-C₂ alkyl, CN, and C₁-C₂ haloalkyl.

9. The compound of any one of the preceding embodiments, wherein L is O, [X]—CH₂—O—, or [X]—CH₂CH₂—O—.

10. The compound of any one of the preceding embodiments, wherein R³ and R⁵ each represent H.

11. The compound of any one of the preceding embodiments, wherein R² is C₁-C₂ alkyl.

12. The compound of any one of the preceding embodiments, wherein R⁶ is C₁-C₂ haloalkyl.

13. The compound of any one of the preceding embodiments, wherein X is (CH₂)₁₋₃ or pyridinyl or phenyl.

14. The compound of any one of embodiments 1-12, wherein X is —CHR^{a}— or —C(Me)₂—

15. The compound of embodiment 1, which is a compound of Formula (IA):

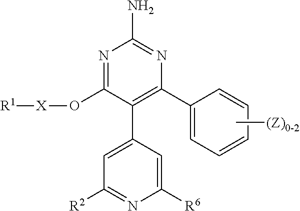

wherein each Z is independently selected from halo, C₁-C₄ alkyl, C₁-C₄ alkoxy, CN, C₁-C₂ haloalkyl, and C₁-C₂haloalkoxy;

R² and R⁶ are independently selected from C₁₋₄ haloalkyl, C₁₋₄ haloalkoxy, and C₁₋₄ alkyl optionally substituted with one or two groups selected from the group consisting of halo, CN, hydroxy and C₁-C₃ alkoxy; and

X and R¹ are as set forth in claim 1;

or a pharmaceutically acceptable salt thereof.

16. The compound of any of the preceding embodiments, wherein R² and R⁶ are different.

17. The compound of any of embodiments 1-16, R⁶ is selected from the group consisting of —CH₂F, CHF₂, —CF₃, and —CF₂CH₃.

18. The compound of any of embodiments 1-17, wherein R² is methyl.

19. The compound of any one of embodiments 15-18, wherein X is (CH₂)₁₋₃ or pyridinyl or phenyl.

20. The compound of any one of embodiments 15-18, wherein X is —C(R^{a})₂— or —C(R^{a})₂—C(R^{a})₂—.

21. The compound of any one of embodiments 1-20, wherein R¹ is R⁷ or —OR⁷.

22. The compound of any one of embodiments 1-20, wherein R¹ is —(CR^{a}₂)₀₋₂—Cy, (CR^{a}₂)₀₋₂—O—Cy, or —O—(CR^{a}₂)₁₋₂—Cy.

23. The compound of embodiment 22, wherein Cy is a cyclic group selected from phenyl, C₃-C₈ cycloalkyl, a 5-6 membered monocyclic heteroaryl group having up to four heteroatoms selected from N, O and S as ring members, and a 3-8 membered heterocyclic ring comprising one or two heteroateoms selected from N, O and S as ring members,
and is optionally fused to a phenyl or a 5-6 membered heteroaryl or a heterocyclic ring having one or two heteroatoms selected from N, O and S as ring members or a C₃-C₈ cycloalkyl ring, to form a bicyclic group;
wherein the cyclic or bicyclic group Cy is optionally substituted as described in Claim 1.

24. The compound of any of embodiments 1-23, wherein at least one atom of the compound is isotopically enriched.

25. The compound of any of embodiments 1-24, which is selected from the compounds in Table 1, or a pharmaceutically acceptable salt thereof.

26. A pharmaceutical composition comprising the compound of any one of embodiments 1-25 or a pharmaceutically acceptable salt thereof, admixed with at least one pharmaceutically acceptable excipient.

27. A method to treat a proliferative disorder, cancer, inflammatory disease, renal disease, diabetes, vascular disease, lung disease, or an autoimmune disease, which comprises administering to a subject in need of such treatment the compound according to any one of embodiments 1-25 or a pharmaceutically acceptable salt thereof, or the pharmaceutical composition of embodiment 26.

28. The method of embodiments 27, which further comprises administering to the subject at least one additional therapeutic agent.

In addition to the embodiments above, the following enumerated embodiments are included within the scope of the invention.

1a. The compound of Embodiment la is a compound of Formula (I) wherein:

L is selected from the group consisting of [X]—(C(R^{a})₂)ₙ—, [X]—(C(R^{a})₂)ₙ—O—, [X]—(C(R^{a})₂)ₙ—NR^{b}—, [X]—(C(R^{a})₂)ₙ—NR^{b}—SO₂—, [X]—(C(R^{a})₂)ₙ—NR^{b}—C(O)—, [X]—(C(R^{a})₂)ₙ—C(O)—, [X]—(C(R^{a})₂)ₙ—C(O)—NR^{b}—, [X]—NR^{b}—(C(R^{a})₂)ₙ—C(O)—, [X]—O—(C(R^{a})₂)ₚ—O—, [X]—NR^{b}—(C(R^{a})₂)ₚ—O—, [X]—(C(R^{a})₂)ₙ—S(O)ₘ— and [X]—(C(R^{a})₂)ₙ−C(O)—;
where [X] indicates which end of L is attached to X in Formula (I); and
each p is independently 1, 2 or 3;
X is absent (i.e., it represents a bond between L and R¹), (CR^{a}₂)ₙ, C(═O), [R¹]—(CR^{a}₂)ₙ—NR^{b}—, [R¹]—(CR^{a}₂)ₙ—O—, [R¹]—O—(CR^{a}₂)ₙ—, [R¹]—NR^{b}—(CR^{a}₂)ₙ—, [R¹]—(CR^{a}₂)ₙ—S(O)ₘ—, [R¹]—S(O)ₘ—(CR^{a}2)ₙ—, [R¹]—C(O)—NR^{b}—, [R¹]—(CR^{a}₂)ₙ—NR^{b}—C(O)—, [R¹]—NR^{b}—C(O)—NR^{b}—, [R¹]—NR^{b}—C(O)—O—, C₃-C₈ cycloalkyl, a 3-8 membered heterocyclic ring, phenyl, or a 5-12 membered heteroaryl ring; where [R¹] indicates which end of X is attached to R¹;
   wherein the C₃-C₈ cycloalkyl, 3-8 membered heterocyclic ring, phenyl, or 5-12 membered heteroaryl ring is optionally substituted with one to three groups selected from C₁-C₃ alkyl, —OH, oxo, COOR¹⁰, C(O)NR⁸R⁹, SO₂R¹¹, SO₂NR⁸R⁹, —S(═O)(═NR^{b})R¹¹, NR^{b}C(O)OR¹¹, NR^{b}C(O)NR⁸R⁹, C₁₋₃ alkyl optionally substituted with OH, OMe, Cx or —O—Cx, and C₁₋₃ alkoxy optionally substituted with OH, OMe, Cx, or —O—Cx;
   wherein each Cx is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
      where each Cx is optionally substituted with one or two groups
   selected from halo, C₁₋₃ alkyl, C₁₋₃ haloalkyl, C₁₋₃ alkoxy, and OH;

each R^{a} and R^{b} is independently H, —COOR^{c}, or C₁-C₃ alkyl optionally substituted with one or two groups selected from halo, oxo, —COOR^{c}, —OR^{c}, and —N(R^{c})₂; where each R^{c} is independently H or C₁-C₃ alkyl optionally substituted with one to three groups independently selected from halo, OH, oxo, and methoxy;

R¹ is selected from the group consisting of H, OH, R⁷, OR⁷, —NR⁸R⁹, —S(O)ₘR⁷, —(CR^{a}₂)₀₋₂—Cy, (CR^{a}₂)₀₋₂—O—Cy, —O—(CR^{a}₂)₁₋₂—Cy, —C(O)R¹⁰, —C(O)OR¹⁰, —C(O)NR⁸_{R}⁹, —NR^{b}C(O)R¹⁰, —NR^{b}COOR¹¹, —NR^{b}C(O)NR⁸R⁹, —NR^{b}SO₂R¹¹, —NR^{b}SO₂NR⁸R⁹, —SO₂R¹¹, —SO₂NR⁸R⁹, OSO₂R¹¹, —OSO₂NR⁸R⁹, —S(═O)(═NR^{b})R¹¹, —OC(O)NR⁸R⁹, —OC(O)R¹¹, —P(O)(R¹¹)₂, —P(O)(OR¹⁰)₂, —P(O)(OR¹⁰)—R¹¹, —P(O)(NR⁸R⁹)₂, —O—P(O)(OR¹⁰)₂, —O—P(O)(OR¹⁰)—R¹¹, and —P(O)(NR⁸R⁹)—R¹¹;
Cy is a cyclic group selected from phenyl, C₃-C₈ cycloalkyl, a 5-12 membered monocyclic heteroaryl group having up to four heteroatoms selected from N, O and S as ring members, and a 3-8 membered heterocyclic ring comprising one or two heteroateoms selected from N, O and S as ring members, and is optionally fused to a phenyl or a 5-12 membered heteroaryl or heterocyclic ring having one or two heteroatoms selected from N, O and S as ring members to form a bicyclic group, wherein the cyclic or bicyclic group Cy is optionally substituted with up to three groups independently selected from R⁷, —OR⁷, oxo, halo, —OH, C₁-C₃ alkyl, C₁-C₃ haloalkyl, C₃-C₈ cycloalkyl, C₁-C₃ alkoxy, C₃-C₈ cycloalkyl, COOR¹⁰, CN, SO₂R¹¹, C(O)R¹⁰, —NR⁸R⁹, —C(O)NR⁸R⁹, NR^{b}COOR¹¹, NR^{b}SO₂R¹¹, and C₁-C₃ alkyl that is substituted with one or two groups selected from OH, OMe, COOR¹⁰, CN, SO₂R¹¹, C(O)R¹⁰, and C(O)NR⁸R⁹;
R⁷ is C₁-C₆ alkyl, C₁-C₃ haloalkyl, C₃-C₈ cycloalkyl, or 3-8 membered heterocyclic group having one or two heteroatoms selected from N, O and S as ring members,
   wherein the C₁-C₆ alkyl, C₁-C₃ haloalkyl, C₃-C₈ cycloalkyl, or 3-8 membered heterocyclic group is optionally substituted with one or two groups selected from —OH, oxo, COOR¹⁰, C(O)R¹⁰, C(O)NR⁸R⁹, SO₂R¹¹, SO₂NR⁸R⁹, —S(═O)(═NR^{b})R¹¹, NR^{b}C(O)OR¹¹, NR^{b}C(O)NR⁸R⁹, C₁₋₃ alkyl optionally substituted with OH, OMe, Cz, or —O—Cz, and C₁₋₃ alkoxy optionally substituted with OH, OMe, Cz, or —O—Cz;
   wherein each Cz is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
      where each Cz is optionally substituted with one or two groups selected from halo, C₁₋₃ alkyl, C₁₋₃ haloalkyl, C₁₋₃ alkoxy, and OH;
R⁸ and R⁹ are independently at each occurrence selected from H, C(O)R¹⁰, C(O)OR¹⁰, haloalkyl, and C₁-C₄ alkyl, C₃-C₈ cycloalkyl or 4-8 membered heterocyclyl having one or two heteroatoms selected from N, O and S as ring members, wherein the C₁-C₄ alkyl, C₃-C₈ cycloalkyl or 4-6 membered heterocyclyl are each optionally substituted with —OH, Me, —OR¹¹, —NR¹²R¹³, —SO₂R¹¹, COOR¹⁰, C(O)NR¹²R¹³, SO₂NR¹²R¹³, NR^{b}C(O)OR¹¹, and NR^{b}C(O)NR¹²R¹³;
   or R⁸ and R⁹ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, C₁-C₃ alkoxy, —C(O)R¹⁰, —COOR¹⁰, NR¹²R¹³, C(O)NR¹²R¹³, and —SO₂R¹¹;
R¹⁰ is independently at each occurrence H, C₁-C₄ alkyl optionally substituted with one to three groups selected from halo, —OH, and C₁-C₃ alkoxy;
R¹¹ is independently at each occurrence C₁-C₄ alkyl optionally substituted with one to three groups selected from halo, —OH, and C₁-C₃ alkoxy;
R¹² and R¹³ are independently at each occurrence selected from H, C(O)R¹⁴, C(O)OR¹⁴, haloalkyl, and C₁-C₄ alkyl optionally substituted with —OH or —OR¹⁴;
   where R¹⁴ is independently at each occurrence C₁-C₄ alkyl optionally substituted with one to three groups selected from halo, —OH, and C₁-C₃ alkoxy;
or R¹² and R¹³ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, and C₁-C₃ alkoxy, and C₁-C₄ alkyl substituted with one or two groups selected from —OH, C₁-C₃ alkoxy, CN, SO₂R¹¹, —COOR¹⁰, —NR¹⁵R¹⁶, —NR^{b}C(O)R¹¹, and —CONR¹⁵R¹⁶;

R² and R⁶ are independently selected from H, halo, C₁₋₄ alkoxy, C₁₋₄ haloalkyl, C₁₋₄ haloalkoxy, CN and C₁₋₄ alkyl optionally substituted with one or two groups selected from the group consisting of halo, CN, hydroxy and C₁-C₃ alkoxy;

R³ and R⁵ are independently selected from H, halo, C₁₋₄ alkyl, C₁₋₄ alkoxy, C₁₋₄ haloalkyl, C₁₋₄ haloalkoxy, and CN;

Ar is phenyl or a 5-12 membered heteroaryl ring, and is optionally substituted by 1-3 groups independently selected from halo, hydroxy, C₁₋₄ alkyl, C₁₋₄ alkoxy, C₁₋₄ haloalkyl, C₁₋₄ haloalkoxy, CN, —SO₂R¹¹, —COOR¹⁰, —NR¹⁵R¹⁶, —NR^{b}C(O)R¹⁰, —CONR¹⁵R¹⁶, and C₁-C₄ alkyl substituted with one or two groups selected from —OH, C₁-C₃ alkoxy, CN, SO₂R¹¹, —COOR¹⁰, —NR¹⁵R¹⁶, —NR^{b}C(O)R¹¹, and —CONR¹⁵R¹⁶;
wherein R¹⁵ and R¹⁶ are independently H or C₁₋₄ alkyl;
   or R¹⁵ and R¹⁶ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, C₁-C₃ alkoxy, —C(O)R¹⁰, —COOR¹⁰, and —SO2R¹¹;

each n is independently an integer selected from 0, 1, 2 and 3; and

each m is independently an integer selected from 0, 1 and 2;

or a pharmaceutically acceptable salt thereof.

In compounds of embodiment 1 or 1a, any or all of the following options may apply:
i. In embodiment 1, when R^{b} is attached to nitrogen, R^{b} is preferably H.
ii. In embodiment 1, each C₃-C₈ cycloalkyl can be C₃-C₆ cycloalkyl.
iii. In embodiment 1, each 3-8 membered heterocyclic ring typically contains one or two heteroatoms selected from N, O and S as ring members, and each 3-8 membered heterocyclic ring is preferably a 4-6 membered heterocyclic ring containing one heteroatom that is N, O or S, or a 6 membered heterocyclic ring, containing two heteroatoms independently selected from N, O and S.
iv. In embodiment 1, each 5-12 membered heteroaryl ring contains 1-4 heteroatoms selected from N, O and S as ring members, and is preferably a 5-6 membered heteroaryl ring, containing one to three heteroatoms selected from N, O and S as ring members.
v. In certain versions of embodiment 1, R¹ is not H. In some of these embodiments, R¹ is R⁷ or —OR⁷. When R¹ is R⁷, it is preferably C₁-C₆ alkyl optionally substituted with one or two groups selected from —OH, COOR¹⁰, C(O)R¹⁰, C(O)NR⁸R⁹, SO2R¹¹, SO₂NR⁸R⁹, —S(═O)(═NR^{b})R¹¹, NR^{b}C(O)OR¹¹, NR^{b}C(O)NR⁸R⁹, C₁₋₃ alkyl optionally substituted with OH, OMe, Cz, or —O—Cz, and C₁₋₃ alkoxy optionally substituted with OH, OMe, Cz, or —O—Cz.
vi. In certain version of embodiment 1, R¹ is selected from the group consisting of —NR⁸R⁹, —S(O)ₘR⁷, —(CR^{a}₂)₀₋₂—Cy, (CR^{a}₂)₀₋₂—O—Cy, or —O—(CR^{a}₂)₁₋₂—Cy. In these embodiments, Cy is typically a 5 or 6 membered heterocyclic group or C₃-C₆ cycloalkyl.

In addition to the compounds of embodiment 1 described above, the following enumerated embodiments are disclosed:

2a. The compound of embodiment 1a, wherein R² is H, halo, C₁₋₄ alkyl, C₁₋₄ alkoxy, C₁₋₄ haloalkyl, or C₁₋₄ haloalkoxy. In some of these embodiments, R² is selected from C₁₋₄ alkyl and C₁₋₄ haloalkyl.

3a. The compound of embodiment 1a or 2a, wherein R⁶ is halo, C₁₋₄ alkyl, C₁₋₄ alkoxy, C₁₋₄ haloalkyl, or C₁₋₄ haloalkoxy. In some of these embodiments, R⁶ is selected from C₁₋₄ alkyl and C₁₋₄ haloalkyl.

4a. The compound of any one of the preceding embodiments 1a-3a, wherein R³ is H, halo, C₁₋₄ alkyl, or C₁₋₄ haloalkyl. In some of these embodiments, R³ is H.

5a. The compound of any one of the preceding embodiments 1a-4a, wherein R⁵ is is H, halo, C₁₋₄ alkyl, or C₁₋₄ haloalkyl. In some of these embodiments, R⁵ is H.

6a. The compound of any one of the preceding embodiments 1a-5a, wherein L is [X]—(C(R^{a})₂)—O—. Frequently, n is 0 or 1 in these embodiments. In some of these embodiments, each R^{a} is independently selected from H and Me. Typically, in these embodiments, L is O, [X]—CH₂—O—, [X]—CH₂—CH₂—O—, or [X]—CH(Me)—O—. In preferred embodiments, L is O.

7a. The compound of any one of the preceding embodiments 1a-6a, wherein Ar is phenyl or furanyl and is optionally substituted with one or two groups selected from halo, hydroxy, C₁₋₄ alkyl, C₁₋₄ alkoxy, C₁₋₄ haloalkyl, C₁₋₄ haloalkoxy, and CN. In preferred embodiments, Ar is phenyl substituted with up to two groups selected from halo, hydroxy, C₁₋₄ alkyl, C₁₋₄ alkoxy, C₁₋₄ haloalkyl, C₁₋₄ haloalkoxy, and CN, and preferably is unsubstituted, or is substituted with halo.

8a. The compound of embodiment 7a, wherein Ar is phenyl optionally substituted with one or two groups selected from halo and C₁-C₂ haloalkyl, or one or two groups selected from halo, C1-2 alkyl, and CN. Suitable examples of A in these embodiments include phenyl substituted with halo, phenyl substituted with CN, phenyl substituted with CN and halo, and phenyl substituted with CN and methyl. Specific examples of Ar in these embodiments include 4-Fluorophenyl, 3-cyanophenyl, 2-methylphenyl, and 2-methyl-3-cyanophenyl.

9a. The compound of any one of the preceding embodiments 1a-8a, wherein L is O, [X]—CH₂—O—, [X]—CH₂CH₂—O—, or [X]—CH(Me)—O—.

10a. The compound of any one of the preceding embodiments 1a-9a, wherein R³ and R⁵ each represent H.

11a. The compound of any one of the preceding embodiments 1a-10-, wherein R² is C₁-C₂ alkyl. In some of these embodiments, R² is Me.

12a. The compound of any one of the preceding embodiments 1a-11a, wherein R⁶ is C₁-C₂ haloalkyl. In some of these embodiments, R⁶ is selected from CF₃, CHF₂, and CH₂F.

13a. The compound of any one of the preceding embodiments, wherein X is (CH₂)₁₋₃ or phenyl. In other embodiments of any one of the preceding embodiments X is a bond. In other embodiments of any one of the preceding embodiments X is a 5 or 6 membered heteroaryl optionally substituted with one to three groups selected from C₁-C₃ alkyl, —OH, oxo, COOR¹⁰, —NR⁸R⁹, C(O)NR⁸R⁹, SO₂R¹¹, SO₂NR⁸R⁹, —S(═O)(═NR⁶)R¹¹, NR⁶C(O)OR¹¹, NR^{b}C(O)NR⁸R⁹, C₁₋₃ alkyl optionally substituted with OH, OMe, Cx or —O—Cx, and C₁₋₃ alkoxy optionally substituted with OH, OMe, Cx, or —O—Cx. Suitable 5-6 membered heteroaryl groups for these embodiments include pyridinyl, pyrazolyl, triazolyl, and imidazolyl.

14a. The compound of any one of embodiments 1a-12a, wherein X is —CHR^{a}—.

15a. The compound of embodiment 1a, which is a compound of Formula (IA):

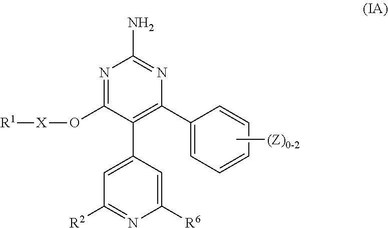

wherein each Z is independently selected from halo, C₁-C₄ alkyl, C₁-C₄ alkoxy, CN, and C₁-C₂ haloalkyl, and C₁-C₂ haloalkoxy;

R² and R⁶ are independently selected from C₁₋₄ haloalkyl, C₁₋₄ haloalkoxy, and C₁₋₄ alkyl optionally substituted with one or two groups selected from the group consisting of halo, CN, hydroxy and C₁-C₃ alkoxy; and

X and R¹ are as set forth in embodiment 1;

or a pharmaceutically acceptable salt thereof.

In some embodiments of Formula (IA), the group R¹—X— represents R⁷—(CH₂)₀₋₂— or R⁷—CHMe-, wherein R⁷ is C₁-C₆ alkyl optionally substituted with one or two groups selected from —OH, COOR¹⁰, C(O)R¹⁰, C(O)NR⁸R⁹, SO₂R¹¹, and SO₂NR⁸R⁹. In other embodiments of Formula (IA), the group R¹—X— represents R⁷—O—phenyl- or R⁷—O-pyridinyl-.

16a. The compound of any of the preceding embodiments 1a-15a, wherein R² and R⁶ are different.

17a. The compound of any of embodiments 1a-16a, R⁶ is selected from the group consisting of —CH₂F, CHF₂, —CF₃, and —CF₂CH₃.

18a. The compound of any of embodiments 1a-17a, wherein R² is methyl.

19a. The compound of any of embodiments 1a-18a, wherein at least one atom of the compound is isotopically enriched. In some of these embodiments, the isotopically enriched atom is a hydrogen atom that is enriched in deuterium. In some of these embodiments, R² is isotopically enriched, typically with 1, 2 or 3 deuterium atoms, i.e., R² is —CH₂D, —CHD₂, or —CD₃.

20a. The compound of any of embodiments 1a-19a, which is selected from the compounds in Table 1, or a pharmaceutically acceptable salt thereof. Each individual compound of Table 1 is a preferred embodiment.

21a. A pharmaceutical composition comprising the compound of any one of embodiments 1a-20a or a pharmaceutically acceptable salt thereof, admixed with at least one pharmaceutically acceptable excipient.

22a. A method to treat a proliferative disorder, cancer, inflammatory disease, renal disease, diabetes, vascular disease, lung disease, or an autoimmune disease, which comprises administering to a subject in need of such treatment the compound according to any one of embodiments 1a-20a or a pharmaceutically acceptable salt thereof, or the pharmaceutical composition of embodiment 21a.

23a. The method of embodiment 22a, which further comprises administering to the subject at least one additional therapeutic agent.

In compounds of Formula (I) and (IA) according to any of the foregoing embodiments, R² is preferably different from R⁶; and preferably at least one of R² and R⁶ is a halomethyl group. In some embodiments, L is selected from [X]—(C(R^{a})₂)—O—, [X]—O—(C(R^{a})₂)ₚ—O—, and [X]—NR⁶—(C(R^{a})₂)ₚ—O—: in these embodiments n is preferably 1 or 2, and each Ra is selected from H and Me.

Any formula given herein, unless otherwise indicated, is intended to encompass compounds having naturally occurring isotopic abundance, as well as isotopically enriched forms of the compounds of the invention, particularly isotopically-enriched compounds having up to three atoms with non-natural isotope distributions, e.g., compounds wherein one or more atoms are enriched in an ordinarily low-abundance (<5%) isotope such as deuterium or ¹³C or ¹⁵N. Isotopically labeled compounds have the structures depicted by the formulas given herein, except that at least one of the atoms is enriched, meaning the natural isotope or isotope distribution is replaced by an atom of the same element having a selected atomic mass or mass distribution other than the natural-abundance mass distribution. Examples of isotopes that can be usefully over-incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes unlabeled compounds (those having natural isotopic abundance for each atom), as well as various isotopically labeled compounds of Formula (I), for example those in which radioactive isotopes, such as ³H and ¹⁴C, or those in which non-radioactive isotopes, such as ²H and ¹³C are present at levels substantially above natural isotope distribution. Such isotopically labelled compounds are useful in metabolic studies (with ¹⁴C, for example), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F labeled compound of the present invention may be particularly desirable for PET or SPECT studies.

Isotopically-labeled compounds, or ‘isotopically enriched’ compounds of the present invention can generally be prepared by conventional techniques known to those skilled in the art or by processes analogous to those described in the accompanying Examples and Preparations using an appropriate isotopically-labeled reagent in place of the non-labeled reagent typically employed. Labeled samples may be useful with quite low isotope incorporation, such as where a radiolabel is used to detect trace amounts of the compound.

Further, more extensive substitution with heavier isotopes, particularly deuterium (i.e., ²H or D), may afford certain therapeutic advantages resulting from greater metabolic stability, for example increased in vivo half-life or reduced dosage requirements or an improvement in therapeutic index.

Where a compound of the invention is enriched in an isotope at a particular atom, the isotope distribution for that atom in a sample of the compound will not correspond to natural abundance; rather, an isotope that is naturally absent or present in low amounts (less than 5%) will be present at a higher-than-normal level. Typically, this means it will be enriched by at least five-fold, and commonly more than 10-fold above natural occurrence. For example, where a particular hydrogen is enriched in the deuterium isotope, typically a sample of the compound will have at least 50% deuterium incorporation at the labeled position(s).

The concentration of such a heavier isotope, specifically deuterium, may be defined by the isotopic enrichment factor. The term “isotopic enrichment factor” as used herein means the ratio between the isotopic abundance and the natural abundance of a specified isotope. If a hydrogen atom in a compound of the invention is enriched in deuterium isotope, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “an optical isomer” or “a stereoisomer” refers to any of the various stereoisomeric configurations which may exist for a given compound of the present invention. The term “chiral” refers to molecules which have the property of non-superimposability on their mirror image partner, while the term “achiral” refers to molecules which are superimposable on their mirror image partner.“Enantiomers” are a pair of stereoisomers that are non-superimposable mirror images of each other. A 1:1 mixture of a pair of enantiomers is a “racemic” mixture. The term is used to designate a racemic mixture where appropriate. The invention includes enantiomers and diastereomers, and it includes separated enantiomers, mixtures of enantiomers, and racemates of the compound. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Compounds of the invention are not necessarily chiral, since the skeleton of Formula (I) is not inherently necessarily chiral, but they may contain one or more chiral centers as a result of the presence of a substituent in the compounds of Formula (I). Moreover, chirality may arise due to restricted rotation of single bonds (atropisomerism), particularly in the biaryl linkages of compounds of the invention. Compounds may also exist as single geometric isomers or mixtures of geometric isomers with respect to geometry of carbon-carbon double bonds. Where compounds of the invention can exist as two or more stereoisomers (including diasteromers), geometric isomers, or atropisomeric forms, the invention includes each such form and combinations or mixtures of those forms, unless otherwise indicated.

The compounds of the invention may be made and used as single stereoisomers, geometric isomers, or atropisomers, or as mixtures. Preferably, a single stereoisomer, geometric isomer, or atropisomer is used. Methods for separating these forms, including diastereomers and enantiomers, are known in the art. In certain embodiments, the compounds of the invention are used as a single substantially pure stereoisomer, geometric isomer, or atropisomer, meaning at least 90% of a sample of the compound is the specified isomer and less than 10% of the sample is any other isomer or mixture of isomers. Preferably, at least 95% of the sample is a single stereoisomer, geometric isomer, or atropisomer. Selection of a suitable isomer is within the ordinary level of skill, as one isomer will frequently be more active in the relevant in vitro assays described herein, and will be the preferred isomer. Where in vitro activity differences between isomers are relatively small, e.g. less than about a factor of 4, a preferred isomer may be selected based on other factors, such as pharmacokinetic behavior or toxicology, as is well understood in the art.

Chiral compounds of the invention may or may not have a second chiral center. Where a second chiral center is present, the preferred diastereomer is typically the one having a greater potency as an antagonist of A2AR, or alternatively the greater potency as an antagonist of A_{2B}R: if the activities of two isomers do not differ by a factor of 4 in in vitro activity, each isomer or a mixture of those isomers may suitably be used for the methods and compositions of the invention.

### General Synthetic Procedures

Compounds of the present invention can be readily prepared from available starting materials using procedures known to those skilled in the art in view of the examples and schemes provided herein.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the compound may be E or Z geometric configuration. If the compound contains a disubstituted cycloalkyl, the cycloalkyl substituents may be in either a cis- or trans-configuration, or a mixture thereof. All tautomeric forms are also intended to be included.

Any resulting mixtures of isomers can be separated on the basis of the physicochemical differences of the constituents, into the pure or substantially pure geometric or optical isomers or diastereomers, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Within the scope of this text, only a readily removable group that is not a constituent of the particular desired end product of the particular example, intermedieate, or compound of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Protein” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Salts of compounds of the present invention having at least one salt-forming group may be prepared in a manner known to those of skill in the art. For example, salts of compounds of the present invention having acid groups may be formed by treating the compounds with basic metal compounds, such as alkali metal salts of suitable organic carboxylic acids, e.g., the sodium salt of 2-ethyl hexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds, or with ammonia or a suitable organic amine, stoichiometric amounts or only a small excess of the salt-forming agent preferably being used.

Acid addition salts of compounds of the present invention are obtained in customary manner, e.g., by treating the compounds with an acid or a suitable anion exchange reagent. Internal salts of compounds of the present invention containing acid and basic salt-forming groups, e.g., a free carboxy group and a free amino group, may be formed, e.g., by the neutralisation of salts, such as acid addition salts, to the isoelectric point, e.g., with weak bases, or by treatment with ion exchangers.

Salts can be converted or interconverted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

The process steps to synthesize the compounds of the invention can be carried out under reaction conditions that are known to those of skill in the art, including those mentioned specifically, in the absence or, customarily, in the presence of solvents or diluents, including, for example, solvents or diluents that are inert towards the reagents used and capable to dissolve them, in the absence or presence of catalysts, condensation or neutralizing agents, for example ion exchangers, such as cation exchangers, e.g., in the 1-1±form, depending on the nature of the reaction and/or of the reactants at reduced, normal or elevated temperature, for example in a temperature range of from about −-100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water; esters, such as lower alkyl-lower alkanoates, for example ethyl acetate; ethers, such as aliphatic ethers, for example diethyl ether, or cyclic ethers, for example tetrahydrofurane or dioxane; liquid aromatic hydrocarbons, such as benzene or toluene; alcohols, such as methanol, ethanol or 1- or 2-propanol; nitriles, such as acetonitrile or propionitrile; halogenated hydrocarbons, such as methylene chloride or chloroform; acid amides, such as dimethylformamide or dimethyl acetamide; bases, such as heterocyclic nitrogen bases, for example pyridine or N-methylpyrrolidin-2-one; carboxylic acid anhydrides, such as lower alkanoic acid anhydrides, for example acetic anhydride; cyclic, linear or branched hydrocarbons, such as cyclohexane, hexane or isopentane; or mixtures of those solvents, for example aqueous solutions, unless otherwise indicated in the description of the processes. Such solvent mixtures may also be used in working up reaction products, for example by chromatography or partitioning.

The compounds, including their salts, may also be obtained in the form of hydrates, or their crystals may, for example, include the solvent used for crystallization. Different crystalline forms may be present.

The invention also provides methods for making compounds of Formula (I) as described herein.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

In accordance with the foregoing the present invention provides in a yet further aspect:
A pharmaceutical combination comprising (a) a first agent which is a compound of the invention, e.g. a compound of Formula I or any subformulae or species thereof, and (b) a co-agent, e.g. a second drug or therapeutic agent as defined above.
A method as defined above comprising co-administration, e.g. concomitantly or in sequence, of a therapeutically effective amount of a compound of the invention, e.g. a compound of Formula I or any subformulae thereof, and a co-agent, e.g. a second therapeutic agent as defined above.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time, but are used in the course of a coordinated therapeutic intervention. Fixed combinations are within the scope of the present invention, as are sequential administrations to provide concurrent therapeutic effects. The administration of a pharmaceutical combination of the invention results in a beneficial effect, e.g. a synergistic therapeutic effect, compared to a monotherapy applying only one of its pharmaceutically active ingredients.

Each component of a combination according to this invention may be administered separately, together, or in any combination thereof.

The compound of the invention and any additional agent may be formulated in separate dosage forms. Alternatively, to decrease the number of dosage forms administered to a patient, the compound of the invention and any additional agent may be formulated together in any combination. For example, the compound of the invention may be formulated in one dosage form and the additional agent may be formulated together in another dosage form. Any separate dosage forms may be administered at the same time or different times.

Alternatively, a composition of this invention comprises an additional agent as described herein. Each component may be present in individual compositions, combination compositions, or in a single composition.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like, or a compound formed by a compound of the invention forming an acid addition salt. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

The compounds and compositions described herein can be administered to a subject in need of treatment for a cell proliferation disorder such as cancer, particularly cancers selected from leukemia, lymphoma, lung cancer, colon cancer, CNS cancer, melanoma, ovarian cancer, renal cancer, prostate cancer, breast cancer, head and neck cancers, and pancreatic cancer. The subject is typically a mammal diagnosed as being in need of treatment for one or more of such proliferative disorders, and frequently the subject is a human. The methods comprise administering an effective amount of at least one compound of the invention; optionally the compound may be administered in combination with one or more additional therapeutic agents, particularly therapeutic agents known to be useful for treating the cancer or proliferative disorder afflicting the particular subject.

### Anti-Cancer Indications

Because blocking A_{2A} receptor activity can increase the anti-tumor activity of lymphocytes, A_{2A} antagonists are useful to treat a broad range of cancers, including in particular solid tumors in which adenosine in the tumor microenvironment may play a strong role in suppressing the anti-tumor immune response. Examples of the cancers treatable by compounds of Formula (I) include, but are not limited to, lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), skin cancer (e.g. melanoma, squamous cell carcinoma, Kaposi sarcoma, Merkel cell skin cancer), bladder cancer, breast cancer, cervical cancer, colorectal cancer, cancer of the small intestine, colon cancer, rectal cancer, cancer of the anus, endometrial cancer, gastric cancer, head and neck cancer (e.g., cancers of the larynx, hypopharynx, nasopharynx, oropharynx, lips, and mouth), liver cancer (e.g., hepatocellular carcinoma, cholangiocellular carcinoma), ovarian cancer, prostate cancer, testicular cancer, uterine cancer, esophageal cancer, gall bladder cancer, pancreatic cancer (e.g. exocrine pancreatic carcinoma), stomach cancer, thyroid cancer, and parathyroid cancer.

Compounds of the disclosure can also be useful in the inhibition of tumor metastasis.

### A2A Antagonist as a Combination Agent

Suitable agents for use in combination with the compounds of the present application for the treatment of cancer include chemotherapeutic agents, targeted cancer therapies, radiation therapy and immunotherapies. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylenemelamine, triethylenethiophosphoramine, busulfan, carmustine, lomustine, streptozocin, dacarbazine, and temozolomide.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-.alpha.), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agents include inhibitors of kinases associated cell proliferative disorder. These kinases include but not limited to Aurora-A, BTK, CDK1, CDK2, CDK3, CDK4, CDKS, CDK6, CDK7, CDK8, CDK9, ephrin receptor kinases, CHK1, CHK2, SRC, Yes, Fyn, Lck, Fer, Fes, Syk, Itk, Bmx, GSK3, JNK, MEK, PAK1, PAK2, PAK3, PAK4, PDK1, PKA, PKC, RAF, Rsk and SGK.

Other anti-cancer agents include PARP inhibitor such as olaparib, rucaparib and niraparib

Other anti-cancer agents include CSF1R inhibitors (PLX3397, LY3022855, etc.) and CSF1R antibodies (IMC-054, RG7155, etc).

Other anti-cancer agents include BET inhibitors (INCB054329, OTX015, CPI-0610, etc.), LSD1 inhibitors (GSK2979552, INCB059872, etc), HDAC inhibitors (panobinostat, vorinostat, etc), DNA methyl transferase inhibitors (azacitidine and decitabine), and other epigenetic modulators.

Other anti-cancer agents include Bc12 inhibitor ABT-199, and other Bcl-2 family protein inhibitors.

Other anti-cancer agents include TGF beta receptor kinase inhibitor such as LY2157299.

Other anti-cancer agents include BTK inhibitor such as ibrutinib.

Other anti-cancer agents include SHP-2 inhibitor such as TNO155.

Other anti-cancer agents include HIF-2a inhibitors such as PT2977 and PT2385.

Other anti-cancer agent(s) include antibody therapeutics such as trastuzumab (Herceptin), Alemtuzumab, blinatumomab, Bev acizumab, and Cetuximab.

Other anti-cancer agents include beta catenin pathway inhibitors, notch pathway inhibitors and hedgehog pathway inhibitors.

Compounds of this application may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present dislcosure. These include anti-androgens including but not limited to flutamide, bicalutamide, and nilutamide, luteinizing hormone-releasing hormone (LHRH) analogs including leuprolide, oserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with A2A inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Other anti-cancer agents/drugs that can be used in combination with the compounds of the invention include, but are not limited to, liver X receptor (LXR) modulators, including LXR agonists and LXR beta-selective agonists; aryl hydrocarbon receptor (AhR) inhibitors;); MEK inhibitors, including cobimetinib; B-Raf enzyme inhibitors, including vemurafenib; inhibitors of the Wnt pathway; inhibitors of epidermal growth factor receptor (EGFR) including AZD9291, (AstraZeneca), erlotinib, gefitinib, panitumumab, and cetuximab; adenosine A_{2B} receptor inhibitors;

Other anti-cancer treatments that can be used in combination with compounds of the invention include surgery and radiation therapy.

One or more additional immune checkpoint inhibitors can also be used in combination with a compound as described herein for treatment of a wide range of tumors. Exemplary immune checkpoint inhibitors include inhibitors (small molecules or biologics) against immune checkpoint molecules such as CD27, CD28, CD40, CD122, CD96, CD73, CD39, CD47, OX40, GITR, CSF1R, HPK1, JAK, PI3K delta, PI3K gamma, TAM, arginase, CD137 (also known as 4-1BB), ICOS, A2BAR, B7-H3, B7-H4, BTLA, CTLA-4, LAG3, TIM3, VISTA, CD96, TIGIT, PD-1, PD-Ll and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from B7-H3, B7-H4, BTLA, CTLA-4, IDO, TDO, arginase, KIR, LAG3, PD-1, TIM3, CD96 TIGIT and VISTA. In some embodiments, the compounds provided herein can be used in combination with one or more agents selected from KIR inhibitors and anti-TIGIT antibodies.

In one embodiment, the combination therapies of the invention include an immunomodulator that is an inhibitor or antagonist of an inhibitory molecule of an immune checkpoint molecule. In another embodiment the immunomodulator binds to a protein that naturally inhibits the immuno-inhibitory checkpoint molecule. Thus a compound of any one of embodiments 1-20 or a pharmaceutical composition of embodiment 21 can be administered to a subject who is being treated with an immunomodulator; the immunomodulator and compound can be administered together or separately, but are simultaneously used to treat a condition treatable with the compounds of Formula (I) as described herein.

The term “immune checkpoints” refers to a group of molecules on the cell surface of CD4 and CD8 T cells. These molecules can effectively serve as “brakes” to down-modulate or inhibit an adaptive immune response Immune checkpoint molecules include, but are not limited to, Programmed Death 1 (PD-1), Cytotoxic T-Lymphocyte Antigen 4 (CTLA-4), B7H1, B7H4, OX-40, CD137, CD40, and LAG3, which directly inhibit immune cells Immunotherapeutic agents which can act as immune checkpoint inhibitors useful in the methods of the present invention, include, but are not limited to, inhibitors of PD-L1, PD-L2, CTLA4, TIM3, LAG3, VISTA, BTLA, TIGIT, LAIR1, CD160, 2B4 and/or TGFR beta. Inhibition of an inhibitory molecule can be performed by inhibition at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide, e.g., a soluble ligand, or an antibody or antigen-binding fragment thereof, that binds to the inhibitory molecule.

The immunomodulator can be administered concurrently with, prior to, or subsequent to, one or more compounds of the invention, and optionally one or more additional therapies or therapeutic agents. The therapeutic agents in the combination can be administered in any order. In general, each agent will be administered at a dose and/or on a time schedule determined for that agent. It will further be appreciated that the therapeutic agents utilized in this combination may be administered together in a single composition or administered separately in different compositions. In general, it is expected that each of the therapeutic agents utilized in combination be utilized at levels that do not exceed the levels at which they are utilized individually. In some embodiments, the levels utilized in combination will be lower than those utilized individually.

In certain embodiments, the compounds described herein are administered in combination with one or more immunomodulators that are inhibitors of PD-1, PD-L1 and/or PD-L2. Each such inhibitor may be an antibody, an antigen binding fragment thereof, an immunoadhesin, a fusion protein, or an oligopeptide. Examples of such immunomodulators are known in the art.

In some embodiments, the immunomodulator is an anti-PD-1 antibody chosen from MDX-1106, Merck 3475 or CT-011.

In some embodiments, the immunomodulator is an immunoadhesin (e.g., an immunoadhesin comprising an extracellular or PD-1 binding portion of PD-L1 or PD-L2 fused to a constant region (e.g., an Fc region of an immunoglobulin sequence).

In some embodiments, the immunomodulator is a PD-1 inhibitor such as AMP-224.

In some embodiments, the the immunomodulator is a PD-L1 inhibitor such as anti-PD-L1 antibody.

In some embodiments, the immunomodulator is an anti-PD-L1 binding antagonist chosen from YW243.55.S70, MPDL3280A, MEDI-4736, MSB-0010718C, or MDX-1105. MDX-1105, also known as BMS-936559, is an anti-PD-Ll antibody described in WO2007/005874. Antibody YW243.55.S70 is an anti-PD-Ll described in WO 2010/077634.

In some embodiments, the immunomodulator is nivolumab (CAS Registry Number: 946414-94-4). Alternative names for nivolumab include MDX-1106, MDX-1106-04, ONO-4538, or BMS-936558. Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449, EP2161336 and WO2006/121168.

In some embodiments, the immunomodulator is an anti-PD-1 antibody Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509, WO2009/114335, and WO2013/079174.

In some embodiments, the immunomodulator is Pidilizumab (CT-011; Cure Tech), a humanized IgGlk monoclonal antibody that binds to PD1. Pidilizumab and other humanized anti-PD-1 monoclonal antibodies are disclosed in WO2009/101611.

Other anti-PD1 antibodies useful as immunomodulators for use in the methods disclosed herein include AMP 514 (Amplimmune), and anti-PD1 antibodies disclosed in U.S. Pat. No. 8,609,089, US 2010028330, and/or US 20120114649. In some embodiments, the anti-PD-L1 antibody is MSB0010718C. MSB0010718C (also referred to as A09-246-2; Merck Serono) is a monoclonal antibody that binds to PD-L1.

In some embodiments, the immunomodulator is MDPL3280A (Genentech/Roche), a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents useful as immunomodulators for methods of the invention include YW243.55.570 (see WO2010/077634), MDX-1105 (also referred to as BMS-936559), and anti-PD-Ll binding agents disclosed in WO2007/005874.

In some embodiments, the immunomodulator is AMP-224 (B7-DCIg; Amplimmune; e.g., disclosed in WO2010/027827 and WO2011/066342), is a PD-L2 Fc fusion soluble receptor that blocks the interaction between PD1 and B7-H1.

In some embodiments, the immunomodulator is an anti-LAG-3 antibody such as BMS-986016. BMS-986016 (also referred to as BMS986016) is a monoclonal antibody that binds to LAG-3. BMS-986016 and other humanized anti-LAG-3 antibodies are disclosed in US 2011/0150892, WO2010/019570, and WO2014/008218

In certain embodiments, the combination therapies disclosed herein include a modulator of a costimulatory molecule or an inhibitory molecule, e.g., a co-inhibitory ligand or receptor.

In one embodiment, the costimulatory modulator, e.g., agonist, of a costimulatory molecule is chosen from an agonist (e.g., an agonistic antibody or antigen-binding fragment thereof, or soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In another embodiment, the combination therapies disclosed herein include an immunomodulator that is a costimulatory molecule, e.g., an agonist associated with a positive signal that includes a costimulatory domain of CD28, CD27, ICOS and/or GITR.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 090505B1, U.S Pat. No.: 8,586,023, PCT Publication Nos.: WO 2010/003118 and 2011/090754, or an anti-GITR antibody described, e.g., in U.S. Pat. No.: 7,025,962, European Patent No.: 1947183B1, U.S. Pat. Nos.: 7,812,135, 8,388,967, 8,591,886.

In one embodiment, the immunomodulator used is a soluble ligand (e.g., a CTLA-4-Ig), or an antibody or antibody fragment that binds to PD-L1, PD-L2 or CTLA4. For example, the anti-PD-1 antibody molecule can be administered in combination with an anti-CTLA-4 antibody, e.g., ipilimumab, for example. Exemplary anti-CTLA4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

In one embodiment, an anti-PD-1 antibody molecule is administered after treatment with a compound of the invention as described herein.

In another embodiment, an anti-PD-1 or PD-L1 antibody molecule is administered in combination with an anti-LAG-3 antibody or an antigen-binding fragment thereof. In another embodiment, the anti-PD-1 or PD-L1 antibody molecule is administered in combination with an anti-TIM-3 antibody or antigen-binding fragment thereof. In yet other embodiments, the anti-PD-1 or PD-L1 antibody molecule is administered in combination with an anti-LAG-3 antibody and an anti-TIM-3 antibody, or antigen-binding fragments thereof. The combination of antibodies recited herein can be administered separately, e.g., as separate antibodies, or linked, e.g., as a bispecific or trispecific antibody molecule. In one embodiment, a bispecific antibody that includes an anti-PD-1 or PD-L1 antibody molecule and an anti-TIM-3 or anti-LAG-3 antibody, or antigen-binding fragment thereof, is administered. In certain embodiments, the combination of antibodies recited herein is used to treat a cancer, immune disorder, diabetes, renal disease, vascular disease or lung disease selected from those described herein. The efficacy of the aforesaid combinations can be tested in animal models known in the art.

Exemplary immunomodulators that can be used in the combination therapies include, but are not limited to, e.g., afutuzumab (available from Roche®); pegfilgrastim (Neulasta®); lenalidomide (CC-5013, Revlimid®); thalidomide (Thalomid®), actimid (CC4047); and cytokines, e.g., IL-21 or IRX-2 (mixture of human cytokines including interleukin 1, interleukin 2, and interferon y, CAS 951209-71-5, available from IRX Therapeutics).

Exemplary doses of such immunomodulators that can be used in combination with the compounds of the invention include a dose of anti-PD-1 antibody molecule of about 1 to 10 mg/kg, e.g., 3 mg/kg, and a dose of an anti-CTLA-4 antibody, e.g., ipilimumab, of about 3 mg/kg.

Examples of embodiments of the methods of using the compounds of the invention in combination with an immunomodulator include these:

i. A method to treat cancer in a subject, comprising administering to the subject a compound of Formula (I) as described herein, and an immunomodulator.

ii. The method of embodiment i, wherein the immunomodulator is an activator of a costimulatory molecule or an inhibitor of an immune checkpoint molecule.

iii. The method of either of embodiments i and ii, wherein the activator of the costimulatory molecule is an agonist of one or more of OX40, CD2, CD27, CDS, ICAM-1, LFA-1 (CD11a/CD18), ICOS (CD278), 4-1BB (CD137), GITR, CD30, CD40, BAH-R, HVEM, CD7, LIGHT, NKG2C, SLAMF7, NKp80, CD160, B7-H3 and CD83 ligand.

iv. The method of any of embodiments i-iii above, wherein the inhibitor of the immune checkpoint molecule is chosen from PD-1, PD-L1, PD-L2, CTLA4, TIM3, LAG3, VISTA, BTLA, TIGIT, LAIR1, CD160, 2B4 and TGFR beta.

v. The method of any of any of embodiments i-iii, wherein the inhibitor of the immune checkpoint molecule is chosen from an inhibitor of PD-1, PD-L1, LAG-3, TIM-3 or CTLA4, or any combination thereof.

vi. The method of any of embodiments i-v, wherein the inhibitor of the immune checkpoint molecule is a soluble ligand or an antibody or antigen-binding fragment thereof, that binds to the immune checkpoint molecule.

vii. The method of any of embodiments i-vi, wherein the antibody or antigen-binding fragment thereof is from an IgG1 or IgG4 (e.g., human IgG1 or IgG4).

viii. The method of any of embodiments i-vii, wherein the antibody or antigen-binding fragment thereof is altered, e.g., mutated, to increase or decrease one or more of: Fc receptor binding, antibody glycosylation, the number of cysteine residues, effector cell function, or complement function.

ix. The method of any of embodiments i-viii, wherein the antibody molecule is a bispecific or multispecific antibody molecule that has a first binding specificity to PD-1 or PD-L1 and a second binding specifity to TIM-3, LAG-3, or PD-L2.

x. The method of any of embodiments i-ix, wherein the immunomodulator is an anti-PD-1 antibody chosen from Nivolumab, Pembrolizumab or Pidilizumab.

xi. The method of any of embodiments i-x, wherein the immunomodulator is an anti-PD-L1 antibody chosen from YW243.55.570, MPDL3280A, MEDI-4736, MSB-0010718C, or MDX-1105.

xii. The method of any of embodiments i-x, wherein the immunomodulator is an anti-LAG-3 antibody molecule.

xiii. The method of embodiment xii, wherein the anti-LAG-3 antibody molecule is BMS-986016,

xiv. The method of any of embodiments i-x, wherein the immunomodulator is an anti-PD-1 antibody molecule administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg., e.g., once a week to once every 2, 3, or 4 weeks.

xv. The method of embodiment xiv, wherein the anti-PD-1 antibody molecule is administered at a dose from about 10 to 20 mg/kg every other week.

xvi. The method of embodiment xv, wherein the anti-PD-1 antibody molecule, e.g., nivolumab, is administered intravenously at a dose from about 1 mg/kg to 3 mg/kg, e.g., about 1 mg/kg, 2 mg/kg or 3 mg/kg, every two weeks.

xvii. The method of embodiment xv, wherein the anti-PD-1 antibody molecule, e.g., nivolumab, is administered intravenously at a dose of about 2 mg/kg at 3-week intervals. inhibitors, LAIR1 inhibitors, CD160 inhibitors, 2B4 inhibitors and TGF beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab.

In some embodiments, the inhibitor of an immune checkpoint molecule is an inhibitor of LAGS, e.g., an anti-LAGS antibody. In some embodiments, the anti-LAGS antibody is BMS-986016 or LAG525.

In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248.

In some embodiments, the inhibitor of an immune checkpoint molecule is an inhibitor of OX40, e.g., an anti-OX40 antibody or OX40L fusion protein. In some embodiments, the anti-OX40 antibody is MEDI0562 or, INCAGN01949, GSK2831781, GSK-3174998, MOXR-0916, PF-04518600 or LAG525. In some embodiments, the OX40L fusion protein is MEDI6383.

Compounds of the invention can also be used to increase or enhance an immune response, including increasing the immune response to an antigen; to improve immunization, including increasing vaccine efficacy; and to increase inflammation. In some embodiments, the compounds of the invention can be used to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine).

Anti-cancer vaccines include dendritic cells, synthetic peptides, DNA vaccines and recombinant viruses

Other immune-modulatory agents also include those that block immune cell migration such as antagonists to chemokine receptors, including CCR2 and CCR4; Sting agonists and Toll receptor agonists.

Other anti-cancer agents also include those that augment the immune system such as adjuvants or adoptive T cell transfer.

Compounds of this application may be effective in combination with CAR (Chimeric antigen receptor) T cell treatment as a booster for T cell activation

### Indications Beyond Cancers

Adenosine acts on a variety of immune cells to induce immunosuppression, and the immunosuppressive effects of ectonucleotidases that enhance adenosine levels are also associated with enhanced infections of mammalian cells by parasites, fungi, bacteria, and viruses. Apart from immunosuppressive effects, adenosine also promotes fibrosis (excess matrix production) in a variety of tissues. A2A is one of the major adenosine receptors involved in these physiological/pathological processes. Therefore, improved treatments targeting A2A would provide therapies for treating a wide range of conditions in addition to cancer, including pulmonary and liver fibrosis, immune and inflammatory disorders, neurological, neurodegenerative and CNS disorders and diseases (e.g., depression, Parkinson's disease).

In one embodiment, compounds of the invention are used to enhance the immune response in an immuno suppressed subject, such as a subject infected with an immunodeficiency virus (e.g., HIV-1 or HIV-2). In another embodiment, compounds of the invention are used to enhance the immune response in a subject infected with a pathogen such as a bacterial, viral, or fungal pathogen, to facilitate destruction of the pathogen in the subject.

Suitable antiviral agents contemplated for use in combination with the compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methyl ethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Compounds of the invention can be used to improve motor impairment due to neurodegenerative diseases such as Parkinson's disease.

Compounds of the invention can be used to treat and prevent hepatic cirrhosis

### Indications Related to Inhibition of A_{2B} Receptor

In addition to antagonizing A_{2A}R, some compounds of the invention also have antagonistic activity against A_{2B} adenosine receptors, which bind to the endogenous ligand adenosine with much lower affinity compared to A_{2A} adenosine receptors. The A_{2B} receptor, in addition to activating adenylate cyclase in a manner similar to A_{2A}R, is believed to be coupled to distinct intracellular signaling pathways and play physiological roles that differ from those of A_{2A}Rs. Numerous studies have demonstrated a critical role of A_{2B}R in the regulation of various human diseases including cancer, renal disease, diabetes, vascular and lung disease. As such, compounds of the invention can be used to treat these human diseases as a single agent or in combination with other treatment modalities. Compounds of the invention with activity on both A_{2A}R and A_{2B}R may be particularly effective against cancer, and should reduce the likelihood that treated cancers will develop resistance.

### Methods of Synthesizing the Compounds of the Invention

Compounds of Formula (I) with R^{A}O-side chains can be prepared using conventional synthetic methods according to the route outlined in Scheme 1. The Suzuki coupling between 4,6-dichloropyrimidin-2-amine and a suitable aryl boronic acid affords the 4-chloro substituted arylated pyrimidine-2-amine The iodine group then is introduced on the pyrimidine ring by reaction with N-iodosuccinimide, followed by coupling with a 4-pyridinyl boronic ester agent to form di-aryl substituted pyrimidine. The final step is the substitution reaction between aryl chloride and RAOH to afford the desired target compound.

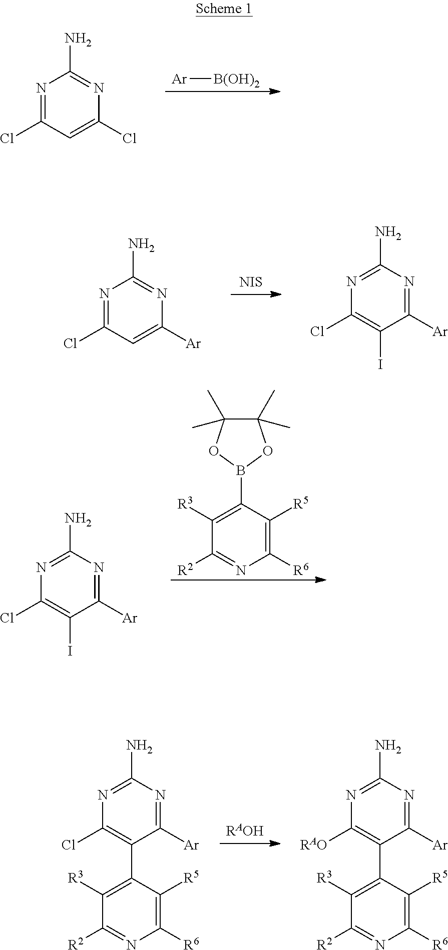

The desired target compounds can also be prepared according to the route shown in Scheme 2. In this approach, the R^{A}O— side chain can be incorporated first by reaction of R^{A}OH with a chloropyrimidine, followed by introduction of the substituted pyridine ring by Suzuki Coupling between aryl iodide and boronic ester.

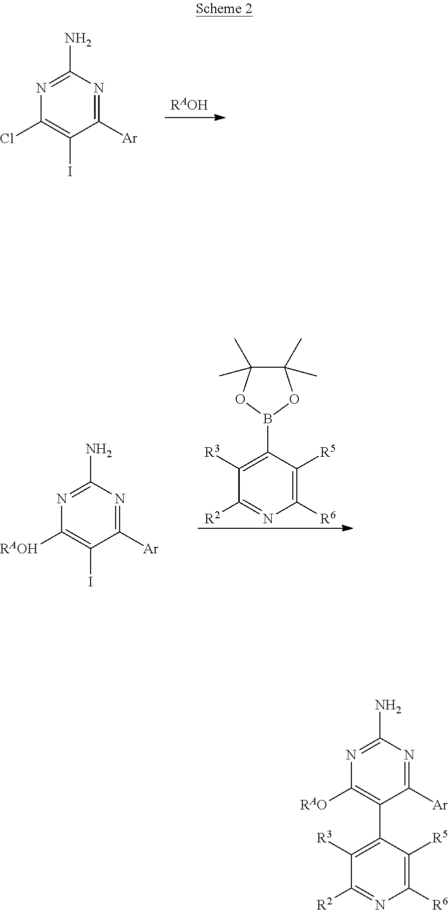

A third way to synthesize target compound is to convert aryl chloride into the corresponding hydroxypyrimidine first, as shown in Scheme 3, which then is converted into R^{A}O side chain by some conventional synthetic methods.

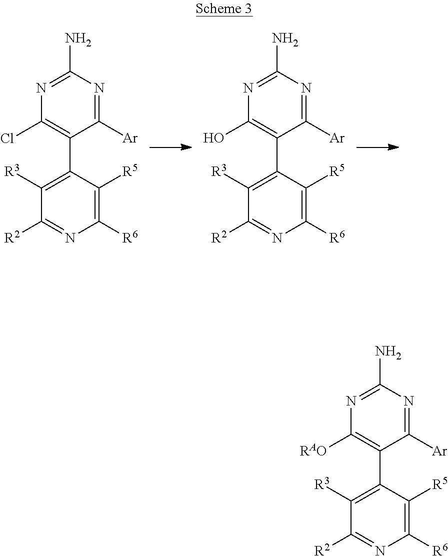

When the side chains are alkyl groups, the desired target compounds can be prepared via the Suzuki coupling of aryl chloride with vinylboronic acid. After hydrogenation the saturated side chain can be established as shown in Scheme 4. The palladium-catalyzed carbonylation reaction with aryl chloride can also incorporate ester group on the pyrimidine ring. Other side chains, such as alkyl and amide groups, then can be constructed after some straightforward functional group transformations.

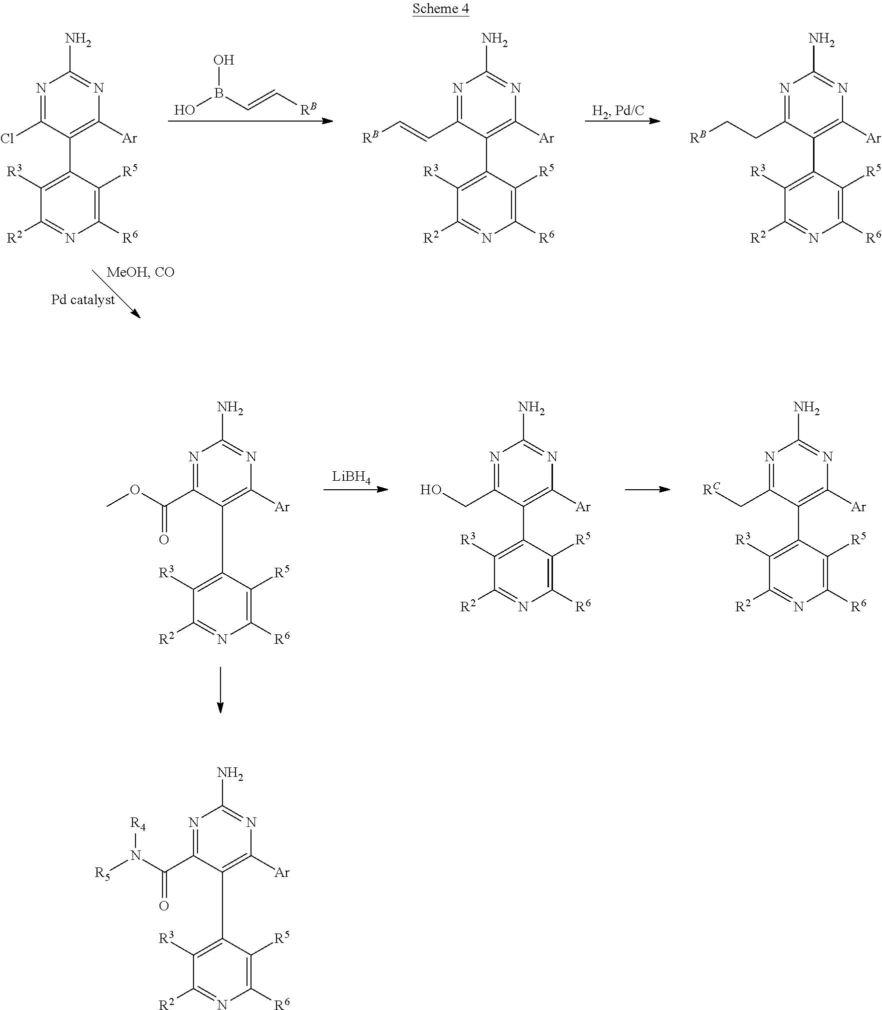

Compounds of Formula (I) where L is an amino group can be synthesized in similar ways to those used for compounds with R^{A}O side chains. The amino group can be incorporated first, followed by the introduction of the pyridine group, or these two chemical transformations can be reversed, and the amino moiety can be introduced in the last step as shown in Scheme 5.

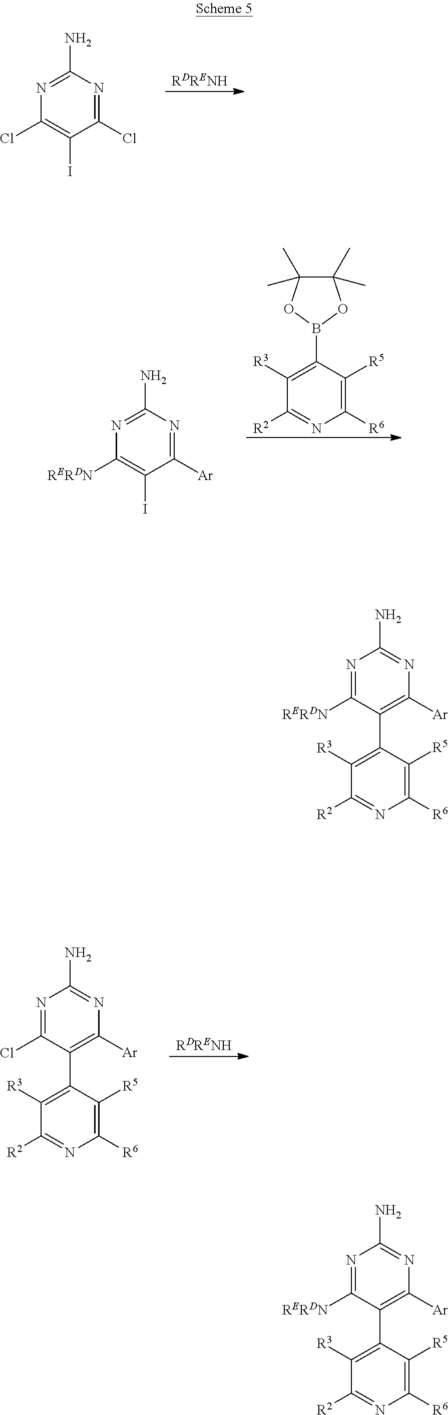

### Pharmaceutical Compositions, Combinations, and Other Related Uses

In still another aspect, the present disclosure provides for a pharmaceutical composition comprising a compound described above admixed with at least one pharmaceutically acceptable carrier or excipient. Pharmaceutically acceptable carriers are well known in the art, and include water and isotonic glucose or saline, each of which is preferably sterile. For solid compositions, pharmaceutically acceptable carriers include, e g , mannitol, sucrose, cellulose, and the like. Suitable diluents, binders, glidants, disintegrants, lubricants, preservatives, and other ingredients are mentioned herein or known to those of ordinary skill in the art.

The above described compounds can be used for any suitable purpose. For example, the present compounds can be used in therapy and/or testing. Thus the invention provides a compound of any of the disclosed embodiments herein for use in therapy, and particularly for use in therapy to treat a proliferative disorder, or a cancer, or a tumor.

In yet another aspect, the present disclosure provides for a method for treating and/or preventing a proliferation disorder, a cancer, or a tumor. The method comprises administering to a subject in need thereof a compound of any of the above compound embodiments. In some embodiments, the method comprises administering an effective amount of the compound. In some embodiments, the subject is one diagnosed as in need of treatment for at least one condition treatable with the compounds of the invention.

In yet another aspect, the present disclosure provides for a use of a compound described above for the manufacture of a medicament, especially a medicament for use in treating a proliferative disorder, tumor or cancer.

In yet another aspect, the present disclosure provides for a combination for treating and/or preventing a proliferation disorder, a cancer, or tumor in a subject, which combination comprises an effective amount of a compound described above, or a pharmaceutically acceptable salt thereof, and an effective amount of a second prophylactic or therapeutic agent for treating and/or preventing a proliferation disorder, a cancer, or a tumor.

In yet another aspect, the present disclosure provides for a method for treating and/or preventing a proliferation disorder, a cancer, or a tumor, which comprises administering to a subject in need of such treatment an effective amount of a compound of Formula (I) as described herein in the form of a pharmaceutical composition as described above.

In yet another aspect, the present disclosure provides for a method for inhibiting an activity of adenosine A2A receptor, either in vitro or in vivo. The method comprises contacting the A2A receptor with a compound of Formula (I) as described herein.

The present methods can be used for any suitable purpose. In some embodiments, the present methods can be used to treat and/or prevent a proliferation disorder, a cancer, or a tumor.

In some embodiments, the invention provides any of the individual compounds selected from the group consisting of the compounds in Table 1, as well as any subset thereof, and including the pharmaceutically acceptable salts of any of the compounds in Table 1.

### Formulations

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

The compounds having Formula (I) as described herein are generally soluble in organic solvents such as chloroform, dichloromethane, ethyl acetate, ethanol, methanol, isopropanol, acetonitrile, glycerol, N,N-dimethylformamide, N,N-dimethylacetamide, dimethylsulfoxide, etc. In one embodiment, the present invention provides formulations prepared by mixing a compound having Formula (I) with a pharmaceutically acceptable carrier. In one aspect, the formulation may be prepared using a method comprising: a) dissolving a described compound in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present methods include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present methods include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present methods include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present methods include but are not limited to oleic acid, monoglycerides, diglycerides, a mono- or di-fatty acid ester of PEG, or a combination thereof.

Illustrative examples of cyclodextrins for use in the present methods include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present methods include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug Combinations

The methods of the embodiments comprise administering an effective amount of at least one exemplary compound of the present disclosure; optionally the compound may be administered in combination with one or more additional therapeutic agents, particularly therapeutic agents known to be useful for treating a proliferation disorder, a cancer, a tumor, an inflammatory disease, an autoimmune disease, psoriasis, dry eye or an immunologically related disease afflicting the subject.

Compounds of the invention inhibit signaling by the A2A receptor, and are thus checkpoint inhibitors useful to treat tumors that cause extracellular adenosine levels to increase. Mediavilla-Varella, et al., Neoplasia, 19(7), July 2017, pp. 530-536. Like PD-1 and CTLA-4 receptors, the adenosine A_{2A} receptor plays a regulatory role by suppressing immunologic response to tumor cells: inhibition of A_{2A}R can thus allow the adaptive immune system to better combat tumors associated with locally elevated adenosine levels, and is expected to be more effective when used in combination with other checkpoint inhibitors. Multiple checkpoint pathway inhibition has been shown to have an additive effect, as shown by an increase in response with blockade to PD-1 and CTLA-4 via monoclonal antibodies as compared to the blockade of a single pathway. Also, inhibition of A_{2A}R in chimeric antigen receptor T-cells (CAR-T cells) was shown to increase tumor clearance through CAR T-cell therapy in mice. Beavis et al., J Clin. Invest. 2017, 127(3):929-941. It is thus expected that A_{2A}R blockade would increase the efficacy of such treatments. Accordingly, the compounds of Formula (I) can advantageously be used in combination with other checkpoint inhibitors, including anti-PD-1 and anti-PD-L1 therapeutic agents, and CAR T-cell therapy.

The additional therapeutic agents may be administered in a separate pharmaceutical composition from at least one exemplary compound of the present disclosure or may be included with at least one exemplary compound of the present disclosure in a single pharmaceutical composition. The additional therapeutic agents may be administered simultaneously with, prior to, or after administration of at least one exemplary compound of the present disclosure.

Suitable therapeutic agents for use in combination with the compounds of Formula (I) as described herein include small molecule and biologic agents that inhibit other checkpoint proteins, including inhibitors or antagonists of CTLA-4, PD11, PD1,2, PD1, B7-H3, I37-H4, IDO, BTLA, HVEM, TIM3, GAL9, LAGS, 0X40, VISTA, KIR, 2B4, CD160, CGEN-15049, CHK 1, CHK2A7aR, and B-7. The compounds of the invention can potentiate activity of various known chemotherapeutic agents such as cyclophosphamide., mechlorethamine, chlorambucil, melphalan, dacarbazine, nitrosoureas, temozolomicle, anthracyclines (daunorubicin, doxorubicin, epirubicin, idarubicin, mitaxantrone, valrubicin), taxanes (paclitaxel, docetaxel, abraxane, taxotere), epotholones, HDac inhibitors (vorinostat, romidepsin), topoisomerase inhibitors (irinotecan, topotecan, etoposide, teniposide, taftuposide), various kinase inhibitors (inhibitors of RAF, MEK, ERK. PIM, VEOF, IGFR, BTK, Bcr-Abl, JAIL, and the like) including afatinib, axitinib, bosutinib, cetuximab, cobiraetinib, crizotinib, cabozantinib, dasatinib, entrectinib, erdafitinib, erlotinib, gefitinib, ihruninih, imatinib, lapatinih, leiivatinila, nilotinib, pazopanib, sorafenib, sunitinib, vandetarab, and vemurafenib, nucleotide analogs such as azacytidine, azathioprine, capecitabine, cytarabine, doxifluridine, fluorouracil, gemcitabine, hydroxyurea, mercaptopurine., a ethotrexate, and tioguanine; bleomycin, actinomycin, platinates (carboplatin, cisplatin, oxaliplatin), retinoids, and vinca alkaloids (vinblastine, vincristine, vindesine, vinorelbine). Particular therapeutic agents for use in such combinations include, but are not limited to, ipilimumab, nivolumab, pembrolizumab, atezolizumab, avelumab, durvalumab, spartalizumab, BGB-A317 (PD-1 inhibitor antibody), PBF-509, BMS-936559, tremelimumab, bevacizumab, bleomycin, bortezomib, brentuximab, capecitabine, ceritinib, carboplatin, cisplatin, everolimus, irinotecan, gemcitabine, cyclophosphamide, dactinomycin, daunorubicin, docetaxel, rituximab, sorafenib, temozolomide, temsirolimus, trastuzumab, taxanes, and various kinase inhibitors. Selection of therapeutic agents for use in combination with the compounds of Formula (I) is based on the condition to be treated.

### Methods of Using the Exemplary Compounds and Pharmaceutical Compositions Thereof

The present invention also provides pharmaceutical compositions for the treatment and/or prevention of a proliferation disorder, a cancer, or a tumor, comprising any compound having Formula (I), or any of the compounds in Table 1, combined with at least one pharmaceutically acceptable excipient or diluent.

To practice the method of the present invention, compounds having formula and pharmaceutical compositions thereof may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In addition, the compounds having Formula (I) may be administered alone or in combination with other therapeutic agents, e.g., anticancer agents such as those mentioned above, for the treatment of various proliferation disorder, cancer, and tumors, as well as agents to treat symptoms of the underlying condition or of administration of the compound of the invention. These include anti-inflammatories, steroids, antihistamines, and pain relievers Combination therapies according to the present invention comprise the administration of at least one exemplary compound of the present disclosure and at least one other pharmaceutically active ingredient. The active ingredient(s) and pharmaceutically active agents may be administered separately or together. The amounts of the active ingredient(s) and pharmaceutically active agent(s) and the relative timings of administration will be selected in order to achieve the desired combined therapeutic effect.

### EXAMPLES

Compounds of the invention can readily be prepared using methods known in the art in view of the following examples.

### Abbreviations

Ac acetyl
Ac₂O Acetic anhydride

### ACN Acetonitrile

AcOEt/EtOAc/EA Ethyl acetate
AcOH acetic acid
aq aqueous
Ar aryl
BINAP (1,1′-Binaphthalene-2,2′-diyl)bis(diphenylphosphine)
Bn benzyl
Bu butyl(nBu=n-butyl, tBu=tert-butyl)
nBuLi n-Butyllithium
tBuOH tert-Butanol
tBuONa Sodium tert-butoxide
CCl₄ Carbon tetrachloride

### CDI Carbonyldiimidazole

### CH₃CN Acetonitrile

CH₃NH₂ Methylamine
CO Carbon monoxide
Cs₂CO₃ Cesium carbonate
DAST (Diethylamino)sulfur trifluoride
DBU 1,8-Diazabicyclo[5.4.0]-undec-7-ene
Boc₂O di-tert-butyl dicarbonate

### DCC Dicyclohexylcarbodiimide

### DCE 1,2-Dichloroethane

### DCM Dichloromethane

### DIBAL-H Diisobutylaluminum Hydride

### DIPEA N-Ethyldiisopropylamine

### DMAP Dimethylaminopyridine

### DMF N,N′-Dimethylformamide

### DMSO Dimethylsulfoxide

EI Electrospray ionization
ES-MS Electrospray mass spectrometry

### Et₂O Diethylether

### Et₃N Triethylamine

### Ether Diethylether

EtMgBr Ethylmagnesium bromide

### EtOH Ethanol

### FC Flash Chromatography

h hour(s)
HATU O-(7-Azabenzotriazole-1-yl)-N,N,N′N′-tetramethyluronium hexafluorophosphate
HBTU O-(Benzotriazol-1-yl)-N,N,N′,N′-tetramethyluronium hexafluorophosphate
HCl Hydrochloric acid

### HMPA Hexamethylphosphoramide

### HOBt 1-Hydroxybenzotriazole

### HPLC High Performance Liquid Chromatography

### H₂O Water

i-PrOH Isopropyl alcohol
K₂CO₃ Potassium carbonate
K₃ PO₄ Potassium phosphate tribasic
L liter(s)

### LC-MS Liquid Chromatography Mass Spectrometry

LiAlH₄ Lithium aluminum hydride
LiHMDS Lithium bis(trimethylsilyl)amide
LiOH Lithium hydroxide
M mol/L
mCPBA 3-Chloroperbenzoic acid

### MgSO₄ Magnesium Sulfate

Me methyl
MeMgBr Methylmagnesium bromide

### MeI Iodomethane

### MeOH Methanol

### MPa Megapascal

mg milligram
min minute(s)
mL milliliter
mmol milimolar

### MS Mass Spectrometry

MsCl Methanesulfonyl chloride
m/z Mass divided by charge number

### N Normality

NaBH₃CN Sodium cyanoborohydride
NaBH(OAc)₃ Sodium triacetoxyborohydride
NaH Sodium hydride

### NaHCO₃ Sodium Bicarbonate

Na₂SO₄ Sodium Sulfate

### NH₃ Ammonia

NH₂OH hydroxylamine
NH₄Cl Ammonium chloride

### NIS N-Iodosuccinimide

Pd/C palladium on charcoal
Pd₂(dba)₃ Tris(dibenzylideneacetone)dipalladium(0)

### Pd(dppf)Cl2 [1,1′-Bis(diphenylphosphino)ferrocene]dichloropalladium(II)

Pd(OH)₂ palladium hydroxide
Pd(PPh₃)₃ Tetrakis(triphenylphosphine)-palladium(O)
PE Petroleum ether
PG protecting group
Ph phenyl
pH Scale of acidity from 0 to 14
Ph₃P triphenyl phosphine

### Prep Preparative

Rf ratio of fronts
RP reverse phase
psi pound per square inch
Rt Retention time
rt Room temperature

### SiO₂ Silica gel

### SOCl₂ Thionyl Chloride

TBAF Tetrabutylammonium fluoride
TBDMS t-Butyldimethylsilyl
TBSCl t-Butyldimethylsilyl chloride

### TEA Triethylamine

TFA Trifluoroacetic acid

### THF Tetrahydrofuran

### TLC Thin Layer Chromatography

TsCl toluene sulfonyl chloride
XPhos 2-Dicyclohexylphosphino-2′,4′,6′-triisopropylbiphenyl
Zn(CN)2 Zinc cyanide

The invention is further illustrated by the following examples, which should not be construed as limiting. The assays used throughout the Examples are well established in the art: demonstration of efficacy in these assays is generally regarded as predictive of efficacy in subjects.

### Example 1

### 2-((2-amino-5-(2-methyl-6-(trifluoromethyl)pyridin-4-yl)-6-phenylpyrimidin-4-yl)oxy)-N-methylacetamide

Step 1: 4-chloro-6-phenylpyrimidin-2-amine. Into a 1000-mL round-bottom flask and under nitrogen atmosphere were placed 4,6-dichloropyrimidin-2-amine (30.0 g, 182.9 mmol), phenylboronic acid (11.2 g, 91.5 mmol), Pd(dppf)Cl₂ (6.69 g, 9.15 mmol, K₂CO₃ (50.6 g, 365.9 mmol), 1,4-dioxane (600 mL) and H₂O (30 mL). The resulting mixture was stirred for 16 h at 100° C. After cooling, the reaction mixture was quenched with 300 mL of water. The resulting mixture was extracted with 3×500 mL of ethyl acetate. The combined organic layers were washed with 2×100 mL of H₂O, 100 mL of brine, then dried over anhydrous sodium sulfate and concentrated. The residue was purified on a silica gel column with ethyl acetate/petroleum ether (1/3) to obtain 9.0 g (23.9%) of 4-chloro-6-phenylpyrimidin-2-amine as a light yellow solid. ES-MS (m/z): [M+1]⁺=206.

Step 2: 4-chloro-5-iodo-6-phenylpyrimidin-2-amine. Into a 250-mL round-bottom flask were placed 4-chloro-6-phenylpyrimidin-2-amine (4.2 g, 20.4 mmol), NIS (9.2 g, 40.9 mmol) and DMF (100 mL). The resulting mixture was stirred for 16 h at 80° C. After cooling, the reaction mixture was quenched with 100 mL of water. The resulting mixture was extracted with 3×50 mL of ethyl acetate. The combined organic layers were washed with 2×50 mL of H₂O, 50 mL of brine, then dried over anhydrous sodium sulfate and concentrated. The residue was purified on silica gel column with ethyl acetate/petroleum ether (1/1) to obtain 2.1 g (31.0%) of 4-chloro-5-iodo-6-phenylpyrimidin-2-amine as a brown solid. ES-MS (m/z): [M+2]⁺=332.

Step 3: methyl 2-((2-amino-5-iodo-6-phenylpyrimidin-4-yl)oxyl acetate. Into a 100-mL round-bottom flask were placed 4-chloro-5-iodo-6-phenylpyrimidin-2-amine (2.30 g, 6.94 mmol), methyl 2-hydroxyacetate (937.4 mg, 10.41 mmol), K₂CO₃ (1.92 g, 13.87 mmol) and DMF (20 mL). The resulting mixture was stirred for 16 h at 80° C. After cooling, the reaction mixture was quenched with 50 mL of water. The resulting mixture was extracted with 3×50 mL of ethyl acetate. The combined organic layers were washed with 2×50 mL of H2O, 50 mL of brine. The resulting organic layer was dried over anhydrous sodium sulfate and concentrated. The residue was purified on a silica gel column with ethyl acetate/petroleum ether (1/1) to obtain 1.20 g (44.9%) of methyl 2-[(2-amino-5-iodo-6-phenylpyrimidin-4-yl)oxy]acetate as a white solid. ES-MS (m/z): [M+1]+=386.

Step 4. methyl 2-[(2-amino-5-[2-methyl-6-(trifluoromethyl)pyridin-4-yl]-6-phenylpyrimidin-4-yl]oxy)acetate. Into a 40-mL sealed tube under nitrogen atmosphere were placed methyl 2-[(2-amino-5-iodo-6-phenylpyrimidin-4-yl)oxy]acetate (700 mg, 1.82 mmol), [2-methyl-6-(trifluoromethyl)pyridin-4-yl]boronic acid (447 mg, 2.18 mmol), Pd(dppf)Cl₂ (66.5 mg, 0.09 mmol), K₂CO₃ (502.4 mg, 3.63 mmol), 1,4-dioxane (10 mL) and H₂O (1 mL). The resulting mixture was stirred for 16 h at 100° C. After cooling, the reaction mixture was quenched with 50 mL of water. The resulting mixture was extracted with 3×50 mL of ethyl acetate. The combined organic extracts were washed with 2×50 mL of H₂O, then 50 mL of brine. The resulting organic layer was dried over anhydrous sodium sulfate and concentrated. The residue was purified on a silica gel column with ethyl acetate/petroleum ether (1/1) to obtain 300 mg (39.5%) of methyl 2-([2-amino-5-P-methyl-6-(trifluoromethyl)pyridin-4-yl]-6-phenylpyrimidin-4-yl)oxy)acetate as a white solid.

Step 5: Synthesis of 24(2-amino-5-(2-methyl-6-(trifluoromethyl)pyridin-4-yl)-6-phenylpyrimidin-4-yl)oxy)-N-methylacetamide. Into a 8-mL sealed tube were placed methyl 2-([2-amino-5-P-methyl-6-(trifluoromethyl)pyridin-4-yl]-6-phenylpyrimidin-4-yl)oxy)acetate (100.0 mg, 0.24 mmol), MeNH₂/THF (2.0 M, 3 mL). The resulting solution was stirred for 16 h at 80° C. After cooling, the reaction mixture was concentrated. The crude was purified by Flash-Prep-HPLC to obtain 47.3 mg (47.4%) of 24(2-amino-5-(2-methyl-6-(trifluoromethyl)pyridin-4-yl)-6-phenylpyrimidin-4-yl)oxy)-N-methylacetamide as a white solid. ES-MS (m/z): [M+1]⁺=418.1.

### Example 2

### 2-((2-amino-5-(2-methyl-6-(trifluoromethyl)pyridin-4-yl)-6-phenylpyrimidin-4-yl)oxy)-N,N-dimethylacetamide

Step 1: 2-(12-amino-5-12-methyl-6-(trifluoromethyl)pyridin-4-yl1-6-phenylpyrimidin-4-yl)oxy)acetic acid. Into a 8-mL sealed tube were placed methyl 2-([2-amino-5-12-methyl-6-(trifluoromethyl)pyridin-4-yl]-6-phenylpyrimidin-4-yl)oxy)acetate (160 mg, 0.38 mmol), LiOH (18.3 mg, 0.76 mmol), MeOH (2 mL) and H₂O (2 mL). The resulting mixture was stirred for 16 h at room temperature. The resulting mixture was concentrated to remove most organic solvent. The pH value of the residual solution was adjusted to 5 with aqueous HCl (1.0 M). The solid was collected by filtration to obtain 120 mg (77.6%) of 2-((2-amino-5-(2-methyl-6-(trifluoromethyl)pyridin-4-yl)-6-phenylpyrimidin-4-yl)oxy)acetic acid as a white solid.

Step 2: 2-((2-amino-5-(2-methyl-6-(trifluoromethyl)pyridin-4-yl)-6-phenylpyrimidin-4-yl)oxy)-N,N-dimethylacetamide. Into a 8-mL sealed tube were placed 2-((2-amino-5-[2-methyl-6-(trifluoromethyl)pyridin-4-yl]-6-phenylpyrimidin-4-yl)oxy)acetic acid (90 mg, 0.22 mmol), dimethylamine (20.1 mg, 0.45 mmol), HATU (126.9 mg, 0.33 mmol), DIPEA (115.1 mg, 0.89 mmol) and DCM (4 mL). The resulting solution was stirred for 16 h at room temperature. The resulting mixture was concentrated. The residue was purified by Flash-Prep-HPLC to obtain 28.1 mg (29.3%) of 2-((2-amino-5-(2-methyl-6-(trifluoromethyl)pyridin-4-yl)-6-phenylpyrimidin-4-yl)oxy)-N,N-dimethylacetamide as a white solid. ES-MS (m/z): [M+1]+=432.1.

### Example 3

### 2-((2-amino-5-(2-methyl-6-(trifluoromethyl)pyridin-4-yl)-6-phenylpyrimidin-4-yl)oxy)-N,N-diethylacetamide

Following step 2, example 2. From 2-([2-amino-5-[-2-methyl-6-(trifluoromethyl)pyridin-4-yl]-6-phenylpyrimidin-4-yl]oxy)acetic acid, diethylamine, DIPEA and HATU in DCM. ES-MS (m/z): [M+1]⁺=460.2.

### Example 4

### (S)-5-(2-methyl-6-(trifluoromethyl)pyridin-4-yl)-6-phenyl-N4-((6-(((tetrahydrofuran-3-yl)oxy)methyl)pyridin-2-yl)methyl)pyrimidine-2,4-diamine

Step 1: (S)-5-iodo-6-phenyl-N⁴-((6-(((tetrahydrofuran-3-yl)oxy)methyl)pyridin-2-yl)methyl)pyrimidine-2,4-diamine Into a 8 mL sealed tube were placed 1-(6-[[(3S)-oxolan-3-yloxy]methyl]pyridin-2-yl)methanamine (201 mg, 0.97 mmol), 4-chloro-5-iodo-6-phenylpyrimidin-2-amine (300 mg, 0.88 mmol), K₂CO₃ (243 mg, 1.76 mmol) and DMF (5 mL) under nitrogen atmosphere. The resulting mixture was stirred for 16 h at 80° C. After cooling to room temperature, the reaction was quenched with 20 mL of water. The resulting mixture was extracted with 3×20 mL of ethyl acetate. The combined organic layers were washed with 2×20 mL of H₂O, 20 mL of brine, then dried over anhydrous Na₂SO₄ and concentrated. The residue was purified on a silica gel column with ethyl acetate/petroleum ether (1/1) to obtain 210 mg (46.1%) of (S)-5-iodo-6-phenyl-N⁴-((6-(((tetrahydrofuran-3-yl)oxy)methyl)pyridin-2-yl)methyl)pyrimidine-2,4-diamine as a white solid. LC-MS (ES, m/z): [M+1]⁺=504.3.

Step 2: (S)-5-(2-methyl-6-(trifluoromethyl)pyridin-4-yl)-6-phenyl-N4-(((6-(((tetrahydrofuran-3-yl)oxy)methyl)pyridin-2-yl)methyl)pyrimidine-2,4-diamine. Into a 8-mL sealed tube were placed 5-iodo-N4-[(6-[[(3S)-oxolan-3-yloxy]methyl]pyridin-2-yl)methyl]-6-phenylpyrimidine-2,4-diamine (100 mg, 0.20 mmol), [2-methyl-6-(trifluoromethyl)pyridin-4-yl]boronic acid (48.9 mg, 0.24 mmol), Pd(dppf)Cl₂ (7.3 mg, 0.01 mmol), K₂CO₃ (54.9 mg, 0.40 mmol), 1,4-dioxane (3 mL) and H₂O (0.3 mL) under nitrogen atmosphere. After stirring for 16 h at 100° C., the reaction mixture was cooled and concentrated. The crude product was purified by Flash-Prep-HPLC to afford 22.6 mg (21.2%) of (S)-5-(2-methyl-6-(trifluoromethyl)pyridin-4-yl)-6-phenyl-N4-((6-(((tetrahydrofuran-3-yl)oxy)methyl)pyridin-2-yl)methyl)pyrimidine-2,4-di amine as a white solid. ES-MS (m/z): [M+1]⁺=537.2.

### Example 5

### (S)-5-(2-methyl-6-(trifluoromethyl)pyridin-4-yl)-4-phenyl-6-46-(((tetrahydrofuran-3-yl)oxy)methyl)pyridin-2-yl)methoxy)pyrimidin-2-amine

Step 1: 5-iodo-4-[(6-(((3S)-oxolan-3-yloxy)methyl]pyridin-2-yl)methoxyl-6-phenylpyrimidin-2-amine Following step 3, example 1. From (6-(((3S)-oxolan-3-yloxy)methyl)pyridin-2-yl)methanol, 4-chloro-5-iodo-6-phenylpyrimidin-2-amine and K₂CO₃ in DMF.

Step 2: (S)-5-(2-methyl-6-(trifluoromethyl)pyridin-4-yl)-4-phenyl-6-((6-(((tetrahydrofuran-3-yl)oxy)methyl)pyridin-2-yl)methoxy)pyrimidin-2-amine. Following step 4, example 1. From 5-iodo-4-[(6-(((3S)-oxolan-3-yloxy)methyl]pyridin-2-yl)methoxyl-6-phenylpyrimidin-2-amine, 2-methyl-4-(4,4,5,5-tetramethyl-1,3,2-dioxaborolan-2-yl)-6-(trifluoromethyl)pyridine, Pd(dppf)Cl₂, K₂CO₃ in 1,4-dioxane and H₂O. ES-MS (m/z): [M+1]⁺=538.1.

### Example 6

### 4-(3-(4-(2,4-difluorophenyl)piperazin-1-yl)propoxy)-5-(2-methyl-6-(trifluoromethyl)pyridin-4-yl)-6-phenylpyrimidin-2-amine

Step 1: 1-(2,4-difluorophenyl)piperazine. To a stirred mixture of 1-bromo-2,4-difluorobenzene (2.0 g, 10.4 mmol) and piperazine (5.4 g, 62.2 mmol) in toluene (20 mL) were added t-BuONa (1.5 g, 15.5 mmol), Pd₂(dba)₃ (0.2 g, 0.21 mmol) and BINAP (0.4 g, 0.62 mmol) at room temperature under nitrogen atmosphere. The resulting mixture was stirred for 24 h at 110° C. After cooling to room temperature, the reaction mixture was diluted with H₂O (50 mL). The resulting mixture was extracted with ethyl acetate (3×30 mL). The combined organic layers were washed with H₂O (2×30 mL) and brine (30 mL). The resulting organic layer was dried over anhydrous Na₂SO₄, and concentrated. The residue was purified on silica gel column with DCM/MeOH (10/1) to obtain 675 mg (32.9%) of 1-(2,4-difluorophenyl)piperazine as a brown solid. ES-MS (m/z): [M+1]⁺=199.

Step 2: 3-[4-(2,4-difluorophenyl)piperazin-1-yl]propan-1-ol. To a stirred mixture of 1-(2,4-difluorophenyl)piperazine (330 mg, 1.66 mmol) and K₂CO₃ (460.2 mg, 3.33 mmol) in DMF (5 mL) was added 3-bromopropan-1-ol (347.1 mg, 2.50 mmol) at room temperature under nitrogen atmosphere. The resulting mixture was stirred for 16 h at 60° C. After cooling to room temperature, the resulting mixture was diluted with ethyl acetate (30 mL). The resulting mixture was washed with 2×10 mL of water and 1×10 mL of brine. The organic phase was dried over Na₂SO₄, filtered and concentrated. The residue was purified on silica gel column with DCM/MeOH (10/1) to obtain 265 mg (62.1%) of 3-[4-(2,4-difluorophenyl)piperazin-1-yl]propan-1-ol as a light brown oil. ES-MS (m/z): [M+1]⁺=257.

Step 3: 4-[3-(4-(2,4-difluorophenyl)piperazin-1-yl]propoxyl-5-iodo-6-phenylpyrimidin-2-amine To a stirred solution of NaH (42.5 mg, 1.06 mmol, 60%) in DMF (3 mL) was added 3-[4-(2,4-difluorophenyl)piperazin-1-yl]propan-1-ol (181.7 mg, 0.71 mmol) dropwise at 0° C. under nitrogen atmosphere. After stirring the mixture for 10 min at 0° C., 4-chloro-5-iodo-6-phenylpyrimidin-2-amine (235 mg, 0.71 mmol) was added. The resulting mixture was stirred for additional 4 h at 40° C. After cooling to room temperature, the reaction mixture was quenched by the addition of sat. aqueous NH₄Cl (0.5 mL). The mixture was purified by Prep-HPLC to obtain 135 mg (34.5%) of 4-(3-(4-(2,4-difluorophenyl)piperazin-1-yl)propoxyl-5-iodo-6-phenylpyrimidin-2-amine as a brown oil. ES-MS (m/z): [M+1]⁺=552.0.

Step 4: 4-(3-(4-(2,4-difluorophenyl)piperazin-1-yl)propoxy)-5-(2-methyl-6-(trifluoromethyl)pyridin-4-yl)-6-phenylpyrimidin-2-amine Following step 4, example 1. From 4-[3-(4-(2,4-difluorophenyl)piperazin-1-yl]propoxyl-5-iodo-6-phenylpyrimidin-2-amine, 2-methyl-4-(4,4,5,5-tetramethyl-1,3,2-dioxaborolan-2-yl)-6-(trifluoromethyl)pyridine, Pd(dppf)Cl₂, K₂CO₃ in 1,4-dioxane and H₂O. ES-MS (m/z): [M+1]⁺=585.2.

### Example 7

### (S)-5-(2-methyl-6-(trifluoromethyl)pyridin-4-yl)-4-(5-methylfuran-2-yl)-6-((6-(((tetrahydrofuran-3-yl)oxy)methyl)pyridin-2-yl)methoxy)pyrimidin-2-amine

Step 1: 4-chloro-6-(5-methylfuran-2-yl)pyrimidin-2-amine. To a stirred mixture of 4,6-dichloropyrimidin-2-amine (1.16 g, 7.09 mmol) and (5-methylfuran-2-yl)boronic acid (446.5 mg, 3.55 mmol) in 1,4-dioxane (1 mL) and H₂O (0.25 mL) were added Pd(PPh₃)₄ (819.5 mg, 0.71 mmol) and K₂CO₃ (2940.4 mg, 21.28 mmol) at room temperature under nitrogen atmosphere. The resulting mixture was stirred for 3 h at 70° C. After cooling to room temperature, the reaction mixture was diluted with ethyl acetate (20 mL). The mixture was filtered and the solid cake was washed with ethyl acetate (3×10 mL). The combined filtrate was concentrated. The residue was purified on silica gel column with DCM/MeOH (10/1) to obtain 458 mg (30.8%) of 4-chloro-6-(5-methylfuran-2-yl)pyrimidin-2-amine as a light brown solid.

Step 2: 4-chloro-5-iodo-6-(5-methylfuran-2-yl)pyrimidin-2-amine To a stirred solution of 4-chloro-6-(5-methylfuran-2-yl)pyrimidin-2-amine (471 mg, 2.25 mmol) in DMF (5 mL) was added NIS (758.2 mg, 3.37 mmol) in portions at room temperature under nitrogen atmosphere. The resulting mixture was stirred for 16 h at 80° C. After cooling to room temperature, the reaction mixture was diluted with ethyl acetate (20 mL). The mixture was washed with 3×10 mL of water, 10 mL of brine. The organic layer was dried over Na₂SO₄, filtered and concentrated. The residue was purified on silica gel column with DCM/MeOH (10/1) to obtain 393 mg (52.1%) of 4-chloro-5-iodo-6-(5-methylfuran-2-yl)pyrimidin-2-amine as a yellow solid.

Step 3: 5-iodo-4-(5-methylfuran-2-yl)-6-((6-[[(3S)-oxolan-3-yloxy]methyl]pyridin-2-yl)methoxyl-pyrimidin-2-amine. Following step 3, example 6. From (6-[[(3S)-oxolan-3-yloxy]methyl]pyridin-2-yl)methanol, 4-chloro-5-iodo-6-(5-methylfuran-2-yl)pyrimidin-2-amine, NaH in DMF.

Step 4: (S)-5-(2-methyl-6-(trifluoromethyl)pyridin-4-yl)-4-(5-methylfuran-2-yl)-6-((6-(((tetrahydrofuran-3-yl)oxy)methyl)pyridin-2-yl)methoxy)pyrimidin-2-amine. Following step 4, example 1. From 5-iodo-4-(5-methylfuran-2-yl)-6-[(6-[[(3S)-oxolan-3-yloxy]methyl]pyridin-2-yl)methoxy]pyrimidin-2-amine, 2-methyl-4-(4,4,5,5-tetramethyl-1,3,2-dioxaborolan-2-yl)-6-(trifluoromethyl)pyridine, Pd(dppf)Cl₂, K₂CO₃ in 1,4-dioxane and H₂O. ES-MS (m/z): [M+1]⁺=542.2.

### Example 8

### 5-(2-methyl-6-(trifluoromethyl)pyridin-4-yl)-4-phenethoxy-6-phenylpyrimidin-2-amine

Step 1: 4-chloro-5-[2-methyl-6-(trifluoromethyl)pyridin-4-yl]-6-phenylpyrimidin-2-amine. To a stirred mixture of 4-chloro-5-iodo-6-phenylpyrimidin-2-amine (1.0 g, 3.02 mmol) and P-methyl-6-(trifluoromethyl)pyridin-4-yl)boronic acid (0.6 g, 3.02 mmol) in 1,4-dioxane (20 mL) and H₂O (2 mL) were added Pd(dppf)Cl₂ (0.1 g, 0.15 mmol) and K₂CO₃ (0.8 g, 6.03 mmol) at room temperature under nitrogen atmosphere. The reaction mixture was stirred for 16 h at 70° C. After cooling to room temperature, the mixture was diluted with H₂O (30 mL), extracted with ethyl acetate (3×30 mL). The combined organic layers were washed with 30 mL of water and 30 mL of brine, dried over anhydrous Na₂SO₄ and concentrated. The residue was purified on silica gel column with PE/EA (3/1) to afford 4-chloro-5-[2-methyl-6-(trifluoromethyl)pyridin-4-yl]-6-phenylpyrimidin-2-amine (793 mg, 72.1%) as a light yellow solid. ES-MS (m/z): [M+1]⁺=365.

Step 2: 5-(2-methyl-6-(trifluoromethyl)pyridin-4-yl)-4-phenethoxy-6-phenylpyrimidin-2-amine. Into a 4 mL sealed tube were placed 2-phenylethan-1-ol (32.2 mg, 0.26 mmol), THF (2 mL), NaH (7.9 mg, 0.33 mmol, 60%) and 4-chloro-5-[2-methyl-6-(trifluoromethyl)pyridin-4-yl]-6-phenylpyrimidin-2-amine (80.0 mg, 0.22 mmol) under nitrogen atmosphere. The resulting mixture was stirred for 4 h at room temperature. After cooling to room temperature, the reaction mixture was quenched with 1 mL of aq. NH₄Cl. The resulting mixture was concentrated and purified by Prep-HPLC to get 35 mg (34.3%) of 5-(2-methyl-6-(trifluoromethyl)pyridin-4-yl)-4-phenethoxy-6-phenylpyrimidin-2-amine. ES-MS (m/z): [M+1]⁺==451.2.

### Example 9

### N-(4-(2-((2-amino-5-(2-methyl-6-(trifluoromethyl)pyridin-4-yl)-6-phenylpyrimidin-4-yl)loxy)ethyl)phenyl)acetamide

Step 1: 2-(4-aminophenyl)ethan-1-ol. To a stirred solution of 2-(4-nitrophenyl)ethan-1-ol (5.0 g, 29.91 mmol) in MeOH (100 mL) was added 10% Pd/C (0.3 g) at room temperature under nitrogen atmosphere. The flask was evacuated and flushed three times with nitrogen, followed by flushing with hydrogen. The mixture was stirred for 4 h at room temperature under hydrogen atmosphere (balloon). The mixture was filtered and the filtrate was concentrated to obtain 2-(4-aminophenyl)ethan-1-ol (3.8 g, 92.6%) as a yellow solid. ES-MS (m/z): [M+1]⁺=138.

Step 2: N-[4-(2-hydroxyethyl)phenyl]acetamide. To a stirred solution of 2-(4-aminophenyl)ethan-1-ol (500 mg, 3.64 mmol) and DIPEA (942.1 mg, 7.29 mmol) in DCM (10 mL) was added Ac₂O (372.1 mg, 3.64 mmol) dropwise at room temperature. The resulting mixture was stirred for 4 h at room temperature, then was concentrated and the residue was purified on silica gel column with EA/PE (1/1) to afford N-[4-(2-hydroxyethyl)phenyl]acetamide (256 mg, 39.2%) as an off-white solid. ES-MS (m/z): [M+1]⁺=180.

Step 3: N-(4-(2-((2-amino-5-(2-methyl-6-(trifluoromethyl)pyridin-4-yl)-6-phenylpyrimidin-4-yl)oxy)ethyl)phenyl)acetamide. To a mixture of N-[4-(2-hydroxyethyl)phenyl]acetamide (35.0 mg, 0.20 mmol) and 4-chloro-5-[2-methyl-6-(trifluoromethyl)pyridin-4-yl]-6-phenylpyrimidin-2-amine(71.2 mg, 0.20 mmol) in DMF (2 mL) was added Cs₂CO₃ (127.3 mg, 0.39 mmol) at room temperature under nitrogen atmosphere. The resulting mixture was stirred for 16 h at 80° C. After cooling to room temperature, the mixture was filtered and the solid cake was washed with DMF (2×0.5 mL). The combined filtrate was purified by Prep-HPLC to obtain N-(4-(2-((2-amino-5-(2-methyl-6-(trifluoromethyl)pyridin-4-yl)-6-phenylpyrimidin-4-yl)oxy)ethyl)phenyl)acetamide (21 mg, 21.2%) as a white solid. ES-MS (m/z): [M+1]⁺=508.2.

### Example 10

### 2-((2-amino-5-(2-methyl-6-(trifluoromethyl)pyridin-4-yl)-6-phenylpyrimidin-4-yl)oxy)-1-morpholinoethan-1-one

Following step 2, example 2. From 2-([2-amino-5-[2-methyl-6-(trifluoromethyl)pyridin-4-yl]-6-phenylpyrimidin-4-yl]oxy)acetic acid, morpholine, Et₃N and HATU in DCM. ES-MS (m/z): [M+1]⁺=474.2.

### Example 11

### 2-((2-amino-6-(furan-2-yl)-5-(2-methyl-6-(trifluoromethyl)pyridin-4-yl)pyrimidin-4-yl)oxy)-N,N-diethylacetamide

Step 1: 4-chloro-6-(furan-2-yl)pyrimidin-2-amine. To a stirred mixture of 4,6-dichloropyrimidin-2-amine (2.0 g, 12.20 mmol) and 2-furanylboronic acid (682.3 mg, 6.10 mmol) in 1,4-dioxane (20 mL) and H₂O (2 mL) were added K₃PO₄ (7.8 g, 36.59 mmol) and Pd(PPh₃)₄ (704.7 mg, 0.61 mmol) at room temperature under nitrogen atmosphere. The resulting mixture was stirred for 16 h at 70° C. After cooling to room temperature, the mixture was diluted with ethyl acetate (50 mL), and washed with 3×30 mL of water, dried over anhydrous Na₂SO₄, and filtered. The filtrate was concentrated. The residue was purified on silica gel column with PE/EA (3/1) to afford 4-chloro-6-(furan-2-yl)pyrimidin-2-amine (814 mg, 34.1%) as a yellow solid. ES-MS (ink): [M+1]⁺=196.

Step 2: 4-chloro-6-(furan-2-yl)-5-iodopyrimidin-2-amine. To a stirred mixture of 4-chloro-6-(furan-2-yl)pyrimidin-2-amine (350.0 mg, 1.79 mmol) in AcOH (214.9 mg, 3.58 mmol) and DMF (5 mL) was added NIS (805.1 mg, 3.58 mmol) in portions at room temperature. The resulting mixture was stirred for 3 days at room temperature. The reaction mixture was poured into 50 mL of water. The mixture was filtered, and the solid cake was washed with water (5×50 mL) to obtain 4-chloro-6-(furan-2-yl)-5-iodopyrimidin-2-amine (136 mg, 23.6%) as a yellow solid. ES-MS (m/z): [M+1]⁺=322.

Step 3: 4-chloro-6-(furan-2-yl)-5-[2-methyl-6-(trifluoromethyl)pyridin-4-yl]pyrimidin-2-amine. Following step 1, example 8. From 4-chloro-6-(furan-2-yl)-5-iodopyrimidin-2-amine, 2-methyl-4-(4,4,5,5-tetramethyl-1,3,2-dioxaborolan-2-yl)-6-(trifluoromethyl)pyridine, Pd(dppf)Cl₂ and K₂CO₃ in 1,4-dioxane and H₂O. ES-MS (m/z): [M+1]⁺=355.

Step 4: 2-(benzyloxy)-N,N-diethylacetamide. To a stirred mixture of 2-(benzyloxy)acetic acid (2.0 g, 12.04 mmol) and diethylamine (0.9 g, 12.04 mmol) in DCM (20 mL) were added HATU (6.9 g, 18.05 mmol) and DIPEA (4.7 g, 36.11 mmol) at room temperature. The resulting mixture was stirred for 16 h at room temperature. The reaction mixture was diluted with ethyl acetate (100 mL), washed with 3×30 mL of water and 30 mL of brine. The organic layer was dried over anhydrous Na₂SO₄, filtered and concentrated. The residue was purified on silica gel column with DCM/MeOH (20/1) to afford 2-(benzyloxy)-N,N-diethylacetamide (1.8 g, 67.6%) as a yellow solid. ES-MS (m/z): [M+1]⁺=222.

Step 5: N,N-diethyl-2-hydroxyacetamide. To a stirred solution of 2-(benzyloxy)-N,N-diethylacetamide (1.8 g, 8.13 mmol) in ethyl acetate (18 mL) was added 10% Pd/C (86.6 mg) at room temperature under nitrogen atmosphere. The flask was evacuated and flushed three times with nitrogen, followed by flushing with hydrogen. The mixture was stirred 16 h at room temperature under an atmosphere of hydrogen (balloon). The resulting mixture was filtered and the filter cake was washed with ethyl acetate (3×10 mL). The filtrate was concentrated to obtain N,N-diethyl-2-hydroxyacetamide (800 mg, 75.0%) as a colorless oil. ES-MS (m/z): [M+1]⁺=132.

Step 6: 2-((2-amino-6-(furan-2-yl)-5-(2-methyl-6-(trifluoromethyl)pyridin-4-yl)pyrimidin-4-yl)oxy)-N,N-diethylacetamide. Following step 2, example 8. From 4-chloro-6-(furan-2-yl)-5-[2-methyl-6-(trifluoromethyl)pyridin-4-yl]pyrimidin-2-amine, N,N-diethyl-2-hydroxyacetamide and 60% NaH in THF. ES-MS (m/z): [M+1]⁺=450.3.

### Example 12

### 2-((2-amino-5-(2-methyl-6-(trifluoromethyl)pyridin-4-yl)-6-(5-methylfuran-2-yl)pyrimidin-4-yl)oxy)-N,N-diethylacetamide

Step 1: 4-chloro-5-[2-methyl-6-(trifluoromethyl)pyridin-4-yl]-6-(5-methylfuran-2-yl)pyrimidin-2-amine. Following step 1, example 8. From 4-chloro-5-iodo-6-(5-methylfuran-2-yl)pyrimidin-2-amine, 2-methyl-4-(4,4,5,5-tetramethyl-1,3,2-dioxaborolan-2-yl)-6-(trifluoromethyl)pyridine, Pd(dppf)Cl₂ and K₂CO₃ in 1,4-dioxane and H₂O. ES-MS (m/z): [M+1]⁺=369.

Step 2: 2-((2-amino-5-(2-methyl-6-(trifluoromethyl)pyridin-4-yl)-6-(5-methylfuran-2-yl)pyrimidin-4-yl)oxy)-N,N-diethylacetamide. Following step 2, example 8. From 4-chloro-5-[2-methyl-6-(trifluoromethyl)pyridin-4-yl]-6-(5-methylfuran-2-yl)pyrimidin-2-amine, N,N-diethyl-2-hydroxyacetamide and 60% NaH in THF. ES-MS (m/z): [M+1]⁺=464.1.

### Examples 13 and 14

### (R)-2-((2-amino-5-(2-methyl-6-(trifluoromethyl)pyridin-4-yl)-6-phenylpyrimidin-4-yl)oxy)-N,N-diethylpropanamide and

### (S)-2-((2-amino-5-(2-methyl-6-(trifluoromethyl)pyridin-4-yl)-6-phenylpyrimidin-4-yl)oxy)-N,N-diethylpropanamide

Step 1: methyl 2-([2-amino-5-[2-methyl-6-(trifluoromethyl)pyridin-4-yl]-6-phenylpyrimidin-4-yl]oxy)propanoate. Following step 3, example 9. From 4-chloro-5-[2-methyl-6-(trifluoromethyl)pyridin-4-yl]-6-phenylpyrimidin-2-amine, methyl 2-hydroxypropanoate, and K₂CO₃ in DMF. ES-MS (m/z): [M+1]⁺=433.

Step 2. 2-([2-amino-5-[2-methyl-6-(trifluoromethyl)pyridin-4-yl]-6-phenylpyrimidin-4-yl]oxy)propanoic acid. To a stirred solution of methyl 2-([2-amino-5-[2-methyl-6-(trifluoromethyl)pyridin-4-yl]-6-phenylpyrimidin-4-yl]oxy)propanoate (132.0 mg, 0.31 mmol) in MeOH (1.0 mL) and H₂O (0.1 mL) was added LiOH (14.6 mg, 0.61 mmol) at room temperature. The resulting mixture was stirred for 3 h at room temperature. The reaction mixture was concentrated and the residue was dissolved in H₂O (2 mL). The aqueous solution was acidified to pH 3 with 1 M aqueous HCl. The resulting mixture was filtered and the filter cake was washed with H₂O (3×3 mL) to obtain 2-([2-amino-5-[2-methyl-6-(trifluoromethyl)pyridin-4-yl]-6-phenylpyrimidin-4-yl]oxy)propanoic acid (109 mg, 85.3%) as a white solid. ES-MS (m/z): [M+1]⁺=419.

Step 3: 2-([2-amino-5-[2-methyl-6-(trifluoromethyl)pyridin-4-yl]-6-phenylpyrimidin-4-yl]oxy)-N,N-diethylpropanamide. To a stirred mixture of 2-([2-amino-5-[2-methyl-6-(trifluoromethyl)pyridin-4-yl]-6-phenylpyrimidin-4-yl]oxy)propanoic acid (108 mg, 0.26 mmol) and diethylamine (28.3 mg, 0.39 mmol) in DCM (2 mL) were added HATU (147.2 mg, 0.39 mmol) and triethylamine (78.4 mg, 0.77 mmol) at room temperature. The resulting mixture was stirred for 2 h at room temperature. The reaction mixture was diluted with ethyl acetate (30 mL), and washed with 3×10 mL of water, 10 mL of brine. The organic layer was dried over anhydrous Na₂SO₄, filtered, and concentrated. The residue was purified on silica gel with DCM/MeOH (20/1) to afford 2-([2-amino-5-[2-methyl-6-(trifluoromethyl)pyridin-4-yl]-6-phenylpyrimidin-4-yl]oxy)-N,N-diethylpropanamide (82 mg, 67.1%) as an off-white solid. ES-MS (m/z): [M+1]⁺=474.

Step 4: (R)-2-((2-amino-5-(2-methyl-6-(trifluoromethyl)pyridin-4-yl)-6-phenylpyrimidin-4-yl)oxy)-N,N-diethylpropanamide and (S)-2-((2-amino-5-(2-methyl-6-(trifluoromethyl)pyridin-4-yl)-6-phenylpyrimidin-4-yl)oxy)-N,N-diethylpropanamide. (2-([2-amino-5-[2-methyl-6-(trifluoromethyl)pyridin-4-yl]-6-phenylpyrimidin-4-yl]oxy)-N,N-diethylpropanamide (82 mg) was resolved by Prep-Chiral-HPLC with the following conditions: eluting with n-hexane/ethanol (9/1) to afford (R)-2-((2-amino-5-(2-methyl-6-(trifluoromethyl)pyridin-4-yl)-6-phenylpyrimidin-4-yl)oxy)-N,N-diethylpropanamide (36 mg, 43.9%) as an off-white solid, ES-MS (m/z): [M+1]⁺=474.0, and (S)-2-((2-amino-5-(2-methyl-6-(trifluoromethyl)pyridin-4-yl)-6-phenylpyrimidin-4-yl)oxy)-N,N-diethylpropanamide (41 mg, 50.0%) as an off-white solid, ES-MS (m/z): [M+1]⁺=474.0.

### Example 15

### 2-((2-amino-5-(2-(difluoromethyl)-6-methylpyridin-4-yl)-6-phenylpyrimidin-4-yl)oxy)-N,N-diethylacetamide

Step 1: 2-[(2-amino-5-iodo-6-phenylpyrimidin-4-yl)oxy]-N,N-diethylacetamide. Following step 3, example I1. From 4-chloro-5-iodo-6-phenylpyrimidin-2-amine, N,N-diethyl-2-hydroxyacetamide, 60% NaH in THF. ES-MS (m/z): [M+1]⁺=427.

Step 2: 2-(difluoromethyl)-6-methylpyridine. To a stirred solution of 6-methylpyridine-2-carbaldehyde (1.0 g, 8.25 mmol) in DCM (10 mL) was added DAST (2.7 g, 16.51 mmol) at 0° C. under nitrogen atmosphere. The resulting mixture was stirred for 3 h at room temperature. The reaction mixture was quenched with saturated aqueous NaHCO₃ at room temperature. The resulting mixture was diluted with DCM (30 mL), washed with 3×20 mL of water, 20 mL of brine. The organic layer was dried over anhydrous Na₂SO₄, filtered, and concentrated. The residue was purified on silica gel column with PE/EA (5/1) to afford 2-(difluoromethyl)-6-methylpyridine (231 mg, 19.6%) as colorless oil. ES-MS (m/z): [M+1]⁺=144.

Step 3: 2-(difluoromethyl)-6-methyl-4-(4,4,5,5-tetramethyl-1,3,2-dioxaborolan-2-yl)pyridine. A mixture of 4,4,5,5-tetramethyl-2-(4,4,5,5-tetramethyl-1,3,2-dioxaborolan-2-yl)-1,3,2-dioxaborolane (601.4 mg, 2.37 mmol), 4,4-di-tert-butyl-2,2-bipyridine (2.9 mg) and methoxy(cyclooctadiene)iridium(I) dimer (6.6 mg) in 1,4-dioxane (3.0 mL) was stirred at 50° C. for 10 min under nitrogen atmosphere, 2-(difluoromethyl)-6-methylpyridine (311.0 mg, 2.17 mmol) then was added. The reaction mixture was stirred for 4 h at 50° C. After cooling to room temperature, the reaction mixture was concentrated. The residue was purified on silica gel column with PE/EA (2/1) to afford 2-(difluoromethyl)-6-methyl-4-(4,4,5,5-tetramethyl-1,3,2-dioxaborolan-2-yl)pyridine (246 mg, 42.1%) as an off-white solid. ES-MS (m/z): [M+1]⁺=269.8.

Step 4: 2-((2-amino-5-(2-(difluoromethyl)-6-methylpyridin-4-yl)-6-phenylpyrimidin-4-yl)oxy)-N,N-diethylacetamide. Following step 4, example 1. From 2-((2-amino-5-iodo-6-phenylpyrimidin-4-yl)oxyl-N,N-diethylacetamide, 2-(difluoromethyl)-6-methyl-4-(4,4,5,5-tetramethyl-1,3,2-dioxaborolan-2-yl)pyridine, Pd(dppf)Cl₂ and K₂CO₃ in 1,4-dioxane and H₂O. ES-MS (m/z): [M+1]⁺=442.1.

### Example 16

2-((2-amino-5-(2-(1,1-difluoroethyl)-6-methylpyridin-4-yl)-6-phenylpyrimidin-4-yl)oxy)-N,N-diethylacetamide Step 1: N-methoxy-N,6-dimethylpyridine-2-carboxamide To a stirred mixture of 6-methylpyridine-2-carboxylic acid (5.0 g, 36.46 mmol) and methoxy(methyl)amine hydrochloride (4.3 g, 43.75 mmol) in DCM (50 mL) were added DIPEA (14.1 g, 109.38 mmol) and HATU (20.8 g, 54.69 mmol) at room temperature. The resulting mixture was stirred for 3 h at room temperature. The reaction mixture was diluted with DCM (100 mL), washed with saturated NaHCO₃ (3×50 mL) and brine (50 mL). The organic layer was dried over anhydrous Na₂SO₄, filtered and concentrated. The residue was purified on silica gel column with PE/EA (0-50%) to afford N-methoxy-N,6-dimethylpyridine-2-carboxamide (3.6 g, 54.8%) as a yellow solid. ES-MS (m/z): [M+1]⁺=181.2.

Step 2: 1-(6-methylpyridin-2-yl) ethan-1-one. To a stirred solution of N-methoxy-N,6-dimethylpyridine-2-carboxamide (1.8 g, 9.99 mmol) in THF (20 mL) was added MeMgBr (6.7 mL, 3M in THF, 20.1 mmol) dropwise at −30° C. under nitrogen atmosphere. The mixture was allowed to warm to room temperature slowly, then stirred for 2 h at room temperature. The reaction was quenched with 2 mL of aqueous NH₄Cl at room temperature. The resulting mixture was extracted with ethyl acetate (3×100 mL). The combined organic layers were washed with brine (100 mL), dried over anhydrous Na₂SO₄. After filtration, the filtrate was concentrated. The residue was purified on silica gel column with PE/EA (0-30%) to afford 1-(6-methylpyridin-2-yl)ethan-1-one (1.4 g, 100%) as a yellow oil. ES-MS (m/z): [M+1]⁺=136.0.

Step 3: 2-(1,1-difluoroethyl)-6-methylpyridine. To a stirred solution of 1-(6-methylpyridin-2-yl)ethan-1-one (1.40 g, 10.36 mmol) in DCM (14 mL) was added DAST (3.34 g, 20.72 mmol) dropwise at 5° C. The reaction mixture was warmed to room temperature and stirred at this temperature for 4 days. The reaction mixture was quenched by the addition of sat. NaHCO₃ (20 mL) at 5° C. The resulting mixture was extracted with CH₂Cl₂ (3×20 mL). The combined organic extracts were washed with brine (30 mL), dried over anhydrous Na₂SO₄, filtered and concentrated. The residue was purified on silica gel column with PE/EtOAc (0-10%) to afford 2-(1,1-difluoroethyl)-6-methylpyridine (448 mg, 27.5%) as a colorless oil.

Step 4: 2-(1,1-difluoroethyl)-6-methyl-4-(4,4,5,5-tetramethyl-1,3,2-dioxaborolan-2-yl)pyridine. Following step 3, example 15. From 4,4,5,5-tetramethyl-2-(4,4,5,5-tetramethyl-1,3,2-dioxaborolan-2-yl)-1,3,2-dioxaborolane, 4,4-di-tert-butyl-2,2-bipyridine, bis(1,5-cyclooctadiene)di-methoxydiiridium(I) and 2-(1,1-difluoroethyl)-6-methylpyridine in 1,4-dioxane.

Step 5: 2-((2-amino-5-(2-(1,1-difluoroethyl)-6-methylpyridin-4-yl)-6-phenylpyrimidin-4-yl)oxy)-N,N-diethylacetamide. Following step 4, example 1. From 2-(1,1-difluoroethyl)-6-methyl-4-(4,4,5,5-tetramethyl-1,3,2-dioxaborolan-2-yl)pyridine, 2-[(2-amino-5-iodo-6-phenylpyrimidin-4-yl)oxy]-N,N-diethylacetamide, K₂CO₃ and Pd(dppf)Cl₂ in 1,4-dioxane and H₂O. ES-MS (m/z): [M+1]⁺=456.4.

### Example 17

### 2-((2-amino-5-(2-(1,1-difluoropropyl)-6-methylpyridin-4-yl)-6-phenylpyrimidin-4-yl)oxy)-N,N-diethylacetamide

Step 1: 1-(6-methylpyridin-2-yl)propan-1-one. Following step 2, example 16. From N-methoxy-N,6-dimethylpyridine-2-carboxamide and EtMgBr in THF. ES-MS (m/z): [M+1]⁺=150.2.

Step 2: 2-(1,1-difluoropropyl)-6-methylpyridine. Following step 3, example 16. From 1-(6-methylpyridin-2-yl)propan-1-one and DAST in DCM. ES-MS (m/z): [M+1]⁺=172.2.

Step 3: 2-(1,1-difluoropropyl)-6-methyl-4-(4,4,5,5-tetramethyl-1,3,2-dioxaborolan-2-yl)pyridine. Following step 3, example 15. From 2-(1,1-difluoropropyl)-6-methylpyridine, 4,4,5,5-tetramethyl-2-(4,4,5,5-tetramethyl-1,3,2-dioxaborolan-2-yl)-1,3,2-dioxaborolane, 4,4-di-tert-butyl-2,2-bipyridine and methoxy(cyclooctadiene)iridium(I) dimer in 1,4-dioxane. ES-MS (m/z): [M+1]⁺=298.

Step 4: 2-((2-amino-5-(2-(1,1-difluoropropyl)-6-methylpyridin-4-yl)-6-phenylpyrimidin-4-yl)oxy)-N,N-diethylacetamide. Following step 4, example 1. From 2-((2-amino-5-iodo-6-phenylpyrimidin-4-yl)oxyl-N,N-diethylacetamide, 2-(1,1-difluoropropyl)-6-methyl-4-(4,4,5,5-tetramethyl-1,3,2-dioxaborolan-2-yl)pyridine, Pd(dppf)Cl₂ and K₂CO₃ in 1,4-dioxane and H₂O. ES-MS (m/z): [M+1]⁺=470.0.

### Example 18

### 3-(2-amino-5-(2-methyl-6-(trifluoromethyl)pyridin-4-yl)-6-phenylpyrimidin-4-yl)-N,N-diethylpropanamide

Step 1: ethyl 3-[2-amino-5-[2-methyl-6-(trifluoromethyl)pyridin-4-yl]-6-phenylpyrimidin-4-yl]prop-2-enoate. Into a 20 mL sealed tube were placed 4-chloro-5-[2-methyl-6-(trifluoromethyl)pyridin-4-yl]-6-phenylpyrimidin-2-amine (100.0 mg, 0.27 mmol), 3-methoxy-3-oxoprop-1-en-1-yl)boronic acid (46.3 mg, 0.36 mmol), Pd(dppf)Cl₂ (10.0 mg, 0.01 mmol), K₂CO₃ (75.8 mg, 0.55 mmol), 1,4-dioxane (5 mL) and H₂O (0.5 mL) under nitrogen atmosphere. The resulting mixture was stirred for 16 h at 100° C. The reaction mixture was cooled to room temperature and diluted with 10 mL of water. The resulting mixture was extracted with 3×20 mL of ethyl acetate. The organic layers were combined, and dried over anhydrous Na₂SO₄, filtrated and concentrated. The residue was purified on silica gel column with ethyl acetate/petroleum ether (1/1) to obtain 60 mg (46.0%) of ethyl 3-[2-amino-5-[2-methyl-6-(trifluoromethyl)pyridin-4-yl]-6-phenylpyrimidin-4-yl]prop-2-enoate as a light yellow solid. ES-MS (m/z): [M+1]⁺=429.2.

Step 2: ethyl 3-[2-amino-5-[2-methyl-6-(trifluoromethyl)pyridin-4-yl]-6-phenylpyrimidin-4-yl]propanoate. Into a 50 mL round-bottom flask were placed ethyl 3-[2-amino-5-[2-methyl-6-(trifluoromethyl)pyridin-4-yl]-6-phenylpyrimidin-4-yl]prop-2-enoate (60.0 mg, 0.14 mmol), EtOH (10 mL), and 10% Pd/C (60 mg). The resulting mixture was stirred for 16 h at room temperature under hydrogen atmosphere (2 atm). The mixture was filtered and concentrated to obtain 55 mg (91.2%) of ethyl 3-[2-amino-5-[2-methyl-6-(trifluoromethyl)pyridin-4-yl]-6-phenylpyrimidin-4-yl]propanoate as light yellow oil. ES-MS (m/z): [M+1]⁺=431.3.

Step 3: 3-[2-amino-5-[2-methyl-6-(trifluoromethyl)pyridin-4-yl]-6-phenylpyrimidin-4-yl]propanoic acid. Into a 40 mL vail were added ethyl 3-[2-amino-5-[2-methyl-6-(trifluoromethyl)pyridin-4-yl]-6-phenylpyrimidin-4-yl]propanoate (45.0 mg, 0.10 mmol), LiOH (5.0 mg, 0.21 mmol), H₂O (1 mL) and MeOH (1 mL). The resulting mixture was stirred for 16 h at room temperature. The organic solvent was removed under vacuum. The pH value of the resulting solution was adjusted to 5 with aqueous HCl (1 N). The resulting solution was extracted with 3×5 mL of DCM. The organic layers were combined, dried over anhydrous Na₂SO₄, filtrated and concentrated to obtain 28 mg (59.2%) of 3-[2-amino-5-[2-methyl-6-(trifluoromethyl)pyridin-4-yl]-6-phenylpyrimidin-4-yl]propanoic acid as a light yellow solid. ES-MS (m/z): [M+1]⁺=403.3.

Step 4: 3-(2-amino-5-(2-methyl-6-(trifluoromethyl)pyridin-4-yl)-6-phenylpyrimidin-4-yl)-N,N-diethylpropanamide. Into a 40 mL vial were placed 3-[2-amino-5-[2-methyl-6-(trifluoromethyl)pyridin-4-yl]-6-phenylpyrimidin-4-yl]propanoic acid (28.0 mg, 0.07 mmol), diethylamine (6.3 mg, 0.14 mmol), Et₃N (21.1 mg, 0.21 mmol), HATU (39.7 mg, 0.10 mmol) and DCM (2 mL). The mixture was stirred for 4 h at room temperature. The resulting mixture was concentrated and purified by Prep-HPLC to get 5.5 mg (16.6%) of 3-(2-amino-5-(2-methyl-6-(trifluoromethyl)pyridin-4-yl)-6-phenylpyrimidin-4-yl)-N,N-diethylpropanamide as a white solid. ES-MS (m/z): [M+1]⁺=458.2.

### Example 19

### (2-amino-5-(2-methyl-6-(trifluoromethyl)pyridin-4-yl)-6-phenylpyrimidin-4-yl)methanol

Step 1: methyl 2-amino-5-[2-methyl-6-(trifluoromethyl)pyridin-4-yl]-6-phenylpyrimidine-4-carboxylate. To a stirred mixture of 4-chloro-5-[2-methyl-6-(trifluoromethyl)pyridin-4-yl]-6-phenylpyrimidin-2-amine (1.0 g, 2.74 mmol) and Pd(dppf)Cl₂ (0.2 g, 0.27 mmol) in MeOH (20 mL) was added TEA (0.6 g, 5.48 mmol) at room temperature. The resulting mixture was stirred for 3 h at 80° C. under 0.1 MPa CO atmosphere. The mixture was cooled to room temperature and concentrated. The residue was purified on silica gel column with PE/EA (2/1) to afford methyl 2-amino-5-[2-methyl-6-(trifluoromethyl)pyridin-4-yl]-6-phenylpyrimidine-4-carboxylate (712 mg, 66.9%) as an off-white solid. ES-MS (m/z): [M+1]⁺=389.1.

Step 2: (2-amino-5-(2-methyl-6-(trifluoromethyl)pyridin-4-yl)-6-phenylpyrimidin-4-yl)methanol. To a stirred solution of methyl 2-amino-5-[2-methyl-6-(trifluoromethyl)pyridin-4-yl]-6-phenylpyrimidine-4-carboxylate (104.0 mg, 0.27 mmol) in THF (1 mL) was added LiBH₄ (11.7 mg, 0.54 mmol) at room temperature. After stirring for 16 h, the reaction mixture was quenched with saturated aqueous NH₄Cl (1 mL) at room temperature. The resulting mixture was filtered and concentrated. The residue was purified by Prep-HPLC to obtain (2-amino-5-(2-methyl-6-(trifluoromethyl)pyridin-4-yl)-6-phenylpyrimidin-4-yl)methanol (14.1 mg, 14.6%) as an off-white solid. ES-MS (m/z): [M+1]⁺=361.0.

### Example 20

### 1-(4-(4-(2-((2-amino-5-(2-methyl-6-(trifluoromethyl)pyridin-4-yl)-6-phenylpyrimidin-4-yl)oxy)ethyl)phenyl)piperazin-1-yl)ethan-1-one

Step 1: tert-butyl 4-(4-(2-42-amino-5-(2-methyl-6-(trifluoromethyl)pyridin-4-yl)-6-phenylpyrimidin-4-yl) oxy)ethyl)phenyl)piperazine-1-carboxylate. To a mixture of tert-butyl 4-(4-(2-hydroxyethyl)phenyl)piperazine-1-carboxylate (353.0 mg, 1.15 mmol) in THF (12 mL) was added NaH (60% in mineral, 99.0 mg, 2.47 mmol) at room temperature under nitrogen atmosphere. The reaction mixture was stirred at 30° C. for 20 min A solution of 4-chloro-5-(2-methyl-6-(trifluoromethyl)pyridin-4-yl)-6-phenylpyrimidin-2-amine (300.0 mg, 0.82 mmol) in THF (3 mL) then was added dropwise at 0° C. The reaction mixture was further stirred at 0° C. for 10 min, then at 30° C. for 6 h. The reaction mixture was quenched with ice water (2 mL) and concentrated. The residue was purified on silica gel column (Petroleum ether : EtOAc=2:1) to afford tert-butyl 4-(4-(2-((2-amino-5-(2-methyl-6-(trifluoromethyl)pyridin-4-yl)-6-phenylpyrimidin-4-yl) oxy)ethyl)phenyl)piperazine-1-carboxylate (470 mg, 90%) as a yellow oil. ES-MS (m/z): [M+1]⁺=635.2.

Step 2: 5-(2-methyl-6-(trifluoromethyl)pyridin-4-yl)-4-phenyl-6-(4-(piperazin-1-yl)phenethoxy)pyrimidin-2-amine hydrochloride. To a mixture of tert-butyl 4-(4-(2-((2-amino-5-(2-methyl-6-(trifluoromethyl)pyridin-4-yl)-6-phenylpyrimidin-4-yl)oxy)ethyl)phenyl)piperazine-1-carboxylate (470.0 mg, 0.74 mmol) in ethyl acetate (4 mL) was added HCl in ethyl acetate (4.0 M, 4 mL). The reaction mixture was stirred at room temperature for 30 min, then was concentrated to afford 5-(2-methyl-6-(trifluoromethyl)pyridin-4-yl)-4-phenyl-6-(4-(piperazin-1-yl)phenethoxy)pyrimidin-2-amine hydrochloride (410.0 mg, 97%) as a yellow solid. ES-MS (m/z): [M+1]⁺=535.2.

Step 3: 1-(4-(4-(2-((2-amino-5-(2-methyl-6-(trifluoromethyl)pyridin-4-yl)-6-phenylpyrimidin-4-yl)oxy)ethyl)phenyl)piperazin-1-yl)ethan-1-one. To a mixture of 5-(2-methyl-6-(trifluoromethyl)pyridin-4-yl)-4-phenyl-6-(4-(piperazin-1-yl)phenethoxy)pyrimidin-2-amine hydrochloride (95.0 mg, 0.17 mmol) and triethylamine (50.0 mg, 0.49 mmol) in DCM (3 mL) was added a solution of acetyl chloride (13.0 mg, 0.17 mmol) in DCM (1 mL) dropwise at room temperature. After stirring at room temperature for 2 h, the reaction mixture was concentrated. The residue was purified by prep-TLC (EtOAc) to afford 1-(4-(4-(2-((2-amino-5-(2-methyl-6-(trifluoromethyl)pyridin-4-yl)-6-phenylpyrimidin-4-yl)oxy)ethyl)phenyl)piperazin-1-yl)ethan-1-one (26 mg, 27%) as a white solid. ES-MS (m/z): [M+1]⁺=577.3.

### Example 21

### (2-amino-5-(2-methyl-6-(trifluoromethyl)pyridin-4-yl)-6-phenylpyrimidin-4-yl)methyl diethylcarbamate

Step 1: (2-amino-5-(2-methyl-6-(trifluoromethyl)pyridin-4-yl)-6-phenylpyrimidin-4-yl)methyl diethylcarbamate. To a stirred mixture of NaH (12.0 mg, 0.30 mmol, 60%) in DCM (1 mL) was added [2-amino-5-[2-methyl-6-(trifluoromethyl)pyridin-4-yl]-6-phenylpyrimidin-4-yl]methanol (54.0 mg, 0.15 mmol) at room temperature under nitrogen atmosphere. After stirring at this temperature for 15 min, N,N-diethylcarbamoyl chloride (20.3 mg, 0.15 mmol) was added. The resulting mixture was stirred for additional 16 h at room temperature. The reaction mixture was acidified to pH 7 with 1 M aqueous HCl. The resulting mixture was concentrated and purified by Prep-HPLC to obtain (2-amino-5-(2-methyl-6-(trifluoromethyl)pyridin-4-yl)-6-phenylpyrimidin-4-yl)methyl diethylcarbamate (30 mg, 43.6%) as an off-white solid. ES-MS (m/z): [M+1]⁺=460.0.

### Example 22

### 5-(2-methyl-6-(trifluoromethyl)pyridin-4-yl)-4-((methylamino)methyl)-6-phenylpyrimidin-2-amine

Step 1: (2-amino-5-(2-methyl-6-(trifluoromethyl)pyridin-4-yl)-6-phenylpyrimidin-4-yl)methyl methanesulfonate. To a stirred solution of [2-amino-5-[2-methyl-6-(trifluoromethyl)pyridin-4-yl]-6-phenylpyrimidin-4-yl]methanol (162.0 mg, 0.45 mmol) and DIPEA (174.3 mg, 1.35 mmol) in DCM (3 mL) was added MsCl (51.5 mg, 0.45 mmol) dropwise at room temperature under nitrogen atmosphere. The resulting mixture was stirred for additional 3 h at this temperature and concentrated. The obtained crude 2-amino-5-(2-methyl-6-(trifluoromethyl)pyridin-4-yl)-6-phenylpyrimidin-4-yl)methyl methanesulfonate was used for next step directly without further purification. ES-MS (m/z): [M+1]⁺=439.2.

Step 2: 5-(2-methyl-6-(trifluoromethyl)pyridin-4-yl)-4-((methylamino)methyl)-6-phenylpyrimidin-2-amine. Into a 8 mL vial were added (2-amino-5-(2-methyl-6-(trifluoromethyl)pyridin-4-yl)-6-phenylpyrimidin-4-yl)methyl methanesulfonate (crude, 0.14 mmol) and CH₃NH₂ (0.347 mL, 2M in THF, 0.69 mmol) at room temperature. The resulting mixture was stirred for 16 h at room temperature, then concentrated. The residue was purified by Prep-HPLC to obtain 5-(2-methyl-6-(trifluoromethyl)pyridin-4-yl)-4-((methylamino)methyl)-6-phenylpyrimidin-2-amine (33 mg, 63.7%) as an off-white solid. ES-MS (m/z): [M+1]⁺=374.2.

### Example 23

### 4-((dimethylamino)methyl)-5-(2-methyl-6-(trifluoromethyl)pyridin-4-yl)-6-phenylpyrimidin-2-amine

Step 1: 4-[(dimethylamino)methyl]-5-[2-methyl-6-(trifluoromethyl)pyridin-4-yl]-6-phenylpyrimidin-2-amine Following step 2, example 22. From 2-amino-5-(2-methyl-6-(trifluoromethyl)pyridin-4-yl)-6-phenylpyrimidin-4-yl)methyl methanesulfonate (crude) and dimethylamine in THF. ES-MS (m/z): [M+1]⁺=388.3.

### Example 24

### 5-(2-methyl-6-(trifluoromethyl)pyridin-4-yl)-4-phenyl-6-(((2,2,2-trifluoroethyl)amino)methyl)pyrimidin-2-amine

Step 1: 5-(2-methyl-6-(trifluoromethyl)pyridin-4-yl)-4-phenyl-6-(((2,2,2-trifluoroethyl)amino)methyl)pyrimidin-2-amine Following step 2, example 22. From 2-amino-5-(2-methyl-6-(trifluoromethyl)pyridin-4-yl)-6-phenylpyrimidin-4-yl)methyl methanesulfonate (crude) and 2,2,2-trifluoroethan-1-amine in acetonitrile. ES-MS (m/z): [M+1]⁺=442.2.

### Example 25

### 1-(3-(4-(2-((2-amino-5-(2-methyl-6-(trifluoromethyl)pyridin-4-yl)-6-phenylpyrimidin-4-yl)oxy)ethyl)phenoxy)pyrrolidin-1-yl)ethan-1-one

Step 1: 1-(3-(4-(2-((2-amino-5-(2-methyl-6-(trifluoromethyl)pyridin-4-yl)-6-phenylpyrimidin-4-yl)oxy)ethyl)phenoxy)pyrrolidin-1-yl)ethan-1-one. To a mixture of 5-(2-methyl-6-(trifluoromethyl)pyridin-4-yl)-4-phenyl-6-(4-(pyrrolidin-3-yloxy)phenethoxy)pyrimidin-2-amine (120.0 mg, 0.22 mmol) and triethylamine (27.2 mg, 0.27 mmol) in DCM (4 mL) was added acetic anhydride (22.8 mg, 0.22 mmol) in DCM (2 mL) dropwise at 0° C. The mixture then was stirred at room temperature for 30 min and concentrated. The crude was purified by prep-HPLC to give 1-(3-(4-(2-((2-amino-5-(2-methyl-6-(trifluoromethyl)pyridin-4-yl)-6-phenylpyrimidin-4-yl)oxy)ethyl)phenoxy)pyrrolidin-1-yl)ethan-1-one (25 mg, 19%) as white solid. ES-MS (m/z): [M+1]⁺=578.0.

### Example 26

### 1-(4-(2-((2-amino-5-(2-methyl-6-(trifluoromethyl)pyridin-4-yl)-6-phenylpyrimidin-4-yl)oxy)ethyl)phenyl)piperidin-4-one

Step 1: (4-bromophenethoxy)(tert-butyl)dimethylsilane. To a mixture of 2-(4-bromophenyl)ethanol (10.0 g, 49.7 mmol) in DCM (100 mL) were added imidazole (10.0 g, 149.2 mmol) and TBSC1 (8.2 g, 54.7 mmol) at 0° C. After stirring at room temperature for 3 h, the reaction mixture was quenched with water, extracted with DCM. The organic layer was washed with brine, dried over Na₂SO₄, filtered and concentrated. The crude was purified by silica gel column (PE:EA=50:1) to get (4-bromophenethoxy)(tert-butyl)dimethylsilane (16.2 g, 99%) as a colorless oil.

Step 2: 1-(4-(2-((tert-butyldimethylsilyl)oxy)ethyl)phenyl)piperidin-4-ol. To a mixture of (4-bromophenethoxy)(tert-butyl)dimethylsilane (3.0 g, 9.5 mmol), piperidin-4-ol (1.4 g, 14.3 mmol), Pd₂(dba)₃ (871.0 mg, 0.95 mmol), XPhos (906.0 mg, 1.9 mmol) in THF (40 mL) under nitrogen was added LiHMDS (28.5 mL, 1.0 M in THF, 28.5 mmol) dropwise at room temperature. The mixture then was stirred at 75° C. overnight. The reaction mixture was cooled and quenched with H₂O, then extracted with ethyl acetate. The organic layer was washed with brine, dried over Na₂SO₄. The mixture was filtered and concentrated. The crude was purified on silica gel column (PE:EA=5:1) to get 1-(4-(2-((tert-butyldimethylsilyl)oxy)ethyl)phenyl)piperidin-4-ol (2.5 g, 78%) as a brown oil. ES-MS (m/z): [M+1]⁺=336.1.

Step 3: 1-(4-(2-((tert-butyldimethylsilyl)oxy)ethyl)phenyl)piperidin-4-one. To a mixture of oxalyl chloride (1.82 g, 14.3 mmol) in DCM (27 mL) at −78° C. was added a solution of DMSO (2.23 g, 28.6 mmol) in DCM (7.7 mL) dropwise. After stirring further at −78° C. for 15 min, a solution of 1-(4-(2-((tert-butyldimethylsilyl)oxy)ethyl)phenyl)piperidin-4-ol (2.4 g, 7.15 mmol) in DCM (14 mL) was added dropwise at −78° C. The mixture was stirred at −78° C. for additional 1 h, TEA (4.3 g, 42.9 mmol) was added dropwise at this temperature. The resulting mixture was slowly warmed to room temperature and stirred at this temperature for 1 h. The reaction was quenched with water and extracted with DCM. The organic layer was washed with brine, dried over Na₂SO₄. The mixture was filtered and concentrated. The crude was purified on silica gel column (PE:EA=2:1) to get 1-(4-(2-((tert-butyldimethylsilyl)oxy)ethyl)phenyl)piperidin-4-one (3.7 g, 76%) as a brown oil. ES-MS (m/z): [M+1]⁺=333.9.

Step 4: 1-(4-(2-hydroxyethyl)phenyl)piperidin-4-one. To a mixture of 1-(4-(2-((tert-butyldimethylsilyl)oxy)ethyl)phenyl)piperidin-4-one (1.0 g, 3.0 mmol) in THF (6.0 mL) was added TBAF in THF (5.94 mL, 1.0 M, 5.94 mmol). The resulting mixture was stirred at room temperature for 3 h. The mixture was concentrated and purified on silica gel column (DCM: MeOH=70:1) to get 1-(4-(2-hydroxyethyl)phenyl)piperidin-4-one (227 mg, 35%) as a brown oil. ES-MS (m/z): [M+1]⁺=220.0.

Step 5: 1-(4-(2-((2-amino-5-(2-methyl-6-(trifluoromethyl)pyridin-4-yl)-6-phenylpyrimidin-4-yl)oxy)ethyl)phenyl)piperidin-4-one. Following step 2, example 8. From 1-(4-(2-hydroxyethyl)phenyl)piperidin-4-one, 60% NaH and 4-chloro-5-(2-methyl-6-(trifluoromethyl)pyridin-4-yl)-6-phenylpyrimidin-2-amine in THF. ES-MS (m/z): [M+1]⁺=548.2.

### Example 27

### 2-[[2-amino-6-(4-fluorophenyl)-5-[2-methyl-6-(trifluoromethyl)pyridin-4-yl]pyrimidin-4-yl]oxy]-N,N-diethylacetamide

Step 1: 2-[[2-amino-6-(4-fluorophenyl)-5-(2-methyl-6-(trifluoromethyl)pyridin-4-yl]pyrimidin-4-yl]oxyl-N,N-diethylacetamide. Following step 2, example 8. From N, N-diethyl-2-hydroxyacetamide, 60% NaH and 4-chloro-6-(4-fluorophenyl)-5-[2-methyl-6-(trifluoromethyl)pyridin-4-yl]pyrimidin-2-amine in THF. ES-MS (m/z): [M+1]⁺=478.1.

### Example 28

### 2-([2-amino-5-[2-(difluoromethyl)-6-methylpyridin-4-yl]-6-(4-fluorophenyl)pyrimidin-4-yl]oxy)-N,N-diethylacetamide

Step 1: 4-chloro-6-(4-fluorophenyl)pyrimidin-2-amine Following step 1, example 1. From 4,6-dichloropyrimidin-2-amine, 4-fluorophenylboronic acid, Pd(dpp1)Cl₂ and K₂CO₃ in 1,4-dioxane and H₂O.

Step 2: 4-chloro-6-(4-fluorophenyl)-5-iodopyrimidin-2-amine Following step 2, example 1. From 4-chloro-6-(4-fluorophenyl)pyrimidin-2-amine and NIS in DMF.

Step 3: 4-chloro-5-l2-(difluoromethyl)-6-methylpyridin-4-yl1-6-(4-fluorophenyl)pyrimidin-2-amine. Following step 3, example 1. From 4-chloro-6-(4-fluorophenyl)-5-iodopyrimidin-2-amine, 2-(difluoromethyl)-6-methylpyridin-4-yl)boronic acid, Pd(dppf)Cl₂ and K₂CO₃ in 1,4-dioxane and H₂O.

Step 4: 2-([2-amino-5-l2-(difluoromethyl)-6-methylpyridin-4-yl]-6-(4-fluorophenyl)pyrimidin-4-yl)oxy)-N,N-diethylacetamide. Following step 2, example 8. From N, N-diethyl-2-hydroxyacetamide, 60% NaH and 4-chloro-5-l2-(difluoromethyl)-6-methylpyridin-4-yl)-6-(4-fluorophenyl)pyrimidin-2-amine in THF. ES-MS (m/z): [M+1]⁺=460.2.

### Example 29

### 2-([2-amino-5-[2-methyl-6-(trifluoromethyl)pyridin-4-yl]-6-phenylpyrimidin-4-yl]oxy)-N-ethylacetamide

Step 1: 2-([2-amino-5-[2-methyl-6-(trifluoromethyl)pyridin-4-yl]-6-phenylpyrimidin-4-yl]oxy)-N-ethylacetamide. Following step 2, example 2. From 2-(12-amino-5-[2-methyl-6-(trifluoromethyl)pyridin-4-yl]-6-phenylpyrimidin-4-yl)oxy)acetic acid, ethylamine, DIPEA and HATU in DMF. ES-MS (m/z): [M+1]⁺=432.1.

### Example 30

### 2-((2-amino-5-(2-methyl-6-(trifluoromethyl)pyridin-4-yl)-6-phenylpyrimidin-4-yl)amino)-N,N-diethylacetamide

Step 1: tert-butyl N-[(diethylcarbamoyl)methyl]carbamate. To a stirred solution of 2-[[(tert-butoxy)carbonyl]amino]acetic acid (2.0 g, 11.42 mmol) in DMF (20 mL) were added HOBt (1.85 g, 13.70 mmol), then DCC (2.83 g, 13.70 mmol) at 0° C. The resulting mixture was stirred for 1 h at 0° C., diethylamine (0.83 g, 11.42 mmol) then was added dropwise at 0° C. After stirring for additional 6 h at room temperature, the reaction mixture was filtered and the filter cake was washed with DCM (50 mL). The organic filtrate was washed with 0.1 M HCl, water (3×25 mL) and brine (25 mL). The organic layer was dried over anhydrous Na₂SO₄, filtered and concentrated. The residue was purified on silica gel column with PE/EA (1/1) to afford tert-butyl N—R(diethylcarbamoyl)methyl)carbamate (1.6 g, 60.9%) as an off-white solid. ES-MS (m/z): [M+1]⁺=231.0.

Step 2: 2-amino-N,N-diethylacetamide hydrochloride. To a stirred solution of tert-butyl N-[(diethylcarbamoyl)methyl]carbamate (100 mg, 0.43 mmol) in 1,4-dioxane (1 mL) was added HCl (2 mL, 4M in 1,4-dioxane) dropwise at room temperature. After stirring for 16 h at room temperature, the reaction mixture was filtered and the solid cake was washed with Et₂O (3×10 mL) to obtain 2-amino-N,N-diethylacetamide hydrochloride (64 mg, 88.5%) as a white solid. ES-MS (m/z): [M+1]⁺=131.1.

Step 3: 2-((2-amino-5-(2-methyl-6-(trifluoromethyl)pyridin-4-yl)-6-phenylpyrimidin-4-yl)amino)-N,N-diethylacetamide. To a stirred mixture of 2-amino-N,N-diethylacetamide hydrochloride (64.0 mg, 0.38 mmol) and triethylamine (77.7 mg, 0.77 mmol) in i-PrOH (3 mL) was added 4-chloro-5-[2-methyl-6-(trifluoromethyl)pyridin-4-yl]-6-phenylpyrimidin-2-amine (140.1 mg, 0.38 mmol) at room temperature. After stirring for 16 h at 110° C., the reaction mixture was cooled and concentrated. The residue was purified by Prep-HPLC to afford 2-((2-amino-5-(2-methyl-6-(trifluoromethyl)pyridin-4-yl)-6-phenylpyrimidin-4-yl)amino)-N,N-diethylacetamide (61 mg, 34.6%) as an off-white solid. ES-MS (m/z): [M+1]⁺=459.1.

### Example 31

### 1-(4-[4-[2-([2-amino-5-[2-(difluoromethyl)-6-methylpyridin-4-yl]-6-(4-fluorophenyl)pyrimidin-4-yl]oxy)ethyl]phenyl]piperazin-1-yl)ethan-1-one

Step 1: [2-(4-bromophenyl)ethoxy](tert-butyl)dimethylsilane. To a stirred solution of 2-(4-bromophenyl)ethan-1-ol (5.0 g, 24.9 mmol) and imidazole (4.23 g, 62.2 mmol) in DCM (50 mL) was added TBSC1 (4.5 g, 29.8 mmol) in portions at 0° C. under nitrogen atmosphere. The resulting mixture was stirred for 16 h at room temperature under nitrogen atmosphere. This mixture was diluted with water (50 mL) and DCM (50 mL), and the aqueous layer was extracted with DCM (3×50 mL). The combined organic layers were washed with brine (30 mL), dried over anhydrous Na₂SO₄. The mixture was filtered and concentrated, and the residue was purified on silica gel column with PE/EtOAc (8:1) to afford [2-(4-bromophenyl)ethoxy](tert-butyl)dimethylsilane (7.6 g, 96.9%) as a colorless oil.

Step 2: 1-[4-(4-(2-[(tert-butyldimethylsilyl)oxylethyl]phenyl)piperazin-1-yl]ethan-1-one. To a stirred solution [2-(4-bromophenyl)ethoxy](tert-butyl)dimethylsilane (1.0 g, 3.17 mmol) and 1-(piperazin-1-yl)ethan-1-one (490 mg, 3.82 mmol) in toluene (10 mL) were added BINAP (100 mg, 0.16 mmol), Pd₂(dba)₃ (146 mg, 0.16 mmol) and t-BuONa (917 mg, 9.54 mmol) at room temperature under nitrogen atmosphere. The resulting mixture was stirred for 16 h at 100° C. under nitrogen atmosphere. This mixture was cooled and filtered. The filter cake was washed with DCM (50 mL). The combined filtrate was concentrated. The residue was purified on silica gel column with PE/EtOAc (5:1) to afford 1-[4-(4-[2-[(tert-butyldimethylsilyl)oxy]ethyl]phenyl)piperazin-1-yl]ethan-1-one (620 mg, 53.9%) as a yellow solid. ES-MS (m/z): [M+1]⁺=363.4.

Step 3: 1-[4-[4-(2-hydroxyethyl)phenyl]piperazin-1-yl]ethan-1-one. To a stirred solution of 1-[4-(4-[2-[(tert-butyldimethylsilyl)oxy]ethyl]phenyl)piperazin-1-yl]ethan-1-one (300 mg, 0.83 mmol) in DCM (2 mL) was added 2 M HCl in ethyl acetate (2 mL, 4.0 mmol). After stirring for 16 h at room temperature under nitrogen atmosphere, the reaction mixture was diluted with DCM (5 mL) and washed with saturated Na₂CO₃. The organic layer was concentrated and the residue was purified on silica gel column with DCM/MeOH (9:1) to afford 1-[4-[4-(2-hydroxyethyl)phenyl]piperazin-1-yl]ethan-1-one (150 mg, 73.0%) as a yellow solid. ES-MS (m/z): [M+1]⁺=249.3.

Step 4: 1-(4-[4-[2-([2-amino-5-[2-(difluoromethyl)-6-methylpyridin-4-yl]-6-(4-fluorophenyl)pyrimidin-4-yl]oxy)ethyl]phenyl]piperazin-1-yl)ethan-1-one. Following step 2, example 8. From 1-[4-[4-(2-hydroxyethyl)phenyl]piperazin-1-yl]ethan-1-one, 60% NaH and 4-chloro-5-[2-(difluoromethyl)-6-methylpyridin-4-yl]-6-(4-fluorophenyl)pyrimidin-2-amine in THF. ES-MS (m/z): [M+1]⁺=577.3.

### Example 32

### 2-((2-amino-5-(2-(difluoromethyl)-6-methylpyridin-4-yl)-6-(4-fluorophenyl)pyrimidin-4-yl)oxy)-N-methylacetamide

Step 1: methyl 2-([2-amino-5-[2-(difluoromethyl)-6-methylpyridin-4-yl]-6-(4-fluorophenyl)pyrimidin-4-yl]oxy)acetate. To a stirred mixture of 4-chloro-5-[2-(difluoromethyl)-6-methylpyridin-4-yl]-6-(4-fluorophenyl)pyrimidin-2-amine (1.1 g, 3.02 mmol) and K₂CO₃ (1.3 g, 9.05 mmol) in DMF (10 mL) was added methyl 2-hydroxyacetate (0.5 g, 6.03 mmol) at room temperature. After stirring for 6 h at 80° C., the reaction mixture was cooled and diluted with ethyl acetate (30 mL). The resulting mixture was washed with water (3×30 mL) and brine (30 mL). The organic layer was dried over anhydrous Na₂SO₄, filtered and concentrated. The residue was purified on silica gel column with PE/EA (3/1) to afford methyl 2-([2-amino-5-[2-(difluoromethyl)-6-methylpyridin-4-yl]-6-(4-fluorophenyl)pyrimidin-4-yl]oxylacetate (461 mg, 36.5%) as a light yellow solid. ES-MS (m/z): [M+1]⁺=419.

Step 2: 2-([2-amino-5-[2-(difluoromethyl)-6-methylpyridin-4-yl]-6-(4-fluorophenyl)pyrimidin-4-yl]oxy)acetic acid. To a stirred solution of methyl 2-([2-amino-5-[2-(difluoromethyl)-6-methylpyridin-4-yl]-6-(4-fluorophenyl)pyrimidin-4-yl]oxy)acetate (461.0 mg, 1.10 mmol) in MeOH (4 mL) was added a solution of LiOH (52.8 mg, 2.20 mmol) in H₂O (4 mL) at room temperature. After stirring for 16 h at room temperature, the reaction mixture was concentrated to remove organic solvent. The residue was acidified to pH 3-4 with 0.5 M aqueous HCl. The resulting mixture was filtered and washed with water (3×10 mL) to afford 2-([2-amino-5-[2-(difluoromethyl)-6-methylpyridin-4-yl]-6-(4-fluorophenyl)pyrimidin-4-yl]oxy)acetic acid (340 mg, 76.3%) as an off-white solid. ES-MS (m/z): [M+1]⁺=404.9.

Step 3: 2-((2-amino-5-(2-(difluoromethyl)-6-methylpyridin-4-yl)-6-(4-fluorophenyl)pyrimidin-4-yl)oxy)-N-methylacetamide. To a stirred mixture of 2-([2-amino-5-[2-(difluoromethyl)-6-methylpyridin-4-yl]-6-(4-fluorophenyl)pyrimidin-4-yl]oxy)acetic acid (35 mg, 0.087 mmol) and methylamine hydrochloride (6 mg, 0.094 mmol) in DCM (0.5 mL) were added DIPEA (34 mg, 0.260 mmol) and HATU (49 mg, 0.130 mmol) at room temperature. After stirring for 2 h at room temperature, the reaction mixture was diluted with DCM (5 mL). The reaction mixture was washed with aqueous NaHCO₃ (3×5 mL). The organic layer was dried over anhydrous Na₂SO₄, filtered and concentrated. The residue was purified by Prep-HPLC to afford 2-((2-amino-5-(2-(difluoromethyl)-6-methylpyridin-4-yl)-6-(4-fluorophenyl)pyrimidin-4-yl)oxy)-N-methylacetamide (17.7 mg, 45.1%) as an off-white solid. ES-MS (m/z): [M+1]⁺=418.0.

### Example 33

### 5-(2-(difluoromethyl)-6-methylpyridin-4-yl)-4-(4-fluorophenyl)-6-((tetrahydro-2H-pyran-4-yl)oxy)pyrimidin-2-amine p Step 1: 5-(2-(difluoromethyl)-6-methylpyridin-4-yl)-4-(4-fluorophenyl)-6-((tetrahydro-2H-pyran-4-yl)oxy)pyrimidin-2-amine Following step 2, example 8. From tetrahydro-2H-pyran-4-ol, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=431.1.

### Example 34

### 5-(2-(difluoromethyl)-6-methylpyridin-4-yl)-4-(4-fluorophenyl)-6-(oxetan-3-yloxy)pyrimidin-2-amine

Step 1: 5-(2-(difluoromethyl)-6-methylpyridin-4-yl)-4-(4-fluorophenyl)-6-(oxetan-3-yloxy)pyrimidin-2-amine Following step 2, example 8. From oxetan-3-ol, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=403.1.

### Example 35

### 5-(2-(difluoromethyl)-6-methylpyridin-4-yl)-4-(4-fluorophenyl)-6-(2-(4-methylpiperazin-1-yl)ethoxy)pyrimidin-2-amine

Step 1: 5-(2-(difluoromethyl)-6-methylpyridin-4-yl)-4-(4-fluorophenyl)-6-(2-(4-methylpiperazin-1-yl)ethoxy)pyrimidin-2-amine Following step 2, example 8. From 2-(4-methylpiperazin-1-yl)ethanol, 60% NaH and 4-chloro-5-(2-(difluoromethyl)-6-methylpyridin-4-yl)-6-(4-fluorophenyl)pyrimidin-2-amine in DMF/DCM. ES-MS (m/z): [M+1]⁺=473.0.

### Example 36

### 5-(2-(difluoromethyl)-6-methylpyridin-4-yl)-4-(4-fluorophenyl)-6-(2-morpholinoethoxy)pyrimidin-2-amine

Step 1: 5-(2-(difluoromethyl)-6-methylpyridin-4-yl)-4-(4-fluorophenyl)-6-(2-morpholinoethoxy)pyrimidin-2-amine Following step 2, example 8. From 2-morpholinoethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=460.2.

### Example 37

### 5-(2-(difluoromethyl)-6-methylpyridin-4-yl)-4-(4-fluorophenyl)-6-(oxetan-3-ylmethoxy)pyrimidin-2-amine

Step 1: 5-(2-(difluoromethyl)-6-methylpyridin-4-yl)-4-(4-fluorophenyl)-6-(oxetan-3-ylmethoxy)pyrimidin-2-amine. Following step 2, example 8. From oxetan-3-ylmethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=417.1.

### Example 38

### 2-((2-amino-5-(2-methyl-6-(trifluoromethyl)pyridin-4-yl)-6-phenylpyrimidin-4-yl)(methyl)amino)-N,N-diethylacetamide

Step 1: tert-butyl N-[(diethylcarbamoyl)methyl]-N-methylcarbamate. To a stirred solution of NaH (121.6 mg, 3.04 mmol, 60%) in THF (5 mL) was added tert-butyl N-[(diethylcarbamoyl)methyl]carbamate (350.0 mg, 1.52 mmol) at room temperature. After stirring for 15 min at room temperature, MeI (215.7 mg, 1.52 mmol) was added dropwise. The resulting mixture was stirred for additional 16 h at room temperature, then was quenched with 0.2 mL of MeOH. The mixture was acidified to pH 7 using aqueous HCl (1.0 N), then diluted with ethyl acetate (10 mL). The resulting mixture was washed with 3×10 mL of water and 10 mL of brine. The organic layer was dried over anhydrous Na₂SO₄, filtered and concentrated. The residue was purified on silica gel column with PE/EA (2/1) to afford tert-butyl N-[(diethylcarbamoyl)methyl]-N-methylcarbamate (221 mg, 59.5%) as a white solid. ES-MS (m/z): [M+1]⁺=245.2.

Step 2: N,N-diethyl-2-(methylamino)acetamide hydrochloride. To a stirred solution of tert-butyl N-[(diethylcarbamoyl)methyl]-N-methylcarbamate (221 mg, 0.904 mmol) in 1,4-dioxane (1 mL) was added HCl (1 mL, 4 M in 1,4-dioxane) at room temperature. After stirring for 6 h at room temperature, the reaction mixture was filtered and washed with Et₂O (3×3 mL) to afford N,N-diethyl-2-(methylamino)acetamide hydrochloride (151 mg, 92.4%) as an off-white solid. ES-MS (m/z): [M+1]⁺=145.2.

Step 3: 24(2-amino-5-(2-methyl-6-(trifluoromethyl)pyridin-4-yl)-6-phenylpyrimidin-4-yl)(methyl)amino)-N,N-diethylacetamide. To a stirred mixture of 4-chloro-5-[2-methyl-6-(trifluoromethyl)pyridin-4-yl]-6-phenylpyrimidin-2-amine (60.6 mg, 0.166 mmol) and K₂CO₃ (45.9 mg, 0.332 mmol) in DMF (1 mL) was added N,N-diethyl-2-(methylamino)acetamide hydrochloride (30.0 mg, 0.166 mmol) at room temperature. After stirring for 16 h at 80° C., the reaction mixture was cooled and filtered, and the filter cake was washed with DMF (2×1 mL). The filtrate was purified by Prep-HPLC to afford 2-([2-amino-5-[2-methyl-6-(trifluoromethyl)pyridin-4-yl]-6-phenylpyrimidin-4-yl](methyl)amino)-N,N-diethylacetamide (27 mg, 34.4%) as an off-white solid. ES-MS (m/z): [M+1]⁺=473.1.

### Example 39

### 2-((2-amino-5-(2-(difluoromethyl)-6-methylpyridin-4-yl)-6-(4-fluorophenyl)pyrimidin-4-yl)oxy)-N-ethylacetamide

Following step 3, example 32. From 2-([2-amino-5-[2-(difluoromethyl)-6-methylpyridin-4-yl]-6-(4-fluorophenyl)pyrimidin-4-yl]oxy)acetic acid, ethylamine hydrochloride, DIPEA and HATU in DCM. ES-MS (m/z): [M+1]⁺=432.0.

### Example 40

### 2-((2-amino-5-(2-(difluoromethyl)-6-methylpyridin-4-yl)-6-(4-fluorophenyl)pyrimidin-4-yl)oxy)-N-(tetrahydro-2H-pyran-4-yl)acetamide

Following step 3, example 32. From 2-([2-amino-5-[2-(difluoromethyl)-6-methylpyridin-4-yl]-6-(4-fluorophenyl)pyrimidin-4-yl]oxy)acetic acid, tetrahydro-2H-pyran-4-amine, DIPEA and HATU in DCM. ES-MS (m/z): [M+1]⁺=488.3.

### Example 41

### 2-((2-amino-5-(2-(difluoromethyl)-6-methylpyridin-4-yl)-6-(4-fluorophenyl)pyrimidin-4-yl)oxy)-N-isopropylacetamide

Following step 3, example 32. From 2-([2-amino-5-[2-(difluoromethyl)-6-methylpyridin-4-yl]-6-(4-fluorophenyl)pyrimidin-4-yl]oxy)acetic acid, isopropylamine, DIPEA and HATU in DCM. ES-MS (m/z): [M+1]⁺=446.1.

### Example 42

### 2-((2-amino-5-(2-(difluoromethyl)-6-methylpyridin-4-yl)-6-(4-fluorophenyl)pyrimidin-4-yl)oxy)-N-cyclopropylacetamide

Following step 3, example 32. From 2-([2-amino-5-[2-(difluoromethyl)-6-methylpyridin-4-yl]-6-(4-fluorophenyl)pyrimidin-4-yl]oxy)acetic acid, cyclopropylamine, DIPEA and HATU in DCM. ES-MS (m/z): [M+1]⁺=444.3.

### Example 43

### 2-((2-amino-5-(2-(difluoromethyl)-6-methylpyridin-4-yl)-6-(4-fluorophenyl)pyrimidin-4-yl)oxy)-N-(2-(dimethylamino)ethyl)acetamide

Following step 3, example 32. From 2-([2-amino-5-[2-(difluoromethyl)-6-methylpyridin-4-yl]-6-(4-fluorophenyl)pyrimidin-4-yl]oxy)acetic acid, (2-aminoethyl)dimethylamine, DIPEA and HATU in DCM. ES-MS (m/z): [M+1]⁺=475.1.

### Example 44

### 2-((2-amino-5-(2-(difluoromethyl)-6-methylpyridin-4-yl)-6-(4-fluorophenyl)pyrimidin-4-yl)oxy)-N-(1-methylpiperidin-4-yl)acetamide

Following step 3, example 32. From 2-([2-amino-5-[2-(difluoromethyl)-6-methylpyridin-4-yl]-6-(4-fluorophenyl)pyrimidin-4-yl]oxy)acetic acid, 1-methylpiperidin-4-amine, DIPEA and HATU in DCM. ES-MS (m/z): [M+1]⁺=501.1.

### Example 45

### 2-((2-amino-5-(2-(difluoromethyl)-6-methylpyridin-4-yl)-6-(4-fluorophenyl)pyrimidin-4-yl)oxy)-N-(2-methoxyethyl)acetamide

Following step 3, example 32. From 2-([2-amino-5-[2-(difluoromethyl)-6-methylpyridin-4-yl]-6-(4-fluorophenyl)pyrimidin-4-yl]oxy)acetic acid, 2-methoxyethan-1-amine, DIPEA and HATU in DCM. ES-MS (m/z): [M+1]⁺=462.1.

### Example 46

### 5-(2-(difluoromethyl)-6-methylpyridin-4-yl)-4-(4-fluorophenyl)-6-(3-(methylsulfonyl)propoxy)pyrimidin-2-amine

Step 1: 5-(2-(difluoromethyl)-6-methylpyridin-4-yl)-4-(4-fluorophenyl)-6-(3-(methylsulfonyl)propoxy)pyrimidin-2-amine Following step 2, example 8. From 3-(methylsulfonyl)propan-1-ol, 60% NaH and 4-chloro-5-(2-(difluoromethyl)-6-methylpyridin-4-yl)-6-(4-fluorophenyl)pyrimidin-2-amine in DMF/DCM. ES-MS (m/z): [M+1]⁺=467.1.

### Example 47

### 5-(2-(difluoromethyl)-6-methylpyridin-4-yl)-4-(4-fluorophenyl)-6-phenoxypyrimidin-2-amine

Step 1: 5-(2-(difluoromethyl)-6-methylpyridin-4-yl)-4-(4-fluorophenyl)-6-phenoxypyrimidin-2-amine

Following step 2, example 8. From phenol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=423.1.

### Example 48

### 5-(2-(difluoromethyl)-6-methylpyridin-4-yl)-4-(4-fluorophenyl)-6-(2-methoxyethoxy)pyrimidin-2-amine

Step 1: 5-(2-(difluoromethyl)-6-methylpyridin-4-yl)-4-(4-fluorophenyl)-6-(2-methoxyethoxy)pyrimidin-2-amine Following step 2, example 8. From tert-butyl 4-(2-hydroxyethyl)piperazine-1-carboxylate, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=405.1.

### Example 49

### 5-(2-(difluoromethyl)-6-methylpyridin-4-yl)-4-(4-fluorophenyl)-6-(2-(4-(methylsulfonyl)piperazin-1-yl)ethoxy)pyrimidin-2-amine

Step 1: benzyl 4-methanesulfonylpiperazine-1-carboxylate. To a stirred solution of benzyl piperazine-1-carboxylate (5.0 g, 22.7 mmol) in DCM (50 mL) were added triethylamine (6.89 g, 68.1 mmol) and MsCl (3.4 g, 29.7 mmol, 1.31) dropwise at 0° C. under nitrogen atmosphere. After stirring for 3 h at room temperature, the reaction mixture was quenched with water (30 mL) at 0° C., extracted with DCM (3×30 mL). The combined organic extracts were washed with brine (20 mL), dried over anhydrous Na₂SO₄, filtered and concentrated. The residue was purified on silica gel column with DCM/MeOH (20:1) to afford benzyl 4-methanesulfonylpiperazine-1-carboxylate (6 g, 88.6%) as a yellow solid. ES-MS (m/z): [M+1]⁺=299.2.

Step 2: 1-methanesulfonylpiperazine. To a solution of benzyl 4-methanesulfonylpiperazine-1-carboxylate (3.0 g, 10.1 mmol) in methanol (150 mL) was added Pd/C (10%, 500 mg). The mixture was hydrogenated at 50° C. under 10 atm of hydrogen pressure for 5 h. The resulting mixture was filtered and the filter cake was washed with MeOH (3×50 mL). The combined filtrate was concentrated to afford 1-methanesulfonylpiperazine (1.1 g, 66.6%) as a white solid.

Step 3: 2-(4-methanesulfonylpiperazin-1-yl)ethan-1-ol. To a stirred solution of 1-methanesulfonylpiperazine (500 mg, 3.05 mmol) in DMF (6 mL) were added 2-bromoethan-1-ol (460 mg, 3.68 mmol) and DIPEA (1.6 mL, 9.19 mmol) at room temperature under nitrogen atmosphere. The resulting mixture was stirred for 7 h at 120° C. under nitrogen atmosphere. After cooling, the reaction mixture was concentrated and the residue was purified on silica gel column with DCM/MeOH (10:1) to afford 2-(4-methanesulfonylpiperazin-1-yl)ethan-1-ol (230 mg, 36.3%) as a yellow oil. ES-MS (m/z): [M+1]⁺=209.2.

Step 4: 5-(2-(difluoromethyl)-6-methylpyridin-4-yl)-4-(4-fluorophenyl)-6-(2-(4-(methylsulfonyl)piperazin-1-yl)ethoxy)pyrimidin-2-amine Following step 2, example 8. From 2-(4-methanesulfonylpiperazin-1-yl)ethan-1-ol, 60% NaH and 4-chloro-5-[2-(difluoromethyl)-6-methylpyridin-4-yl]-6-(4-fluorophenyl)pyrimidin-2-amine in DMF. ES-MS (m/z): [M+1]⁺=537.2.

### Example 50

### 2-((2-amino-5-(2-(difluoromethyl)-6-methylpyridin-4-yl)-6-(4-fluorophenyl)pyrimidin-4-yl)oxy)-N-(3-(methylsulfonyl)propyl)acetamide

Following step 3, example 32. From 2-([2-amino-5-[2-(difluoromethyl)-6-methylpyridin-4-yl]-6-(4-fluorophenyl)pyrimidin-4-yl]oxy)acetic acid, 3-methanesulfonylpropan-1-amine hydrochloride, DIPEA and HATU in DCM. ES-MS (m/z): [M+1]⁺=524.1.

### Example 51

### methyl 4-(2-((2-amino-5-(2-(difluoromethyl)-6-methylpyridin-4-yl)-6-(4-fluorophenyl)pyrimidin-4-yl)oxy)ethyl)piperazine-1-carboxylate

Step 1: tert-butyl 4-(2-((2-amino-5-(2-(difluoromethyl)-6-methylpyridin-4-yl)-6-(4-fluorophenyl)pyrimidin-4-yl)oxy)ethyl)piperazine-1-carboxylate. Following step 2, example 8. From 2-methoxyethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=559.0.

Step 2: 5-(2-(difluoromethyl)-6-methylpyridin-4-yl)-4-(4-fluorophenyl)-6-(2-(piperazin-1-yl)ethoxy)pyrimidin-2-amine hydrochloride. To a mixture of tert-butyl 4-(2-((2-amino-5-(2-(difluoromethyl)-6-methylpyridin-4-yl)-6-(4-fluorophenyl)pyrimidin-4-yl)oxy)ethyl)piperazine-1-carboxylate (180 mg, 0.32 mmol) in ethyl acetate (4 mL) was added HCl in ethyl acetate (4.0 M, 10 mL). After stirring at room temperature for 3 h, the reaction mixture was concentrated to afford 5-(2-(difluoromethyl)-6-methylpyridin-4-yl)-4-(4-fluorophenyl)-6-(2-(piperazin-1-yl)ethoxy)pyrimidin-2-amine hydrochloride (160 mg, 100%) as a yellow solid. ES-MS (m/z): [M+1]⁺=459.

Step 3: methyl 4-(2-((2-amino-5-(2-(difluoromethyl)-6-methylpyridin-4-yl)-6-(4-fluorophenyl)pyrimidin-4-yl)oxy)ethyl)piperazine-1-carboxylate. To a mixture of 5-(2-(difluoromethyl)-6-methylpyridin-4-yl)-4-(4-fluorophenyl)-6-(2-(piperazin-1-yl)ethoxy)pyrimidin-2-amine hydrochloride (70 mg, 0.14 mmol), triethylamine (43 mg, 0.43 mmol) in DCM (4 mL) was added a solution of methyl chloroformate (13 mg, 0.14 mmol) in DCM (1 mL) dropwise at room temperature under nitrogen atmosphere. After stirring at room temperature for 2 h, the reaction mixture was concentrated. The residue was purified by prep-HPLC to afford methyl 4-(2-((2-amino-5-(2-(difluoromethyl)-6-methylpyridin-4-yl)-6-(4-fluorophenyl)pyrimidin-4-yl)oxy)ethyl)piperazine-1-carboxylate (41.5 mg, 57%) as a white solid. ES-MS (m/z): [M+1]⁺=517.2.

### Example 52

### 5-(2-(difluoromethyl)-6-methylpyridin-4-yl)-4-(4-fluorophenyl)-6-(2-(4-(oxetan-3-yl)piperazin-1-yl)ethoxy)pyrimidin-2-amine

Step 1: 5-(2-(difluoromethyl)-6-methylpyridin-4-yl)-4-(4-fluorophenyl)-6-(2-(4-(oxetan-3-yl)piperazin-1-yl)ethoxy)pyrimidin-2-amine A mixture of 5-(2-(difluoromethyl)-6-methylpyridin-4-yl)-4-(4-fluorophenyl)-6-(2-(piperazin-1-yl)ethoxy)pyrimidin-2-amine hydrochloride (80 mg, 0.16 mmol), oxetan-3-one (12 mg, 0.16 mmol) and acetic acid (19 mg, 0.32 mmol) in MeOH (10 mL) was stirred at room temperature for 30 min NaBH₃CN (37 mg, 0.32 mmol) then was added. The reaction mixture was stirred at room temperature overnight under nitrogen atmosphere, then was concentrated. The residue was purified by prep-HPLC to give 5-(2-(difluoromethyl)-6-methylpyridin-4-yl)-4-(4-fluorophenyl)-6-(2-(4-(oxetan-3-yl)piperazin-1-yl)ethoxy)pyrimidin-2-amine (21.6 mg, 26%) as a white solid. ES-MS (m/z): [M+1]⁺=515.0.

### Example 53

### 5-(2-(difluoromethyl)-6-methylpyridin-4-yl)-4-(4-fluorophenyl)-6-(2-(4-(oxetan-3-yl)piperazin-1-yl)ethoxy)pyrimidin-2-amine

Step 1: 4-(4-hydroxy-phenyl)-piperazine-1-carboxylic acid tert-butyl ester. To a mixture of 4-piperazin-1-yl-phenol (5.0 g, 28.1 mmol) in DCM (100 mL) were added triethylamine (5.7 g, 56.1 mmol) and (Boc)₂O (6.4 g, 30.9 mmol) at room temperature. After stirring at room temperature overnight, the reaction mixture was concentrated and purified on silica gel column (PE:EA=1:1) to obtain 4-(4-hydroxy-phenyl)-piperazine-1-carboxylic acid tert-butyl ester (7.0 g, 89%) as an off-white solid. ES-MS (m/z): [M+1]⁺=279.

Step 2: 4-[4-(2-methoxy-ethoxy)-phenyl]-piperazine-1-carboxylic acid tert-butyl ester. To a solution of 4-(4-hydroxy-phenyl)-piperazine-1-carboxylic acid tert-butyl ester (2.0 g, 7.2 mmol) in DMF (30 mL) were added 1-bromo-2-methoxy-ethane (2.0 g, 14.4 mmol) and Cs₂CO₃ (7.0 g, 21.6 mmol) at room temperature. After stirring at room temperature overnight, the reaction mixture was quenched with H₂O (90 mL), extracted with ethyl acetate (50 mL×3). The combined organic extracts were washed with brine (100 mL), dried over Na₂SO₄, filtered and concentrated. The residue was purified on silica gel column (PE:EA=2:1) to give 4-[4-(2-methoxy-ethoxy)-phenyl]-piperazine-1-carboxylic acid tert-butyl ester (2.1 g, 87%) as a light yellow solid. ES-MS (m/z): [M+1]⁺=337.

Step 3: 1-[4-(2-methoxy-ethoxy)-phenyl]-piperazine hydrochloride. To a mixture of 4-[4-(2-methoxy-ethoxy)-phenyl]-piperazine-1-carboxylic acid tert-butyl ester (1.0 g, 2.97 mmol) in MeOH (5 mL) was added HCl/MeOH (1.0 M, 10 mL) at 0° C. After stirring at room temperature for 2 h, the reaction mixture was concentrated and petroleum ether (20 mL) was added. The resulting mixture was stirred at room temperature for 30 min and filtered to get 1-[4-(2-methoxy-ethoxy)-phenyl]-piperazine hydrochloride (450 mg, 55%) as a yellow solid. ES-MS (m/z): [M+1]⁺=237.

Step 4: 2-(4-(4-(2-methoxyethoxy)phenyl)piperazin-1-yl)ethan-1-ol. To a mixture of 1-[4-(2-methoxy-ethoxy)-phenyl]-piperazine hydrochloride (200 mg, 0.73 mmol) in DMF (5 mL) were added K₂CO₃ (303 mg, 2.20 mmol) and 2-chloro-ethanol (71 mg, 0.88 mmol). This mixture was stirred at 50° C. overnight, cooled to room temperature and quenched with H₂O (15 mL). The mixture was extracted with ethyl acetate (20 mL×3). The combined organic extracts were washed with brine (50 mL), dried over Na₂SO₄, filtered and concentrated. The residue was purified by silica gel column (PE:EA=2:1) to afford 2-(4-(4-(2-methoxyethoxy)phenyl)piperazin-1-yl)ethan-1-ol (80 mg, 39%) as a white solid. ES-MS (m/z): [M+1]⁺=281.

Step 5: 5-(2-Difluoromethyl-6-methyl-pyridin-4-yl)-4-(4-fluoro-phenyl)-6-(2-{4-[4-(2-methoxy-ethoxy)-phenyl]-piperazin-1-yl}-ethoxy)-pyrimidin-2-ylamine Following step 2, example 8. From 2-(4-(4-(2-methoxyethoxy)phenyl)piperazin-1-yl)ethan-1-ol, 60% NaH and 4-chloro-5-(2-difluoromethyl-6-methyl-pyridin-4-yl)-6-(4-fluoro-phenyl)-pyrimidin-2-ylamine in DMF. ES-MS (m/z): [M+1]⁺=609.3.

### Example 54

### ethyl(2-((2-amino-5-(2-(difluoromethyl)-6-methylpyridin-4-yl)-6-(4-fluorophenyl)pyrimidin-4-yl)oxy)ethyl)carbamate

Step 1: tert-butyl(2-((2-amino-5-(2-(difluoromethyl)-6-methylpyridin-4-yl)-6-(4-fluorophenyl)pyrimidin-4-yl)oxy)ethyl)carbamate. Following step 2, example 8. From tert-butyl (2-hydroxyethyl)carbamate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=490.3.

Step 2: 4-(2-aminoethoxy)-5-(2-(difluoromethyl)-6-methylpyridin-4-yl)-6-(4-fluorophenyl)pyrimidin-2-amine. A solution of tert-butyl(2-((2-amino-5-(2-(difluoromethyl)-6-methylpyridin-4-yl)-6-(4-fluorophenyl)pyrimidin-4-yl)oxy)ethyl)carbamate (100 mg, 0.21 mmol) and MeOH/HCl (1.0 M, 2.0 mL) was stirred at 50° C. for 30 min The mixture was cooled and concentrated, and the residue was triturated with NH₃ in methanol (1.0 M, 10 mL). The mixture was concentrated to give 4-(2-aminoethoxy)-5-(2-(difluoromethyl)-6-methylpyridin-4-yl)-6-(4-fluorophenyl)pyrimidin-2-amine (100 mg, crude) as a white solid. ES-MS (m/z): [M+1]⁺=390.3.

Step 3: ethyl (2-((2-amino-5-(2-(difluoromethyl)-6-methylpyridin-4-yl)-6-(4-fluorophenyl)pyrimidin-4-yl)oxy)ethyl)carbamate. To a mixture of 4-(2-aminoethoxy)-5-(2-(difluoromethyl)-6-methylpyridin-4-yl)-6-(4-fluorophenyl)pyrimidin-2-amine (100 mg, 0.21 mmol), DIPEA (81 mg, 0.63 mmol) in DCM (2 mL) was added ethyl chloroformate (35 mg, 0.32 mmol) at room temperature. After stirring at room temperature for 1 h, the mixture was concentrated and the residue was purified by C18 reverse phase silica gel column (ACN: H₂O) to give ethyl (2-((2-amino-5-(2-(difluoromethyl)-6-methylpyridin-4-yl)-6-(4-fluorophenyl)pyrimidin-4-yl)oxy)ethyl)carbamate (12 mg, 13%) as a white solid. ES-MS (m/z): [M+1]⁺=462.1.

### Example 55

### 5-(2-(difluoromethyl)-6-methylpyridin-4-yl)-4-(4-fluorophenyl)-6-((tetrahydro-2H-pyran-4-yl)methoxy)pyrimidin-2-amine

Step 1: 5-(2-(difluoromethyl)-6-methylpyridin-4-yl)-4-(4-fluorophenyl)-6-((tetrahydro-2H-pyran-4-yl)methoxy)pyrimidin-2-amine Following step 2, example 8. From (tetrahydro-2H-pyran-4-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=445.0.

### Example 56

### 5-(2-(difluoromethyl)-6-methylpyridin-4-yl)-4-(4-fluorophenyl)-6-(2-(4-(4-(methylsulfonyl)phenyl)piperazin-1-yl)ethoxy)pyrimidin-2-amine

Step 1: benzyl 4-(4-methanesulfonylphenyl)piperazine-1-carboxylate. To a solution of benzyl piperazine-1-carboxylate (5.00 g, 22.7 mmol) and 1-bromo-4-methanesulfonylbenzene (5.34 g, 22.699 mmol) in DMF (10 mL) were added Cs₂CO₃ (14.79 g, 45.4 mmol), Pd(OAc)₂ (509.6 mg, 2.27 mmol,) and XPhos (1082.1 mg, 2.27 mmol) under a nitrogen atmosphere. The reaction mixture was stirred under microwave irradiation for 3 h at 150° C. The mixture was cooled and diluted with ethyl acetate (50 mL). The resulting mixture was washed with 3×30 mL of water, then 30 mL of brine. The organic phase was dried over anhydrous Na₂SO₄, filtered and concentrated. The residue was purified on silica gel column with DCM/MeOH (2/1) to afford benzyl 4-(4-methanesulfonylphenyl)piperazine-1-carboxylate (2.1 g, 24.7%) as a light brown solid. ES-MS (m/z): [M+1]⁺=375.5.

Step 2: 1-(4-methanesulfonylphenyl)piperazine. To a solution of benzyl 4-(4-methanesulfonylphenyl)piperazine-1-carboxylate (2.1 g, 5.61 mmol) in MeOH (20 mL) and DCM (4 mL) was added Pd/C (10%, 59.7 mg, 0.56 mmol). The mixture was stirred at 50° C. for 5 h under hydrogen atmosphere (1 atm). The reaction mixture was cooled and filtered through a celite pad and concentrated to obtain 1-(4-methanesulfonylphenyl)piperazine (1.1 g, 81.6%) as a light yellow solid. ES-MS (m/z): [N4+1]+=241.2.

Step 3: 2-[4-(4-methanesulfonylphenyl)piperazin-1-yl]ethan-1-ol. Following step 2, example 49. From 1-(4-methanesulfonylphenyl)piperazine, 2-bromoethan-1-ol and K₂CO₃ in DMF. ES-MS (m/z): [M+1]⁺=285.2.

Step 4: 5-(2-(difluoromethyl)-6-methylpyridin-4-yl)-4-(4-fluorophenyl)-6-(2-(4-(4-(methylsulfonyl)phenyl)piperazin-1-yl)ethoxy)pyrimidin-2-amine Following step 2, example 8. From 2-[4-(4-methanesulfonylphenyl)piperazin-1-yl]ethan-1-ol, 60% NaH and 4-chloro-5-[2-(difluoromethyl)-6-methylpyridin-4-yl]-6-(4-fluorophenyl)pyrimidin-2-amine in DMF. ES-MS (m/z): =613.3.

### Example 57

### 5-(2-(difluoromethyl)-6-methylpyridin-4-yl)-4-(4-fluorophenyl)-6-(4-(4-methylpiperazin-1-yl)phenethoxy)pyrimidin-2-amine

Step 1: 1-(4-[2-(tert-butyldimethylsilyl)oxy]ethyl)phenyl)piperazine. To a stirred solution [2-(4-bromophenyl)ethoxy](tert-butyl)dimethylsilane (1.00 g, 3.17 mmol) and piperazine (0.33 g, 3.81 mmol) in toluene (10 mL) were added BINAP (100 mg, 0.16 mmol), Pd₂(dba)₃ (146 mg, 0.16 mmol) and t-BuONa (917 mg, 9.54 mmol) at room temperature under nitrogen atmosphere. After stirring for 16 h at 100° C. under nitrogen atmosphere, the mixture was cooled and filtered, and the filter cake was washed with DCM (50 mL). The combined filtrate was concentrated and the residue was purified on silica gel column with DCM/MeOH (20:1) to afford 1-(4-[2-[(tert-butyldimethylsilyl)oxy]ethyl]phenyl)piperazine (631 mg, 62.1%) as a brown oil. ES-MS (m/z): [M+1]⁺=321.3.

Step 2: 1-(4-[2-[(tert-butyldimethylsilyl)oxy]ethyl]phenyl)-4-methylpiperazine. To a stirred mixture of 1-(4-(2-[(tert-butyldimethylsilyl)oxylethyl]phenyl)piperazine (300.0 mg, 0.94 mmol) and K₂CO₃ (258.7 mg, 1.87 mmol) in ACN (10 mL) was added MeI (132.8 mg, 0.94 mmol) dropwise at room temperature under nitrogen atmosphere. The resulting mixture was stirred for 16 h at room temperature, then was quenched with EtOH (0.5 mL). The resulting mixture was filtered and the filter cake was washed with DCM (2×10 mL). The combined filtrate was concentrated and the residue was purified on silica gel column with DCM/MeOH (20/1) to afford 1-(4-(2-[(tert-butyldimethylsilyl)oxylethyl]phenyl)-4-methylpiperazine (186 mg, 59.4%) as a brown oil. ES-MS (m/z): [M+1]⁺=335.4.

Step 3: 2-[4-(4-methylpiperazin-1-yl)phenyl]ethan-1-ol. To a stirred solution of 1-(4-[2-1(tert-butyldimethylsilyl)oxylethyl]phenyl)-4-methylpiperazine (186.0 mg, 0.56 mmol) in THF (3 mL) was added TBAF (290.7 mg, 1.11 mmol) in portions at room temperature. After stirring for 5 h at room temperature, the reaction mixture was diluted with ethyl acetate (10 mL), washed with 3×10 mL of water, then 10 mL of brine. The organic layer was dried and concentrated. The residue was purified on silica gel column with DCM/MeOH (20/1) to afford 2-[4-(4-methylpiperazin-1-yl)phenyl]ethan-1-ol (50 mg, 40.8%) as a light yellow oil. ES-MS (m/z): [M+1]⁺=221.2.

Step 4: 5-(2-(difluoromethyl)-6-methylpyridin-4-yl)-4-(4-fluorophenyl)-6-(4-(4-methylpiperazin-1-yl)phenethoxy)pyrimidin-2-amine Following step 2, example 8. From 2-[4-(4-methylpiperazin-1-yl)phenyl]ethan-1-ol, 60% NaH and 4-chloro-5-[2-(difluoromethyl)-6-methylpyridin-4-yl]-6-(4-fluorophenyl)pyrimidin-2-amine in DMF. ES-MS (m/z): [M+1]⁺=549.5.

### Example 58

### 5-(2-(difluoromethyl)-6-methylpyridin-4-yl)-4-(4-fluorophenyl)-6-(4-(4-(methylsulfonyl)piperazin-1-yl)phenethoxy)pyrimidin-2-amine

Step 1: 1-(4-(2-[(tert-butyldimethylsilyl)oxylethyl]phenyl)-4-methanesulfonylpiperazine. To a stirred mixture of 1-(4-[2-1(tert-butyldimethylsilyl)oxylethyl]phenyl)piperazine hydrochloride (136.0 mg, 0.38 mmol) and DIPEA (98.5 mg, 0.76 mmol) in DCM (2 mL) was added MsCl (52.4 mg, 0.46 mmol) dropwise at room temperature under nitrogen atmosphere. After stirring for 4 h at room temperature, the reaction mixture was quenched with H₂O (0.1 mL) and concentrated. The residue was purified on silica gel column with DCM/MeOH (20/1) to afford 1-(4-[2-[(tert-butyldimethylsilyl)oxy]ethyl]phenyl)-4-methanesulfonylpiperazine (136 mg, 89.6%) as a light oil . ES-MS (m/z): [M+1]⁺=421.3.

Step 2: 2-[4-(4-methanesulfonylpiperazin-1-yl)phenyl]ethan-1-ol. To a stirred solution of 1-(4-[2-[(tert-butyldimethylsilyl)oxy]ethyl]phenyl)-4-methanesulfonylpiperazine (138.0 mg, 0.35 mmol) in THF (2 mL) was added TBAF (181.0 mg, 0.69 mmol) in portions at room temperature. After stirring for 2 h at room temperature, the reaction mixture was diluted with ethyl acetate (20 mL), washed with 3×10 mL of water, then 10 mL of brine. The organic layer was dried and concentrated. The residue was purified on silica gel column with DCM/MeOH (20/1) to afford 2-[4-(4-methanesulfonylpiperazin-1-yl)phenyl]ethan-1-ol (68 mg, 69.1%) as a yellow oil. ES-MS (m/z): [M+1]⁺=285.2.

Step 3: 5-(2-(difluoromethyl)-6-methylpyridin-4-yl)-4-(4-fluorophenyl)-6-(4-(4-(methylsulfonyl)piperazin-1-yl)phenethoxy)pyrimidin-2-amine. Following step 2, example 8. From 2-[4-(4-methanesulfonylpiperazin-1-yl)phenyl]ethan-1-ol, 60% NaH and 4-chloro-5-[2-(difluoromethyl)-6-methylpyridin-4-yl]-6-(4-fluorophenyl)pyrimidin-2-amine in DMF. ES-MS (m/z): [M+1]⁺=613.2.

### Example 59

### (2-amino-5-(2-(difluoromethyl)-6-methylpyridin-4-yl)-6-(4-fluorophenyl)pyrimidin-4-yl)oxy)ethyl)sulfuric diamide

Step 1: tert-butyl N-(2-hydroxyethyl)sulfamoylcarbamate. To a solution of sulfurisocyanatidic chloride (2.0 g, 14.2 mmol) in DCM (20 mL) was added a solution of t-BuOH (1.5 g, 21.3 mmol) in DCM (10 mL) at 0° C. After stirring for 30 min at 0° C., TEA (2.1 g, 21.3 mmol was added. The reaction mixture was stirred continually for 30 min at 0° C., 2-aminoethanol (865.0 mg, 14.2 mmol) then was added at 0° C. The reaction mixture was warmed to room temperature slowly and stirred overnight. The reaction mixture was diluted with DCM (30 mL) and washed with 0.1 N aqueous HCl. The aqueous phase was adjusted to pH=4 with 1 N aqueous HCl and extracted with ethyl acetate (30 mL×2). The combined organic extracts were washed with brine (30 mL), dried over Na₂SO₄, filtered and concentrated to get tert-butyl N-(2-hydroxyethyl)sulfamoylcarbamate (2.5 g, 73%) as a white solid.

Step 2: tert-butyl N-(2-((2-amino-5-(2-(difluoromethyl)-6-methylpyridin-4-yl)-6-(4-fluorophenyl)pyrimidin-4-yl)oxy)ethyl)sulfamoylcarbamate. Following step 2, example 8. From tert-butyl N-(2-hydroxyethyl)sulfamoylcarbamate, 60% NaH and 4-chloro-5-(2-(difluoromethyl)-6-methylpyridin-4-yl)-6-(4-fluorophenyl)pyrimidin-2-amine in DMF.

Step 3: (2-amino-5-(2-(difluoromethyl)-6-methylpyridin-4-yl)-6-(4-fluorophenyl)pyrimidin-4-y)oxy)ethyl)sulfuric diamide. To a solution of tert-butyl N-(2-((2-amino-5-(2-(difluoromethyl)-6-methylpyridin-4-yl)-6-(4-fluorophenyl)pyrimidin-4-yl)oxy)ethyl)sulfamoylcarbamate (150.0 mg, 0.26 mmol) in MeOH (2 mL) was added HCl/MeOH (1.0 mL, 1 M, 1.0 mmol). After stirring at 30° C. for 2 h, the reaction mixture was neutralized with NH₃ in MeOH, then concentrated. The residue was purified by flash chromatography (DCM:MeOH=30:1) to obtain (2-amino-5-(2-(difluoromethyl)-6-methylpyridin-4-yl)-6-(4-fluorophenyl)pyrimidin-4-yl)oxy)ethyl)sulfuric diamide (26.0 mg, 21%) as an off-white solid. ES-MS (m/z): [M+1]⁺=469.1.

### Example 60

### 1-(2-((2-amino-5-(2-(difluoromethyl)-6-methylpyridin-4-yl)-6-(4-fluorophenyl)pyrimidin-4-yl)oxy)ethyl)-3 -ethylurea

Step 1: 1-ethyl-3-(2-hydroxyethyl)urea. To a mixture of 2-aminoethanol (2.0 g, 32.7 mmol) in DCM (100 mL) was added DIPEA (8.45 g, 65.5 mmol) and isocyanatoethane (3.03 g, 42.6 mmol) at 0° C. After stirring for 30 min at 0° C., the reaction mixture was warmed slowly to room temperature, then stirred overnight. The reaction mixture was concentrated and the residue was purified by reverse phase silica gel column to get 1-ethyl-3-(2-hydroxyethyl)urea (2.8 g, 65%) as a colorless oil.

Step 2: 1-(2-((2-amino-5-(2-(difluoromethyl)-6-methylpyridin-4-yl)-6-(4-fluorophenyl)pyrimidin-4-yl)oxy)ethyl)-3-ethylurea. Following step 2, example 8. From 1-ethyl-3-(2-hydroxyethyl)urea, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=461.0.

### Example 61

### 3-((2-amino-5-(2-(difluoromethyl)-6-methylpyridin-4-yl)-6-(4-fluorophenyl)pyrimidin-4-yl)oxy)-2,2-dimethylpropanoic acid

Step 1: 3-((2-amino-5-(2-(difluoromethyl)-6-methylpyridin-4-yl)-6-(4-fluorophenyl)pyrimidin-4-yl)oxy)-2,2-dimethylpropanoic acid. Following step 2, example 8. From 3-hydroxy-2,2-dimethylpropanoic acid, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=447.0.

### Example 62

### 1-((2-amino-5-(2-(difluoromethyl)-6-methylpyridin-4-yl)-6-(4-fluorophenyl)pyrimidin-4-yl)oxy)-2-methylpropan-2-ol

Step 1: 1-((2-amino-5-(2-(difluoromethyl)-6-methylpyridin-4-yl)-6-(4-fluorophenyl)pyrimidin-4-yl)oxy)-2-methylpropan-2-ol. Following step 2, example 18. From 2-methylpropane-1,2-diol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=419.1.

### Example 63

### Ethyl((2-amino-5-(2-methyl-6-(trifluoromethyl)pyridin-4-yl)-6-phenylpyrimidin-4-yl)methyl)carbamate

Step 1: 2-amino-5-[2-methyl-6-(trifluoromethyl)pyridin-4-yl]-6-phenylpyrimidine-4-carbonitrile. To a solution of 4-chloro-5-[2-methyl-6-(trifluoromethyl)pyridin-4-yl]-6-phenylpyrimidin-2-amine (365.0 mg, 1.00 mmol) and Zn(CN)₂ (141.0 mg, 1.20 mmol) in DMF (3 mL) was added Pd(PPh₃)₄ (115.6 mg, 0.10 mmol) under nitrogen atmosphere at room temperature. The reaction mixture was stirred under microwave irradiation for 2 h at 120° C., then was cooled and diluted with ethyl acetate (30 mL). The resulting mixture was washed with 3×10 mL of water, then 10 mL of brine. The organic phase was dried over anhydrous Na₂SO₄, filtered and concentrated. The residue was purified on silica gel column with PE/EA (3/1) to afford 2-amino-5-[2-methyl-6-(trifluoromethyl)pyridin-4-yl]-6-phenylpyrimidine-4-carbonitrile (307 mg, 86.3%) as an off-white solid. ES-MS (m/z): [M+1]⁺=356.0.

Step 2: 4-(aminomethyl)-5-[2-methyl-6-(trifluoromethyl)pyridin-4-yl]-6-phenylpyrimidin-2-amine To a solution of 2-amino-5-[2-methyl-6-(trifluoromethyl)pyridin-4-yl]-6-phenylpyrimidine-4-carbonitrile (307 mg, 0.86 mmol) in MeOH (5 mL) and NH₃ (7 M in MeOH, 0.5 mL) was added nickel (30 mg, 0.51 mmol) under nitrogen atmosphere. The mixture was stirred at room temperature for 16 h under hydrogen atmosphere (1 atm). The reaction solution was filtered through a celite pad and concentrated to afford 4-(aminomethyl)-5-[2-methyl-6-(trifluoromethyl)pyridin-4-yl]-6-phenylpyrimidin-2-amine (306 mg, 98.6%) as a light green solid. ES-MS (m/z): [M+1]⁺=360.2.

Step 3: ethyl N-([2-amino-5-[2-methyl-6-(trifluoromethyl)pyridin-4-yl]-6-phenylpyrimidin-4-yl]methyl)carbamate. To a stirred mixture of 4-(aminomethyl)-5-[2-methyl-6-(trifluoromethyl)pyridin-4-yl]-6-phenylpyrimidin-2-amine (100 mg, 0.28 mmol) and triethylamine (84.5 mg, 0.84 mmol) in DCM (3 mL) was added ethyl chloroformate (30.2 mg, 0.28 mmol) dropwise at 0° C. under nitrogen atmosphere. After stirring for 16 h at room temperature, the reaction mixture was quenched with EtOH (0.5 mL). The resulting mixture was concentrated and the residue was purified by C18 silica gel reverse phase chromatography to afford ethyl N-([2-amino-5-[2-methyl-6-(trifluoromethyl)pyridin-4-yl]-6-phenylpyrimidin-4-yl]methyl)carbamate (10 mg, 8.3%) as an off-white solid. ES-MS (m/z): [M+1]⁺=432.0.

### Example 64

### 5-[2-(difluoromethyl)-6-methylpyridin-4-yl]-4-(4-fluorophenyl)-6-[2-[4-(4-methanesulfonylpiperazin-1-yl)phenyl]ethoxy]pyrimidin-2-amine

Step 1: 1-(4-[2-[(tert-butyldimethylsilyl)oxy]ethyl]phenyl)-4-(oxetan-3-yl)piperazine. To a stirred mixture of 1-(4-[2-[(tert-butyldimethylsilyl)oxy]ethyl]phenyl)piperazine (300.0 mg, 0.94 mmol) and oxetan-3-one (134.9 mg, 1.87 mmol) in DCE (3 mL) were added NaBH(OAc)₃ (297.5 mg, 1.40 mmol) and AcOH (11.2 mg, 0.19 mmol) at room temperature under nitrogen atmosphere. After stirring for 2 h at room temperature, the reaction mixture was diluted with ethyl acetate (10 mL), then washed with 3×10 mL of water, followed by 10 mL of brine. The organic layer was dried and concentrated. The residue was purified on silica gel column with PE/EA (4/1) to afford 1-(4-[2-(tert-butyldimethylsilyl)oxy)ethyl]phenyl)-4-(oxetan-3-yl)piperazine (186 mg, 52.8%) as a light yellow oil. ES-MS (m/z): [M+1]⁺=377.2.

Step 2: 2-[4-[4-(oxetan-3-yl)piperazin-1-yl]phenyl]ethan-1-ol. Following step 2, example 58. From 1-(4-[2-(tert-butyldimethylsilyl)oxy]ethyl)phenyl)-4-(oxetan-3-yl)piperazine and TBAF in THF. ES-MS (m/z): [M+1]⁺=263.2.

Step 3: 5-[2-(difluoromethyl)-6-methylpyridin-4-yl]-4-(4-fluorophenyl)-6-(2-[4-[4-(oxetan-3-yl)piperazin-1-yl]phenyl]ethoxy)pyrimidin-2-amine. Following step 2, example 8. From 2-[4-[4-(oxetan-3-yl)piperazin-1-yl]phenyl]ethan-1-ol, 60% NaH and 4-chloro-5-[2-(difluoromethyl)-6-methylpyridin-4-yl]-6-(4-fluorophenyl)pyrimidin-2-amine in DMF. ES-MS (m/z): [M+1]⁺=591.2.

### Example 65

### 5-(2-(difluoromethyl)-6-methylpyridin-4-yl)-4-(2-(ethylamino)ethoxy)-6-(4-fluorophenyl)pyrimidin-2-amine

Step 1: tert-butyl N-ethyl-N-(2-hydroxyethyl)carbamate. To a stirred mixture of 2-(ethylamino)ethan-1-ol (500.0 mg, 5.61 mmol) and triethylamine (1135.2 mg, 11.22 mmol) in DCM (5 mL) was added di-tert-butyl dicarbonate (1836.3 mg, 8.41 mmol) at room temperature. After stirring for 16 h, the reaction mixture was diluted with DCM (30 mL), then washed with 2×30 mL of water, followed by 30 mL of brine. The organic layer was dried, filtered and concentrated. The residue was purified on silica gel column with DCM/MeOH (20/1) to afford tert-butyl N-ethyl-N-(2-hydroxyethyl)carbamate (432 mg, 40.7%) as an off-white solid.

Step 2. 5-(2-(difluoromethyl)-6-methylpyridin-4-yl)-4-(2-(ethylamino)ethoxy)-6-(4-fluorophenyl)pyrimidin-2-amine. Following step 2, example 8. From tert-butyl N-ethyl-N-(2-hydroxyethyl)carbamate, 60% NaH and 4-chloro-5-[2-(difluoromethyl)-6-methylpyridin-4-yl]-6-(4-fluorophenyl)pyrimidin-2-amine in DMF. The crude product was treated with HCl in 1,4-dioxane (0.5 mL, 4 M) for 3 h at room temperature. The resulting mixture was concentrated and purified by Prep-HPLC to afford 5-[2-(difluoromethyl)-6-methylpyridin-4-yl]-4-[2-(ethylamino)ethoxy]-6-(4-fluorophenyl)pyrimidin-2-amine (18.8 mg, 17.1%) as an off-white solid. ES-MS (m/z): [M+1]⁺=418.0.

### Example 66

### 5-(2-(difluoromethyl)-6-methylpyridin-4-yl)-4-(4-fluorophenyl)-6-(4-((1-(methylsulfonyl)pyrrolidin-3-yl)oxy)phenethoxy)pyrimidin-2-amine

Step 1: tert-butyl 3-[4-(2-hydroxyethyl)phenoxy]pyrrolidine-1-carboxylate. To a solution of tert-butyl 3-hydroxypyrrolidine-1-carboxylate (1.10 g, 5.88 mmol) and triethylamine (1.78 g, 17.63 mmol) in DCM (15 mL) was added MsCl (0.81 g, 7.05 mmol) at room temperature. After stirring for 1 h at room temperature, the reaction mixture was diluted with DCM (20 mL), washed with 3×20 mL of water, followed by 20 mL of brine. The organic layer was dried over anhydrous Na₂SO₄, filtered and concentrated. The residue was dissolved in DMF (10 mL), and to the resulting solution were added 4-(2-hydroxyethyl)phenol (0.97 g, 7.05 mmol) and Cs₂CO₃ (3.83 g, 11.75 mmol). The resulting mixture was stirred for additional 16 h at 80° C. The mixture was cooled and diluted with ethyl acetate (30 mL). The resulting mixture was washed with 3×20 mL of aq. Na₂CO₃, followed by 20 mL of brine. The organic layer was dried over anhydrous Na₂SO₄, filtered and concentrated. The residue was purified on silica gel column with DCM/MeOH (20/1) to afford tert-butyl 3-[4-(2-hydroxyethyl)phenoxy]pyrrolidine-1-carboxylate (400 mg, 22.2%) as a yellow solid. ES-MS (m/z): [M+1]⁺=330.4.

Step 2: tert-butyl 3-[4-[2-([2-amino-5-[2-(difluoromethyl)-6-methylpyridin-4-yl]-6-(4-fluorophenyl)pyrimidin-4-yl]oxy)ethyl]phenoxy]pyrrolidine-1-carboxylate. Following step 2, example 8. From tert-butyl 3-[4-(2-hydroxyethyl)phenoxy]pyrrolidine-1-carboxylate, 60% NaH and 4-chloro-5-[2-(difluoromethyl)-6-methylpyridin-4-yl]-6-(4-fluorophenyl)pyrimidin-2-amine in DMF. ES-MS (m/z): [M+1]⁺=636.5.

Step 3: 5-[2-(difluoromethyl)-6-methylpyridin-4-yl]-4-(4-fluorophenyl)-6-(2-[4-(pyrrolidin-3-yloxy)phenyl]ethoxylpyrimidin-2-amine hydrochloride. To a stirred solution of tert-butyl 3-[4-[2-([2-amino-5-[2-(difluoromethyl)-6-methylpyridin-4-yl]-6-(4-fluorophenyl)pyrimidin-4-yl]oxy)ethyl]phenoxy]pyrrolidine-1-carboxylate (233.0 mg, 0.37 mmol) in 1,4-dioxane (1 mL) was added HCl in 1,4-dioxane (5 mL, 4 M) dropwise at room temperature. After stirring for 1 h at room temperature, the reaction mixture was concentrated and the residue was triturated with Et₂O (2 mL) to obtain 5-[2-(difluoromethyl)-6-methylpyridin-4-yl]-4-(4-fluorophenyl)-6-[2-[4-(pyrrolidin-3-yloxy)phenyl]ethoxy]pyrimidin-2-amine hydrochloride (56 mg, 26.7%) as an off-white solid. ES-MS (m/z): [M+1]⁺=536.2.

Step 4: 5-(2-(difluoromethyl)-6-methylpyridin-4-yl)-4-(4-fluorophenyl)-6-(4-((1-(methylsulfonyl)pyrrolidin-3-yl)oxy)phenethoxy)pyrimidin-2-amine. To a stirred mixture of 5-[2-(difluoromethyl)-6-methylpyridin-4-yl]-4-(4-fluorophenyl)-6-[2-[4-(pyrrolidin-3-yloxy)phenyl]ethoxy]pyrimidin-2-amine hydrochloride (56.0 mg, 0.098 mmol) and DIPEA (50.6 mg, 0.39 mmol) in DCM (1 mL) was added MsCl (13.5 mg, 0.12 mmol) at room temperature. After stirring for 3 h at room temperature, the reaction mixture was concentrated under vacuum and the residue was purified by Prep-HPLC to afford 5[-2-(difluoromethyl)-6-methylpyridin-4-yl]-4-(4-fluorophenyl)-6-(2-[4-[(1-methanesulfonylpyrrolidin-3-yl)oxy]phenyl]ethoxy)pyrimidin-2-amine (12 mg, 20.0%) as an off-white solid. ES-MS (m/z): [M+1]⁺=614.2.

### Example 67

### 1-(4-(2-((2-amino-5 -(2-(difluoromethyl)-6-methylpyridin-4-yl)-6-(4-fluorophenyl)pyrimidin-4-yl)oxy)ethyl)phenyl)-3 -ethylurea

Step 1: 1-ethyl-3-(4-(2-hydroxyethyl)phenyl)urea. To a solution of 2-(4-aminophenyl)ethanol (200 mg, 1.46 mmol) and DIPEA (376 mg, 2.92 mmol) in DCM (10 mL) was added ethyl isocyanate (135 mg, 1.90 mmol) at 0° C. After stirring at 0° C. for 30 min, the reaction mixture was warmed slowly to room temperature, then stirred at this temperature overnight. The reaction mixture was filtered to obtain 1-ethyl-3-(4-(2-hydroxyethyl)phenyl)urea (220 mg, 72.6%) as a white solid. ES-MS (m/z): [M+1]⁺=208.9.

Step 2: 1-(4-(2-((2-amino-5-(2-(difluoromethyl)-6-methylpyridin-4-yl)-6-(4-fluorophenyl)pyrimidin-4-yl)oxy)ethyl)phenyl)-3-ethylurea. Following step 2, example 8. From 1-ethyl-3-(4-(2-hydroxyethyl)phenyl)urea, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=537.2.

### Example 68

### 2-(4-(2-((2-amino-5-(2-(difluoromethyl)-6-methylpyridin-4-yl)-6-(4-fluorophenyl)pyrimidin-4-yl)oxy)ethyl)phenyl)propan-2-ol

Step 1: (4-bromophenethoxy)(tert-butyl)dimethylsilane. To a stirred solution of 2-(4-bromophenyl)ethanol (5.0 g, 24.9 mmol) in DCM (50 mL) were added imidazole (5.0 g, 74.6 mmol) and TBSC1 (4.1 g, 27.4 mmol) at 0° C. under nitrogen atmosphere. After stirring at room temperature for 3 h, the reaction mixture was washed with water (20 mL), followed by brine (20 mL). The organic layer was dried over Na₂SO₄, filtered and concentrated. The residue was purified on silica gel column (PE:EA=50:1) to obtain (4-bromophenethoxy)(tert-butyl)dimethylsilane (6.2 g, 79.2%) as a colorless oil.

Step 2: 2-(4-(2-((tert-butyldimethylsilyl)oxy)ethyl)phenyl)propan-2-ol. To a stirred solution of (4-bromophenethoxy)(tert-butyl)dimethylsilane (500 mg, 1.59 mmol) in anhydrous THF (5 mL) was added n-BuLi (2.5M in THF, 0.77 mL, 1.90 mmol) dropwise at −78° C. Anhydrous acetone (184 mg, 3.17 mmol) then was added slowly at −78° C. The reaction mixture was stirred and warmed slowly to room temperature. The reaction mixture was quenched with water (10 mL), extracted with DCM. The organic layer was dried over Na₂SO₄, filtered and concentrated to obtain crude 2-(4-(2-((tert-butyldimethylsilyl)oxy)ethyl)phenyl)propan-2-ol (400 mg, 85.7%) as a colorless oil.

Step 3: 2-(4-(2-hydroxyethyl)phenyl)propan-2-ol. To a stirred solution of 2-(4-(2-((tert-butyldimethylsilyl)oxy)ethyl)phenyl)propan-2-ol (400 mg, 1.36 mmol) in THF (3 mL) was added TBAF (1 M in THF, 3.0 mL, 3.0 mmol) at room temperature. After stirring at room temperature for 2 h, the reaction mixture was concentrated and the residue was purified on silica gel column (PE:EA=2:1) to give 2-(4-(2-hydroxyethyl)phenyl)propan-2-ol (120mg, 49.0%) as a yellow oil.

Step 4: 2-(4-(2-((2-amino-5-(2-(difluoromethyl)-6-methylpyridin-4-yl)-6-(4-fluorophenyl)pyrimidin-4-yl)oxy)ethyl)phenyl)propan-2-ol. Following step 2, example 8. From 2-(4-(2-hydroxyethyl)phenyl)propan-2-ol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=509.2.

### Example 69

### N-(4-(2-((2-amino-5-(2-(difluoromethyl)-6-methylpyridin-4-yl)-6-(4-fluorophenyl)pyrimidin-4-yl)oxy)ethyl)phenyl)methanesulfonamide

Step 1: N-(4-(2-hydroxyethyl)phenyl)methanesulfonamide. To a stirred solution of 2-(4-aminophenyl)ethanol (500 mg, 3.65 mmol) and pyridine (433 mg, 5.47 mmol) in DCM (5 mL) was added MsCl (418 mg, 3.65 mmol) dropwise at 0° C. under nitrogen atmosphere. After stirring at room temperature overnight, the mixture was diluted with DCM (10 mL), washed with 1M HCl, followed by aqueous NaHCO₃. The organic layer was dried over Na₂SO₄, filtered and concentrated. The residue was purified on silica gel column (PE:EA=5:1 to 2:1) to give N-(4-(2-hydroxyethyl)phenyl)methanesulfonamide (60 mg, 7.7%) as a yellow oil.

Step 2: N-(4-(2-((2-amino-5-(2-(difluoromethyl)-6-methylpyridin-4-yl)-6-(4-fluorophenyl)pyrimidin-4-yl)oxy)ethyl)phenyl)methanesulfonamide Following step 2, example 8. From N-(4-(2-hydroxyethyl)phenyl)methanesulfonamide, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=544.1.

### Example 70

### 4-((2-amino-5-(2-(difluoromethyl)-6-methylpyridin-4-yl)-6-(4-fluorophenyl)pyrimidin-4-yl)oxy)-2,2-dimethylbutanoic acid

Step 1: 4-hydroxy-2,2-dimethylbutanoic acid. A mixture of 3,3-dimethyldihydrofuran-2(3H)-one (200 mg, 1.75 mmol), KOH (98 mg, 1.75 mmol) in H₂O (4 mL) was stirred at 110° C. for 2 h. The reaction mixture was cooled to 0° C., adjusted pH to about 5 with aqueous HCl solution. The resulting mixture was extracted with EtOAc (40 mL×3). The combined organic extracts were washed with brine (30 mL×2), dried over Na₂SO₄, filtered and concentrated to afford crude 4-hydroxy-2,2-dimethylbutanoic acid as a colorless oil (180 mg, yield: 78%).

Step 2: 4-((2-amino-5-(2-(difluoromethyl)-6-methylpyridin-4-yl)-6-(4-fluorophenyl)pyrimidin-4-yl)oxy)-2,2-dimethylbutanoic acid. Following step 2, example 8. From 4-hydroxy-2,2-dimethylbutanoic acid, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=461.1.

### Example 71

### 2-((2-amino-5-(2-(difluoromethyl)-6-methylpyridin-4-yl)-6-(4-fluorophenyl)pyrimidin-4-yl)oxy)acetic acid

This compound was obtained from step 2, example 32. ES-MS (m/z): [M+1]⁺=404.9.

### Example 72

### Methyl(4-(2-((2-amino-5-(2-(difluoromethyl)-6-methylpyridin-4-yl)-6-(4-fluorophenyl)pyrimidin-4-yl)oxy)ethyl)phenyl)carbamate

Step 1: methyl(4-(2-hydroxyethyl)phenyl)carbamate. To a stirred solution of 2-(4-aminophenyl)ethanol (500 mg, 3.65 mmol) and pyridine (433 mg, 5.47 mmol) in DCM (5 mL) was added methyl chloroformate (344 mg, 3.65 mmol) dropwise at 0° C. under nitrogen atmosphere. After stirring at room temperature overnight, the reaction mixture was diluted with DCM (10 mL), washed with 1 M aqueous HCl, followed by aqueous NaHCO₃. The organic layer was dried over NaSO₄, filtered and concentrated. The residue was purified on silica gel column (PE:EA=5:1 to 2:1) to give methyl(4-(2-hydroxyethyl)phenyl)carbamate (200 mg, 28.2%) as a yellow solid.

Step 2: methyl(4-(2-((2-amino-5-(2-(difluoromethyl)-6-methylpyridin-4-yl)-6-(4-fluorophenyl)pyrimidin-4-yl)oxy)ethyl)phenyl)carbamate. Following step 2, example 8. From methyl(4-(2-hydroxyethyl)phenyl)carbamate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=524.1.

### Examples 73 and 74

(R)-2-((2-amino-5-(2-(difluoromethyl)-6-methylpyridin-4-yl)-6-(4-fluorophenyl)pyrimidin-4-yl)oxy)propan-1-ol and (R)-1-((2-amino-5-(2-(difluoromethyl)-6-methylpyridin-4-yl)-6-(4-fluorophenyl)pyrimidin-4-yl)oxy)propan-2-ol

Step 1: (R)-2-((2-amino-5-(2-(difluoromethyl)-6-methylpyridin-4-yl)-6-(4-fluorophenyl)pyrimidin-4-yl)oxy)propan-1-ol and (R)-1-((2-amino-5-(2-(difluoromethyl)-6-methylpyridin-4-yl)-6-(4-fluorophenyl)pyrimidin-4-yl)oxy)propan-2-ol. Following step 2, example 8. From (R)-propane-1,2-diol, 60% NaH and 4-chloro-5-(2-(difluoromethyl)-6-methylpyridin-4-yl)-6-(4-fluorophenyl)pyrimidin-2-amine in DMF. Two products were separated by prep-HPLC to obtain (R)-2-((2-amino-5-(2-(difluoromethyl)-6-methylpyridin-4-yl)-6-(4-fluorophenyl)pyrimidin-4-yl)oxy)propan-1-ol, ES-MS (m/z): [M+1]⁺=405.1. and (R)-1-((2-amino-5-(2-(difluoromethyl)-6-methylpyridin-4-yl)-6-(4-fluorophenyl)pyrimidin-4-yl)oxy)propan-2-ol, ES-MS (m/z): [M+1]⁺=405.1.

### Example 75

### 4-(azetidin-3-ylmethoxy)-5-(2-(difluoromethyl)-6-methylpyridin-4-yl)-6-(4-fluorophenyl)pyrimidin-2-amine

Step 1: tert-butyl 3-4(2-amino-5-(2-(difluoromethyl)-6-methylpyridin-4-yl)-6-(4-fluorophenyl)pyrimidin-4-yl)oxy)methyl)azetidine-1-carboxylate. Following step 2, example 8. From tert-butyl 3-(hydroxymethyl)azetidine-1-carboxylate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=517.2.

Step 2: 4-(azetidin-3-ylmethoxy)-5-(2-(difluoromethyl)-6-methylpyridin-4-yl)-6-(4-fluorophenyl)pyrimidin-2-amine. A mixture of tert-butyl 3-4(2-amino-5-(2-(difluoromethyl)-6-methylpyridin-4-yl)-6-(4-fluorophenyl)pyrimidin-4-yl)oxy)methyl)azetidine-1-carboxylate (150 mg, 0.29 mmol) in HCl/MeOH (20 mL, 1.0 M) was stirred at room temperature for 2 h. The reaction mixture was concentrated and the residue was purified by prep-HPLC to obtain 4-(azetidin-3-ylmethoxy)-5-(2-(difluoromethyl)-6-methylpyridin-4-yl)-6-(4-fluorophenyl)pyrimidin-2-amine (33 mg, 27.3%) as a white solid. ES-MS (m/z): [M+1]⁺=416.0.

### Examples 76 and 77

### (S)-2-((2-amino-5-(2-(difluoromethyl)-6-methylpyridin-4-yl)-6-(4-fluorophenyl)pyrimidin-4-yl)oxy)propan-1-ol and (S)-1-((2-amino-5-(2-(difluoromethyl)-6-methylpyridin-4-yl)-6-(4-fluorophenyl)pyrimidin-4-yl)oxy)propan-2-ol

Step 1: (S)-2-((2-amino-5-(2-(difluoromethyl)-6-methylpyridin-4-yl)-6-(4-fluorophenyl)pyrimidin-4-yl)oxy)propan-1-ol and (S)-1-((2-amino-5-(2-(difluoromethyl)-6-methylpyridin-4-yl)-6-(4-fluorophenyl)pyrimidin-4-yl)oxy)propan-2-ol.

Following step 2, example 8. From (S)-propane-1,2-diol, 60% NaH and 4-chloro-5-(2-(difluoromethyl)-6-methylpyridin-4-yl)-6-(4-fluorophenyl)pyrimidin-2-amine in DMF. Two products were separated by prep-HPLC to obtain (S)-2-((2-amino-5-(2-(difluoromethyl)-6-methylpyridin-4-yl)-6-(4-fluorophenyl)pyrimidin-4-yl)oxy)propan-1-ol, ES-MS (m/z): [M+1]⁺=405.1 and (S)-1-((2-amino-5-(2-(difluoromethyl)-6-methylpyridin-4-yl)-6-(4-fluorophenyl)pyrimidin-4-yl)oxy)propan-2-ol, ES-MS (m/z): [M+1]⁺=405.1.

### Example 78

### 3-((2-amino-5-(2-(difluoromethyl)-6-methylpyridin-4-yl)-6-(4-fluorophenyl)pyrimidin-4-yl)methyl)-1,1-dimethylurea

Step 1: 3-((2-amino-5-(2-(difluoromethyl)-6-methylpyridin-4-yl)-6-(4-fluorophenyl)pyrimidin-4-yl)methyl)-1,1-dimethylurea. To a stirred mixture of 4-(aminomethyl)-5-[2-methyl-6-(trifluoromethyl)pyridin-4-yl]-6-phenylpyrimidin-2-amine (100 mg, 0.28 mmol) and triethylamine (84.5 mg, 0.84 mmol) in DCM (3 mL) was added ethyl chloroformate (30.2 mg, 0.28 mmol) dropwise at 0° C. under nitrogen atmosphere. After stirring at room temperature for 16 h, the reaction mixture was quenched with EtOH (0.5 mL). The resulting mixture was concentrated and the residue was purified by C18 reverse phase silica gel chromatography to obtain 3-((2-amino-5-(2-(difluoromethyl)-6-methylpyridin-4-yl)-6-(4-fluorophenyl)pyrimidin-4-yl)methyl)-1,1-dimethylurea (10 mg, 8.3%) as an off-white solid. ES-MS (m/z): [M+1]⁺=432.0.

### Example 79

### 5-(2-(difluoromethyl)-6-methylpyridin-4-yl)-4-(4-fluorophenyl)-6-(2-(methyl(pyridin-2-yl)amino)ethoxy)pyrimidin-2-amine

Step 1: 5-(2-(difluoromethyl)-6-methylpyridin-4-yl)-4-(4-fluorophenyl)-6-(2-(methyl(pyridin-2-yl)amino)ethoxy)pyrimidin-2-amine Following step 2, example 8. From 2-(methyl(pyridin-2-yl)amino)ethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=481.0.

### Example 80

### 5-(2-(difluoromethyl)-6-methylpyridin-4-yl)-4-(4-fluorophenyl)-6-(3-morpholinopropoxy)pyrimidin-2-amine

Step 1: 5-(2-(difluoromethyl)-6-methylpyridin-4-yl)-4-(4-fluorophenyl)-6-(3-morpholinopropoxy)pyrimidin-2-amine. Following step 2, example 8. From 3-morpholinopropan-1-ol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=474.1.

### Example 81

### 4-((2-amino-5-(2-(difluoromethyl)-6-methylpyridin-4-yl)-6-(4-fluorophenyl)pyrimidin-4-yl)oxy)butane-1,3-diol

Step 1: (2-(4-methoxyphenyl)-1,3-dioxan-4-yl)methanol. To a solution of 1-dimethoxymethyl-4-methoxy-benzene (10 mL) and butane-1,2,4-triol (5.0 g, 47.2 mmol) in DCM (60 mL) was added toluene-4-sulfonic acid (0.49 g, 2.83 mmol) at room temperature. After stirring at 40° C. for 20 h under nitrogen atmosphere, the reaction mixture was cooled and concentrated. The residue was purified on silica gel column (PE:EA=10:1) to get (2-(4-methoxyphenyl)-1,3-dioxan-4-yl)methanol (6.8 g, 65%) as a colorless oil. ES-MS (m/z): [M+1]⁺225.2.

Step 2: 5-(2-(difluoromethyl)-6-methylpyridin-4-yl)-4-(4-fluorophenyl)-6-((2-(4-methoxyphenyl)-1,3-dioxan-5-yl)methoxy)pyrimidin-2-amine Following step 2, example 8. From (2-(4-methoxyphenyl)-1,3-dioxan-4-yl)methanol, 60% NaH and 4-chloro-5-(2-difluoromethyl-6-methyl-pyridin-4-yl)-6-(4-fluoro-phenyl)-pyrimidin-2-ylamine in DMF. ES-MS (m/z): [M+1]⁺=553.0.

Step 3: 4-((2-amino-5-(2-(difluoromethyl)-6-methylpyridin-4-yl)-6-(4-fluorophenyl)pyrimidin-4-yl)oxy)butane-1,3-diol. A solution of 5-(2-(difluoromethyl)-6-methylpyridin-4-yl)-4-(4-fluorophenyl)-6-((2-(4-methoxyphenyl)-1,3-dioxan-5-yl)methoxy)pyrimidin-2-amine (100 mg, 0.18 mmol) and HCl/CH₃OH (2 M, 2 mL) was stirred at room temperature for 1 h. The reaction mixture was concentrated and the residue was purified by prep-HPLC to obtain 4-((2-amino-5-(2-(difluoromethyl)-6-methylpyridin-4-yl)-6-(4-fluorophenyl)pyrimidin-4-yl)oxy)butane-1,3-diol (26.8mg, 34%) as a white solid. ES-MS (m/z): [M+1]⁺=435.1.

### Example 82

### 5-(2-(difluoromethyl)-6-methylpyridin-4-yl)-4-(4-fluorophenyl)-6-(2-(phenylamino)ethoxy)pyrimidin-2-amine

Step 1: 5-(2-(difluoromethyl)-6-methylpyridin-4-yl)-4-(4-fluorophenyl)-6-(2-(phenylamino)ethoxy)pyrimidin-2-amine Following step 2, example 8. From 2-(phenylamino)ethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=466.0.

### Example 83

### (S)-3-((2-amino-5-(2-(difluoromethyl)-6-methylpyridin-4-yl)-6-(4-fluorophenyl)pyrimidin-4-yl)oxy)propane-1,2-diol

Step 1: (R)-5-(2-(difluoromethyl)-6-methylpyridin-4-yl)-4-((2,2-dimethyl-1,3-dioxolan-4-yl)methoxy)-6-(4-fluorophenyl)pyrimidin-2-amine Following step 2, example 8. From (R)-(2,2-dimethyl-1,3-dioxolan-4-yemethanol, 60% NaH and 4-chloro-5-(2-(difluoromethyl)-6-methylpyridin-4-yl)-6-(4-fluorophenyl)pyrimidin-2-amine in DMF to obtain crude (R)-5-(2-(difluoromethyl)-6-methylpyridin-4-yl)-4-((2,2-dimethyl-1,3-dioxolan-4-yl)methoxy)-6-(4-fluorophenyl)pyrimidin-2-amine (80 mg, 64.5%) as a colorless oil.

Step 2: (S)-3-((2-amino-5-(2-(difluoromethyl)-6-methylpyridin-4-yl)-6-(4-fluorophenyl)pyrimidin-4-yl)oxy)propane-1,2-diol. A mixture of (R)-5-(2-(difluoromethyl)-6-methylpyridin-4-yl)-4-((2,2-dimethyl-1,3-dioxolan-4-yl)methoxy)-6-(4-fluorophenyl)pyrimidin-2-amine (80 mg, 0.17 mmol), aqueous HCl (1 M, 1.0 mL) and acetone (1.0 mL) was stirred at 70° C. for 1 h. The reaction mixture was cooled and concentrated. The residue was dissolved in NH₃/MeOH (4.0 m, 5 mL), then concentrated again. The residue was purified by prep-HPLC to obtain (S)-3-((2-amino-5-(2-(difluoromethyl)-6-methylpyridin-4-yl)-6-(4-fluorophenyl)pyrimidin-4-yl)oxy)propane-1,2-diol (24 mg, 28%) as a white solid. ES-MS (m/z): [M+1]⁺=421.0.

### Example 84

### 2-(2-amino-5-(2-(difluoromethyl)-6-methylpyridin-4-yl)-6-(4-fluorophenyl)pyrimidin-4-yl)oxy)-N-benzylacetamide

Step 1: N-benzyl-2-hydroxyacetamide. To a mixture of benzylamine (500 mg, 4.67 mmol), 2-hydroxyacetic acid (355 mg, 4.67 mmol) and DIPEA (1.80 g, 14.0 mmol) in DMF (10 mL) was added HATU (2.70 g, 7.0 mmol) at room temperature. After stirring at room temperature for 2 h, the reaction mixture was quenched with H₂O and concentrated. The residue was purified by C18 reverse phase silica gel chromatography to give N-benzyl-2-hydroxyacetamide (100 mg, 12.9%) as a white solid.

Step 2: 2-((2-amino-5-(2-(difluoromethyl)-6-methylpyridin-4-yl)-6-(4-fluorophenyl)pyrimidin-4-yl)oxy)-N-benzylacetamide. Following step 2, example 8. From N-benzyl-2-hydroxyacetamide, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=494.0.

### Example 85

### 1-((2-amino-5-(2-(difluoromethyl)-6-methylpyridin-4-yl)-6-(4-fluorophenyl)pyrimidin-4-yl)oxy)-3-methoxypropan-2-ol

Step 1: 1-((2-amino-5-(2-(difluoromethyl)-6-methylpyridin-4-yl)-6-(4-fluorophenyl)pyrimidin-4-yl)oxy)-3-methoxypropan-2-ol. Following step 2, example 8. From 3-methoxypropane-1,2-diol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=435.0.

### Example 86

### (R)-3-((2-amino-5-(2-(difluoromethyl)-6-methylpyridin-4-yl)-6-(4-fluorophenyl)pyrimidin-4-yl)oxy)propane-1,2-diol

Step 1: of (S)-5-(2-(difluoromethyl)-6-methylpyridin-4-yl)-4-((2,2-dimethyl-1,3-dioxolan-4-yl)methoxy)-6-(4-fluorophenyl)pyrimidin-2-amine Following step 2, example 8. From (S)-(2,2-dimethyl-1,3-dioxolan-4-yl)methanol, 60% NaH and 4-chloro-5-(2-(difluoromethyl)-6-methylpyridin-4-yl)-6-(4-fluorophenyl)pyrimidin-2-amine in DMF.

Step 2: (R)-3-((2-amino-5-(2-(difluoromethyl)-6-methylpyridin-4-yl)-6-(4-fluorophenyl)pyrimidin-4-yl)oxy)propane-1,2-diol. Following step 2, example 83. From (S)-5-(2-(difluoromethyl)-6-methylpyridin-4-yl)-4-((2,2-dimethyl-1,3-dioxolan-4-yl)methoxy)-6-(4-fluorophenyl)pyrimidin-2-amine, 1.0 M aqueous HCl and acetone. ES-MS (m/z): [M+1]⁺=421.0.

### Example 87

### 5-(2-(difluoromethyl)-6-methylpyridin-4-yl)-4-(4-fluorophenyl)-6-(2-(methyl(phenyl)amino)ethoxy)pyrimidin-2-amine

Step 1: 5-(2-(difluoromethyl)-6-methylpyridin-4-yl)-4-(4-fluorophenyl)-6-(2-(methyl(phenyl)amino)ethoxy)pyrimidin-2-amine Following step 2, example 8. From 2-(methyl(phenyl)amino)ethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=480.0.

### Example 88

### 2-(2-amino-5-(2-(difluoromethyl)-6-methylpyridin-4-yl)-6-(4-fluorophenyl)pyrimidin-4-yl)oxy)-N,N-bis(2-hydroxyethyl)acetamide

Step 1: bis(2-((tert-butyldimethylsilyl)oxy)ethyl)amine. To a mixture of 2,2′-azanediyldiethanol (3.0 g, 28.5 mmol) and imidazole (4.0 g, 58.8 mmol) in DCM (30 mL) was added TBSC1 (6.7 g, 44.5 mmol) at 0° C. under nitrogen atmosphere. After stirring at room temperature for 2 h, the reaction mixture was poured into H₂O (60 mL), extracted with DCM (30 mL×2). The combined organic extracts were washed with brine, dried over Na₂SO₄, filtered and concentrated. The residue was purified on silica gel column (PE:EA=1:1) to obtain bis(2-((tert-butyldimethylsilyl)oxy)ethyl)amine (9.0 g, 94.5%) as a white solid. ES-MS (m/z): [M+1]⁺=334.6.

Step 2: N,N-bis(2-((tert-butyldimethylsilyl)oxy)ethyl)-2-hydroxyacetamide. A mixture of bis(2-((tert-butyldimethylsilyl)oxy)ethyl)amine (1.3 g, 3.9 mmol), 2-hydroxyacetic acid (200 mg, 2.6 mmol), HATU (1.1 g, 2.9 mmol) and DIPEA (1.0 g, 7.8 mmol) in DCM (10 mL) was stirred at room temperature for 1 h. The reaction mixture was poured into H₂O (20 mL), extracted with DCM (20 mL×2). The combined organic extracts were washed with brine, dried over Na₂SO₄, filtered and concentrated. The residue was purified on silica gel column (DCM:MeOH=20:1) to obtain N,N-bis(2-((tert-butyldimethylsilyl)oxy)ethyl)-2-hydroxyacetamide (500 mg, 49.0%) as a white solid. ES-MS (m/z): [M+1]⁺=392.4.

Step 3: 2-((2-amino-5-(2-(difluoromethyl)-6-methylpyridin-4-yl)-6-(4-fluorophenyl)pyrimidin-4-yl)oxy)-N,N-bis(2-((tert-butyldimethylsilyl)oxy)ethyeacetamide. Following step 2, example 8. From N,N-bis(2-((tert-butyldimethylsilyl)oxy)ethyl)-2-hydroxyacetamide, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=720.4.

Step 4: 2-((2-amino-5-(2-(difluoromethyl)-6-methylpyridin-4-yl)-6-(4-fluorophenyl)pyrimidin-4-yl)oxy)-N,N-bis(2-hydroxyethyl)acetamide. A mixture of 2-((2-amino-5-(2-(difluoromethyl)-6-methylpyridin-4-yl)-6-(4-fluorophenyl)pyrimidin-4-yl)oxy)-N,N-bis(2-((tert-butyldimethylsilyl)oxy)ethyl)acetamide (100 mg, crude) and TBAF/THF (1.0 M, 1.0 mL) was stirred at room temperature for 1 h. The reaction mixture was concentrated and purified by C18 reverse phase silica gel column to obtain 2-((2-amino-5-(2-(difluoromethyl)-6-methylpyridin-4-yl)-6-(4-fluorophenyl)pyrimidin-4-yl)oxy)-N,N-bis(2-hydroxyethyeacetamide (6.0 mg, 9.0% over two steps) as a white solid. ES-MS (m/z): [M+1]⁺=492.1.

### Example 89

### 2-((2-amino-5-(2-(difluoromethyl)-6-methylpyridin-4-yl)-6-(4-fluorophenyl)pyrimidin-4-yl)oxy)-N-ethyl-N-(2-hydroxyethyl)acetamide

Step 1: 2-((tert-butyldimethylsilyl)oxy)-N-ethylethanamine. Following step 1, example 88. From 2-(ethylamino)ethanol, imidazole and TBSC1 in DCM. ES-MS (m/z): [M+1]⁺=204.5.

Step 2: N-(2-((tert-butyldimethylsilyl)oxy)ethyl)-N-ethyl-2-hydroxyacetamide. Following step 2, example 88. From 2-hydroxyacetic acid, 2-((tert-butyldimethylsilyl)oxy)-N-ethylethanamine, HATU and DIPEA in DCM. ES-MS (m/z): [M+1]⁺=262.2.

Step 3: 2-((2-amino-5-(2-(difluoromethyl)-6-methylpyridin-4-yl)-6-(4-fluorophenyl)pyrimidin-4-yl)oxy)-N-(2-((tert-butyldimethylsilyl)oxy)ethyl)-N-ethylacetamide. Following step 2, example 8. From N-(2-((tert-butyldimethylsilyl)oxy)ethyl)-N-ethyl-2-hydroxyacetamide, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=590.2.

Step 4: 2-((2-amino-5-(2-(difluoromethyl)-6-methylpyridin-4-yl)-6-(4-fluorophenyl)pyrimidin-4-yl)oxy)-N-ethyl-N-(2-hydroxyethyl)acetamide. Following step 2, example 88. From 2-42-amino-5-(2-(difluoromethyl)-6-methylpyridin-4-yl)-6-(4-fluorophenyl)pyrimidin-4-yl)oxy)-N-(2-((tert-butyldimethylsilyl)oxy)ethyl)-N-ethylacetamide and 1.0 M TBAF in THF. ES-MS (m/z): [M+1]⁺=476.0.

### Example 90

### 2-(2-amino-5-(2-(difluoromethyl)-6-methylpyridin-4-yl)-6-(4-fluorophenyl)pyrimidin-4-yl)oxy)-N-ethyl-N-phenylacetamide

Step 1: 2-((2-amino-5-(2-(difluoromethyl)-6-methylpyridin-4-yl)-6-(4-fluorophenyl)pyrimidin-4-yl)oxy)-N-ethyl-N-phenylacetamide. Following step 2, example 2. From 2-((2-amino-5-(2-(difluoromethyl)-6-methylpyridin-4-yl)-6-(4-fluorophenyl)pyrimidin-4-yl)oxy)acetic acid, N-ethylaniline, DIPEA and HATU in DMF. ES-MS (m/z): [M+1]⁺=508.1.

### Example 91

### 1-((2-amino-5-(2-(difluoromethyl)-6-methylpyridin-4-yl)-6-(4-fluorophenyl)pyrimidin-4-yl)oxy)-3-methylbutane-2,3-diol

Step 1: (3,3-dimethyl-oxiranyl)-methanol. To a solution of 3-methyl-but-2-en-1-ol (2.0 g, 23.0 mmol) in DCM (20 mL) was added mCPBA (4.7 g, 27.6 mmol) in portions at 0° C. under nitrogen atmosphere. After stirring for 2 h at room temperature, the reaction mixture was poured into water (40 mL), extracted with DCM (100 mL×3). The combined organic extracts were dried over Na₂SO₄, filtered and concentrated. The residue was purified on silica gel column (PE:EA=201) to obtain (3,3-Dimethyl-oxiranyl)-methanol (1.8 g, 78%) as a white oil.

Step 2: 5-(2-difluoromethyl-6-methyl-pyridin-4-yl)-4-(3,3-dimethyl-oxiranylmethoxy)-6-(4-fluoro-phenyl)-pyrimidin-2-ylamine Following step 2, example 8. From (3,3-dimethyl-oxiranyl)-methanol, 60% NaH and 4-Chloro-5-(2-difluoromethyl-6-methyl-pyridin-4-yl)-6-(4-fluoro-phenyl)-pyrimidin-2-ylamine in DCM. ES-MS (m/z): [m+1]⁺=431.1.

Step 3: 1-((2-amino-5-(2-(difluoromethyl)-6-methylpyridin-4-yl)-6-(4-fluorophenyl)pyrimidin-4-yl)oxy)-3-methylbutane-2,3-diol. A mixture of 5-(2-difluoromethyl-6-methyl-pyridin-4-yl)-4-(3,3-dimethyl-oxiranylmethoxy)-6-(4-fluoro-phenyl)-pyrimidin-2-ylamine (50 mg, 0.12 mmol) and HCl/CH₃OH (2 M, 2.0 mL) was stirred at room temperature for 1 h. The reaction mixture was concentrated, and to the residue was added NH₃/CH₃OH (2 M, 2.0 mL). After stirring for 0.5 h, the mixture was concentrated again. The residue was purified by prep-HPLC to obtain 1-((2-amino-5-(2-(difluoromethyl)-6-methylpyridin-4-yl)-6-(4-fluorophenyl)pyrimidin-4-yl)oxy)-3-methylbutane-2,3-diol (1.9 mg, 4%) as a white solid. ES-MS (m/z): [M+1]⁺=449.1.

### Example 92

### 2-((2-amino-5-(2-(difluoromethyl)-6-methylpyridin-4-yl)-6-(4-fluorophenyl)pyrimidin-4-yl)oxy)-N-phenylacetamide

Step 1: 2-hydroxy-N-phenylacetamide. To a solution of 2-hydroxyacetic acid (1.0 g, 0.013 mol) in DCM (10 mL) were added HATU (6.0 g, 0.016 mol), DIPEA (5.1 g, 0.019 mmol) and aniline (2.5 g, 0.026 mol). After stirring at room temperature overnight, H₂O (15 mL) was added to the reaction mixture. The resulting mixture was extracted with DCM. The organic extract was washed with brine, dried over Na₂SO₄, filtered and concentrated. The residue was purified on silica gel column (PE:EA=1:1) to obtain 2-hydroxy-N-phenylacetamide (440 mg, 22%) as a white solid. ES-MS (m/z): [M+1]⁺=151.9.

Step 2: 2-((2-amino-5-(2-(difluoromethyl)-6-methylpyridin-4-yl)-6-(4-fluorophenyl)pyrimidin-4-yl)oxy)-N-phenylacetamide. Following step 2, example 8. From 2-hydroxy-N-phenylacetamide, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=480.1.

### Example 93

### 4-(2-((2-amino-5-(2-(difluoromethyl)-6-methylpyridin-4-yl)-6-(4-fluorophenyl)pyrimidin-4-yl)oxy)ethyl)benzonitrile

Step 1: 5-(2-Difluoromethyl-6-methyl-pyridin-4-yl)-4-(4-fluoro-phenyl)-6-(2-(4-iodo-phenyl)-ethoxyl-pyrimidin-2-ylamine Following step 2, example 8. From 2-(4-Iodo-phenyl)-ethanol, 60% NaH and 4-chloro-5-(2-methyl-6-trifluoromethyl-pyridin-4-yl)-6-phenyl-pyrimidin-2-ylamine in DMF. ES-MS (m/z): [M+1]⁺=577.6.

Step 2: 4-(2-((2-amino-5-(2-(difluoromethyl)-6-methylpyridin-4-yl)-6-(4-fluorophenyl)pyrimidin-4-yl)oxy)ethyl)benzonitrile. To a solution of 5-(2-difluoromethyl-6-methyl-pyridin-4-yl)-4-(4-fluoro-phenyl)-6-[2-(4-iodo-phenyl)-ethoxy]-pyrimidin-2-ylamine (40 mg, 0.069 mmol) in DMF (1.0 mL) was added Zn(CN)₂ (11 mg, 0.090 mmol), Pd(PPh₃)₄ (8 mg, 0.007 mmol) under nitrogen atmosphere. This resulting mixture was stirred at 120° C. under nitrogen atmosphere for 4 h. The reaction mixture was cooled and concentrated. The residue was first purified on silica gel column (PE:EA=2:1), followed by prep-HPLC to obtain 4-(2-((2-amino-5-(2-(difluoromethyl)-6-methylpyridin-4-yl)-6-(4-fluorophenyl)pyrimidin-4-yl)oxy)ethyl)benzonitrile (12.5 mg, 37%) as a white solid. ES-MS (m/z): [M+1]⁺=476.1.

### Example 94

### 5-(2-(difluoromethyl)-6-methylpyridin-4-yl)-4-(4-fluorophenyl)-6-(2-(pyridin-2-ylamino)ethoxy)pyrimidin-2-amine

Step 1: 2-(pyridin-2-ylamino)ethanol. A mixture of 2-fluoropyridine (340 mg, 3.5 mmol), 2-aminoethanol (2.1 mL, 35 mmol) and pyridine (1.0 mL) was stirred at 210° C. under microwave irradiation for 1 h. The reaction mixture was cooled and diluted with aqueous NaHCO₃ (10 mL), extracted with ethyl acetate. The organic extract was dried over Na₂SO₄, filtered and concentrated. The residue was purified on silica gel column (DCM:MeOH=50:1) to obtain 2-(pyridin-2-ylamino)ethanol (300 mg, 62.1%) as a white solid. ES-MS (m/z): [M+1]⁺=139.0.

Step 2: 5-(2-(difluoromethyl)-6-methylpyridin-4-yl)-4-(4-fluorophenyl)-6-(2-(pyridin-2-ylamino)ethoxy)pyrimidin-2-amine Following step 2, example 8. From 2-(pyridin-2-ylamino)ethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=467.1.

### Example 95

### 5-(2-(difluoromethyl)-6-methylpyridin-4-yl)-4-(4-fluorophenyl)-6-(pyridin-2-ylmethoxy)pyrimidin-2-amine

Step 1: 5-(2-(difluoromethyl)-6-methylpyridin-4-yl)-4-(4-fluorophenyl)-6-(pyridin-2-ylmethoxy)pyrimidin-2-amine. Following step 2, example 8. From pyridin-2-ylmethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=438.1.

### Example 96

### 5-(2-(difluoromethyl)-6-methylpyridin-4-yl)-4-(4-fluorophenyl)-6-((1-(methylsulfonyl)piperidin-4-yl)methoxy)pyrimidin-2-amine

Step 1: tert-butyl 4-(((2-amino-5-(2-(difluoromethyl)-6-methylpyridin-4-yl)-6-(4-fluorophenyl)pyrimidin-4-yl)oxy)methyl)piperidine-1-carboxylate. Following step 2, example 8. From tert-butyl 4-(hydroxymethyl)piperidine-1-carboxylate, 60% NaH and 4-chloro-5-(2-(difluoromethyl)-6-methylpyridin-4-yl)-6-(4-fluorophenyl)pyrimidin-2-amine in DMF.

Step 2: 5-(2-(difluoromethyl)-6-methylpyridin-4-yl)-4-(4-fluorophenyl)-6-(piperidin-4-ylmethoxy)pyrimidin-2-amine. A mixture of tert-butyl 4-4(2-amino-5-(2-(difluoromethyl)-6-methylpyridin-4-yl)-6-(4-fluorophenyl)pyrimidin-4-yloxy)methyl)piperidine-1-carboxylate (250 mg, 0.46 mmol) in HCl/MeOH (1.0 M, 3.0 mL) was stirred at room temperature for 30 min. The reaction mixture was concentrated to obtained crude 5-(2-(difluoromethyl)-6-methylpyridin-4-yl)-4-(4-fluorophenyl)-6-(piperidin-4-ylmethoxy)pyrimidin-2-amine (200 mg, 98%) as a yellow solid. ES-MS (m/z): [M+1]⁺=444.3.

Step 3: 5-(2-(difluoromethyl)-6-methylpyridin-4-yl)-4-(4-fluorophenyl)-6-((1-(methylsulfonyl)piperidin-4-yl)methoxy)pyrimidin-2-amine To a stirred mixture of 5-(2-(difluoromethyl)-6-methylpyridin-4-yl)-4-(4-fluorophenyl)-6-(piperidin-4-ylmethoxy)pyrimidin-2-amine (100 mg, 0.225 mmol) and triethylamine (70 mg, 0.675 mmol) in DCM (2.0 mL) was added MsCl (50 mg, 0.45 mmol) at 0° C. under nitrogen atmosphere. After stirring at 0° C. for 2 h, water (15 mL) was added, and the resulting mixture was extracted with DCM (30 mL). The organic layer was concentrated and purified by prep-HPLC to obtain 5-(2-(difluoromethyl)-6-methylpyridin-4-yl)-4-(4-fluorophenyl)-6-((1-(methylsulfonyl)piperidin-4-yl)methoxy)pyrimidin-2-amine (15 mg, 12.8%) as a white solid. ES-MS (m/z): [M+1]⁺=522.1.

### Example 97

### 5-(2-(difluoromethyl)-6-methylpyridin-4-yl)-4-(4-fluorophenyl)-6-((3 -methyl-1H-1,2,4-triazol-5-yl)methoxy)pyrimidin-2-amine

Step 1: 2-([2-amino-5-[2-(difluoromethyl)-6-methylpyridin-4-yl]-6-(4-fluorophenyl)pyrimidin-4-yl]oxy)acetohydrazide. A mixture of methyl 2-([2-amino-5-[2-(difluoromethyl)-6-methylpyridin-4-yl]-6-(4-fluorophenyl)pyrimidin-4-yl]oxy)acetate (113.0 mg, 0.27 mmol) and hydrazine monohydrate (50.0 mg, 1.0 mmol) in EtOH (1.0 mL) was stirred for 24 h at 80° C. The reaction mixture was cooled and concentrated. The residue was purified on silica gel column with DCM/MeOH (10/1) to afford 2-([2-amino-5-[2-(difluoromethyl)-6-methylpyridin-4-yl]-6-(4-fluorophenyl)pyrimidin-4-yl]oxy)acetohydrazide (81 mg, 71.7%) as a white solid. ES-MS (m/z): [M+1]⁺=419.2.

Step 2: 5-(2-(difluoromethyl)-6-methylpyridin-4-yl)-4-(4-fluorophenyl)-6-((3-methyl-1H-1,2,4-triazol-5-yl)methoxy)pyrimidin-2-amine To a stirred mixture of 2-([2-amino-5-[2-(difluoromethyl)-6-methylpyridin-4-yl]-6-(4-fluorophenyl)pyrimidin-4-yl]oxy)acetohydrazide (81.0 mg, 0.19 mmol) and ethanimidamide hydrochloride (18.0 mg, 0.19 mmol) in MeOH (1 mL) was added NaOMe (58 mg, 0.19 mmol, 18% in MeOH) dropwise at room temperature. After stirring for 16 h at 70° C., the reaction mixture was cooled and diluted with MeOH (3 mL). The resulting mixture was filtered. The filtrate was purified by Prep-HPLC to afford 5-(2-(difluoromethyl)-6-methylpyridin-4-yl)-4-(4-fluorophenyl)-6-((3-methyl-1H-1,2,4-triazol-5-yl)methoxy)pyrimidin-2-amine (14 mg, 16.4%) as an off-white solid. ES-MS (m/z): [M+1]⁺=442.1.

### Example 98

### 2-((2-amino-5-(2-(difluoromethyl)-6-methylpyridin-4-yl)-6-(4-fluorophenyl)pyrimidin-4-yl)oxy)-N-methyl-N-phenylacetamide

Step 1: 2-((2-amino-5-(2-(difluoromethyl)-6-methylpyridin-4-yl)-6-(4-fluorophenyl)pyrimidin-4-yl)oxy)-N-methyl-N-phenylacetamide. Following step 2, example 2. From 2-((2-amino-5-(2-(difluoromethyl)-6-methylpyridin-4-yl)-6-(4-fluorophenyl)pyrimidin-4-yl)oxy)acetic acid, N-methylaniline, DIPEA and HATU in DMF. ES-MS (m/z): [M+1]⁺=494.1.

### Example 99

### 5-(2-(difluoromethyl)-6-methylpyridin-4-yl)-4-(4-fluorophenyl)-6-((1-isopropyl-1H-imidazol-2-yl)methoxy)pyrimidin-2-amine

Following step 2, example 8. From (1-Isopropyl-1H-imidazol-2-yl)-methanol, 60% NaH and 4-chloro-5-(2-difluoromethyl-6-methyl-pyridin-4-yl)-6-(4-fluoro-phenyl)-pyrimidin-2-ylamine in DMF. ES-MS (m/z): [M+1]⁺=469.1.

### Example 100

### 2-amino-5-(2-(difluoromethyl)-6-methylpyridin-4-yl)-6-(4-fluorophenyl)-N-(5,6,7,8-tetrahydroquinolin-8-yl)pyrimidine-4-carboxamide

Step 1: 2-amino-5-(2-(difluoromethyl)-6-methylpyridin-4-yl)-6-(4-fluorophenyl)pyrimidine-4-carboxylate. A mixture of 4-chloro-5-(2-(difluoromethyl)-6-methylpyridin-4-yl)-6-(4-fluorophenyl)pyrimidin-2-amine (500 mg, 1.4 mmol), Pd(dppf)Cl₂ (205 mg, 0.28 mmol) and TEA (424 mg, 4.2 mmol) in i-PrOH (10 mL) was stirred at 120° C. overnight under 5 MPa of CO. After cooling, the reaction mixture was concentrated and the residue was purified by silica gel flash chromatography (PE:EA=4:1) to obtain isopropyl 2-amino-5-(2-(difluoromethyl)-6-methylpyridin-4-yl)-6-(4-fluorophenyl)pyrimidine-4-carboxylate (260 mg, 44%) as a yellow solid. ES-MS (m/z): [M+1]⁺=416.7.

Step 2: lithium 2-amino-5-(2-(difluoromethyl)-6-methylpyridin-4-yl)-6-(4-fluorophenyl)pyrimidine-4-carboxylate. To a mixture of isopropyl 2-amino-5-(2-(difluoromethyl)-6-methylpyridin-4-yl)-6-(4-fluorophenyl)pyrimidine-4-carboxylate (260 mg, 0.6 mmol) in THF/MeOH/H20=2/2/1 (5.0 mL) was added LiOHH₂O (76 mg, 1.8 mmol). After stirring at room temperature for 1 h, the reaction mixture was concentrated to get crude lithium 2-amino-5-(2-(difluoromethyl)-6-methylpyridin-4-yl)-6-(4-fluorophenyl)pyrimidine-4-carboxylate (300 mg, 100%) as a light-yellow solid. ES-MS (m/z): [M+1]⁺=374.7.

Step 3: 2-amino-5-(2-(difluoromethyl)-6-methylpyridin-4-yl)-6-(4-fluorophenyl)-N-(5,6,7,8-tetrahydroquinolin-8-yl)pyrimidine-4-carboxamide. To a mixture of lithium 2-amino-5-(2-(difluoromethyl)-6-methylpyridin-4-yl)-6-(4-fluorophenyl)pyrimidine-4-carboxylate (80 mg, 0.21 mmol) and 5,6,7,8-tetrahydroquinolin-8-amine (37 mg, 0.25 mmol) in DMF (2 mL) were added HATU (120 mg, 0.31 mmol) and DIPEA (54 mg, 0.42 mmol). After stirring at room temperature for 1 h, the reaction mixture was concentrated. The residue was purified by silica gel flash chromatography (DCM:MeOH=50:1) to obtain 2-amino-5-(2-(difluoromethyl)-6-methylpyridin-4-yl)-6-(4-fluorophenyl)-N-(5,6,7,8-tetrahydroquinolin-8-yl)pyrimidine-4-carboxamide (17 mg, 16%) as a brown solid. ES-MS (m/z): [M+1]⁺=505.1.

### Example 101

### 2-((2-amino-5-(2-(difluoromethyl)-6-methylpyridin-4-yl)-6-(4-fluorophenyl)pyrimidin-4-yl)oxy)-N-methyl-N-(oxetan-3-yl)acetamide

Step 1: 2-((2-amino-5-(2-(difluoromethyl)-6-methylpyridin-4-yl)-6-(4-fluorophenyl)pyrimidin-4-yl)oxy)-N-methyl-N-(oxetan-3-yl)acetamide. Following step 2, example 2. From 2-((2-amino-5-(2-(difluoromethyl)-6-methylpyridin-4-yl)-6-(4-fluorophenyl)pyrimidin-4-yl)oxy)acetic acid, N-methyloxetan-3-amine, DIPEA and HATU in DMF. ES-MS (m/z): [M+1]⁺=474.0.

### Example 102

### 5-(2-(difluoromethyl)-6-methylpyridin-4-yl)-4-(4-fluorophenyl)-6-((4-fluorotetrahydro-2H-pyran-4-yl)methoxy)pyrimidin-2-amine

Step 1: 5-(2-(difluoromethyl)-6-methylpyridin-4-yl)-4-(4-fluorophenyl)-6-((4-fluorotetrahydro-2H-pyran-4-yl)methoxy)pyrimidin-2-amine Following step 2, example 8. From (4-fluorotetrahydro-2H-pyran-4-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=463.0.

### Example 103

### methyl((2-amino-5-(2-(difluoromethyl)-6-methylpyridin-4-yl)-6-(4-fluorophenyl)pyrimidin-4-yl)methyl)carbamate

Step 1: 2-amino-5-(2-(difluoromethyl)-6-methylpyridin-4-yl)-6-(4-fluorophenyl)pyrimidine-4-carbonitrile. A mixture of 4-chloro-5-(2-(difluoromethyl)-6-methylpyridin-4-yl)-6-(4-fluorophenyl)pyrimidin-2-amine (1.0 g, 2.7 mmol), Zn(CN)₂ (632 mg, 5.4 mmol) and Pd(PPh₃)₄ (312 mg, 0.27 mmol) in DMF (15 mL) was stirred at 120° C. overnight under nitrogen atmosphere. After cooling, the reaction mixture was diluted with H₂O (20 mL) and extracted with ethyl acetate (20 mL×2). The combined organic extracts were washed with brine (20 mL), dried over Na₂SO₄, filtered and concentrated. The residue was purified by silica gel flash chromatography (PE:EA=3:1) to obtain 2-amino-5-(2-(difluoromethyl)-6-methylpyridin-4-yl)-6-(4-fluorophenyl)pyrimidine-4-carbonitrile (700 mg, 73%) as a yellow solid.

Step 2: 4-(aminomethyl)-5-(2-(difluoromethyl)-6-methylpyridin-4-yl)-6-(4-fluorophenyl)pyrimidin-2-amine. To a solution of 2-amino-5-(2-(difluoromethyl)-6-methylpyridin-4-yl)-6-(4-fluorophenyl)pyrimidine-4-carbonitrile (150 mg, 0.42 mmol) in acetic acid (5.0 mL) was added Pd/C (20 mg, 10% in activated carbon). The mixture was stirred at room temperature for 2 h under hydrogen atmosphere. The reaction mixture was filtered and concentrated. The residue was purified by silica gel flash chromatography (DCM:MeOH=20:1) to get 4-(aminomethyl)-5-(2-(difluoromethyl)-6-methylpyridin-4-yl)-6-(4-fluorophenyl)pyrimidin-2-amine (120 mg, 79%) as a purple solid. ES-MS (m/z): [M+1]⁺=359.8.

Step 3: methyl((2-amino-5-(2-(difluoromethyl)-6-methylpyridin-4-yl)-6-(4-fluorophenyl)pyrimidin-4-yl)methyl)carbamate. To a stirred solution of 4-(aminomethyl)-5-(2-(difluoromethyl)-6-methylpyridin-4-yl)-6-(4-fluorophenyl)pyrimidin-2-amine (50 mg, 0.14 mmol) and triethylamine (28 mg, 0.28 mmol) in DCM (3 mL) was added methyl chloroformate (16 mg, 0.17 mmol) at 0° C. under nitrogen atmosphere. After further stirring at 0° C. for 2 h, the reaction mixture was diluted with H₂O (15 mL) and extracted with DCM (15 mL×2). The combined organic extracts were washed with brine (15 mL), dried over Na₂SO₄, filtered and concentrated. The residue was purified by silica gel flash chromatography (PE:=1:1) to get methyl((2-amino-5-(2-(difluoromethyl)-6-methylpyridin-4-yl)-6-(4-fluorophenyl)pyrimidin-4-yl)methyl)carbamate (17 mg, 29%) as a yellow solid. ES-MS (m/z): [M+1]⁺=418.0.

### Example 104

### 4-(((2-amino-5-(2-(difluoromethyl)-6-methylpyridin-4-yl)-6-(4-fluorophenyl)pyrimidin-4-yl)oxy)methyl)tetrahydro-2H-pyran-4-ol

Step 1: 2-Amino-5-(2-difluoromethyl-6-methyl-pyridin-4-yl)-6-(4-fluoro-phenyl)-pyrimidin-4-ol. A mixture of 4-chloro-5-(2-difluoromethyl-6-methyl-pyridin-4-yl)-6-(4-fluoro-phenyl)-pyrimidin-2-ylamine (500 mg, 1.37 mmol) and 6 M aqueous HCl (10 mL) was stirred at 90° C. for 1 h. The reaction mixture was concentrated to get crude 2-amino-5-(2-difluoromethyl-6-methyl-pyridin-4-yl)-6-(4-fluoro-phenyl)-pyrimidin-4-ol (500 mg, 100%) as a light yellow solid. ES-MS (m/z): [M+1]⁺=347.8.

Step 2: 4-(((2-amino-5-(2-(difluoromethyl)-6-methylpyridin-4-yl)-6-(4-fluorophenyl)pyrimidin-4-yl)oxy)methyl)tetrahydro-2H-pyran-4-ol. A mixture of 2-amino-5-(2-difluoromethyl-6-methyl-pyridin-4-yl)-6-(4-fluoro-phenyl)-pyrimidin-4-ol (100 mg, 0.29 mmol), 1,6-dioxa-spiro[2.5]octane (33 mg, 0.29 mmol) and Cs₂CO₃ (282 mg, 0.86 mmol) in DMF (6 mL) was stirred at 100° C. under nitrogen atmosphere overnight. After cooling, the reaction mixture was filtered and the filtrate was purified by prep-HPLC to obtain 4-(((2-amino-5-(2-(difluoromethyl)-6-methylpyridin-4-yl)-6-(4-fluorophenyl)pyrimidin-4-yl)oxy)methyl)tetrahydro-2H-pyran-4-ol (8.3 mg, 6.3%) as a pale yellow solid. ES-MS (m/z): [M+1]⁺=461.2.

### Example 105

### 5-(2-(difluoromethyl)-6-methylpyridin-4-yl)-4-((1-ethyl-1H-imidazol-2-yl)methoxy)-6-(4-fluorophenyl)pyrimidin-2-amine

Step 1: (1-ethyl-1H-imidazol-2-yl)methanol. To a solution of 1-ethyl-1H-imidazole-2-carbaldehyde (300 mg, 2.42 mmol) in THF (5.0 mL) was added LAH (184 mg, 4.84 mmol) at 0° C. under nitrogen atmosphere. After further stirring at room temperature for 3 h, the reaction mixture was filtered and concentrated to give crude (1-ethyl-1H-imidazol-2-yl)methanol (120 mg, 39.5%) as a yellow solid. ES-MS (m/z): [M+1]⁺=127.0.

Step 2: 5-(2-(difluoromethyl)-6-methylpyridin-4-yl)-4-((1-ethyl-1H-imidazol-2-yl)methoxy)-6-(4-fluorophenyl)pyrimidin-2-amine Following step 2, example 8. From (1-ethyl-1H-imidazol-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=455.1.

### Example 106

### methyl 4-(((2-amino-5-(2-(difluoromethyl)-6-methylpyridin-4-yl)-6-(4-fluorophenyl)pyrimidin-4-yl)oxy)methyl)piperidine-1-carboxylate

Step 1: methyl 4-(((2-amino-5-(2-(difluoromethyl)-6-methylpyridin-4-yl)-6-(4-fluorophenyl)pyrimidin-4-yl)oxy)methyl)piperidine-1-carboxylate. To a stirred mixture of 5-(2-(difluoromethyl)-6-methylpyridin-4-yl)-4-(4-fluorophenyl)-6-(piperidin-4-ylmethoxy)pyrimidin-2-amine (100 mg, 0.23 mmol) and triethylamine (70 mg, 0.68 mmol) in DCM (2 mL) was added methyl chloroformate (43 mg, 0.45 mmol) at 0° C. under nitrogen atmosphere. After stirring further at 0° C. for 2 h, the reaction mixture was quenched with H₂O (15 mL), extracted with DCM (30 mL). The organic extract was concentrated and purified by prep-HPLC to obtain methyl 4-(((2-amino-5-(2-(difluoromethyl)-6-methylpyridin-4-yl)-6-(4-fluorophenyl)pyrimidin-4-yl)oxy)methyl)piperidine-1-carboxylate (28 mg, 24.8%) as a white solid. ES-MS (m/z): [M+1]⁺=502.0.

### Example 107

### 2-((2-amino-5-(2-(difluoromethyl)-6-methylpyridin-4-yl)-6-(4-fluorophenyl)pyrimidin-4-yl)oxy)-N-(pyridin-2-yl)acetamide

Step 1: 2-((2-amino-5-(2-(difluoromethyl)-6-methylpyridin-4-yl)-6-(4-fluorophenyl)pyrimidin-4-yl)oxy)-N-(pyridin-2-yl)acetamide. Following step 2, example 2. From 2-((2-amino-5-(2-(difluoromethyl)-6-methylpyridin-4-yl)-6-(4-fluorophenyl)pyrimidin-4-yl)oxy)acetic acid, pyridin-2-amine, HATU and TEA in THF. ES-MS (m/z): [M+1]⁺=481.1.

### Example 108

### 2-amino-5-(2-(difluoromethyl)-6-methylpyridin-4-yl)-6-(4-fluorophenyl)-N-((6-isopropylpyridin-2-yl)methyl)pyrimidine-4-carboxamide

Step 1: 6-(prop-1-en-2-yl)picolinonitrile. A mixture of 6-bromopicolinonitrile (500 mg, 2.75 mmol), 4,4,5,5-tetramethyl-2-(prop-1-en-2-yl)-1,3,2-dioxaborolane (462 mg, 2.75 mmol), potassium carbonate (1.14 g, 8.24 mmol) and Pd(dppf)Cl₂ (201mg, 0.27 mmol) in 1,4-dioxane (10 mL) and water (1 mL) was stirred at 100° C. overnight under nitrogen atmosphere. The reaction mixture was cooled concentrated. The residue was purified on a silica gel column (ethyl acetate/petroleum ether=1/20) to afford 6-(prop-1-en-2-yl)picolinonitrile as a yellow oil (320 mg, 81%). ES-MS (m/z): [M+1]⁺=145.0.

Step 2: tert-butyl((6-isopropylpyridin-2-yl)methyl)carbamate. To a stirred mixture of 6-(prop-1-en-2-yl)picolinonitrile (170 mg, 1.18 mmol), TEA (358 mg, 3.54 mmol) and (Boc)₂O (386 mg, 1.77 mmol) in MeOH (10 mL) was added 10% Pd/C (40 mg). The reaction mixture was stirred at room temperature for 4 h under hydrogen atmosphere (15 psi). The reaction mixture was filtered and concentrated to afford tert-butyl((6-isopropylpyridin-2-yl)methyl)carbamate as a light yellow oil (180 mg, 61%). ES-MS (m/z): [M+1]⁺=251.1.

Step 3: (6-isopropylpyridin-2-yl)methanamine hydrochloride. To a stirred mixture of tert-butyl((6-isopropylpyridin-2-yl)methyl)carbamate (180 mg, 0.72 mmol) in ethyl acetate (2.0 mL) was added HCl/ethyl acetate (4 M, 4.0 mL). After stirring at room temperature for 1 h, the reaction mixture was concentrated to afford (6-isopropylpyridin-2-yl)methanamine hydrochloride as a light yellow solid (130 mg). ES-MS (m/z): [M+1]⁺=151.2.

Step 4: 2-amino-5-(2-(difluoromethyl)-6-methylpyridin-4-yl)-6-(4-fluorophenyl)-N-((6-isopropylpyridin-2-yl)methyl)pyrimidine-4-carboxamide Following step 3, example 100. From (6-isopropylpyridin-2-yl)methanamine, lithium 2-amino-5-(2-(difluoromethyl)-6-methylpyridin-4-yl)-6-(4-fluorophenyl)pyrimidine-4-carboxylate, DIPEA and HATU in DMF. ES-MS (m/z): [M+1]⁺=507.1.

### Example 109

### 2-amino-N-(((6-cyclopropylpyridin-2-yl)methyl)-5-(2-(difluoromethyl)-6-methylpyridin-4-yl)-6-(4-fluorophenyl)pyrimidine-4-carboxamide

Step 1: 6-cyclopropylpicolinonitrile. Following step 1, example 108. From 6-bromopicolinonitrile, cyclopropylboronic acid, potassium carbonate and Pd(dppf)Cl₂ in 1,4-dioxane and water. ES-MS (m/z): [M+1]⁺=145.0.

Step 2: tert-butyl((6-cyclopropylpyridin-2-yl)methyl)carbamate. Following step 2, example 108. From 6-cyclopropylpicolinonitrile, TEA, (Boc)₂O and 10% Pd/C in MeOH under H₂ atmosphere (15 psi). ES-MS (m/z): [M+1]⁺=249.3.

Step 3: (6-cyclopropylpyridin-2-yl)methanamine hydrochloride. Following step 3, example 108. From tert-butyl((6-cyclopropylpyridin-2-yl)methyl)carbamate and HCl in ethyl acetate. ES-MS (m/z): [M+1]⁺=149.1.

Step 4: 2-amino-N-((6-cyclopropylpyridin-2-yl)methyl)-5-(2-(difluoromethyl)-6-methylpyridin-4-yl)-6-(4-fluorophenyl)pyrimidine-4-carboxamide Following step 3, example 100. From (6-cyclopropylpyridin-2-yl)methanamine hydrochloride, lithium 2-amino-5-(2-(difluoromethyl)-6-methylpyridin-4-yl)-6-(4-fluorophenyl)pyrimidine-4-carboxylate, DIPEA, and HATU in DMF. ES-MS (m/z): [M+1]⁺=505.1.

### Example 110

### 2-amino-5-(2-(difluoromethyl)-6-methylpyridin-4-yl)-6-(4-fluorophenyl)-N-((6-(2-hydroxypropan-2-yl)pyridin-2-yl)methyl)pyrimidine-4-carboxamide

Step 1: 2-(6-(hydroxymethyl)pyridin-2-yl)propan-2-ol. A solution of methylmagnesium bromide (in ether, 3.0 M, 16 mL) was added dropwise into methyl 6-(hydroxymethyl)picolinate (2.0 g, 12.0 mmol) in anhydrous THF (20 mL) at 0° C. under nitrogen atmosphere. The reaction mixture then was warmed slowly to room temperature and stirred at this temperature for 3 h. After cooling to 0° C., the reaction mixture was quenched by dropwise addition of 10% aqueous HCl. The resulting mixture was extracted with ethyl acetate (10 mL×2). The combined organic extracts were washed with saturated aqueous NaHCO₃ (10 mL), followed by brine (10 mL). The organic layer was dried over Na₂SO₄, filtered and concentrated. The residue was purified on silica gel column with 15% EA in PE to obtain 2-(6-(hydroxymethyl)pyridin-2-yl)propan-2-ol (1.0 g, 50%) as colorless oil. ES-MS (m/z): [M+1]⁺=168.2.

Step 2: 2-(6-(azidomethyl)pyridin-2-yl)propan-2-ol. A mixture of 2-(6-(hydroxymethyl)pyridin-2-yl)propan-2-ol (800 mg, 4.8 mmol), NaN₃ (368 mg, 5.8 mmol) and PPh₃ (2.5 g, 12.0 mmol) in 25 mL of DMF/CCl₄=4:1 was stirred and heated to 90° C. under nitrogen atmosphere. The reaction mixture then was cooled and quenched with 5 mL of water. The resulting mixture was poured into water (20 mL), extracted with DCM (40 mL×3). The combined organic extracts were dried over Na₂SO₄, filtered and concentrated. The residue was purified on silica gel column (PE:EA=20:1) to give 2-(6-(azidomethyl)pyridin-2-yl)propan-2-ol (300 mg, 32%) as yellow solid. ES-MS (m/z): [M+1]⁺=193.1.

Step 3: 2-(6-(aminomethyl)pyridin-2-yl)propan-2-ol. A mixture of 2-(6-(azidomethyl)pyridin-2-yl)propan-2-ol (300 mg, 1.56mmol) and 10% Pd/C (100 mg) in CH₃OH (10 mL) was stirred under hydrogen atmosphere (1 atm) at room temperature for 4 h. The reaction mixture was filtered and concentrated. The residue was purified on a silica gel column (CH₃OH:DCM=10:1) to get 2-(6-(aminomethyl)pyridin-2-yl)propan-2-ol (200 mg, 77%) as yellow solid. ES-MS (m/z): [M+1]⁺=167.2.

Step 4: 2-amino-5-(2-(difluoromethyl)-6-methylpyridin-4-yl)-6-(4-fluorophenyl)-N-((6-(2-hydroxypropan-2-yl)pyridin-2-yl)methyl)pyrimidine-4-carboxamide. Following step 3, example 100. From 2-(6-(aminomethyl)pyridin-2-yl)propan-2-ol, DIPEA, HATU and lithium 2-amino-5-(2-(difluoromethyl)-6-methylpyridin-4-yl)-6-(4-fluorophenyl)pyrimidine-4-carboxylate in DMF. ES-MS (m/z): [M+1]⁺=523.2.

### Example 111

### (2-amino-5-(2-(trifluoromethyl)-6-methylpyridin-4-yl)-6-(4-fluorophenyl)pyrimidin-4-yl)oxy)ethyl)sulfuric diamide

Step 1: tert-butyl N-(2-((2-amino-6-(4-fluorophenyl)-5-(2-methyl-6-(trifluoromethyl)pyridin-4-yl)pyrimidin-4-yl)oxy)ethyl)sulfamoylcarbamate. Following step 2, example 8. From tert-butyl N-(2-hydroxyethyl)sulfamoylcarbamate, 60% NaH and 4-chloro-6-(4-fluorophenyl)-5-(2-methyl-6-(trifluoromethyl)pyridin-4-yl)pyrimidin-2-amine in DMF. ES-MS (m/z): [M+1]⁺=587.3.

Step 2: (2-amino-5-(2-(trifluoromethyl)-6-methylpyridin-4-yl)-6-(4-fluorophenyl)pyrimidin-4-yl)oxy)ethyl)sulfuric diamide A mixture of tert-butyl N-(2-((2-amino-6-(4-fluorophenyl)-5-(2-methyl-6-(trifluoromethyl)pyridin-4-yl)pyrimidin-4-yl)oxy)ethyl)sulfamoylcarbamate (100 mg, 0.17 mmol) in HCl/CH₃OH (6.0 M, 2.0 mL) was stirred at room temperature for 1 h. The reaction mixture was concentrated, triturated with NH₃/CH₃OH (2.0 M, 2.0 mL). The mixture was concentrated again and purified by prep-HPLC to give (2-amino-5-(2-(trifluoromethyl)-6-methylpyridin-4-yl)-6-(4-fluorophenyl)pyrimidin-4-yl)oxy)ethyl)sulfuric diamide (26.7 mg, 33%) as white solid. ES-MS (m/z): [M+1]⁺=487.0.

### Example 112

### (2-amino-5-(2-(trifluoromethyl)-6-methylpyridin-4-yl)-6-phenyl)pyrimidin-4-yl)oxy)ethyl)sulfuric diamide

Step 1: tert-butyl N-(2-((2-amino-5-(2-(difluoromethyl)-6-methylpyridin-4-yl)-6-phenylpyrimidin-4-yl)oxy)ethyl)sulfamoylcarbamate. Following step 2, example 8. From tert-butyl N-(2-hydroxyethyl)sulfamoylcarbamate, 60% NaH and 4-chloro-5-(2-methyl-6-(trifluoromethyl)pyridin-4-yl)-6-phenylpyrimidin-2-amine in DMF. ES-MS (m/z): [M+1]⁺=568.7.

Step 2: (2-amino-5-(2-(trifluoromethyl)-6-methylpyridin-4-yl)-6-phenyl)pyrimidin-4-yl)oxy)ethyl)sulfuric diamide. Following step 2, example 111. From tert-butyl N-(2-((2-amino-5-(2-(difluoromethyl)-6-methylpyridin-4-yl)-6-phenylpyrimidin-4-yl)oxy)ethyl)sulfamoylcarbamate and HCl/MeOH (6 M). ES-MS (m/z): [M+1]⁺=469.0.

### Example 113

### 1-(2-((2-amino-5-(2-(difluoromethyl)-6-methylpyridin-4-yl)-6-(4-fluorophenyl)pyrimidin-4-yl)oxy)ethyl)-3-methylurea

Step 1: 1-(2-((2-amino-5-(2-(difluoromethyl)-6-methylpyridin-4-yl)-6-(4-fluorophenyl)pyrimidin-4-yl)oxy)ethyl)-3-methylurea. Following step 3, example 54. From 4-(2-aminoethoxy)-5-(2-(difluoromethyl)-6-methylpyridin-4-yl)-6-(4-fluorophenyl)pyrimidin-2-amine, triethylamine and N-methylcarbamoyl chloride in DCM. ES-MS (m/z): [M+1]⁺=447.0.

### Example 114

### 4-((1,4-dioxan-2-yemethoxy)-5-(2-(difluoromethyl)-6-methylpyridin-4-yl)-6-(4-fluorophenyl)pyrimidin-2-amine

Step 1: 4-((1,4-dioxan-2-yl)methoxy)-5-(2-(difluoromethyl)-6-methylpyridin-4-yl)-6-(4-fluorophenyl)pyrimidin-2-amine Following step 2, example 8. From (1,4-dioxan-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=447.1.

### Example 115

### 1-(2-amino-5 -(2-(difluoromethyl)-6-methylpyridin-4-yl)-6-(4-fluorophenyl)pyrimidin-4-yl)oxy)ethyl)-3-methyl-sulfuric diamide

Step 1: 1-(2-amino-5-(2-(difluoromethyl)-6-methylpyridin-4-yl)-6-(4-fluorophenyl)pyrimidin-4-yl)oxy)ethyl)-3-methyl-sulfuric diamide. Following step 3, example 54. From 4-(2-aminoethoxy)-5-(2-(difluoromethyl)-6-methylpyridin-4-yl)-6-(4-fluorophenyl)pyrimidin-2-amine, triethylamine and methylsulfamoyl chloride in DCM. ES-MS (m/z): [M+1]⁺=482.9.

### Example 116

### methyl(2-((2-amino-5-(2-(difluoromethyl)-6-methylpyridin-4-yl)-6-(4-fluorophenyl)pyrimidin-4-yl)oxy)ethyl)carbamate

Step 1: methyl(2-((2-amino-5-(2-(difluoromethyl)-6-methylpyridin-4-yl)-6-(4-fluorophenyl)pyrimidin-4-yl)oxy)ethyl)carbamate. Following step 3, example 54. From 4-(2-aminoethoxy)-5-(2-(difluoromethyl)-6-methylpyridin-4-yl)-6-(4-fluorophenyl)pyrimidin-2-amine, triethylamine and methyl chloroformate in DCM. ES-MS (m/z): [M+1]⁺=448.0.

### Example 117

### 2-((2-amino-5-(2-(difluoromethyl)-6-methylpyridin-4-yl)-6-(4-fluorophenyl)pyrimidin-4-yl)oxy)-N-ethyl-N-(pyridin-2-yl)acetamide

Step 1: 2-((4-methoxybenzyl)oxy)-N-(pyridin-2-yl)acetamide. To a mixture of 2-((4-methoxybenzyl)oxy)acetic acid (200 mg, 1.02 mmol) and DIPEA (395 mg, 3.06 mol) in DMF (2 mL) was added HATU (584 mg, 1.53 mmol) at room temperature. After stirring at room temperature for 10 min, pyridin-2-amine (200 mg, 1.02 mmol) was added. The resulting mixture was stirred at room temperature further for 2 h, then was quenched with water (50 ml). The mixture was extracted with ethyl acetate (100 mL×2). The combined organic extracts were washed with brine (100 mL), dried over Na₂SO₄, filtered and concentrated. The residue was purified on a silica gel column (DCM/MeOH=20/1) to give 2-((4-methoxybenzyl)oxy)-N-(pyridin-2-yl)acetamide (160 mg, 57.7%) as a colorless oil.

Step 2: N-ethyl-2-((4-methoxybenzyl)oxy)-N-(pyridin-2-yl)acetamide. To a mixture of 2-((4-methoxybenzyl)oxy)-N-(pyridin-2-yl)acetamide (160 mg, 0.59 mmol) in DMF (2 ml) was added 60% NaH (21.2 mg, 0.88 mmol) at 0° C. After stirring at room temperature for 30 min, iodoethane (119 mg, 0.76 mmol) was added. After further stirring for 30 min, the reaction mixture was quenched with water (50 mL). The mixture was extracted with EA (50 mL×2). The combined extracts were washed with brine (50 mL), dried over Na₂SO₄, filtered and concentrated. The residue was purified on a silica gel column (PE/EA=5/1) to give N-ethyl-2-((4-methoxybenzyl)oxy)-N-(pyridin-2-yl)acetamide (30 mg, 16.9%) as a colorless oil.

Step 3: N-ethyl-2-hydroxy-N-(pyridin-2-yl)acetamide. A solution of N-ethyl-2-((4-methoxybenzyl)oxy)-N-(pyridin-2-yl)acetamide (30 mg, 0.10 mmol) in DCM/TFA=1/1 (2 mL) was stirred at room temperature for 1 h. The reaction mixture was concentrated and the residue was purified by C18 reverse phase silica gel chromatograph to give N-ethyl-2-hydroxy-N-(pyridin-2-yl)acetamide (16 mg, 88.8%) as a colorless oil.

Step 4: 2-((2-amino-5-(2-(difluoromethyl)-6-methylpyridin-4-yl)-6-(4-fluorophenyl)pyrimidin-4-yl)oxy)-N-ethyl-N-(pyridin-2-yl)acetamide. Following step 2, example 8. From N-ethyl-2-hydroxy-N-(pyridin-2-yl)acetamide, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=509.0.

### Example 118

### 2-((2-amino-5-(2-(difluoromethyl)-6-methylpyridin-4-yl)-6-(4-fluorophenyl)pyrimidin-4-yl)oxy)-N-ethyl-N-(oxetan-3-yl)acetamide

Step 1: 2-((2-amino-5-(2-(difluoromethyl)-6-methylpyridin-4-yl)-6-(4-fluorophenyl)pyrimidin-4-yl)oxy)-N-ethyl-N-(oxetan-3-yl)acetamide. Following step 2, example 2. From lithium 2-((2-amino-5-(2-(difluoromethyl)-6-methylpyridin-4-yl)-6-(4-fluorophenyl)pyrimidin-4-yl)oxy)acetate, N-ethyloxetan-3-amine, HATU and DIPEA in DMF. ES-MS (m/z): [M+1]⁺=488.0.

### Example 119

### methyl(2-((2-amino-5-(2-methyl-6-(trifluoromethyl)pyridin-4-yl)-6-phenylpyrimidin-4-yl)oxy)ethyl)carbamate

Step 1: methyl(2-((2-amino-5-(2-methyl-6-(trifluoromethyl)pyridin-4-yl)-6-phenylpyrimidin-4-yl)oxy)ethyl)carbamate. Following step 3, example 54. From 4-(2-aminoethoxy)-5-(2-methyl-6-(trifluoromethyl)pyridin-4-yl)-6-phenylpyrimidin-2-amine, triethylamine and methyl chloroformate in DCM. ES-MS (m/z): [M+1]⁺=448.2.

### Example 120

### 1-(2-((2-amino-5-(2-methyl-6-(trifluoromethyl)pyridin-4-yl)-6-phenylpyrimidin-4-yl)oxy)ethyl)-3-methylurea

Step 1: 1-(2-((2-amino-5-(2-methyl-6-(trifluoromethyl)pyridin-4-yl)-6-phenylpyrimidin-4-yl)oxy)ethyl)-3-methylurea. Following step 3, example 54. From 4-(2-aminoethoxy)-5-(2-methyl-6-(trifluoromethyl)pyridin-4-yl)-6-phenylpyrimidin-2-amine, triethylamine and N-methylcarbamoyl chloride in DCM. ES-MS (m/z): [M+1]⁺=447.2.

### Example 121

### 1-(2-amino-5-(2-(trifluoromethyl)-6-methylpyridin-4-yl)-6-phenylpyrimidin-4-yl)oxy)ethyl)-3-methyl-sulfuric diamide

Step 1: 1-(2-amino-5-(2-(trifluoromethyl)-6-methylpyridin-4-yl)-6-phenylpyrimidin-4-yl)oxy)ethyl)-3-methyl-sulfuric diamide. Following step 3, example 54. From 4-(2-aminoethoxy)-5-(2-methyl-6-(trifluoromethyl)pyridin-4-yl)-6-phenylpyrimidin-2-amine, triethylamine and methylsulfamoyl chloride in DCM. ES-MS (m/z): [M+1]⁺=483.2.

### Example 122

### 1-(2-((2-amino-5-(2-(difluoromethyl)-6-methylpyridin-4-yl)-6-(4-fluorophenyl)pyrimidin-4-yl)oxy)ethyl)pyridin-2(1H)-one

Step 1: 1-(2-hydroxyethyl)pyridin-2(1H)-one. A mixture of pyridin-2(1H)-one (950 mg, 10.0 mmol), 2-bromoethanol (1.25 g, 10.0 mmol) and K₂CO₃ (4.20 g, 30.0 mmol) in acetone (30 mL) was stirred at 50° C. for 16 h. The reaction mixture was cooled, filtered and concentrated. The residue was purified on a silica gel column with PE/EtOAc (5:1) to give 1-(2-hydroxyethyl)pyridin-2(1H)-one (970 mg, 70%) as yellow solid. ES-MS (m/z): [M+1]⁺=140.0.

Step 2: 1-(2-((2-amino-5-(2-(difluoromethyl)-6-methylpyridin-4-yl)-6-(4-fluorophenyl)pyrimidin-4-yl)oxy)ethyl)pyridin-2(1H)-one. Following step 2, example 8. From 1-(2-hydroxyethyl)pyridin-2(1H)-one, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=468.0.

### Example 123

### 1-(2-((2-amino-5-(2-(difluoromethyl)-6-methylpyridin-4-yl)-6-(4-fluorophenyl)pyrimidin-4-yl)oxy)ethyl)-1,3-diethylurea

Step 1: tert-butyl(2-((2-amino-5-(2-(difluoromethyl)-6-methylpyridin-4-yl)-6-(4-fluorophenyl)pyrimidin-4-yl)oxy)ethyl)(ethyl)carbamate. Following step 2, example 8. From tert-butyl ethyl(2-hydroxyethyl)carbamate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=518.3.

Step 2: 5-(2-(difluoromethyl)-6-methylpyridin-4-yl)-4-(2-(ethylamino)ethoxy)-6-(4-fluorophenyl)pyrimidin-2-amine. A mixture of tert-butyl(24(2-amino-5-(2-(difluoromethyl)-6-methylpyridin-4-yl)-6-(4-fluorophenyl)pyrimidin-4-yl)oxy)ethyl)(ethyl)carbamate (100 mg, 0.19 mmol) and HCl/CH₃OH (2 M, 2.0 mL) was stirred at room temperature for 1 h. The reaction mixture was concentrated, triturated with NH₃/CH₃OH (2 M, 2.0 mL), followed by concentration again. Water (20 mL) was added to the residue, and the mixture was extracted with CH₂Cl₂ (10 mL×3). The combined organic extracts were dried over Na₂SO₄, filtered and concentrated to give crude 5-(2-(difluoromethyl)-6-methylpyridin-4-yl)-4-(2-(ethylamino)ethoxy)-6-(4-fluorophenyl)pyrimidin-2-amine (200 mg, 125%) as yellow solid. ES-MS (m/z): [M+1]⁺=418.3.

Step 3: 1-(2-((2-amino-5-(2-(difluoromethyl)-6-methylpyridin-4-yl)-6-(4-fluorophenyl)pyrimidin-4-yl)oxy)ethyl)-1-ethyl-3-methylurea. To a mixture of 5-(2-(difluoromethyl)-6-methylpyridin-4-yl)-4-(2-(ethylamino)ethoxy)-6-(4-fluorophenyl)pyrimidin-2-amine (50 mg, 0.12 mmol) in DCM (2 mL) were added DIPEA (31 mg, 0.24 mmol), followed by isocyanatoethane (11.1 mg, 0.16 mmol) at 0° C. After stirring at room temperature for 0.5 h, the reaction mixture was concentrated and purified by prep-HPLC to give 1-(2-((2-amino-5-(2-(difluoromethyl)-6-methylpyridin-4-yl)-6-(4-fluorophenyl)pyrimidin-4-yl)oxy)ethyl)-1,3-diethylurea (13 mg, 21%) as white solid. ES-MS (m/z): [M+b 1]⁺=489.1.

### Example 124

### 1-(2-((2-amino-5-(2-(difluoromethyl)-6-methylpyridin-4-yl)-6-(4-fluorophenyl)pyrimidin-4-yl)oxy)ethyl)-1-ethyl-3-methylurea

Step 1: 1-(2-((2-amino-5-(2-(difluoromethyl)-6-methylpyridin-4-yl)-6-(4-fluorophenyl)pyrimidin-4-yl)oxy)ethyl)-1-ethyl-3-methylurea. Following step 3, example 123. From 5-(2-(difluoromethyl)-6-methylpyridin-4-yl)-4-(2-(ethylamino)ethoxy)-6-(4-fluorophenyl)pyrimidin-2-amine, triethylamine and N-methylcarbamoyl chloride in DCM. ES-MS (m/z): [M+1]⁺=475.3.

### Example 125

### (4-(((2-amino-5-(2-(difluoromethyl)-6-methylpyridin-4-yl)-6-(4-fluorophenyl)pyrimidin-4-yl)oxy)methyl)tetrahydro-2H-pyran-4-yl)methanol

Step 1: (tetrahydro-2H-pyran-4,4-diyl)dimethanol. To a suspension of LiAlH₄ (752 mg, 19.8 mmol) in anhydrous THF (20 mL) was added dimethyl dihydro-2H-pyran-4,4(3H)-dicarboxylate (1.0 g, 4.95 mmol) at 0° C. under nitrogen atmosphere. After stirring at room temperature overnight, the reaction mixture was quenched with H₂O (1 mL). After further stirring at room temperature for 30 min, the mixture was filtered and concentrated to get crude (tetrahydro-2H-pyran-4,4-diyl)dimethanol (700 mg, 97%) as a colorless oil.

Step 2: (4-4(2-amino-5-(2-(difluoromethyl)-6-methylpyridin-4-yl)-6-(4-fluorophenyl)pyrimidin-4-yl)oxy)methyl)tetrahydro-2H-pyran-4-yemethanol. Following step 2, example 8. From (tetrahydro-2H-pyran-4,4-diyl)dimethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=475.0.

### Example 135

### 4-((1,5-diethyl-1H-1,2,3-triazol-4-yl)methoxy)-5-(2-(difluoromethyl)-6-methylpyridin-4-yl)-6-(4-fluorophenyl)pyrimidin-2-amine

Step 1: 4-(((tert-butyldimethylsilyl)oxy)methyl)-1,5-diethyl-1H-1,2,3-triazole and 5-(((tert-butyldimethylsilyl)oxy)methyl)-1,4-diethyl-1H-1,2,3-triazole

A mixture of 4-(((tert-butyldimethylsilyl)oxy)methyl)-5-ethyl-1H-1,2,3-triazole (from example 136) (500 mg, 2.1 mmol), iodoethane (418.5 mg, 2.7 mmol) and K₂CO₃ (372.6 mg, 2.7 mmol) in DMF (10 mL) was stirred at 130° C. under microwave for 1 h. The reaction mixture was concentrated and the residue was purified by reverse-phase chromatograph to give 4-(((tert-butyldimethylsilyl)oxy)methyl)-1,5-diethyl-1H-1,2,3-triazole and 5 -(((tert-butyldimethylsilyl)oxy)methyl)-1,4-diethyl-1H-1,2,3-triazole (200 mg, 35%) as a colorless oil. ES-MS (m/z): [M+1]⁺=270.2.

Step 2: (1,5-diethyl-1H-1,2,3-triazol-4-yl)methanol and (1,4-diethyl-1H-1,2,3-triazol-5-yl)methanol

A solution of 4-(((tert-butyldimethylsilyl)oxy)methyl)-1,5-diethyl-1H-1,2,3-triazole and 5-(((tert-butyldimethylsilyl)oxy)methyl)-1,4-diethyl-1H-1,2,3-triazole (200 mg, 0.74 mmol) in HCl/MeOH (1.0 M, 2.0 mL) was stirred at room temperature for 2 h. The reaction mixture was concentrated, dissolved in MeOH, then treated with Amberlyst A-21 ion-exchange resin for 30 min. The mixture was filtered and concentrated to give crude (1,5-diethyl-1H-1,2,3-triazol-4-yl)methanol and (1,4-diethyl-1H-1,2,3-triazol-5-yl)methanol (75 mg, 65%) as a colorless oil.

Step 3: 4-((1,5-diethyl-1H-1,2,3-triazol-4-yl)methoxy)-5-(2-(difluoromethyl)-6-methylpyridin-4-yl)-6-(4-fluorophenyl)pyrimidin-2-amine

Following step 2, example 8. From (1,5-diethyl-1H-1,2,3-triazol-4-yl)methanol and (1,4-diethyl-1H-1,2,3-triazol-5-yl)methanol, 60% NaH and 4-chloro-5-(2-(difluoromethyl)-6-methylpyridin-4-yl)-6-(4-fluorophenyl)pyrimidin-2-amine in DMF. The crude was purified by prep-HPLC to obtain 4-((1,5-diethyl-1H-1,2,3-triazol-4-yl)methoxy)-5-(2-(difluoromethyl)-6-methylpyridin-4-yl)-6-(4-fluorophenyl)pyrimidin-2-amine ES-MS (m/z): [M+1]⁺=484.2.

### Example 136

### 5-(2-(difluoromethyl)-6-methylpyridin-4-yl)-4-((5-ethyl-1-methyl-1H-1,2,3-triazol-4-yl)methoxy)-6-(4-fluorophenyl)pyrimidin-2-amine

Step 1: tert-butyldimethyl(pent-2-yn-1-yloxy)silane

To a stirring solution of pent-2-yn-1-ol (4.0 g, 47.6 mmol), triethylamine (9.6 g, 94.9 mmol) in DCM (50 mL) was added TBSC1 (9.2 g, 62.0 mmol) at room temperature. After stirring at room temperature overnight, the reaction mixture was diluted with water (50 mL), and the layers were separated. The aqueous layer was extracted with DCM (80 mL×3). The combined organic layers were washed with brine, dried over Na₂SO₄, filtered and concentrated to give crude tert-butyldimethyl(pent-2-yn-1-yloxy)silane (8.0 g, 85%) as a yellow oil, which was used for next step without further purification.

Step 2: 1-benzyl-4-(((tert-butyldimethylsilyl)oxy)methyl)-5-ethyl-1H-1,2,3-triazole and 1-benzyl-5-(((tert-butyldimethylsilyl)oxy)methyl)-4-ethyl-1H-1,2,3-triazole

A mixture of tert-butyldimethyl(pent-2-yn-1-yloxy)silane (3.0 g, 22.5 mmol), (azidomethyl)benzene (3.6 g, 27 mmol) and RuClCp*(PPh₃)₂ (1.1 g, 1.4 mmol) in DMA (60 ml) was stirred at 110° C. under N₂ for 2 h. The reaction mixture was cooled and diluted with water (50 mL), extracted with ethyl acetate (50 mL×3). The combined organic layers were washed with brine (50 mL×3), dried over Na₂SO₄, filtered and concentrated. The residue was purified by chromatograph on silica gel to give 1-benzyl-4-(((tert-butyldimethylsilyl)oxy)methyl)-5-ethyl-1H-1,2,3-triazole and 1-benzyl-5-(((tert-butyldimethylsilyl)oxy)methyl)-4-ethyl-1H-1,2,3-triazole (5.6 g, 75%) as a yellow oil.

Step 3: (5 -ethyl-1H-1,2,3-triazol-4-yl)methanol

A mixture of 1-benzyl-5-(((tert-butyldimethylsilyl)oxy)methyl)-4-ethyl-1H-1,2,3-triazole and 1-benzyl-4-(((tert-butyldimethylsilyl)oxy)methyl)-5-ethyl-1H-1,2,3-triazole (5.6 g, 16.9 mmol), 20% Pd/C (1.0 g) and aqueous HCl (12.0 M, 12 drops) in MeOH (60 mL) was stirred at 50° C. under 50 psi hydrogen atmosphere for 4 days. The reaction mixture was filtered and concentrated to give crude (5-ethyl-1H-1,2,3-triazol-4-yl)methanol (2.4 g) as a yellow oil, which was used for next step without further purification.

Step 4: 4-(((tert-butyldimethylsilyl)oxy)methyl)-5-ethyl-1H-1,2,3-triazole

To a stirring solution of (5-ethyl-1H-1,2,3-triazol-4-yl)methanol (2.4 g, 18.9 mmol) and triethylamine (5.7 g, 56.7 mmol) in DCM (50 mL) was added TBSC1 (4.3 g, 28.4 mmol) at room temperature. After stirring at room temperature overnight, the reaction mixture was quenched with water (50 mL) and the layers were separated. The aqueous layer was extracted with DCM (80 mL×3). The combined organic layers were washed with brine (50 mL×3), dried over Na₂SO₄, filtered and concentrated. The residue was purified on silica gel chromatography to give 4-(((tert-butyldimethylsilyl)oxy)methyl)-5-ethyl-1H-1,2,3-triazole (2.4 g, 52%) as a yellow oil.

Step 5: 4-(((tert-butyldimethylsilyl)oxy)methyl)-5-ethyl-1-methyl-1H-1,2,3-triazole and 5-(((tert-butyldimethylsilyl)oxy)methyl)-4-ethyl-1-methyl-1H-1,2,3-triazole

A mixture of 4-(((tert-butyldimethylsilyl)oxy)methyl)-5-ethyl-1H-1,2,3-triazole (500 mg, 2.1 mmol), iodomethane (380.7 mg, 2.7 mmol) and K₂CO₃ (869.4 mg, 6.3 mmol) in DMF (10 ml) was stirred at 130° C. under microwave for 1 h. The reaction mixture was concentrated and the residue was purified by silica gel chromatography to give 4-(((tert-butyldimethylsilyl)oxy)methyl)-5-ethyl-1-methyl-1H-1,2,3-triazole and 5-(((tert-butyldimethylsilyl)oxy)methyl)-4-ethyl-1-methyl-1H-1,2,3-triazole (260 mg, 48%) as a colorless oil. ES-MS (m/z): [M+1]⁺=256.2

Step 6: (5-ethyl-1-methyl-1H-1,2,3-triazol-4-yemethanol and (4-ethyl-1-methyl-1H-1,2,3-triazol-5-yl)methanol

A solution of 4-(((tert-butyldimethylsilyl)oxy)methyl)-5-ethyl-1-methyl-1H-1,2,3-triazole and 5-(((tert-butyldimethylsilyl)oxy)methyl)-4-ethyl-1-methyl-1H-1,2,3-triazole (160 mg, 0.63 mmol) in 1.0 M HCl/MeOH (2.0 mL) was stirred at room temperature for 2 h. The reaction mixture was concentrated and the residue was triturated with 1.0 M NH₃/MeOH, and concentrated again. The residue was purified by reverse-phase chromatography to give (5-ethyl-1-methyl-1H-1,2,3-triazol-4-yl)methanol and (4-ethyl-1-methyl-1H-1,2,3-triazol-5-yl)methanol (70 mg, 79%) a colorless oil.

Step 7: 5-(2-(difluoromethyl)-6-methylpyridin-4-yl)-4-((5-ethyl-1-methyl-1H-1,2,3-triazol-4-yl)methoxy)-6-(4-fluorophenyl)pyrimidin-2-amine

Following step 2, example 8. From (5-ethyl-1-methyl-1H-1,2,3-triazol-4-yl)methanol and (4-ethyl-1-methyl-1H-1,2,3-triazol-5-yl)methanol, 60% NaH and 4-chloro-5-(2-(difluoromethyl)-6-methylpyridin-4-yl)-6-(4-fluorophenyl)pyrimidin-2-amine in DMF. The crude was purified by prep-HPLC to obtain 5-(2-(difluoromethyl)-6-methylpyridin-4-yl)-4-((5-ethyl-1-methyl-1H-1,2,3-triazol-4-yl)methoxy)-6-(4-fluorophenyl)pyrimidin-2-amine. ES-MS (m/z): [M+1]⁺=470.2.

### Examples 154

### 2-((6-((2-((2-amino-5-(2-(difluoromethyl)-6-methylpyridin-4-yl)-6-(4-fluorophenyl)pyrimidin-4-yl)oxy)ethyl)(methyl)amino)pyridin-2-yl)oxy)ethan-1-ol

Step 1: 2-((6-bromopyridin-2-yl)oxy)ethanol

A mixture of 6-bromopyridin-2-ol (500 mg, 2.874 mmol), 2-bromoethanol (503 mg, 4.023 mmol) and K₂CO₃ (595 mg, 4.311 mmol) in DMF (5 mL) was stirred at 70° C. under nitrogen atmosphere overnight. The reaction mixture was concentrated and the residue was purified by silica gel column (PE:EA=2:1) to give 2-((6-bromopyridin-2-yl)oxy)ethanol (300 mg, 48%) as a yellow oil. ES-MS (m/z): [M+1]⁺=220.0.

Step 2: 2-bromo-6-(2-((tert-butyldimethylsilyl)oxy)ethoxy)pyridine

To a stirring solution of 2-((6-bromopyridin-2-yl)oxy)ethanol (300 mg, 1.376 mmol) in DCM (10 mL) was added imidazole (280 mg, 4.128 mmol), followed by TBSC1 (228 mg, 1.514 mmol) at 0° C. After stirring at room temperature for 2 h, the reaction mixture was diluted with DCM (20 mL), washed with water (10 mL×3) and brine (10 mL×3). The organic layer was dried over Na₂SO₄, filtered and concentrated to give crude 2-bromo-6-(2-((tert-butyldimethylsilyl)oxy)ethoxy)pyridine (300 mg, 66%) as a yellow oil, which was used for next step with further purification. ES-MS (m/z): [M+1]⁺=332.0.

### Step 3: 2-((6-(2-((tert-butyldimethylsilyl)oxy)ethoxy)pyridin-2-yl)(methyeamino)ethanol

A solution of 2-bromo-6-(2-((tert-butyldimethylsilyl)oxy)ethoxy)pyridine (300 mg, 0.903 mmol) and 2-(methylamino)ethanol (339 mg, 4.52 mmol) in pyridine (5 mL) was stirred at 90° C. under nitrogen atmosphere overnight. The reaction mixture was concentrated and the residue was purified by silica gel column (PE:EA=2:1) to give 2-((6-(2-((tert-butyldimethylsilyl)oxy)ethoxy)pyridin-2-yl)(methyl)amino)ethanol (140 mg, 48%) as a colorless oil. ES-MS (m/z): [M+1]⁺=327.2.

Step 4: 2-((6-((2-((2-amino-5-(2-(difluoromethyl)-6-methylpyridin-4-yl)-6-(4-fluorophenyl)pyrimidin-4-yl)oxy)ethyl)(methyl)amino)pyridin-2-yl)oxy)ethan-1-ol

Following step 2, example 8. From 24(6-(2-((tert-butyldimethylsilyl)oxy)ethoxy)pyridin-2-yl)(methyl)amino)ethanol, 60% NaH and 4-chloro-5-(2-(difluoromethyl)-6-methylpyridin-4-yl)-6-(4-fluorophenyl)pyrimidin-2-amine in DMF. The crude was purified by prep-HPLC to obtain 2-((6-((2-((2-amino-5-(2-(difluoromethyl)-6-methylpyridin-4-yl)-6-(4-fluorophenyl)pyrimidin-4-yl)oxy)ethyl)(methyl)amino)pyridin-2-yl)oxy)ethan-1-ol, ES-MS (m/z): [M+1]⁺=541.3.

### Example 209

### 2-(2-(((2-amino-5-(2-(difluoromethyl)-6-methylpyridin-4-yl)-6-(4-fluorophenyl)pyrimidin-4-yl)oxy)methyl)-1H-imidazol-1-yl)propan-1-ol

Step 1: ethyl 2-(2-formyl-1H-imidazol-1-yl)propanoate

A mixture of 1H-imidazole-2-carbaldehyde (500 mg, 5.20 mmol), ethyl 2-bromopropanoate (1.3 g, 6.77 mmol), Cs₂CO₃ (2.1 g, 6.25 mmol) in DMF (4 mL) was stirred at 100° C. for 4 h. The reaction mixture was concentrated in vacuo, and the residue was purified by silica gel column (PE:EA=10:1) to give ethyl 2-(2-formyl-1H-imidazol-1-yl)propanoate (500 mg, 49%) as a colorless oil. ES-MS (m/z): [M+1]⁺=197.2

Step 2: ethyl 2-(2-(hydroxymethyl)-1H-imidazol-1-yl)propanoate

A solution of ethyl 2-(2-formyl-1H-imidazol-1-yl)propanoate (500 mg, 2.55 mmol) in CH₃OH (5 mL) was stirred at −78° C., NaBH₄ (198 mg, 5.2 mmol) then was added. The resulting mixture was stirred at −78° C. for 12 min, and quenched with aqueous HCl (1 M, 1 mL). The resulting mixture was concentrated in vacuo, and the residue was purified by silica gel column (DCM:MeOH=10:1) to give ethyl 2-(2-(hydroxymethyl)-1H-imidazol-1-yl)propanoate (300 mg, 59%) as a colorless oil. ES-MS (m/z): [M+1]⁺=199.2.

Step 3: 2-(2-4(2-amino-5-(2-(difluoromethyl)-6-methylpyridin-4-yl)-6-(4-fluorophenyl)pyrimidin-4-yl)oxy)methyl)-1H-imidazol-1-yl)propanoic acid

Following step 2, example 8. From ethyl 2-(2-(hydroxymethyl)-1H-imidazol-1-yl)propanoate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=499.2.

Step 4: 2-(2-4(2-amino-5-(2-(difluoromethyl)-6-methylpyridin-4-yl)-6-(4-fluorophenyl)pyrimidin-4-yl)oxy)methyl)-1H-imidazol-1-yl)propan-1-ol

To a solution of 2-(2-4(2-amino-5-(2-(difluoromethyl)-6-methylpyridin-4-yl)-6-(4-fluorophenyl)pyrimidin-4-yl)oxy)methyl)-1H-imidazol-1-yl)propanoic acid (50 mg, 0.10 mmol) in THF (2 mL) at 0° C. was added LiAlH₄ (1 M in THF, 0.4 mL, 0.4 mmol) dropwise under nitrogen. The resulting mixture then was stirred at room temperature for 1 h, followed by quenching with 2 drops of water. The resulting mixture was purified by prep-HPLC to give 2-(2-(((2-amino-5-(2-(difluoromethyl)-6-methylpyridin-4-yl)-6-(4-fluorophenyl)pyrimidin-4-yl)oxy)methyl)-1H-imidazol-1-yl)propan-1-ol as a white solid. ES-MS (m/z): [M+1]⁺=485.0.

### Example 225

### 2-(6-4(2-amino-5-(2-(difluoromethyl)-6-methylpyridin-4-yl)-6-(4-fluorophenyepyrimidin-4-yl)oxy)methyl)pyridin-2-yl)propan-2-ol

### Step 1: 2-(6-(hydroxymethyl)pyridin-2-yl)propan-2-ol

To a stirring solution of methyl 6-(hydroxymethyl)picolinate (200 mg, 1.196 mmol) in THF (3 mL) was added CH₃BrMg (3M in diethyl ether, 2.0 mL, 6.0 mmol) at 0° C. After stirring at room temperature for 16 h, the reaction mixture was quenched with saturated aqueous NH₄Cl (50 mL) carefully, then extracted with ethyl acetate (50 mL×3). The combined organic layer was dried over Na₂SO₄, filtered and concentrated. The residue was purified by silica gel column (PE:EA=5:1) to get 2-(6-(hydroxymethyl)pyridin-2-yl)propan-2-ol (92 mg, 46%) as a yellow oil. ES-MS (m/z): [M+1]⁺=168.1.

Step 2: 2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-ol

Following step 2, example 8. From 2-(6-(hydroxymethyl)pyridin-2-yl)propan-2-ol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=496.2.

### Examples 227

### 5-(2-(difluoromethyl)-6-methylpyridin-4-yl)-4-((1-ethyl-1H-pyrazol-5-yl)methoxy)-6-(4-fluorophenyl)pyrimidin-2-amine

Step 1: (1H-pyrazol-5-yl)methanol

To a stirring solution of ethyl 1H-pyrazole-5-carboxylate (2.0 g, 14.3 mmol) in THF (20 mL) was added LiAlH₄ (1 M in THF, 43.0 mL, 43.0 mmol) slowly under nitrogen atmosphere at 0° C. After stirring at room temperature for 3 h, the reaction mixture was quenched by 10 drops of water, filtered and concentrated to give crude (1H-pyrazol-5-yl)methanol (1.5 g, 107%) as colorless oil, which was used for next step without further purification. ES-MS (m/z): [M+1]⁺=99.3.

Step 2: 5-(((tert-butyldimethylsilyl)oxy)methyl)-1H-pyrazole

A mixture of (1H-pyrazol-5-yl)methanol (1.50 g, 15.3 mmol), TBSCl (3.44 g, 22.9 mmol), imidazole (1.56 g, 22.9 mmol) and K₂CO₃ (1.06 g, 7.65 mmol) in DMF (10 mL) was stirred at 0° C. for 20 min The reaction mixture was poured in to water (30 mL), extracted with ethyl acetate (10 mL×3), and the combined organic layers were dried over Na₂SO₄, concentrated and purified by column chromatography on silica gel with DCM:MeOH=20:1 to give 5-(((tert-butyldimethylsilyl)oxy)methyl)-1H-pyrazole (1.5 g, 46%) as colorless oil. ES-MS (m/z): [M+1]⁺=213.3.

Step 3: 5-(((tert-butyldimethylsilyl)oxy)methyl)-1-ethyl-1H-pyrazole and 3-(((tert-butyldimethylsilyl)oxy)methyl)-1-ethyl-1H-pyrazole

A mixture of 5-(((tert-butyldimethylsilyl)oxy)methyl)-1H-pyrazole (0.500 g, 2.35 mmol), iodoethane (0.478 g, 3.07 mmol) and K₂CO₃ (0.977 g, 7.08 mmol) in DMF (2 mL) was stirred at 130° C. for 5 h. The reaction mixture was cooled and triturated with DCM (10 mL×3). The combined organic phases were dried over Na₂SO₄, concentrated and purified by column chromatography on silica gel with PE:EA=10:1 to give a mixture of 5-(((tert-butyldimethylsilyl)oxy)methyl)-1-ethyl-1H-pyrazole and 3-(((tert-butyldimethylsilyl)oxy)methyl)-1-ethyl-1H-pyrazole (360 mg, 64%) as yellow solid. ES-MS (m/z): [M+1]⁺=241.4.

### Step 4: (1-ethyl-1H-pyrazol-5-yemethanol and (1-ethyl-1H-pyrazol-3-yemethanol

A mixture of 5-(((tert-butyldimethylsilyl)oxy)methyl)-1-ethyl-1H-pyrazole and 3-(((tert-butyldimethylsilyl)oxy)methyl)-1-ethyl-1H-pyrazole (360 mg, 1.5 mmol) and NH₄F (278 mg, 7.5 mmol) in CH₃OH (5 mL) was stirred at room temperature overnight. The mixture was concentrated and purified by column chromatography on silica gel with DCM: CH₃OH=10:1 to give (1-ethyl-1H-pyrazol-5-yl)methanol and (1-ethyl-1H-pyrazol-3-yl)methanol (180 mg, 95%) as colorless oil. ES-MS (m/z): [M+1]⁺=127.2.

Step 5: 5-(2-(difluoromethyl)-6-methylpyridin-4-yl)-4-((1-ethyl-1H-pyrazol-5-yl)methoxy)-6-(4-fluorophenyl)pyrimidin-2-amine

Following step 2, example 8. From (1-ethyl-1H-pyrazol-5-yl)methanol and (1-ethyl-1H-pyrazol-3-yl)nethanol, 60% NaH and 4-chloro-5-(2-(difluoromethyl)-6-methylpyridin-4-yl)-6-(4-fluorophenyl)pyrimidin-2-amine in DMF. The crude was purified by prep-HPLC to obtain 5-(2-(difluoromethyl)-6-methylpyridin-4-yl)-4-((1-ethyl-1H-pyrazol-5-yl)methoxy)-6-(4-fluorophenyl)pyrimidin-2-amine, ES-MS (m/z): [M+1]⁺=455.2.

### Example 230

### 2-(6-(((2-amino-5-(2-(difluoromethyl)-6-methylpyridin-4-yl)-6-(4-fluorophenyl)pyrimidin-4-yl)oxy)methyl)pyridin-2-yl)-2-methylpropanenitrile

Step 1: 2-bromo-6-(((tert-butyldimethylsilyeoxy)methyl)pyridine

A mixture of (6-bromopyridin-2-yl)methanol (5.0 g, 26.59 mmol), TBSC1 (4.8 g, 31.91 mmol), imidazole (2.3 g, 34.57 mmol) and DMAP (650 mg, 5.32 mmol) in DCM (100 mL) was stirred at room temperature overnight under nitrogen atmosphere. The reaction mixture was diluted with H₂O (100 mL), extracted with DCM (100 mL×2), dried over Na₂SO₄, filtered and concentrated. The residue was purified by silica gel column with ethyl acetate/petroleum ether (10/1) to afford 2-bromo-6-(((tert-butyldimethylsilyl)oxy)methyl)pyridine as colorless oil (8.3 g, 103%). ES-MS (m/z): [M+1]⁺=302.1.

Step 2: 2-(6-(((tert-butyldimethylsilyl)oxy)methyl)pyridin-2-yl)-2-methylpropanenitrile

To a stirring mixture of 2-bromo-6-(((tert-butyldimethylsilyl)oxy)methyl)pyridine (300 mg, 0.99 mmol) and isobutyronitrile (274 mg, 3.97 mmol) in toluene (10 mL) was added KHMDS (1.0 M in THF, 1.49 mL, 1.49 mmol) under nitrogen atmosphere. The resulting mixture was stirred at 80° C. for 1 h, cooled, poured into saturated aqueous NH₄Cl (30 mL) and extracted with toluene (30 mL×3). The combined organic layer was washed with H₂O (20 mL×3), dried over Na₂SO₄, filtered and concentrated. The residue was purified by silica gel column with ethyl acetate/petroleum ether (1/60) to afford 2-(6-(((tert-butyldimethylsilyl)oxy)methyl)pyridin-2-yl)-2-methylpropanenitrile as colorless oil (93 mg, 32%). ES-MS (m/z): [M+1]⁺=291.1.

Step 3: 2-(6-(hydroxymethyl)pyridin-2-yl)-2-methylpropanenitrile

A mixture of 2-(6-(((tert-butyldimethylsilyl)oxy)methyl)pyridin-2-yl)-2-methylpropanenitrile (93 mg, 0.32 mmol) and NH₄F (59 mg, 1.60 mmol) in MeOH (5 mL) was stirred at room temperature for 24 h. The reaction mixture was concentrated and the residue was purified by silica gel column with ethyl acetate/petroleum ether (1/10) to afford 2-(6-(hydroxymethyl)pyridin-2-yl)-2-methylpropanenitrile as colorless oil (44 mg, 78%). ES-MS (m/z): [M+1]⁺=177.2.

Step 4: 2-(6-(((2-amino-5-(2-(difluoromethyl)-6-methylpyridin-4-yl)-6-(4-fluorophenyl)pyrimidin-4-yl)oxy)methyl)pyridin-2-yl)-2-methylpropanenitrile

Following step 2, example 8. From 2-(6-(hydroxymethyl)pyridin-2-yl)-2-methylpropanenitrile, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=505.2.

### Example 238

### 2-(2-4(2-amino-5-(2-(difluoromethyl)-6-methylpyridin-4-yl)-6-(4-fluorophenyl)pyrimidin-4-yl)oxy)methyl)pyridin-3-yl)propan-2-ol

### Step 1: 2-(hydroxymethyl)nicotinic acid

To a solution of furo[3,4-b]pyridine-5,7-dione (3.0 g, 20.1 mmol) in THF (24 mL) was added NaBH₄ (0.76 g, 20.1 mmol) at 15° C. under nitrogen, followed by HOAc (2.4 g, 40.2 mmol) dropwise. After stirring at 15° C. for 4 h, the reaction mixture was concentrated in vacuo. To the residue was added H₂O (20 mL), and the solution was adjusted to pH=1.5 with 4N aqueous H₂SO₄ (11 mL). The mixture was charged onto an ion-exchange resin column, eluted with 1N aqueous NH₄OH (50 mL). The fractions containing 2-(hydroxymethyl)nicotinic acid were concentrated in vacuo. The residue was purified by silica gel chromatography (PE:EA=1:10) to give 2-(hydroxymethyl)nicotinic acid (450 mg, 15%) as a yellow solid.

### Step 2: furo[3,4-b]pyridin-5(7H)-one

A mixture of 2-(hydroxymethyl)nicotinic acid (350 mg, 2.28 mmol) and Ac₂O (0.7 mL, 7.41 mmol) in HOAc (35 mL) was stirred at 100° C. for 3 h. The reaction mixture was cooled and concentrated in vacuo. The residue was purified by silica gel chromatography (PE:EA=3:1) to give furo[3,4-b]pyridin-5(7H)-one (202 mg, 65%) as a white solid.

### Step 3: 2-(2-(hydroxymethyl)pyridin-3-yl)propan-2-ol

To a stirring solution of CH₃MgBr (3 M in ether, 1.97 mL, 5.91 mmol) in THF (10 mL) at 0° C. was added furo[3,4-b]pyridin-5(7H)-one (200 mg, 1.48 mmol) in THF (4 mL) dropwise under nitrogen. After stirring at room temperature for 1 h, the reaction mixture was quenched by H₂O (20 mL), extracted with ethyl acetate (20 mL×4). The combined organic extracts were concentrated in vacuo, and the residue was purified by silica gel chromatography (PE:EA=1:2) to give 2-(2-(hydroxymethyl)pyridin-3-yl)propan-2-ol (110 mg, 45%) as a colorless oil.

### Step 4: 2-(2-(((2-amino-5-(2-(difluoromethyl)-6-methylpyridin-4-yl)-6-(4-fluorophenyl)pyrimidin-4-yl)oxy)methyl)pyridin-3-yl)propan-2-ol

Following step 2, example 8. From 2-(2-(hydroxymethyl)pyridin-3-yl)propan-2-ol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=496.1.

### Example 247

### 2-(((6-(((2-amino-5-(2-(difluoromethyl)-6-methylpyridin-4-yl)-6-(4-fluorophenyl)pyrimidin-4-yl)oxy)methyl)pyridin-2-yl)oxy)acetic acid

### Step 1: 6-hydroxymethyl-pyridin-2-ol

To a stirring solution of 6-hydroxy-pyridine-2-carboxylic acid (2.0 g, 14.38 mmol) in THF (10 mL) was added (CH₃)₂S.BH₃ (10 M, 7.2 mL, 71.89 mmol) at room temperature under nitrogen. The resulting mixture was stirred at 50° C. overnight, then cooled and quenched with MeOH (30 mL) slowly and carefully. After stirring at 50° C. for 4 h, the mixture was concentrated and purified by column chromatography on silica gel (DCM:MeOH=10:1) to give 6-hydroxymethyl-pyridin-2-ol (1.3 g, 72%) as a white solid. ES-MS (m/z): [M+1]⁺=126.1.

### Step 2: (6-hydroxymethyl-pyridin-2-yloxy)-acetic acid tert-butyl ester

A mixture of 6-hydroxymethyl-pyridin-2-ol (500 mg, 4.00 mmol), tert-butyl 2-bromoacetate (1.1 g, 5.59 mmol) and K₂CO₃ (827 mg, 5.99 mmol) in DMF (5 mL) was stirred at 70° C. overnight. The reaction mixture was cooled, quenched with H₂O (20 mL), extracted with EtOAc (20 mL×3). The combined organic extract was washed with brine (20 mL), dried over Na₂SO₄, filtered and concentrated. The residue was purified by column chromatography on silica gel (PE:EA=5:1) to give (6-hydroxymethyl-pyridin-2-yloxy)-acetic acid tert-butyl ester (500 mg, 52%) as a yellow oil. ES-MS (m/z): [M+1]⁺=240.1.

### Step 3: 2-(((6-(((2-amino-5-(2-(difluoromethyl)-6-methylpyridin-4-yl)-6-(4-fluorophenyl)pyrimidin-4-yl)oxy)methyl)pyridin-2-yl)oxy)acetic acid

Following step 2, example 8. From (6-hydroxymethyl-pyridin-2-yloxy)-acetic acid tent-butyl ester, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=512.2.

### Example 248

### 2-((6-((2-((2-amino-5-(2-(difluoromethyl)-6-methylpyridin-4-yl)-6-(4-fluorophenyl)pyrimidin-4-yl)oxy)ethyl)(methyl)amino)pyridin-2-yl)oxy)acetic acid

Step 1: tert-butyl 2-((6-bromopyridin-2-yl)oxy)acetate

A mixture of 6-bromopyridin-2-ol (220 mg, 1.149 mmol), tert-butyl 2-bromoacetate (314 mg, 1.609 mmol) and K₂CO₃ (238 mg, 1.724 mmol) in DMF (2 mL) was stirred at 70° C. for 4 h. The reaction mixture was cooled and diluted with DCM (20 mL), washed with water followed by brine. The organic layer was dried over Na₂SO₄, filtered and concentrated to give tert-butyl 2-((6-bromopyridin-2-yl)oxy)acetate (240 mg, 73%) as a yellow solid. ES-MS (m/z): [M+1−56]⁺=232.0.

Step 2: tert-butyl 2-((6-((2-hydroxyethyl)(methyl)amino)pyridin-2-yl)oxy)acetate

A mixture of tert-butyl 2-((6-bromopyridin-2-yl)oxy)acetate (240 mg, 0.833 mmol) and 2-(methylamino)ethanol (625 mg, 8.33 mmol) in pyridine (2 mL) was stirred at 90° C. under nitrogen atmosphere for 48 h. The reaction mixture was concentrated in vacuo and the residue was purified by column chromatography on silica gel (PE:EA=5:1) to give tert-butyl 2-((6-((2-hydroxyethyl)(methyl)amino)pyridin-2-yl)oxy)acetate (50 mg, 21%) as a yellow solid. ES-MS (m/z): [M+1]⁺=283.1.

Step 3: 2-(((6-((2-((2-amino-5-(2-(difluoromethyl)-6-methylpyridin-4-yl)-6-(4-fluorophenyl)pyrimidin-4-yl)oxy)ethyl)(methyl)amino)pyridin-2-yl)oxy)acetic acid

Following step 2, example 8. From tert-butyl 2-((6-((2-hydroxyethyl)(methyl)amino)pyridin-2-yl)oxy)acetate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=555.3.

### Example 257

### 2-(2-(((2-amino-5-(2-(difluoromethyl)-6-methylpyridin-4-yl)-6-(4-fluorophenyl)pyrimidin-4-yl)oxy)methyl)pyridin-4-yl)-2-methylpropanenitrile

Step 1: 4-bromo-2-(((tert-butyldimethylsilyl)oxy)methyl)pyridine

A mixture of (4-bromopyridin-2-yl)methanol (1.0 g, 5.32 mmol), imidazole (470 mg, 6.91 mmol), DMAP (130 mg, 1.06 mmol) and TBSC1 (957 mg, 6.38 mmol) in DCM (20 mL) was stirred at room temperature overnight under nitrogen atmosphere. The reaction mixture was diluted with DCM (100 mL), washed with H₂O (30 mL×3). The organic layer was dried over Na₂SO₄, filtered and concentrated. The residue was purified by silica gel column with ethyl acetate/petroleum ether (1:100) to afford 4-bromo-2-(((tert-butyldimethylsilyl)oxy)methyl)pyridine as colorless oil (1.4 g, 87%). ES-MS (m/z): [M+1]⁺=301.9.

Step 2: 2-(2-(((tert-butyldimethylsilyl)oxy)methyl)pyridin-4-yl)-2-methylpropanenitrile

To a stirring mixture of 4-bromo-2-(((tert-butyldimethylsilyl)oxy)methyl)pyridine (1.3 g, 4.30 mmol), isobutyronitrile (1.18 g, 17.22 mmol) in toluene (20 mL) was added KHMDS (1.0 M in THF, 6.5 mL, 6.5 mmol) at room temperature under nitrogen atmosphere. The reaction mixture was stirred at 80° C. for 2 h, cooled, and quenched with saturated aqueous NH₄Cl (50 mL). The layers were separated and the aqueous layer was extracted with ethyl acetate (30 mL×3). The combined organic layer was dried over Na₂SO₄, filtered and concentrated. The residue was purified by silica gel column with ethyl acetate/petroleum ether (1:20) to afford 2-(2-(((tert-butyldimethylsilyl)oxy)methyl)pyridin-4-yl)-2-methylpropanenitrile as colorless oil (610 mg, 49%). ES-MS (m/z): [M+1]⁺=291.2.

Step 3: 2-(2-(hydroxymethyl)pyridin-4-yl)-2-methylpropanenitrile

A mixture of 2-(2-(((tert-butyldimethylsilyl)oxy)methyl)pyridin-4-yl)-2-methylpropanenitrile (610 mg, 2.10 mmol) and NH₄F (389 mg, 10.52 mmol) in MeOH (10 mL) was stirred at room temperature overnight under nitrogen atmosphere. The reaction mixture was concentrated, and the residue was purified by silica gel column with ethyl acetate/petroleum ether (1:2) to afford 2-(2-(hydroxymethyl)pyridin-4-yl)-2-methylpropanenitrile as a light yellow solid (310 mg, 84%). ES-MS (m/z): [M+1]⁺=177.0.

Step 4: 2-(2-(((2-amino-5-(2-(difluoromethyl)-6-methylpyridin-4-yl)-6-(4-fluorophenyl)pyrimidin-4-yl)oxy)methyl)pyridin-4-yl)-2-methylpropanenitrile

Following step 2, example 8. From 2-(2-(hydroxymethyl)pyridin-4-yl)-2-methylpropanenitrile, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=505.2.

### Example 263

### 2-(6-(((2-amino-5-(2-(difluoromethyl)-6-methylpyridin-4-yl)-6-(4-fluorophenyl)pyrimidin-4-yl)oxy)methyl)pyridin-2-yl)-2-methylpropanoic acid

Step 1: 2-(6-(((2-amino-5-(2-(difluoromethyl)-6-methylpyridin-4-yl)-6-(4-fluorophenyl)pyrimidin-4-yl)oxy)methyl)pyridin-2-yl)-2-methylpropanoic acid

Following step 2, example 8. From 2-(6-(hydroxymethyl)pyridin-2-yl)-2-methylpropanoic acid, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=524.2.

### Example 282

### 5-(2-(difluoromethyl)-6-methylpyridin-4-yl)-4-((6-(dimethylamino)pyridin-2-yl)methoxy)-6-(4-fluorophenyl)pyrimidin-2-amine

Step 1: 6-(((tert-butyldimethylsilyl)oxy)methyl)-N,N-dimethylpyridin-2-amine

A solution of 2-bromo-6-(((tert-butyldimethylsilyl)oxy)methyl)pyridine (300 mg, 0.99 mmol) and dimethylamine (2M in THF, 5 mL, 10 mmol) in pyridine (5 mL) was stirred at 120° C. under microwave for 4 h. The reaction mixture was cooled and diluted with DCM (20 mL), washed with water (10 mL×3) and brine (10 mL×3). The organic layer was dried over Na₂SO₄, filtered and concentrated to give 6-(((tert-butyldimethylsilyl)oxy)methyl)-N,N-dimethylpyridin-2-amine (100 mg, 38%) as a yellow solid. ES-MS (m/z): [M+1]⁺=267.2.

Step 2: (6-(dimethylamino)pyridin-2-yl)methanol

To a solution of 6-(((tert-butyldimethylsilyl)oxy)methyl)-N,N-dimethylpyridin-2-amine (100 mg, 0.376 mmol) and NH₄F (70 mg, 1.88 mmol) in MeOH (5 mL) was stirred at room temperature overnight. The reaction mixture was concentrated and the residue was purified by column chromatography on silica gel (DCM:MeOH=50:1) to give (6-(dimethylamino)pyridin-2-yl)methanol (40 mg, 70%) as a yellow solid. ES-MS (m/z): [M+1]⁺=153.1.

Step 3: 5-(2-(difluoromethyl)-6-methylpyridin-4-yl)-4-(((6-(dimethylamino)pyridin-2-yl)methoxy)-6-(4-fluorophenyl)pyrimidin-2-amine

Following step 2, example 8. From (6-(dimethylamino)pyridin-2-yl)methanol, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=481.2.

### Example 285

### 5-(2-(difluoromethyl)-6-methylpyridin-4-yl)-4-(4-fluorophenyl)-6-(pyridazin-4-ylmethoxy)pyrimidin-2-amine

Step 1: 5-(2-(difluoromethyl)-6-methylpyridin-4-yl)-4-(4-fluorophenyl)-6-(pyridazin-4-ylmethoxy)pyrimidin-2-amine

Following step 2, example 8. From pyridazin-4-ylmethanol, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=439.1.

### Examples 305

### 2-(((6-(((2-amino-5-(2-(difluoromethyl)-6-methylpyridin-4-yl)-6-(4-fluorophenyl)pyrimidin-4-yl)oxy)methyl)pyridin-2-yl)oxy)ethan-1-ol

Step 1: 6-hydroxymethyl-pyridin-2-ol

To a stirring solution of 6-hydroxy-pyridine-2-carboxylic acid (2.0 g, 14.38 mmol) in THF (10 mL) was added (CH₃)₂S. BH₃ (10 M, 7.2 mL, 72.0 mmol) at room temperature. The resulting mixture was stirred at 50° C. overnight. The reaction mixture was cooled, and quenched with MeOH (30 mL) carefully. The resulting mixture then was stirred at 50° C. for 4 h, and concentrated. The residue was purified by silica gel column (DCM:MeOH=10:1) to give 6-hydroxymethyl-pyridin-2-ol (1.3 g, 72%) as a white solid. ES-MS (m/z): [M+1]⁺=126.1

Step 2: (6-(2-((tert-butyldimethylsilyl)oxy)ethoxy)pyridin-2-yl)methanol

A mixture of 6-hydroxymethyl-pyridin-2-ol (400 mg, 3.20 mmol), (2-bromoethoxy)(tert-butyl)dimethylsilane (1.0 g, 4.47 mmol) and K₂CO₃ (661 mg, 4.79 mmol) in DMF (5 mL) was stirred at 70° C. under nitrogen atmosphere overnight. The reaction mixture was cooled, quenched with H₂O (30 mL), and extracted with DCM (30 mL×3). The combined organic extract was dried over Na₂SO₄, filtered and concentrated. The residue was purified with silica gel column (PE:EA=5:1) to give (6-(2-((tert-butyldimethylsilyl)oxy)ethoxy)pyridin-2-yl)methanol (700 mg, 77%) as a yellow oil. ES-MS (m/z): [M+1]⁺=284.2

Step 3: 2-((6-(((2-amino-5-(2-(difluoromethyl)-6-methylpyridin-4-yl)-6-(4-fluorophenyl)pyrimidin-4-yl)oxy)methyl)pyridin-2-yl)oxy)ethan-1-ol

Following step 2, example 8. From (6-(2-((tert-butyldimethylsilyl)oxy)ethoxy)pyridin-2-yl)methanol, 60% NaH and 4-chloro-5-(2-(difluoromethyl)-6-methylpyridin-4-yl)-6-(4-fluorophenyl)pyrimidin-2-amine in DMF. The crude was purified by prep-HPLC to obtain 2-((6-(((2-amino-5-(2-(difluoromethyl)-6-methylpyridin-4-yl)-6-(4-fluorophenyl)pyrimidin-4-yl)oxy)methyl)pyridin-2-yl)oxy)ethan-1-ol, ES-MS (m/z): [M+1]⁺=498.2.

### Example 306

### 4-((1-(difluoromethyl)-1H-imidazol-2-yl)methoxy)-5-(2-(difluoromethyl)-6-methylpyridin-4-yl)-6-(4-fluorophenyl)pyrimidin-2-amine

Step 1: 2-(((tert-butyldiphenylsilyl)oxy)methyl)-1-(difluoromethyl)-1H-imidazole

To a stirring mixture of NaH (59 mg, 1.486 mmol, 60% in mineral oil) in THF (15 mL) at 0° C. was added a solution of 2-(((tert-butyldiphenylsilyl)oxy)methyl)-1H-imidazole (500 mg, 1.486 mmol) in THF (5 mL). After stirring at room temperature for 30 min, chlorodifluoromethane was bubbled into the reaction mixture until saturation. The resulting mixture was stirred overnight, quenched with H₂O (20 mL) and extracted with ethyl acetate (30 mL). The organic layer was concentrated and purified by silica gel column (PE:EA=10:1) to get 2-(((tert-butyldiphenylsilyl)oxy)methyl)-1-(difluoromethyl)-1H-imidazole (276 mg, 48%) as a yellow oil. ES-MS (m/z): [M+1]⁺=387.2.

Step 2: (1-(difluoromethyl)-1H-imidazol-2-yemethanol

A solution of 2-(((tert-butyldiphenylsilyl)oxy)methyl)-1-(difluoromethyl)-1H-imidazole (276 mg, 0.714 mmol) and TBAF (1 M in THF, 1.1 mL, 1.1 mmol) in THF (4 mL) was stirred at room temperature for 1 h. The reaction mixture was concentrated and purified by silica gel column (PE:EA=1:1) to get (1-(difluoromethyl)-1H-imidazol-2-yl)methanol (67.8 mg, 64%) as a white solid. ES-MS (m/z): [M+1]⁺=149.1.

Step 3: 4-((1-(difluoromethyl)-1H-imidazol-2-yemethoxy)-5-(2-(difluoromethyl)-6-methylpyridin-4-yl)-6-(4-fluorophenyl)pyrimidin-2-amine

Following step 2, example 8. From (1-(difluoromethyl)-1H-imidazol-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=477.2.

### Example 308

### 2-(4-(((2-amino-5-(2-(difluoromethyl)-6-methylpyridin-4-yl)-6-(4-fluorophenyepyrimidin-4-yl)oxy)methyl)pyrimidin-2-yl)propan-2-ol

Step 1: methyl 4-(((tert-butyldimethylsilyl)oxy)methyl)pyrimidine-2-carboxylate

A mixture of 4-(((tert-butyldimethylsilyl)oxy)methyl)-2-chloropyrimidine (500 mg, 1.93 mmol), Pd(dppf)Cl₂ (28 mg, 0.039 mmol), triethylamine (390 mg, 3.86 mmol) in CH₃OH (10 mL) was stirred at 100° C. under CO (50 PSI) overnight. The reaction mixture was concentrated in vacuo, and the residue was purified by silica gel column (DCM: CH₃OH=20:1) to give methyl 4-(((tert-butyldimethylsilypoxy)methyl)pyrimidine-2-carboxylate (188 mg, 35%) as yellow oil. ES-MS (m/z): [M+1]⁺=283.6.

Step 2: methyl 4-(hydroxymethyl)pyrimidine-2-carboxylate

A solution of methyl 4-(((tert-butyldimethylsilyl)oxy)methyl)pyrimidine-2-carboxylate (188 mg, 0.67 mmol) and NH₄F (124 mg, 3.33 mmol) in CH₃OH (5 mL) was stirred at room temperature overnight. The reaction mixture was concentrated in vacuo, and the residue was purified by silica gel column (DCM:CH₃OH=20:1) to give methyl 4-(hydroxymethyl)pyrimidine-2-carboxylate (188 mg, 167%) as yellow solid. ES-MS (m/z): [M+1]⁺=169.3.

Step 3: 2-(4-(hydroxymethyl)pyrimidin-2-yl)propan-2-ol

To a stirring solution of methylmagnesium bromide (3 M in ether, 0.6 mL, 1.67 mmol) in THF (2 mL) at 0° C. was added methyl 4-(((tert-butyldimethylsilyl)oxy)methyl)pyrimidine-2-carboxylate (70 mg, 0.42 mmol) in THF (2 mL) dropwise under nitrogen. After stirring at room temperature for 1 h, the reaction mixture was quenched by 2 drops of water, then 2 drops of aqueous HCl (1 M), followed by aqueous NaHCO₃ to pH=7. The mixture was concentrated in vacuo, and the residue was purified by silica gel column (DCM: CH₃OH=20:1) to give 2-(4-(hydroxymethyl)pyrimidin-2-yl)propan-2-ol (35 mg, 50%) as yellow oil. ES-MS (m/z): [M+1]⁺=169.4.

Step 4: 2-(4-4(2-amino-5-(2-(difluoromethyl)-6-methylpyridin-4-yl)-6-(4-fluorophenyl)pyrimidin-4-yl)oxy)methyl)pyrimidin-2-yepropan-2-ol

Following step 2, example 8. From 2-(4-(hydroxymethyl)pyrimidin-2-yl)propan-2-ol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=497.2.

### Example 313

### 5-(2-(difluoromethyl)-6-methylpyridin-4-yl)-4-(4-fluorophenyl)-6-(2-(pyridin-2-yl)ethoxy)pyrimidin-2-amine

Step 1: 5-(2-(difluoromethyl)-6-methylpyridin-4-yl)-4-(4-fluorophenyl)-6-(2-(pyridin-2-yl)ethoxy)pyrimidin-2-amine

Following step 2, example 8. From 2-pyridineethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=452.1.

### Example 314

### 5-(2-(difluoromethyl)-6-methylpyridin-4-yl)-4-(4-fluorophenyl)-6-((1-methyl-1H-benzo[d]imidazol-2-yl)methoxy)pyrimidin-2-amine

Step 1: ethyl 1H-benzo[d]imidazole-2-carboxylate

A solution of 1H-benzo[d]imidazole-2-carboxylic acid (500 mg, 3.08 mmol), thionyl chloride (1836 mg, 15.43 mmol) in CH₃CH₂OH (5 mL) was stirred under refluxing overnight. After cooling, the reaction mixture was concentrated in vacuo to get crude ethyl 1H-benzo[d]imidazole-2-carboxylate (520 mg, 89%) as yellow solid. ES-MS (m/z): [M+1]⁺=191.2.

Step 2: ethyl 1-methyl-1H-benzo1dlimidazole-2-carboxylate

To a stirring solution of ethyl 1H-benzo1dlimidazole-2-carboxylate (200 mg, 1.05 mmol) in DMF (5 mL) at 0° C. was added NaH (60% in mineral oil, 50 mg, 1.58 mmol) under nitrogen. After stirring at room temperature for 0.5 h, iodomethane (223 mg, 1.58 mmol) was added. The resulting mixture was stirred continually at room temperature for 1 h, then was quenched with 2 drops of water. The mixture was purified by silica gel column (DCM: MeOH=40:1) to give ethyl 1-methyl-1H-benzo kflimidazole-2-carboxylate (150 mg, 70%) as yellow oil. ES-MS (m/z): [M+1]⁺=205.3.

Step 3: (1-methyl-1H-benzo[d]imidazol-2-yemethanol

To a stirring solution of ethyl 1-methyl-1H-benzo kflimidazole-2-carboxylate (150 mg, 0.74 mmol) in THF (4 mL) at 0° C. was added LiAlH₄ (1 M, 2.21mL, 2.21 mmol) under nitrogen. After stirring at room temperature for 1 h, the reaction mixture was quenched by 4 drops of water. The mixture was filtered, and concentrated to get crude (1-methyl-1H-benzo[d]imidazol-2-yl)methanol (125 mg, 104%) as yellow oil. ES-MS (m/z): [M+1]⁺=163.2.

Step 4: 5-(2-(difluoromethyl)-6-methylpyridin-4-yl)-4-(4-fluorophenyl)-6-((1-methyl-1H-benzo[d]imidazol-2-yl)methoxy)pyrimidin-2-amine

Following step 2, example 8. From (1-methyl-1H-benzo[d]imidazol-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=491.2.

### Example 315

### 5-(2-(difluoromethyl)-6-methylpyridin-4-yl)-4-((1-ethyl-1H-benzo[d]imidazol-2-yl)methoxy)-6-(4-fluorophenyl)pyrimidin-2-amine

Step 1: ethyl 1-ethyl-1H-benzo[d]imidazole-2-carboxylate

Following step 2, example 283. From ethyl 1H-benzoldlimidazole-2-carboxylate, 60% NaH and iodoethane in DMF. ES-MS (m/z): [M+1]⁺=219.4.

Step 2: (1-ethyl-1H-benzo[d]imidazol-2-yl)methanol

Following step 3, example 283. From ethyl 1-ethyl-1H-benzo[d]imidazole-2-carboxylate and 1 M LiAlH₄ in THF. ES-MS (m/z): [M+1]⁺=177.2.

Step 3: 5-(2-(difluoromethyl)-6-methylpyridin-4-yl)-4-(4-fluorophenyl)-6-((1-ethyl-1H-benzo[d]imidazol-2-yl)methoxy)pyrimidin-2-amine

Following step 2, example 8. From (1-ethyl-1H-benzoldlimidazol-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=505.2.

### Example 320

### 2-(6-((2-((2-amino-5-(2-(difluoromethyl)-6-methylpyridin-4-yl)-6-(4-fluorophenyl)pyrimidin-4-yl)oxy)ethyl)(methyeamino)pyridin-2-yl)-2-methylpropanenitrile

Step 1: 2-(6-bromopyridin-2-yl)-2-methylpropanenitrile

To a stirring solution of 2,6-dibromopyridine (1.0 g, 4.22 mmol) and isobutyronitrile (300 mg, 4.34 mmol) in toluene (10 mL) was added KHMDS (1.0 M in THF, 12.6 mL, 12.6 mmol) under N₂ at room temperature. This reaction was stirred continually at room temperature for 1 h, quenched by aqueous NH4Cl, concentrated, and the residue was purified by column chromatography on silica gel (PE:EA=30:1) to give 2-(6-bromopyridin-2-yl)-2-methylpropanenitrile (540 mg, 57%) as a yellow oil. ES-MS (m/z): [M+1]⁺=225.1.

Step 2: 2-(6-((2-hydroxyethyl)(methyl)amino)pyridin-2-yl)-2-methylpropanenitrile

A mixture of 2-(6-bromopyridin-2-yl)-2-methylpropanenitrile (400 mg, 1.78 mmol) and 2-(methylamino)ethanol (664 mg, 8.84 mmol) in pyridine (4 mL) was stirred at 90° C. for 6 h. The mixture was concentrated and the residue was purified by column chromatography on silica gel (DCM:MeOH=40:1) to give 2-(6-((2-hydroxyethyl)(methyl)amino)pyridin-2-yl)-2-methylpropanenitrile (400 mg, 102%) as a yellow oil. ES-MS (m/z): [M+1]⁺=220.4.

Step 3: 2-(6-((2-((2-amino-5-(2-(difluoromethyl)-6-methylpyridin-4-yl)-6-(4-fluorophenyl)pyrimidin-4-yl)oxy)ethyl)(methyl)amino)pyridin-2-yl)-2-methylpropanenitrile

Following step 2, example 8. From 2-(6-((2-hydroxyethyl)(methyl)amino)pyridin-2-yl)-2-methylpropanenitrile, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=548.2.

### Example 326

### 4-((6-cyclopropylpyridin-2-yl)methoxy)-5-(2-(difluoromethyl)-6-methylpyridin-4-yl)-6-(4-fluorophenyl)pyrimidin-2-amine

Step 1: 2-bromo-6-(((tert-butyldimethylsilyeoxy)methyl)pyridine

To a stirring solution of (6-bromopyridin-2-yl)methanol (4.0 g, 21.3 mmol) in DMF (30 mL) was added imidazole (5.78 g, 85.1 mmol) at room temperature. After stirring continually at room temperature for 10 min, TBSC1 (4.7 g, 31.9 mmol) and DMAP (0.51 g, 4.2 mmol) were added at 0° C. The resulting mixture was stirred at room temperature for 12 h, diluted with H₂O (50 mL) and extracted with ethyl acetate (50 mL×3). The combined organic layers were dried over Na₂SO₄, filtered and concentrated. The residue was purified by column chromatography on silica gel (PE:EA=20:1) to get 2-bromo-6-(((tert-butyldimethylsilyl)oxy)methyl)pyridine (6.31 g, 98%) as a colorless oil.

Step 2: 2-(((tert-butyldimethylsilyl)oxy)methyl)-6-cyclopropylpyridine

A mixture of 2-bromo-6-(((tert-butyldimethylsilyeoxy)methyl)pyridine (500 mg, 1.65 mmol), cyclopropylboronic acid (284 mg, 3.31 mmol), PCy₃ (46 mg, 0.165 mmol), Pd(OAc)₂ (37 mg, 0.165 mmol) and K₃PO₄ (1.0 g, 4.96 mmol) in toluene/H₂O (10 mL/0.5 mL) was stirred at 100° C. under N₂ overnight. The reaction mixture was cooled, concentrated and the residue was purified by column chromatograph on silica gel (PE:EA=10:1) to give 2-(((tert-butyldimethylsilyl)oxy)methyl)-6-cyclopropylpyridine (300 mg, 69%) as a yellow solid. ES-MS (m/z): [M+1]⁺=264.2.

Step 3: (6-cyclopropylpyridin-2-yl)methanol

A mixture of 2-(((tert-butyldimethylsilyl)oxy)methyl)-6-cyclopropylpyridine (150 mg, 0.57 mmol) and NH₄F (105 mg, 2.85 mmol) in MeOH (3 mL) was stirred at room temperature overnight. The reaction mixture was concentrated and purified by column chromatograph on silica gel (PE:EA=2:1) to give (6-cyclopropylpyridin-2-yl)methanol (60 mg, 70%) as a yellow oil. ES-MS (m/z): [M+1]⁺=150.1.

Step 4: 4-((6-cyclopropylpyridin-2-yl)methoxy)-5-(2-(difluoromethyl)-6-methylpyridin-4-yl)-6-(4-fluorophenyl)pyrimidin-2-amine

Following step 2, example 8. From (6-cyclopropylpyridin-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=478.2.

### Example 329

### 5-(2-(difluoromethyl)-6-methylpyridin-4-yl)-4-((3-ethyl-3H-imidazo[4,5-b]pyridin-2-yl)methoxy)-6-(4-fluorophenyl)pyrimidin-2-amine

Step 1: (3H-imidazo[4,5-b]pyridin-2-yl)methanol

A mixture of pyridine-2,3-diamine (2.0 g, 18.3 mmol) and 2-hydroxyacetic acid (3.0 g, 40.3 mmol) was stirred at 145° C. for 6 h. The reaction mixture was cooled and basified with saturated aqueous NaHCO₃ to pH=8, then concentrated. The residue was purified by silica gel column (DCM:MeOH=20:1 to 10:1) to get 3H-imidazo[4,5-b]pyridin-2-yl)methanol (786 mg, 29%) as a red solid. ES-MS (m/z): [M+1]⁺=150.1.

Step 2: 2-(((tert-butyldimethylsilyl)oxy)methyl)-3H-imidazo[4,5-b]pyridine

To a stirring solution of 3H-imidazo[4,5-b]pyridin-2-yl)methanol (786 mg, 5.25 mmol) in DMF (5 mL) at 0° C. was added imidazole (535 mg, 7.87 mmol), K₂CO₃ (362 mg, 2.62 mmol) and TBSC1 (1.18 g, 7.87 mmol). The resulting mixture was stirred at 0° C. for 1 h, diluted with H₂O (50 mL) and extracted with ethyl acetate (50 mL×3). The combined organic layer was dried over Na₂SO₄, filtered and concentrated. The residue was purified by silica gel column (PE:EA=2:1) to get 2-(((tert-butyldimethylsilyl)oxy)methyl)-3H-imidazo[4,5-b]pyridine (1.29 g, 93%) as a white solid. ES-MS (m/z): [M+1]⁺=264.1.

Step 3: 2-(((tert-butyldimethylsilyl)oxy)methyl)-3-ethyl-3H-imidazo[4,5-b]pyridine

To a stirring solution of 2-(((tert-butyldimethylsilyl)oxy)methyl)-3H-imidazo[4,5-b]pyridine (400 mg, 1.52 mmol) in DMF (5 mL) at 0° C. was added NaH (60% in mineral oil, 121 mg, 3.04 mmol). After stirring at 0° C. for 0.5 h, iodoethane (261 mg, 1.67 mmol) was added at 0° C., and the mixture was stirred continually at 0° C. for 1 h. The reaction mixture was quenched with H₂O (2.0 mL) carefully. The resulting mixture was concentrated and the residue was purified by silica gel column (PE:EA=5:1) to get 2-(((tert-butyldimethylsilyl)oxy)methyl)-3-ethyl-3H-imidazo[4,5-b]pyridine (149 mg, 34%) as a white solid. ES-MS (m/z): [M+1]⁺=292.2.

Step 4: (3-ethyl-3H-imidazo[4,5-b]pyridin-2-yemethanol

A solution of 2-(((cert-butyldimethylsilyl)oxy)methyl)-3-ethyl-3H-imidazo[4,5-b]pyridine (149 mg, 0.51 mmol) and NH₄F (95 mg, 2.56 mmol) in MeOH (5 mL) was stirred at room temperature for 16 h. The reaction mixture was concentrated, and the residue was triturated with DCM: MeOH (10: 1, 10 mL×3). The combined organic layer was concentrated to get crude (3-ethyl-3H-imidazo[4,5-b]pyridin-2-yl)methanol (87.7 mg, 96%) as a yellow solid, which was used for next step without further purification. ES-MS (m/z): [M+1]⁺=178.1.

Step 5: 5-(2-(difluoromethyl)-6-methylpyridin-4-yl)-4-((3-ethyl-3H-imidazo[4,5-b]pyridin-2-yl)methoxy)-6-(4-fluorophenyl)pyrimidin-2-amine

Following step 2, example 8. From (3-ethyl-3H-imidazo[4,5-b]pyridin-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=506.2.

### Example 349

### 2-(6-(2-((2-amino-5-(2-(difluoromethyl)-6-methylpyridin-4-yl)-6-(4-fluorophenyl)pyrimidin-4-yl)oxy)ethyl)pyridin-2-yl)propan-2-ol

Step 1: 2-(6-(2-((tetrahydro-2H-pyran-2-yl)oxy)ethyl)pyridin-2-yl)propan-2-ol

To a stirring solution of 2-bromo-6-(2-((tetrahydro-2H-pyran-2-yl)oxy)ethyl)pyridine (300.0 mg, 1.048 mmol) in THF (3.0 mL) was added n-BuLi (1.6 M in hexanes, 0.66 mL, 1.056 mmol) dropwise at −78° C. under nitrogen atmosphere. After stirring for 2 h at −78° C., acetone (304.4 mg, 5.242 mmol) was added dropwise over 15 min at −78° C. The resulting mixture was warmed slowly to room temperature. The reaction mixture was quenched with saturated aqueous NH₄Cl solution (0.1 mL), dried over Na₂SO₄, filtered and concentrated. The residue was purified by silica gel column with PE/EA (5/1) to afford 2-(6-(2-((tetrahydro-2H-pyran-2-yl)oxy)ethyl)pyridin-2-yl)propan-2-ol (183 mg, 66%) as a light brown solid. ES-MS (m/z): [M+1]⁺=266.1.

Step 2: 2-(6-(2-hydroxyethyl)pyridin-2-yl)propan-2-ol

A mixture of 2-(6-(2-((tetrahydro-2H-pyran-2-yl)oxy)ethyl)pyridin-2-yl)propan-2-ol (183.0 mg, 0.690 mmol) and PPTS (17.33 mg, 0.069 mmol) in EtOH (2.0 mL) was stirred at 80° C. for 3 h. The reaction mixture was cooled and concentrated. The residue was purified by Prep-HPLC to afford 2-(6-(2-hydroxyethyl)pyridin-2-yl)propan-2-ol (84 mg, 67%) as colorless oil. ES-MS (m/z): [M+1]⁺=182.1.

Step 3: 2-(6-(2-((2-amino-5-(2-(difluoromethyl)-6-methylpyridin-4-yl)-6-(4-fluorophenyl)pyrimidin-4-yl)oxy)ethyl)pyridin-2-yl)propan-2-ol

To a stirring mixture of 2-(6-(2-hydroxyethyl)pyridin-2-yl)propan-2-ol (30.0 mg, 0.166 mmol) and 4-chloro-5-(2-(difluoromethyl)-6-methylpyridin-4-yl)-6-(4-fluorophenyl)pyrimidin-2-amine (60.4 mg, 0.166 mmol) in DMF (0.3 mL) was added Ag₂O (191.8 mg, 0.828 mmol) at room temperature. The resulting mixture was stirred for 4 h at 80° C., then was cooled and filtered. The filtrate was purified by Prep-HPLC to afford 2-(6-(2-((2-amino-5 -(2-(difluoromethyl)-6-methylpyridin-4-yl)-6-(4-fluorophenyl)pyrimidin-4-yl)oxy)ethyl)pyridin-2-yepropan-2-ol (5 mg, 6%) as a light yellow solid. ES-MS (m/z): [M+1]⁺=510.2.

### Example 350

### 5-(2-(difluoromethyl)-6-methylpyridin-4-yl)-4-(4-fluorophenyl)-6-(thiazol-2-ylmethoxy)pyrimidin-2-amine

Following step 2, example 8. From thiazol-2-ylmethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=444.1.

### Example 355

### 4-((1-(difluoromethyl)-1H-benzo[d]imidazol-2-yl)methoxy)-5-(2-(difluoromethyl)-6-methylpyridin-4-yl)-6-(4-fluorophenyl)pyrimidin-2-amine

Step 1: (1H-benzo kflimidazol-2-yl)methanol

To a stirring solution of 1H-benzo[d]imidazole-2-carboxylic acid (500 mg, 3.08 mmol) in THF (5.0 mL) was added LiAlH₄ (1.0 M, 9.26 mL, 9.26 mmol) at 0° C. under N₂. This reaction was stirred continually at room temperature for 1 h, quenched by water (10 drops), filtered and concentrated to give crude (1H-benzoldlimidazol-2-yl)methanol (212 mg, 46%) as a yellow solid, which was used for next step without further purification. ES-MS (m/z): [M+1]⁺=149.1.

Step 2: (1-(difluoromethyl)-1H-benzoldlimidazol-2-yl)methanol

To a stirring solution of (1H-benzoldlimidazol-2-yl)methanol (200 mg, 1.36 mmol), dibenzo-18-crown-6 (8 mg, 0.027 mmol) in 1,4-dioxane (1.0 mL) at 70° C. was bubbled with CHClF₂, meanwhile 35% KOH(aq) was gradually added to keep pH at 9-11 until disappearance of starting (1H-benzoldlimidazol-2-yl)methanol by TLC. The reaction mixture was cooled, and the organic layer was separated and concentrated. The residue was triturated with DCM (10 mL×3). The combined DCM layers were dried over Na₂SO₄, filtered and concentrated. The residue was purified by column chromatography on silica gel with DCM: MeOH=40:1 to give (1-(difluoromethyl)-1H-benzoldlimidazol-2-yl)methanol (100 mg, 37%) as yellow oil. ES-MS (m/z): [M+1]⁺=199.1.

Step 3: 4-((1-(difluoromethyl)-1H-benzoldlimidazol-2-yemethoxy)-5-(2-(difluoromethyl)-6-methylpyridin-4-yl)-6-(4-fluorophenyl)pyrimidin-2-amine

Following step 2, example 8. From (1-(difluoromethyl)-1H-benzoldlimidazol-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=527.1.

### Example 376

### 4-(((6-aminopyridin-2-yl)methoxy)-5-(2-(difluoromethyl)-6-methylpyridin-4-yl)-6-(4-fluorophenyl)pyrimidin-2-amine

Step 1: (6-aminopyridin-2-yl)methanol

To a stirring solution of methyl 6-aminopicolinate (2.0 g, 13.1 mmol) in THF (30 mL) was added AlLiH₄ (1.5 g, 39.5 mmol) was added at room temperature. The reaction mixture was stirred continually at room temperature for 12 h, quenched by H₂O (2 mL) carefully. After stirring at room temperature for 2 h, the mixture was filtered and concentrated. The residue was purified by chromatograph on silica gel (DCM/MeOH=20/1) to give (6-aminopyridin-2-yl)methanol (1.5 g, 92%) as a white solid.

Step 2: 4-((6-aminopyridin-2-yemethoxy)-5-(2-(difluoromethyl)-6-methylpyridin-4-yl)-6-(4-fluorophenyl)pyrimidin-2-amine

Following step 2, example 8. From (6-aminopyridin-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=453.2.

### Example 379

### 5-(2-(difluoromethyl)-6-methylpyridin-4-yl)-4-(4-fluorophenyl)-6-((6-(methylamino)pyridin-2-yl)methoxy)pyrimidin-2-amine

Step 1: methyl 6-((tert-butoxycarbonyl)amino)picolinate

To a stirring solution of methyl 6-aminopicolinate (5.0 g, 32.9 mmol), triethylamine (10.0 g, 98.8 mmol) and DMAP (805 mg, 6.6 mmol) in DCM (60 mL) was added (Boc)₂O (8.5 g, 38.9 mmol) at room temperature. After stirring continually at room temperature for 3 h, the reaction mixture was concentrated and the residue was purified by chromatograph on silica gel (PE/EA=10/1) to give methyl 6-((tert-butoxycarbonyl)amino)picolinate (3.0 g, 36%) as a white solid.

Step 2: (6-(methylamino)pyridin-2-yl)methanol

To a stirring solution of methyl 6-((tert-butoxycarbonyl)amino)picolinate (0.5 g, 4.0 mmol) in THF (10 mL) was added AlLiH₄ (456 mg, 12.0 mmol) at room temperature. The reaction mixture was refluxed for 3 h, cooled and quenched with H₂O (10 mL) carefully. After stirring at room temperature for 1 h, the resulting mixture was filtrated and concentrated. The residue was purified by chromatograph on silica gel (PE/EA=3/1) to give (6-(methylamino)pyridin-2-yl)methanol (24 mg, 4.3%) as a yellow oil. ES-MS (m/z): [M+1]⁺=139.1.

Step 3: 5-(2-(difluoromethyl)-6-methylpyridin-4-yl)-4-(4-fluorophenyl)-6-((6-(methylamino)pyridin-2-yl)methoxy)pyrimidin-2-amine

Following step 2, example 8. From (6-(methylamino)pyridin-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=467.2.

### Example 381

### 5-(2-(difluoromethyl)-6-methylpyridin-4-yl)-4-(4-fluorophenyl)-6-(isoquinolin-1-ylmethoxy)pyrimidin-2-amine

Step 1: methyl isoquinoline-1-carboxylate

To a stirring solution of isoquinoline-1-carboxylic acid (5.2 g, 30.0 mmol) in MeOH (100 mL) was added concentrated H₂SO0₄ (300 mg, about 3.0 mmol) at room temperature. The resulting mixture was heated at 60° C. for 16 h, concentrated, and partitioned between EtOAc (50 mL×3) and H₂O (70 mL). The combined organic layers were dried over Na₂SO₄, filtered and concentrated to give crude methyl isoquinoline-1-carboxylate (4.0 g, 71%) as brown oil, which was used for next step without further purification. ES-MS (m/z): [M+1]⁺=188.1.

Step 2: isoquinolin-1-ylmethanol

To a stirring solution of methyl isoquinoline-1-carboxylate (935 mg, 5.0 mmol) in MeOH (20 mL) was added NaBH₄ (570 mg, 15.0 mmol) in portionwise at room temperature. After stirring continually at room temperature for 5 h, the reaction mixture was quenched with saturated aqueous NH₄Cl, filtered and concentrated. The residue was purified by column chromatography on silica gel eluting with PE/EtOAc (4:1) to give isoquinolin-1-ylmethanol (430 mg, 54%) as yellow oil. ES-MS (m/z): [M+1]⁺=160.1.

Step 3: 5-(2-(difluoromethyl)-6-methylpyridin-4-yl)-4-(4-fluorophenyl)-6-(isoquinolin-1-ylmethoxy)pyrimidin-2-amine

Following step 2, example 8. From isoquinolin-1-ylmethanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=488.2.

### Example 389

### 5-(2-(difluoromethyl)-6-methylpyridin-4-yl)-4-(4-fluorophenyl)-6-((6-(2-(methylamino)propan-2-yl)pyridin-2-yl)methoxy)pyrimidin-2-amine

Step 1: 6-(((tert-butyldimethylsilyl)oxy)methyl)picolinonitrile

A mixture of 2-bromo-6-(((tert-butyldimethylsilyeoxy)methyl)pyridine (1.5 g, 4.96 mmol), Zn(CN)₂ (1.1 g, 9.37 mmol) and Pd(PPh₃)₄ (577 mg, 0.50 mmol) in DMF (20 mL) was stirred at 90° C. for 2 h. The reaction mixture was filtered and concentrated. The residue was purified by flash chromatography on silica gel (PE:EA=20:1) to get 6-(((tert-butyldimethylsilyl)oxy)methyl)picolinonitrile (1.18 g, 96%) as a white solid.

Step 2: 2-(6-(((tert-butyldimethylsilyl)oxy)methyl)pyridin-2-yl)propan-2-amine

To a stirring solution of 6-(((tert-butyldimethylsilyl)oxy)methyl)picolinonitrile (900 mg, 3.6 mmol) in toluene (12 mL) at 0° C. was added CH₃MgBr (3.5 mL, 9.1 mmol, 2.6 M) slowly under nitrogen atmosphere. The resulting mixture was refluxed overnight, cooled and quenched with saturated aqueous NH₄Cl (20 mL), then extracted with ethyl acetate (20 mL×2). The combined organic phases were washed with brine (20 mL), dried over Na₂SO₄, filtered and concentrated to get crude 2-(6-(((tert-butyldimethylsilyl)oxy)methyl)pyridin-2-yl)propan-2-amine (600 mg, 59%) as a brown oil, which was used for next step without further purification. ES-MS (m/z): [M+1]⁺=281.2.

Step 3: N-benzyl-2-(6-(((tert-butyldimethylsilyl)oxy)methyl)pyridin-2-yl)-N-methylpropan-2-amine

To a stirring solution of 2-(6-(((tert-butyldimethylsilyl)oxy)methyl)pyridin-2-yl)propan-2-amine (450 mg, 1.6 mmol), benzaldehyde (254 mg, 2.4 mmol), triethylamine (162 mg, 1.6 mmol) and AcOH (96 mg, 1.6 mmol) in MeOH (8 mL) was added NaBH₃CN (302 mg, 4.8 mmol). After stirring the reaction mixture at 30° C. overnight, paraformaldehyde (144 mg, 4.8 mmol) was added. The resulting mixture was stirred continually at 30° C. for 1 h, diluted with H₂O (20 mL) and extracted with ethyl acetate (20 mL×2). The combined organic phases were washed with brine (20 mL), dried over Na₂SO₄, filtered and concentrated. The residue was purified by flash chromatography on silica gel (PE:EA=2:1) to get N-benzyl-2-(6-(((tert-butyldimethylsilyl)oxy)methyl)pyridin-2-yl)-N-methylpropan-2-amine (350 mg, 56%) as a yellow oil.

Step 4: (6-(2-(benzyl(methyl)amino)propan-2-yl)pyridin-2-yl)methanol

A mixture of N-benzyl-2-(6-(((tert-butyldimethylsilyl)oxy)methyl)pyridin-2-yl)-N-methylpropan-2-amine (350 mg, 0.91 mmol) and NH₄F (168 mg, 4.54 mmol) in MeOH (8 mL) was stirred at 30° C. overnight. The mixture was concentrated and the solid was triturated with DCM:MeOH=20:1 (20 mL). The mixture was filtered and concentrated to get crude (6-(2-(benzyl(methyl)amino)propan-2-yl)pyridin-2-yl)methanol (250 mg, 101%) as a yellow oil, which was used for next step without further purification. ES-MS (m/z): [M+1]⁺=271.2.

Step 5: (6-(2-(methylamino)propan-2-yl)pyridin-2-yl)methanol

To a stirring solution of (6-(2-(benzyl(methyl)amino)propan-2-yl)pyridin-2-yl)methanol (250 mg, 0.92mmol) in MeOH (6 mL) was added 10% Pd/C (100 mg). The resulting mixture was stirred at 30° C. overnight under 1 atm hydrogen atmosphere. The reaction mixture was filtered and concentrated to get crude (6-(2-(methylamino)propan-2-yl)pyridin-2-yl)methanol (170 mg, 102%) as an off-white solid, which was used for next step without further purification. ES-MS (m/z): [M+1]⁺=181.2.

Step 6: 5-(2-(difluoromethyl)-6-methylpyridin-4-yl)-4-(4-fluorophenyl)-6-((6-(2-(methylamino)propan-2-yl)pyridin-2-yl)methoxy)pyrimidin-2-amine

Following step 2, example 8. From (6-(2-(methylamino)propan-2-yl)pyridin-2-yl)methanol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=509.3.

### Example 393

### (2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-yl)glycine

Step 1: ethyl 2-((2-(6-(((tert-butyldimethylsilyl)oxy)methyl)pyridin-2-yl)propan-2-yl)amino)acetate

A mixture of 2-(6-(((tert-butyldimethylsilyl)oxy)methyl)pyridin-2-yl)propan-2-amine (500 mg, 1.78 mmol), ethyl 2-bromoacetate (451 mg, 2.70 mmol) and K₂CO₃ (497 mg, 3.60 mmol) in acetonitrile (10 mL) was stirred at 80° C. for 4 h. The resulting mixture was cooled, filtered and concentrated. The residue was purified by flash chromatography on silica gel (PE:EA=2:1) to get ethyl 2-((2-(6-(((tert-butyldimethylsilyl)oxy)methyl)pyridin-2-yl)propan-2-yl)amino)acetate (170 mg, 26%) as a yellow oil. ES-MS (m/z): [M+1]⁺=367.3.

Step 2: ethyl 2-((2-(6-(hydroxymethyl)pyridin-2-yl)propan-2-yl)amino)acetate

A mixture of ethyl 2-((2-(6-(((tert-butyldimethylsilyl)oxy)methyl)pyridin-2-yl)propan-2-yl)amino)acetate (170 mg, 0.46 mmol) and NH₄F (85 mg, 2.29 mmol) in MeOH (5 mL) was stirred at 30° C. overnight. The reaction mixture was concentrated and the residual solid was triturated with DCM:MeOH=20:1 (20 mL). The mixture was filtered and concentrated to get crude ethyl 2-((2-(6-(hydroxymethyl)pyridin-2-yl)propan-2-yl)amino)acetate (100 mg, 87%) as a yellow oil, which was used for next step without further purification. ES-MS (m/z): [M+1]⁺=253.2.

Step 3: (2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-yl)glycine

Following step 2, example 8. From ethyl 2-((2-(6-(hydroxymethyl)pyridin-2-yl)propan-2-yl)amino)acetate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=553.3.

### Example 398

### 2-(6-(((2-amino-5-(2-(difluoromethyl)-6-methylpyridin-4-yl)-6-(4-fluorophenyl)pyrimidin-4-yl)oxy)methyl)pyridin-2-yl)-2-ethylbutanoic acid

Step 1: [6-(tert-Butyl-dimethyl-silanyloxymethyl)-pyridin-2-yl]-acetonitrile

n-BuLi (2.5 M in hexanes, 3.3 mL, 8.25 mmol) was slowly added to anhydrous THF (15 mL) at −78° C., followed by anhydrous CH₃CN (407 mg, 9.92 mmol) at −78° C. The resulting mixture was stirred at −78° C. for 30 min, a solution of 2-bromo-6-(tert-butyl-dimethyl-silanyloxymethyl)-pyridine (500 mg, 1.65 mmol) in anhydrous THF (15 mL) was added dropwise at −78° C. After stirring at −78° C. under nitrogen atmosphere for 5 h, the reaction mixture was quenched with saturated aqueous NH₄Cl, extracted with EtOAc (50 mL×2). The combined organic layers were washed with brine (50 mL), dried over Na₂SO₄, filtered and concentrated. The residue was purified by column chromatography on silica gel (PE:EA=20:1) to give [6-(tert-butyl-dimethyl-silanyloxymethyl)-pyridin-2-yl]-acetonitrile (95 mg, 22%) as a yellow oil. ES-MS (m/z): [M+1]⁺=263.1.

Step 2: 2-[6-(tert-Butyl-dimethyl-silanyloxymethyl)-pyridin-2-yl]-2-ethyl-butyronitrile

To a stirring solution of [6-(tert-Butyl-dimethyl-silanyloxymethyl)-pyridin-2-yl]-acetonitrile (180 mg, 0.686 mmol) in DMF (3.0 mL) was added NaH (60% in mineral oil, 55 mg, 1.372 mmol) at 0° C. After stirring at 0° C. for 30 min, iodoethane (428 mg, 2.744 mmol) was added dropwise at 0° C. The resulting mixture was stirred at 0° C. for 1 h, quenched with H₂O (0.5 mL), diluted with DCM (10 mL), dried over Na₂SO₄ and concentrated. The residue was purified by column chromatography on silica gel (PE:EA=20:1) to give 2-[6-(tert-Butyl-dimethyl-silanyloxymethyl)-pyridin-2-yl]-2-ethyl-butyronitrile (124 mg, 57%) as a yellow oil. ES-MS (m/z): [M+1]⁺=319.2.

Step 3: 2-ethyl-2-(6-hydroxymethyl-pyridin-2-yl)-butyronitrile

A mixture of 2-[6-(tert-Butyl-dimethyl-silanyloxymethyl)-pyridin-2-yl]-2-ethyl-butyronitrile (124 mg, 0.389 mmol) and NH₄F (144 mg, 3.890 mmol) in MeOH (2.0 mL) was stirred at 30° C. overnight. The reaction mixture was concentrated and the residue was purified by column chromatography on silica gel (PE:EA=5:1) to give 2-ethyl-2-(6-hydroxymethyl-pyridin-2-yl)-butyronitrile (60 mg, 76%) as a yellow oil. ES-MS (m/z): [M+1]⁺=205.1.

Step 4: 2-ethyl-2-(6-hydroxymethyl-pyridin-2-yl)-butyric acid

A mixture of 2-ethyl-2-(6-hydroxymethyl-pyridin-2-yl)-butyronitrile (50 mg, 0.245 mmol) and KOH (1.0 M in H₂O, 2.0 mL) was stirred at 100° C. for 2 days. The reaction mixture was cooled, adjusted to pH=7-8 with 1.0 M HCl at 0° C., concentrated and the residue was purified by column chromatography on silica gel (PE:EA=2:1) to give 2-ethyl-2-(6-hydroxymethyl-pyridin-2-yl)-butyric acid (23 mg, 42%) as a yellow oil. ES-MS (m/z): [M+1]⁺=224.2.

Step 5: 2-(6-4(2-amino-5-(2-(difluoromethyl)-6-methylpyridin-4-yl)-6-(4-fluorophenyl)pyrimidin-4-yl)oxy)methyl)pyridin-2-yl)-2-ethylbutanoic acid

Following step 2, example 8. From 2-ethyl-2-(6-hydroxymethyl-pyridin-2-yl)-butyric acid, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=551.3.

### Example 403

### 5-(2-(difluoromethyl)-6-methylpyridin-4-yl)-4-(4-fluorophenyl)-6-((6-((methylamino)methyl)pyridin-2-yl)methoxy)pyrimidin-2-amine

Step 1: 6-(tert-butyl-dimethyl-silanyloxymethyl)-pyridine-2-carbonitrile

To a stirring mixture of 2-bromo-6-(tert-butyl-dimethyl-silanyloxymethyl)-pyridine (5.0 g, 16.5 mmol) in DMF (50 mL) was added Zn(CN)₂ (3.9 g , 33.2 mmol) and Pd(PPh₃)₄ (1.9 g, 1.64 mmol). The resulting mixture was stirred at 90° C. under nitrogen atmosphere for 4 h. The reaction mixture was cooled, filtered and concentrated. The residue was purified by column chromatography on silica gel (PE:EA=20:1) to obtain 6-(tert-butyl-dimethyl-silanyloxymethyl)-pyridine-2-carbonitrile (4.0 g, 98%) as a white solid. ES-MS (m/z): [M+1]⁺=249.1.

Step 2: (6-(((tert-butyldimethylsilyl)oxy)methyl)pyridin-2-yl)methanamine

To a stirring mixture of 6-(tert-Butyl-dimethyl-silanyloxymethyl)-pyridine-2-carbonitrile (1.2 g, 4.83 mmol) in NH₃/MeOH (2.0 M, 30 mL) was added Raney-Ni (2.4 g). The resulting mixture was stirred at 20° C. under hydrogen atmosphere for 3 h, filtered and concentrated. The residue was purified by column chromatography on silica gel (DCM:MeOH=20:1) to give (6-(((tert-butyldimethylsilyl)oxy)methyl)pyridin-2-yl)methanamine (930 mg, 76%). ES-MS (m/z): [M+1]⁺=253.1.

Step 3: tert-butyl((6-(((tert-butyldimethylsilyl)oxy)methyl)pyridin-2-yl)methyl)carbamate

To a stirring mixture of (6-(((tert-butyldimethylsilyl)oxy)methyl)pyridin-2-yl)methanamine (830 mg, 3.29 mmol) in DCM (80 mL) was added (Boc)₂O (781 mg, 3.62 mmol) at room temperature. The resulting mixture was stirred continually at room temperature for 2 h, then concentrated and the residue was purified by column chromatography on silica gel (DCM:MeOH=20:1) to give tert-butyl((6-(((tert-butyldimethylsilyl)oxy)methyl)pyridin-2-yl)methyl)carbamate (1.1 g, 95%) as a yellow solid. ES-MS (m/z): [M+1]⁺=353.2.

Step 4: tert-butyl((6-(((tert-butyldimethylsilyl)oxy)methyl)pyridin-2-yl)methyl)(methyl)carbamate

To a stirring mixture of tert-butyl((6-(((tert-butyldimethylsilyl)oxy)methyl)pyridin-2-yl)methyl)carbamate (100 mg, 0.284 mmol) in THF (1.0 mL) was added NaH (60% in mineral oil, 17 mg, 0.425 mmol) under nitrogen atmosphere at 0° C. After stirring at room temperature for 30 min, Iodomethane (81 mg, 0.571 mmol) was added at 0° C. The resulting mixture was stirred at room temperature for 3 h, quenched with saturated aqueous NH₄Cl (0.2 mL), diluted with DCM (10 mL), dried over Na₂SO₄, filtered and concentrated. The residue was purified by column chromatography on silica gel (DCM:MeOH=20:1) to give tert-butyl((6-(((tert-butyldimethylsilyl)oxy)methyl)pyridin-2-yl)methyl)(methyl)carbamate (85 mg, 82%) as a yellow oil. ES-MS (m/z): [M+1]⁺=367.2.

Step 5: (6-hydroxymethyl-pyridin-2-ylmethyl)-methyl-carbamic acid tert-butyl ester

A mixture of tert-butyl((6-(((tert-butyldimethylsilyl)oxy)methyl)pyridin-2-yl)methyl)(methyl)carbamate (85 mg, 0.232 mmol) and NH₄F (86 mg, 2.320 mmol) in MeOH (2.0 mL) was stirred at room temperature overnight. The reaction mixture was concentrated and purified by column chromatography on silica gel (DCM:MeOH=20:1) to give (6-hydroxymethyl-pyridin-2-ylmethyl)-methyl-carbamic acid tert-butyl ester (50 mg, 85%) as a yellow oil. ES-MS (m/z): [M+1]⁺=253.2.

Step 6: tert-butyl((6-(((2-amino-5-(2-(difluoromethyl)-6-methylpyridin-4-yl)-6-(4-fluorophenyl)pyrimidin-4-yl)oxy)methyl)pyridin-2-yl)methyl)(methyl)carbamate

Following step 2, example 8. From (6-hydroxymethyl-pyridin-2-ylmethyl)-methyl-carbamic acid tert-butyl ester, 60% NaH and 4-chloro-5-(2-(difluoromethyl)-6-methylpyridin-4-yl)-6-(4-fluorophenyl)pyrimidin-2-amine in DMF. The crude was purified by column chromatography on silica gel (DCM:MeOH=20:1) to give tert-butyl((6-(((2-amino-5-(2-(difluoromethyl)-6-methylpyridin-4-yl)-6-(4-fluorophenyl)pyrimidin-4-yl)oxy)methyl)pyridin-2-yl)methyl)(methyl)carbamate (40 mg, 52%) as a yellow oil. ES-MS (m/z): [M+1]⁺=581.3.

Step 7: 5-(2-(difluoromethyl)-6-methylpyridin-4-yl)-4-(4-fluorophenyl)-6-((6-((methylamino)methyl)pyridin-2-yemethoxy)pyrimidin-2-amine

A mixture of tert-butyl((6-(((2-amino-5-(2-(difluoromethyl)-6-methylpyridin-4-yl)-6-(4-fluorophenyl)pyrimidin-4-yl)oxy)methyl)pyridin-2-yl)methyl)(methyl)carbamate (30 mg, 0.052 mmol) and TFA (0.6 mL) in DCM (3.0 mL) was stirred at 0° C. for 3 h. The reaction mixture was adjusted to pH=7-8 with sat. aqueous NaHCO₃, then concentrated. The residue was purified by prep-HPLC to give 5-(2-(difluoromethyl)-6-methylpyridin-4-yl)-4-(4-fluorophenyl)-6-((6-((methylamino)methyl)pyridin-2-yl)methoxy)pyrimidin-2-amine as a TFA salt (32.2 mg, 100%) as a white solid. ES-MS (m/z): [M+1]⁺=481.2.

### Example 407

### 2-((2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-yl)amino)ethan-1-ol

Step 1: N-(2-((tert-butyldimethylsilyl)oxy)ethyl)-2-(6-(((tert-butyldimethylsilyl)oxy)methyl)pyridin-2-yl)propan-2-amine

A mixture of 2-(6-(((tert-butyldimethylsilyl)oxy)methyl)pyridin-2-yl)propan-2-amine (400 mg, 1.43 mmol), (2-bromoethoxy)(tert-butyl)dimethylsilane (509 mg, 2.13 mmol) and K₂CO₃ (592 mg, 4.29 mmol) in DMF (10 mL) was stirred at 80° C. overnight. The mixture was cooled and diluted with H₂O (20 mL) and extracted with ethyl acetate (20 mL×2). The combined organic phases were washed with brine (20 mL), dried over Na₂SO₄, filtered and concentrated. The residue was purified by flash chromatography on silica gel (PE:EA=1:1) to get N-(2-((tert-butyldimethylsilyl)oxy)ethyl)-2-(6-(((tert-butyldimethylsilyl)oxy)methyl)pyridin-2-yl)propan-2-amine (350 mg, 56%) as a yellow oil. ES-MS (m/z): [M+1]⁺=439.3.

Step 2: 2-((2-(6-(hydroxymethyl)pyridin-2-yl)propan-2-yl)amino)ethanol

A solution of N-(2-((tert-butyldimethylsilyl)oxy)ethyl)-2-(6-(((tert-butyldimethylsilyl)oxy)methyl)pyridin-2-yl)propan-2-amine (350 mg, 0.80 mmol) and NH₄F (118 mg, 3.19 mmol) in MeOH (8.0 mL) was stirred at 30° C. overnight. The reaction mixture was concentrated and the residue was triturated with DCM:MeOH=20:1. The mixture was filtered and concentrated to get crude 2-((2-(6-(hydroxymethyl)pyridin-2-yl)propan-2-yl)amino)ethanol (200 mg) as a yellow solid, which was used for next step without further purification. ES-MS (m/z): [M+1]⁺=211.1.

Step 3: 24(2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-yl)amino)ethan-1-ol

Following step 2, example 8. From 2-((2-(6-(hydroxymethyl)pyridin-2-yl)propan-2-yl)amino)ethanol, 60% NaH and 4-chloro-5-(2-(difluoromethyl)-6-methylpyridin-4-yl)-6-(4-fluorophenyl)pyrimidin-2-amine in DMF. The crude was purified by preparative TLC (DCM:MeOH=15:1) to get 2-((2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-yl)amino)ethan-1-ol as a white solid. ES-MS (m/z): [M+1]⁺=539.2.

### Example 408

### 5-(2-(difluoromethyl)-6-methylpyridin-4-yl)-4-(2-(3,4-dihydro-1,8-naphthyridin-1 (2H)-yl)ethoxy)-6-(4-fluorophenyl)pyrimidin-2-amine

Step 1: 2-(3,4-dihydro-1,8-naphthyridin-1(2H)-yl)ethan-1-ol

To a stirring solution of 1,2,3,4-tetrahydro-1,8-naphthyridine (60 mg, 0.45 mmol) in DMF (3.0 mL) was added NaH (90 mg, 2.25 mmol, 60% in mineral oil) at room temperature. The resulting mixture was stirred at 30° C. for 30 min, 2-iodoethanol (231 mg, 1.34 mmol) then was added. The reaction mixture was heated at 60° C. for 2 h, cooled and quenched with H₂O (15 mL), extracted with DCM (15 mL×2). The combined organic phases were washed with brine (15 mL), dried over Na₂SO₄, filtered and concentrated. The residue was purified by flash chromatography on silica gel (DCM:MeOH=20:1) to get 2-(3,4-dihydro-1,8-naphthyridin-1(2H)-yl)ethan-1-ol (20 mg, 25%) as a yellow oil.

Step 2: 5-(2-(difluoromethyl)-6-methylpyridin-4-yl)-4-(2-(3,4-dihydro-1,8-naphthyridin-1(2H)-yl)ethoxy)-6-(4-fluorophenyl)pyrimidin-2-amine

Following step 2, example 8. From 2-(3,4-dihydro-1,8-naphthyridin-1(2H)-yl)ethan-1-ol, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=507.2.

### Example 410

### methyl((6-(((2-amino-5-(2-(difluoromethyl)-6-methylpyridin-4-yl)-6-(4-fluorophenyl)pyrimidin-4-yl)oxy)methyl)pyridin-2-yl)methyl)carbamate

Step 1: methyl 46-(((tert-butyldimethylsilyl)oxy)methyl)pyridin-2-yl)methyl)carbamate

To a stirring solution of (6-(((tert-butyldimethylsilyl)oxy)methyl)pyridin-2-yl)methanamine (150 mg, 0.594 mmol) and triethylamine (180 mg, 1.785 mmol) in DCM (2.0 mL) was added methyl chloroformate (68 mg, 0.720 mmol) at 0° C. The resulting mixture was stirred at room temperature for 2 h, diluted with DCM (20 mL), washed with water (10 mL×3) and brine (10 mL). The organic layer was dried over Na₂SO₄, filtered and concentrated to give crude methyl((6-(((tert-butyldimethylsilyl)oxy)methyl)pyridin-2-yl)methyl)carbamate (190 mg, 103%) as a yellow oil, which was used for next step without further purification. ES-MS (m/z): [M+1]⁺=311.2.

Step 2: methyl((6-(hydroxymethyl)pyridin-2-yl)methyl)carbamate

A mixture of methyl((6-(((tert-butyldimethylsilyl)oxy)methyl)pyridin-2-yl)methyl)carbamate (190 mg, 0.612 mmol) and NH₄F (227 mg, 6.13 mmol) in MeOH (3.0 mL) was stirred at room temperature overnight. The reaction mixture was concentrated and the residue was triturated with DCM (5.0 mL), filtered and the filtrate was concentrated to give crude methyl((6-(hydroxymethyl)pyridin-2-yl)methyl)carbamate (110 mg, 92%) as a yellow oil, which was used for next step without further purification. ES-MS (m/z): [M+1]⁺=197.2.

Step 3: methyl ((6-(((2-amino-5-(2-(difluoromethyl)-6-methylpyridin-4-yl)-6-(4-fluorophenyl)pyrimidin-4-yl)oxy)methyl)pyridin-2-yl)methyl)carbamate

Following step 2, example 8. From methyl((6-(hydroxymethyl)pyridin-2-yl)methyl)carbamate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=525.2.

### Example 413

### 4-((2-aminopyrimidin-4-yl)methoxy)-5-(2-(difluoromethyl)-6-methylpyridin-4-yl)-6-(4-fluorophenyl)pyrimidin-2-amine

Step 1: (2-aminopyrimidin-4-yl)methanol

A solution of (2-chloropyrimidin-4-yl)methanol (300 mg, 2.08 mmol) and aqueous NH₃ (30%, 1.0 mL) in isopropanol (0.5 mL) was heated at 80° C. under sealed condition for 2 h. The reaction mixture was concentrated and purified by column chromatography on silica gel (DCM:MeOH=100:1) to give (2-aminopyrimidin-4-yl)methanol (60 mg, 23%) as yellow oil. ES-MS (m/z): [M+1]⁺=125.9.

Step 2: 4-((2-aminopyrimidin-4-yl)methoxy)-5-(2-(difluoromethyl)-6-methylpyridin-4-yl)-6-(4-fluorophenyl)pyrimidin-2-amine

Following step 2, example 8. From (2-aminopyrimidin-4-yl)methanol, 60% NaH and 4-chloro-5-(2-(difluoromethyl)-6-methylpyridin-4-yl)-6-(4-fluorophenyl)pyrimidin-2-amine in THF. ES-MS (m/z): [M+1]⁺=454.1.

### Example 415

### 1-(((6-(((2-amino-5-(2-(difluoromethyl)-6-methylpyridin-4-yl)-6-(4-fluorophenyl)pyrimidin-4-yl)oxy)methyl)pyridin-2-yl)methyl)-3 -methylurea

Step 1: 1-((6-(hydroxymethyl)pyridin-2-yl)methyl)-3-methylurea

To a stirring solution of (6-(((tert-butyldimethylsilyl)oxy)methyl)pyridin-2-yl)methanamine (100 mg, 0.396 mmol) in DCM (3 mL) was added CDI (71 mg, 0.438 mmol) at room temperature. After stirring at room temperature for 10 min, MeNH₂ (2.0 M in THF, 0.4 mL, 0.800 mmol) was added. The resulting mixture was stirred continually at room temperature for 3 h, diluted with DCM (20 mL), washed with water (10 mL), dried over Na₂SO₄, and concentrated. The residue was dissolved in MeOH (3 mL), and NH₄F (60 mg, 1.620 mmol) was added. The resulting mixture was stirred at room temperature overnight, concentrated and the residue was purified by column chromatography on silica gel (DCM:MeOH=20:1) to give 1-((6-(hydroxymethyl)pyridin-2-yl)methyl)-3-methylurea (55 mg, 71%) as a yellow oil. ES-MS (m/z): [M+1]⁺=196.2.

Step 2: 1-((6-(((2-amino-5-(2-(difluoromethyl)-6-methylpyridin-4-yl)-6-(4-fluorophenyl)pyrimidin-4-yl)oxy)methyl)pyridin-2-yl)methyl)-3-methylurea

Following step 2, example 8. From 1-((6-(hydroxymethyl)pyridin-2-yl)methyl)-3-methylurea, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=524.2.

### Example 417

### 2-((2-(6-(((2-amino-5-(2-(difluoromethyl)-6-methylpyridin-4-yl)-6-(4-fluorophenyl)pyrimidin-4-yl)oxy)methyl)pyridin-2-yepropan-2-yl)amino)propane-1,3-diol

Step 1: 1,3-bis((tetrahydro-2H-pyran-2-yl)oxy)propan-2-one

A solution of 1,3-dihydroxypropan-2-one (3.0 g, 33.3 mmol), 3,4-dihydro-2H-pyran (11.1 g, 132.0 mmol) and PPTS (836 mg, 3.3 mmol) in DCM (40 mL) was stirred at 30° C. overnight. The mixture was concentrated and the residue was dissolved in ether (30 mL), washed with saturated aqueous NaHCO₃ (30 mL) and brine (20 mL). The organic layer was dried over Na₂SO₄, filtered and concentrated. The residue was purified by flash chromatography on silica gel (PE:EA=5:1) to get 1,3-bis((tetrahydro-2H-pyran-2-yl)oxy)propan-2-one (5.0 g, 58%) as a colorless oil.

Step 2: N-(2-(6-(((tert-butyldimethylsilyl)oxy)methyl)pyridin-2-yl)propan-2-yl)-1,3-bis((tetrahydro-2H-pyran-2-yl)oxy)propan-2-amine

A mixture of 2-(6-(((tert-butyldimethylsilyl)oxy)methyl)pyridin-2-yl)propan-2-amine (300 mg, 1.07 mmol), 1,3-bis((tetrahydro-2H-pyran-2-yl)oxy)propan-2-one (1.1 g, 4.26 mmol), NaBH₃CN (270 mg, 4.30 mmol) and 2.0 M HCl in DMF (5.0 mL) was stirred at 70° C. overnight. The reaction mixture was cooled and diluted with H₂O (20 mL), extracted with ethyl acetate (20 mL×2). The combined organic phases were washed with brine (20 mL), dried over Na₂SO₄, filtered and concentrated. The residue was purified by flash chromatography on silica gel (PE:EA=3:1) to get N-(2-(6-(((tert-butyldimethylsilyl)oxy)methyl)pyridin-2-yl)propan-2-yl)-1,3-bis((tetrahydro-2H-pyran-2-yl)oxy)propan-2-amine (300 mg, 53%) as a yellow oil. ES-MS (m/z): [M+1]⁺=523.3.

Step 3: (6-(2-((1,3-bis((tetrahydro-2H-pyran-2-yl)oxy)propan-2-yl)amino)propan-2-yl)pyridin-2-yl)methanol

A mixture of N-(2-(6-(((tert-butyldimethylsilyl)oxy)methyl)pyridin-2-yl)propan-2-yl)-1,3-bis((tetrahydro-2H-pyran-2-yl)oxy)propan-2-amine (300 mg, 0.57 mmol) and NH₄F (63 mg, 1.70 mmol) in MeOH (5.0 mL) was stirred at 30° C. overnight. The reaction mixture was concentrated and the residue was triturated with DCM:MeOH=20:1 (20 mL). The mixture was filtered and concentrated to get crude (6-(2-((1,3-bis((tetrahydro-2H-pyran-2-yl)oxy)propan-2-yl)amino)propan-2-yl)pyridin-2-yl)methanol (280 mg) as a brown oil, which was used for next step without further purification. ES-MS (m/z): [M+1]⁺=409.2.

Step 4: 4-((6-(2-(1,3-bis((tetrahydro-2H-pyran-2-yl)oxy)propan-2-yl)amino)propan-2-yl)pyridin-2-yl)methoxy)-5-(2-(difluoromethyl)-6-methylpyridin-4-yl)-6-(4-fluorophenyl)pyrimidin-2-amine

Following step 2, example 8. From (6-(2-((1,3-bis((tetrahydro-2H-pyran-2-yl)oxy)propan-2-yl)amino)propan-2-yl)pyridin-2-yl)methanol, 60% NaH and 4-chloro-5-(2-(difluoromethyl)-6-methylpyridin-4-yl)-6-(4-fluorophenyl)pyrimidin-2-amine in DMF. The crude was purified by flash chromatography on silica gel (DCM:MeOH=30:1) to get 4-((6-(2-((1,3-bis((tetrahydro-2H-pyran-2-yl)oxy)propan-2-yl)amino)propan-2-yl)pyridin-2-yl)methoxy)-5-(2-(difluoromethyl)-6-methylpyridin-4-yl)-6-(4-fluorophenyl)pyrimidin-2-amine (40 mg, 25%) as a yellow solid. ES-MS (m/z): [M+1]⁺=737.3.

Step 5: 2-((2-(6-(((2-amino-5-(2-(difluoromethyl)-6-methylpyridin-4-yl)-6-(4-fluorophenyl)pyrimidin-4-yl)oxy)methyl)pyridin-2-yl)propan-2-yl)amino)propane-1,3-diol

A mixture of 4-46-(2-41,3-bis((tetrahydro-2H-pyran-2-yl)oxy)propan-2-yl)amino)propan-2-yl)pyridin-2-yemethoxy)-5-(2-(difluoromethyl)-6-methylpyridin-4-yl)-6-(4-fluorophenyl)pyrimidin-2-amine (40 mg, 0.054 mmol) and HCl/MeOH (1.0 M, 1.0 mL) in MeOH (2.0 mL) was stirred at room temperature for 30 min The reaction mixture was concentrated and the residue was purified by Prep-HPLC to get 24(2-(6-(42-amino-5-(2-(difluoromethyl)-6-methylpyridin-4-yl)-6-(4-fluorophenyl)pyrimidin-4-yl)oxy)methyl)pyridin-2-yl)propan-2-yl)amino)propane-1,3-diol (5 mg, 16%) as a white solid. ES-MS (m/z): [M+1]⁺=569.4.

### Example 418

### 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)ethan-1-ol

Step 1: 6-(((tert-butyldimethylsilyl)oxy)methyl)picolinaldehyde

To a stirring solution of 2-bromo-6-(((tert-butyldimethylsilyl)oxy)methyl)pyridine (1.0 g, 3.308 mmol) in THF (20 mL) was added dropwise n-BuLi (2.5 M in hexanes, 2.4 mL, 6.0 mmol) at −78° C. under nitrogen atmosphere. This resulting mixture was stirred at −78° C. for 30 min, DMF (364 mg, 4.980 mmol) was added dropwise at −78° C. After stirring at −78° C. for 3 h, the reaction mixture was quenched with saturated aqueous NH₄Cl, extracted with EtOAc (30 mL×2). The combined organic layers were washed with brine (30 mL), dried over Na₂SO₄, filtered and concentrated. The residue was purified by column chromatography on silica gel (PE:EA=20:1) to give 6-(((tert-butyldimethylsilyl)oxy)methyl)picolinaldehyde (350 mg, 42%) as a yellow oil. ES-MS (m/z): [M+1]⁺=252.1.

Step 2: 2-(((6-(((tert-butyldimethylsilyl)oxy)methyl)pyridin-2-yl)methyl)amino)ethan-1-ol

To a stirring solution of 6-(((tert-butyldimethylsilyl)oxy)methyl)picolinaldehyde (350 mg, 1.392 mmol) in DCE/MeOH (8 mL/2 mL) was added 2-amino-ethanol (128 mg, 2.096 mmol) and NaBH3CN (175 mg, 2.785 mmol) under nitrogen atmosphere. This resulting mixture was stirred at room temperature overnight, quenched with H₂O (10 mL), extracted with DCM (10 mL×3). The combined organic layers were dried over Na₂SO₄, filtered and concentrated. The residue was purified by column chromatography on silica gel (DCM:MeOH=10:1) to give 2-(((6-(((tert-butyldimethylsilyl)oxy)methyl)pyridin-2-yl)methyl)amino)ethan-1-ol (150 mg, 36%) as a colorless oil. ES-MS (m/z): [M+1]⁺=297.3.

Step 3: tert-butyl((6-(((tert-butyldimethylsilyl)oxy)methyl)pyridin-2-yl)methyl)(2-hydroxyethyl)carbamate

To a stirring solution of 2-(((6-(((tert-butyldimethylsilyl)oxy)methyl)pyridin-2-yl)methyl)amino)ethan-1-ol (150 mg, 0.506 mmol) in DCM (3.0 mL) was added (BOC)20 (164 mg, 0.751 mmol) and triethylamine (154 mg, 1.522 mmol). The resulting mixture was stirred at room temperature under nitrogen atmosphere for 3 h, concentrated and the residue was purified by column chromatography on silica gel (PE:EA=1:1) to give tert-butyl((6-(((tert-butyldimethylsilyl)oxy)methyl)pyridin-2-yl)methyl)(2-hydroxyethyl)carbamate (150 mg, 75%) as a yellow oil. ES-MS (m/z): [M+1]⁺=397.3.

Step 4: tert-butyl ((6-(((tert-butyldimethylsilyl)oxy)methyl)pyridin-2-yl)methyl)(2-((tetrahydro-2H-pyran-2-yl)oxy)ethyl)carbamate

To a stirring solution of tert-butyl((6-(((tert-butyldimethylsilyl)oxy)methyl)pyridin-2-yl)methyl)(2-hydroxyethyl)carbamate (150 mg, 0.378 mmol) in DCM (3 mL) was added PPTS (20 mg, 0.0796 mmol) and DHP (129 mg, 1.534 mmol) under nitrogen atmosphere. The resulting mixture was stirred at 30° C. overnight, concentrated and the residue was purified by column chromatography on silica gel (PE:EA=1:1) to give tent-butyl((6-(((tert-butyldimethylsilyl)oxy)methyl)pyridin-2-yl)methyl)(2-((tetrahydro-2H-pyran-2-yl)oxy)ethyl)carbamate (150 mg, 83%) as a colorless oil. ES-MS (m/z): [M+1]⁺=481.3.

Step 5: tert-butyl((6-(hydroxymethyl)pyridin-2-yl)methyl)(2-((tetrahydro-2H-pyran-2-yl)oxy)ethyl)carbamate

A mixture of [tert-butyl((6-(((tert-butyldimethylsilyl)oxy)methyl)pyridin-2-yl]methyl)(2-((tetrahydro-2H-pyran-2-yl)oxy)ethyl)carbamate (150 mg, 0.312 mmol) and NH₄F (116 mg, 3.132 mmol) in MeOH (3.0 mL) was stirred at room temperature overnight. The reaction mixture was concentrated and the residue was purified by column chromatography on silica gel (PE:EA=1:1) to give tert-butyl((6-(hydroxymethyl)pyridin-2-yl)methyl)(2-((tetrahydro-2H-pyran-2-yl)oxy)ethyl)carbamate (73 mg, 64%) as a colorless oil. ES-MS (m/z): [M+1]⁺=367.2.

Step 6: tert-butyl ((6-(((2-amino-5-(2-(difluoromethyl)-6-methylpyridin-4-yl)-6-(4-fluorophenyl)pyrimidin-4-yl)oxy)methyl)pyridin-2-yl)methyl)(2-((tetrahydro-2H-pyran-2-yl)oxy)ethyl)carbamate

Following step 2, example 8. From tert-butyl((6-(hydroxymethyl)pyridin-2-yl)methyl)(2-((tetrahydro-2H-pyran-2-yl)oxy)ethyl)carbamate, 60% NaH and 4-chloro-5-(2-(difluoromethyl)-6-methylpyridin-4-yl)-6-(4-fluorophenyl)pyrimidin-2-amine in DMF. The crude was purified by column chromatography on silica gel (PE:EA=1:1) to give tert-butyl((6-4(2-amino-5-(2-(difluoromethyl)-6-methylpyridin-4-yl)-6-(4-fluorophenyl)pyrimidin-4-yl)oxy)methyl)pyridin-2-yl)methyl)(2-((tetrahydro-2H-pyran-2-yl)oxy)ethyl)carbamate (60 mg, 68%) as a colorless oil. ES-MS (m/z): [M+1]⁺=695.2.

Step 7: 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)ethan-1-ol

A mixture of tert-butyl((6-((2-amino-5-(2-(difluoromethyl)-6-methylpyridin-4-yl)-6-(4-fluorophenyl)pyrimidin-4-yl)oxy)methyl)pyridin-2-yl)methyl)(2-((tetrahydro-2H-pyran-2-yl)oxy)ethyl)carbamate (60 mg, 0.0864 mmol) and TFA (0.5 mL) in DCM (2.0 mL) was stirred at room temperature for 1 h. The reaction mixture was diluted with MeOH (5 mL), adjusted to pH=7-8 with aqueous saturated NaHCO₃ and filtered. The organic layer was separated and concentrated. The residue was purified by Prep-HPLC to give 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)ethan-1-ol (36.8 mg, 83%) as a pale yellow solid. ES-MS (m/z): [M+1]⁺=511.2.

### Example 419

### 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)propane-1,3-diol

Step 1: 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)propane-1,3-diol

A mixture of 4-((6-(aminomethyl)pyridin-2-yl)methoxy)-5-(2-(difluoromethyl)-6-methylpyridin-4-yl)-6-(4-fluorophenyl)pyrimidin-2-amine (310 mg, 0.665 mmol), 2-aminopropane-1,3-diol (10 mg, 0.110 mmol) and CH₃COOH (13.2 mg, 0.220 mmol) in DCE (2.0 mL) was stirred under nitrogen atmosphere at room temperature overnight. The reaction mixture was concentrated and purified by Prep-HPLC to give 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)propane-1,3-diol (2.7 mg, 5%) as a white solid. ES-MS (m/z): [M+1]⁺541.3

### Example 420

### 2-((6-(((2-amino-5-(2-(difluoromethyl)-6-methylpyridin-4-yl)-6-(4-fluorophenyl)pyrimidin-4-yl)oxy)methyl)pyridin-2-yl)oxy)propane-1,3 -diol

Step 1: 2-bromo-6-((2-(4-methoxyphenyl)-1,3-dioxan-5-yl)oxy)pyridine

To a stirring mixture of NaH (60.0 mg, 1.500 mmol, 60% in mineral oil) in DMF (3.0 mL) was added 2-(4-methoxyphenyl)-1,3-dioxan-5-ol (300.0 mg, 1.427 mmol) and 2,6-dibromopyridine (331.8 mg, 1.400 mmol) at 0° C. The resulting mixture was stirred at 100° C. for 2 h, cooled, quenched with water (50 mL) and extracted with ethyl acetate (50 mL×2). The combined organic layers were washed with brine, dried over Na₂SO₄, filtered and concentrated. The residue was purified by chromatograph on silica gel (PE/EA=10/1) to give 2-bromo-6-((2-(4-methoxyphenyl)-1,3-dioxan-5-yl)oxy)pyridine (315 mg, 61%) as a white solid.

Step 2: (6-((2-(4-methoxyphenyl)-1,3-dioxan-5-yl)oxy)pyridin-2-yl)methanol

To a stirring solution of 2-bromo-6-((2-(4-methoxyphenyl)-1,3-dioxan-5-yl)oxy)pyridine (315 mg, 0.860 mmol) in anhydrous THF (4.0 mL) was added n-BuLi (2.5 M, 0.5 mL, 1.25 mmol) at −70° C. dropwise. The resulting mixture was stirred at −70° C. for 0.5 h, anhydrous DMF (100.4 mg, 1.374 mmol) then was added dropwise at −70° C. After stirring continually at −70° C. for 1 h, the reaction mixture was quenched with MeOH (4.0 mL) at −30° C., followed by the addition of NaBH₄ (65.4 mg, 1.729 mmol) at 0° C. The resulting mixture was stirred at room temperature for 1 h, concentrated and the residue was purified by silica gel chromatograph (PE/EA=3/1) to give (6-((2-(4-methoxyphenyl)-1,3-dioxan-5-yl)oxy)pyridin-2-yl)methanol (120 mg, 44%) as a white solid.

Step 3: 5-(2-(difluoromethyl)-6-methylpyridin-4-yl)-4-(4-fluorophenyl)-6-((6-((2-(4-methoxyphenyl)-1,3-dioxan-5-yl)oxy)pyridin-2-yl)methoxy)pyrimidin-2-amine

To a stirring solution of (6-((2-(4-methoxyphenyl)-1,3-dioxan-5-yl)oxy)pyridin-2-yl)methanol (120 mg, 0.378 mmol) in DMF (2.0 ml) was added NaH (60% in mineral oil, 22.8 mg, 0.570 mmol) at 0° C. .The resulting mixture was stirred at room temperature for 30 min, 4-chloro-5-(2-(difluoromethyl)-6-methylpyridin-4-yl)-6-(4-fluorophenyl)pyrimidin-2-amine (138.7 mg, 0.380 mmol) then was added at room temperature. The reaction mixture was stirred continually at room temperature for 2 h, quenched with H₂O (20 mL) and extracted with ethyl acetate (50 mL×3). The combined organic layers were washed with brine, dried over Na₂SO₄, filtered and concentrated. The residue was purified by chromatograph on silica gel (DCM/MeOH=50/1) to give 5-(2-(difluoromethyl)-6-methylpyridin-4-yl)-4-(4-fluorophenyl)-6-((6-((2-(4-methoxyphenyl)-1,3-dioxan-5-yl)oxy)pyridin-2-yl)methoxy)pyrimidin-2-amine (170 mg, 70%) as a white solid. ES-MS (m/z): [M+1]⁺=646.5.

Step 4: 2-((6-(((2-amino-5-(2-(difluoromethyl)-6-methylpyridin-4-yl)-6-(4-fluorophenyl)pyrimidin-4-yl)oxy)methyl)pyridin-2-yl)oxy)propane-1,3-diol

A mixture of 5-(2-(difluoromethyl)-6-methylpyridin-4-yl)-4-(4-fluorophenyl)-6-((6-((2-(4-methoxyphenyl)-1,3-dioxan-5-yl)oxy)pyridin-2-yl)methoxy)pyrimidin-2-amine (90 mg, 0.139 mmol) in HC1/MeOH (1.0 M, 3.0 mL) was stirred at room temperature for 1 h. The reaction mixture was quenched with aqueous NaHCO₃ (saturated) and extracted with ethyl acetate (50 mL×2). The combined organic layers were washed with brine, dried over Na₂SO₄, filtered and concentrated. The residue was purified by prep-HPLC to give 2-((6-(((2-amino-5-(2-(difluoromethyl)-6-methylpyridin-4-yl)-6-(4-fluorophenyl)pyrimidin-4-yl)oxy)methyl)pyridin-2-yl)oxy)propane-1,3-diol (35 mg, 48%) as a white solid. ES-MS (m/z): [M+1]⁺=528.2.

### Example 425

### (6-(((2-amino-5-(2-(difluoromethyl)-6-methylpyridin-4-yl)-6-(4-fluorophenyl)pyrimidin-4-yl)oxy)methyl)pyridin-2-yl)methyl methylcarbamate

Step 1: (6-(((tert-butyldimethylsilyl)oxy)methyl)pyridin-2-yl)methyl methylcarbamate

A mixture of (6-(((tert-butyldimethylsilyl)oxy)methyl)pyridin-2-yemethanol (150 mg, 0.592 mmol), CDI (144 mg, 0.888 mmol), DIPEA (229 mg, 1.772 mmol) and DMAP (36 mg, 0.295 mmol) in THF (4.0 mL)) was stirred at room temperature for 30 min under nitrogen atmosphere, MeNH₂ (2.0 M in THF, 1.48 mL, 2.96 mmol) then was added, and the resulting mixture was stirred continually at room temperature for 1 h. The reaction mixture was concentrated and the residue was purified by silica gel column with ethyl acetate/petroleum ether (1:5) to afford (6-(((tert-butyldimethylsilyl)oxy)methyl)pyridin-2-yl)methyl methylcarbamate as a colorless oil (160 mg, 87%). ES-MS (m/z): [M+1]⁺=311.3.

Step 2: (6-(hydroxymethyl)pyridin-2-yl)methyl methylcarbamate

A mixture of (6-(((tert-butyldimethylsilyl)oxy)methyl)pyridin-2-yl)methyl methylcarbamate (160 mg, 0.515 mmol) and NH₄F (95 mg, 2.565 mmol) in MeOH (8.0 mL) was stirred at room temperature for 24 h. The reaction mixture was concentrated and the residue was purified by silica gel column with DCM/MeOH (40:1) to afford (6-(hydroxymethyl)pyridin-2-yl)methyl methylcarbamate as a white solid (95 mg, 94%). ES-MS (m/z): [M+Na]⁺=219.1.

Step 3: (6-(((2-amino-5-(2-(difluoromethyl)-6-methylpyridin-4-yl)-6-(4-fluorophenyl)pyrimidin-4-yl)oxy)methyl)pyridin-2-yl)methyl methylcarbamate

Following step 2, example 8. From (6-(hydroxymethyl)pyridin-2-yl)methyl methylcarbamate, 60% NaH and 4-chloro-5-(2-(difluoromethyl)-6-methylpyridin-4-yl)-6-(4-fluorophenyl)pyrimidin-2-amine in DMF. ES-MS (m/z): [M+1]⁺=525.2.

### Example 429

### (6-(((2-amino-5-(2-(difluoromethyl)-6-methylpyridin-4-yl)-6-(4-fluorophenyl)pyrimidin-4-yl)oxy)methyl)pyridin-2-yl)methyl carbamate

Step 1: 4-((6-(((tert-butyldimethylsilyl)oxy)methyl)pyridin-2-yl)methoxy)-5-(2-(difluoromethyl)-6-methylpyridin-4-yl)-6-(4-fluorophenyl)pyrimidin-2-amine

To a stirring mixture of (6-(((tert-butyldimethylsilyl)oxy)methyl)pyridin-2-yl)methanol (104 mg, 0.410 mmol) in DMF (3.0 mL) was added NaH (60% in mineral oil, 33 mg, 0.825 mmol) at 0° C. After stirring at 0° C. for 30 min, 4-chloro-5-(2-(difluoromethyl)-6-methylpyridin-4-yl)-6-(4-fluorophenyl)pyrimidin-2-amine (100 mg, 0.274 mmol) was added and the resulting mixture was stirred continually at 0° C. for 3 h. The reaction mixture was quenched with aqueous saturated NH₄Cl (1.0 mL) at 0° C. and concentrated. The residue was purified by silica gel column with ethyl acetate/petroleum ether (1:5) to afford 4-((6-(((tert-butyldimethylsilyl)oxy)methyl)pyridin-2-yl)methoxy)-5-(2-(difluoromethyl)-6-methylpyridin-4-yl)-6-(4-fluorophenyl)pyrimidin-2-amine as a yellow solid (65 mg, 41%). ES-MS (m/z): [M+1]⁺=582.5.

Step 2: (6-4(2-amino-5-(2-(difluoromethyl)-6-methylpyridin-4-yl)-6-(4-fluorophenyl)pyrimidin-4-yl)oxy)methyl)pyridin-2-yl)methanol

A mixture of 4-((6-(((tert-butyldimethylsilyl)oxy)methyl)pyridin-2-yemethoxy)-5-(2-(difluoromethyl)-6-methylpyridin-4-yl)-6-(4-fluorophenyl)pyrimidin-2-amine (65 mg, 0.112 mmol) and NH₄F (21 mg, 0.567 mmol) in MeOH (5 mL) was stirred at room temperature for 24 h. The reaction mixture was concentrated and the residue was purified by silica gel column with DCM/MeOH (60:1) to afford (6-((2-amino-5-(2-(difluoromethyl)-6-methylpyridin-4-yl)-6-(4-fluorophenyl)pyrimidin-4-yl)oxy)methyl)pyridin-2-yl)methanol as a white solid (45 mg, 86%). ES-MS (m/z): [M+1]⁺=468.4.

Step 3: (6-4(2-amino-5-(2-(difluoromethyl)-6-methylpyridin-4-yl)-6-(4-fluorophenyl)pyrimidin-4-yl)oxy)methyl)pyridin-2-yl)methyl carbamate

A mixture of (6-((2-amino-5-(2-(difluoromethyl)-6-methylpyridin-4-yl)-6-(4-fluorophenyl)pyrimidin-4-yl)oxy)methyl)pyridin-2-yl)methanol (20 mg, 0.0428 mmol), CDI (10 mg, 0.0617 mmol) and DIPEA (17 mg, 0.132 mmol) in DMF (3.0 mL)) was stirred at room temperature for 30 min, concentrated aqueous NH₃ (3.0 mL) then was added, and the resulting mixture was stirred at room temperature overnight. The reaction mixture was concentrated and the residue was purified by Prep-HPLC to afford (6-((2-amino-5-(2-(difluoromethyl)-6-methylpyridin-4-yl)-6-(4-fluorophenyl)pyrimidin-4-yl)oxy)methyl)pyridin-2-yl)methyl carbamate as a white solid (10.3 mg, 47%). ES-MS (m/z): [M+1]⁺=511.1.

### Example 433

### 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)acetamide

Step 1: 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)acetamide

A mixture of 4-((6-(aminomethyl)pyridin-2-yl)methoxy)-5-(2-(difluoromethyl)-6-methylpyridin-4-yl)-6-(4-fluorophenyl)pyrimidin-2-amine (60 mg, 0.129 mmol), 2-bromoacetamide (18 mg, 0.130 mmol) and DIPEA (17 mg, 0.132 mmol) in ACN (5.0 mL) was stirred at 60° C. for 2 h. The reaction mixture was concentrated and the residue was purified by preparative TLC (DCM:MeOH=15:1) to get 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)acetamide (9 mg, 13%) as a white solid. ES-MS (m/z): [M+1]⁺=524.2.

### Example 440

### 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)-N-methylacetamide

Step 1: 2-(((6-(((2-amino-5-(2-(difluoromethyl)-6-methylpyridin-4-yl)-6-(4-fluorophenyl)pyrimidin-4-yl)oxy)methyl)pyridin-2-yl)methyl)amino)-N-methylacetamide

A mixture of 4-((6-(aminomethyl)pyridin-2-yl)methoxy)-5-(2-(difluoromethyl)-6-methylpyridin-4-yl)-6-(4-fluorophenyl)pyrimidin-2-amine (60 mg, 0.129 mmol), 2-bromo-N-methylacetamide (20 mg, 0.132 mmol) and DIPEA (17 mg, 0.132 mmol) in ACN (5.0 mL) was stirred at 60° C. for 2 h. The reaction mixture was concentrated and the residue was purified by preparative TLC (DCM:MeOH=15:1) to give 2-(((6-(((2-amino-5-(2-(difluoromethyl)-6-methylpyridin-4-yl)-6-(4-fluorophenyl)pyrimidin-4-yl)oxy)methyl)pyridin-2-yl)methyeamino)-N-methylacetamide (15 mg, 20%) as a white solid. ES-MS (m/z): [M+1]⁺=538.3.

### Biological Screening and Anticancer Activity:

Some exemplary assays and examples for assessing therapeutic efficacy, e.g., anti-cancer effects, of exemplary compounds of the invention are described below.

### Adenosine A_{2A} Receptor Competitive Binding Assay

The Tag-lite adenosine A_{2A} receptor ligand binding assay is a homogeneous alternative to radioligand binding assay and developed by Cisbio (Bedford, Mass.). We carried out this assay following Cisbio's instructions with minor modification. Pre-transfected, fluorescent donor-labelled cells (Cisbio Cat#: C1TT1A2A, 200 testes) was stored at −80° C. and thawed right before use. Cells were washed once with 5 ml TLB and gently resuspended in 2 ml TLB. 4 μl suspended cells was dispensed into each well of white low-volume 384-well plate (Greiner Bio-One). Serial dilutions of compounds were prepared in Tag-lite buffer (TLB, Cisbio Cat #: LABMED) to a DMSO concentration of 0.6%. After adding 4 μl of compound dilutions to each well, 4 μl60 nM Adenosine A_{2A} receptor red antagonist fluorescent ligand (Cisbio Cat #: L0058RED) was dispensed to each well. The 384-well assay plate was centrifuged briefly and incubated at room temperature for 2 h. HTRF signals were measured using CIARIOstar plate reader (BMG Labtech).

cAMP Assay

A 293A cell line (Invitrogen, Cat# R705-07) stably expressing the adenosine A_{2A} receptor was generated in house. Briefly, 293A cells were seeded in 6-well plate and infected with A_{2A} expressing lentivirus (custom packaged by Biosettia) and selected with puromycin (1 ug/ml). Cells survived selection were confirmed for A2A expression by RT-qPCR (RT Reagent: High-Capacity cDNA Reverse Transcription Kit, Applied Biosystems/Thermo Fisher Cat# 4368814, qPCR reagent: SYBR™ Green Master Mix, Applied Biosystems/Thermo Fisher Cat# 4309155). The cells were maintained in DMEM/F12 medium containing 10% FBS, 100 units/ml Penicillin, 100 μg/ml Streptomycin, and 1 μg/ml puromycin at 37° C., 5% CO₂. 293A-A2AR cells were harvested on the day of assay and resuspended at 0.6 million cells/ml in DMEM/F12 with 0.1% BSA and 30 U/ml denosine deaminase(Roche, Cat #: 10102121001). Serial dilutions of compounds were prepared in DMEM/F12 with 0.1% BSA to a DMSO concentration of 0.6%. After 4 μl of compound dilutions were added, 4 μl suspended cells were dispensed into low-volume white 384-well plates (Greiner Bio-One). The assay plate was incubated for 2 h, before 4 μl of stimulation buffer (DMEM/F12, 0.1% BSA, 150 μM Ro 20-1724, 24 nM CGS21680) was added to each well. The assay plate was then incubated at room temperature for 30 min. 3 μl of d2 conjugated cAMP and 3 μl cryptate conjugated anti-cAMP antibody were then added. D2 conjugated cAMP and cryptate conjugated anti-cAMP antibody (Cisbio, Cat #: 62AM6PEC) were both prepared in cell lysis buffer according to the manufacturer's instruction. After 1 h incubation at room temperature, plates were read in CIARIOstar plate reader (BMG Labtech) using the HTRF detection mode.

p-CREB Assay

Assay protocol for p-CREB (phosphorylated cAMP response-element binding protein) in human whole blood by flow cytometry

Fresh human whole blood (Biological Specialty Corporation) was added at 100 ul per wellinto a 96-well plate and treated with test compounds (Serial dilutions of 1:3 with starting concentration at 30 uM, 11 concentrations total, in the presence of adenosine deaminase) for one hour at room temperature on a plate shaker. Then 10 ul of 10× NECA (50 uM stock in PBS) and 10 ul of 10 × Rolipram (50 uM stock in PBS) were added, with designated wells for unstimulated (Rolipram+DMSO) and stimulated (Rolipram+NECA+DMSO) conditions. The plates were mixed well and incubated at room temperature for 30 min on a plate shaker. 1× lyse/Fix buffer (5× stock, BD #558049) was warmed to 37° in a water bath. 1200 ul of pre-warmed 1× lyse/Fix buffer was then added to the plate, which was then sealed and mixed by inverting the plate a few times. The plate was then incubated for 30 min at 37° in a water bath. After incubation, the plate was centrifuged at 1600 rpm for 5 min and the supernatant was decanted, then 1200 ul PBS was added and the plate was mixed by inverting a few times followed by centrifuging at 1600 rpm for 5 min and again decanting the supernatant. Then, 200 ul FIX PERM buffer II (BD #558052) was added and the plate was mixed well, then kept on ice for 45 min After spinning the plate at 1600 rpm for 5 min the supernatant was again decanted. Then was added 200 ul staining buffer (BD #554657), and the plate was mixed well and spun at 1600 rpm for 5 min, then the supernatant was again decanted. 200 ul staining buffer was added, the plate was mixed well and the plate was sealed at this step for storage at 4° overnight or until staining. Before staining, the plate was spun at 1600 rpm for 5 min and the supernatant was again decanted. Antibody mix in staining buffer was prepared (75 ul/well, 1:50 dilution for p-CREB antibody from Cell Signaling Tech #14001S, 1:200 dilution for CD4 antibody from BD #566320) and the cells were resuspended in the above antibody mix solution. After mixing well, the plate was incubated (shielded from light) at room temperature for two hours on a plate shaker. It was then spun at 1600 rpm for 5 min and the supernatant was decanted. 200 ul staining buffer was then added to wash cells, and the plate was then mixed by shaking or pipetting. The wash step was repeated at least one more time, and after the final wash, cells were resusptended in 200 ul staining buffer in U bottom plate for flow cytometry analysis.

TABLE 1
Activity of exemplary compounds of the invention.

| | | | | | | | p- |
|---|---|---|---|---|---|---|---|
| | | | | | cAMP | cAMP | CREB |
| | | | | | (nM) | (nM) | assay, |
| | | | LC | | 0.1% | 0.1% | (nM) |
| | | | MS | Binding | BSA, 8 | BSA, 1 | human |
| Exp | | | (M + | assay | nM | uM | whole |
| # | Chemical structure | Chemical name | 1) | (nM) | CGS | CGS | blood) |
| 1 | 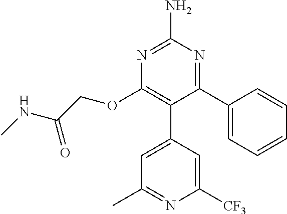 [SMILES: CNC(=O)COc1nc(N)nc(-c2ccccc2)c1-c1cc(C)nc(C(F)(F)F)c1] | 2-((2-amino-5-(2-methyl- 6-(trifluoromethyl)pyridin- 4-yl)-6-phenylpyrimidin-4- yl)oxy)-N- methylacetamide | 418.1 | 11.14 | 40/17 | 4038 | |
| 2 | 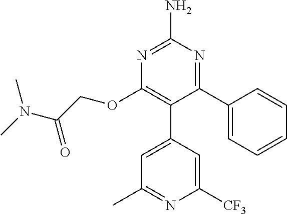 [SMILES: Cc1cc(-c2c(OCC(=O)N(C)C)nc(N)nc2-c2ccccc2)cc(C(F)(F)F)n1] | 2-((2-amino-5-(2-methyl- 6-(trifluoromethyl)pyridin- 4-yl)-6-phenylpyrimidin-4- yl)oxy)-N,N- dimethylacetamide | 432.1 | 30.49 | 6.8/3.4 | | |
| 3 | 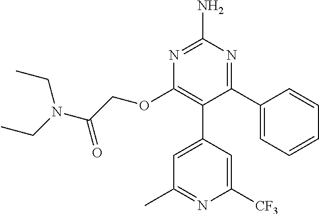 [SMILES: CCN(CC)C(=O)COc1nc(N)nc(-c2ccccc2)c1-c1cc(C)nc(C(F)(F)F)c1] | 2-((2-amino-5-(2-methyl- 6-(trifluoromethyl)pyridin- 4-yl)-6-phenylpyrimidin-4- yl)oxy)-N,N- diethylacetamide | 460.2 | 0.52/ 0.76/ 2.85/ 0.85/ 8.3 | 0.6 | 69/24 | 47 |
| 4 | 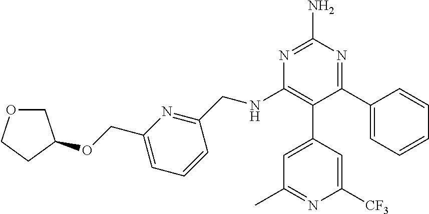 [SMILES: Cc1cc(-c2c(NCc3cccc(CO[C@H]4CCOC4)n3)nc(N)nc2-c2ccccc2)cc(C(F)(F)F)n1] | (S)-5-(2-methyl-6- (trifluoromethyl)pyridin-4- yl)-6-phenyl-N4-((6- (((tetrahydrofuran-3- yl)oxy)methyl)pyridin-2- yl)methyl)pyrimidine-2,4- diamine | 537.2 | 139.1 | 482/288 | 23051 | |
| 5 | 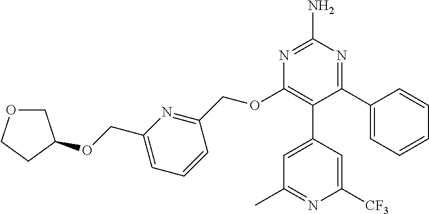 [SMILES: Cc1cc(-c2c(OCc3cccc(CO[C@H]4CCOC4)n3)nc(N)nc2-c2ccccc2)cc(C(F)(F)F)n1] | (S)-5-(2-methyl-6- (trifluoromethyl)pyridin-4- yl)-4-phenyl-6-((6- (((tetrahydrofuran-3- yl)oxy)methyl)pyridin-2- yl)methoxy)pyrimidin-2- amine | 538.1 | 8.97 | 11.5 | | |
| 6 | 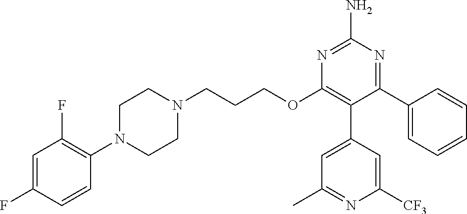 [SMILES: Cc1cc(-c2c(OCCCN3CCN(c4ccc(F)cc4F)CC3)nc(N)nc2-c2ccccc2)cc(C(F)(F)F)n1] | 4-(3-(4-(2,4- difluoropnenyl)piperazin- 1-yl)propoxy)-5-(2-methyl- 6-(trifluoromethyl)pyridin- 4-yl)-6-phenylpyrimidin-2- amine | 585.2 | 74.29 | 182.6 | | |
| 7 | 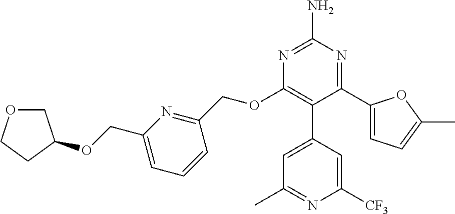 [SMILES: Cc1cc(-c2c(OCc3cccc(CO[C@H]4CCOC4)n3)nc(N)nc2-c2ccc(C)o2)cc(C(F)(F)F)n1] | (S)-5-(2-methyl-6- (trifluoromethyl)pyridin-4- yl)-4-(5-methylfuran-2-yl)- 6-((6-(((tetrahydrofuran-3- yl)oxy)methyl)pyridin-2- yl)methoxy)pyrimidin-2- amine | 542.2 | 58 | 380 | | |
| 8 | 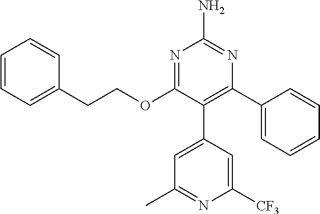 [SMILES: Cc1cc(-c2c(OCCc3ccccc3)nc(N)nc2-c2ccccc2)cc(C(F)(F)F)n1] | 5-(2-methyl-6- (trifluoromethyl)pyridin-4- yl)-4-phenethoxy-6- phenylpyrimidin-2-amine | 451.2 | 10.17/ 18 | 7.4/7.7 | | |
| 9 | 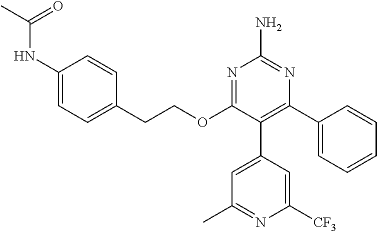 [SMILES: CC(=O)Nc1ccc(CCOc2nc(N)nc(-c3ccccc3)c2-c2cc(C)nc(C(F)(F)F)c2)cc1] | N-(4-(2-((2-amino-5-(2- methyl-6- (trifluoromethyl)pyridin-4- yl)-6-phenylpyrimidin-4- yl)oxy)ethyl)phenyl) acetamide | 508.2 | 0.15/ 2.57 | 5.7/14.0 | | |
| 10 | 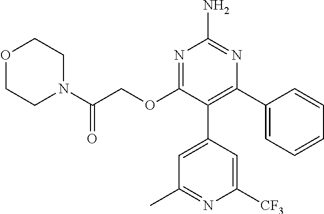 [SMILES: Cc1cc(-c2c(OCC(=O)N3CCOCC3)nc(N)nc2-c2ccccc2)cc(C(F)(F)F)n1] | 2-((2-amino-5-(2-methyl- 6-(trifluoromethyl)pyridin- 4-yl)-6-phenylpyrimidin-4- yl)oxy)-1- morpholinoethan-1-one | 474.2 | 25.25 | 8.8/51.5/ 24.2 | | |
| 11 | 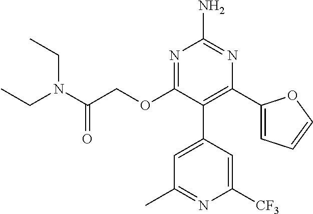 [SMILES: CCN(CC)C(=O)COc1nc(N)nc(-c2ccco2)c1-c1cc(C)nc(C(F)(F)F)c1] | 2-((2-amino-6-(furan-2- yl)-5-(2-methyl-6- (trifluoromethyl)pyridin-4- yl)pyrimidin-4-yl)oxy)- N,N-diethylacetamide | 450.3 | 7.21 | 3.2 | | |
| 12 | 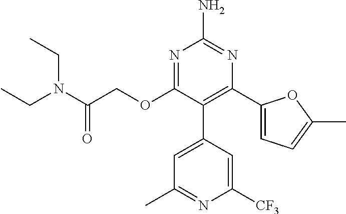 [SMILES: CCN(CC)C(=O)COc1nc(N)nc(-c2ccc(C)o2)c1-c1cc(C)nc(C(F)(F)F)c1] | 2-((2-amino-5-(2-methyl- 6-(trifluoromethyl)pyridin- 4-yl)-6-(5-methylfuran-2- yl)pyrimidin-4-yl)oxy)- N,N-diethylacetamide | 464.1 | 13.81 | 2.0 | | |
| 13 | 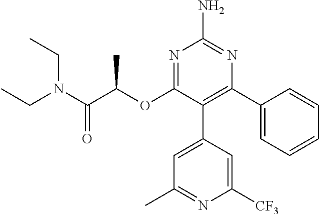 [SMILES: CCN(CC)C(=O)[C@@H](C)Oc1nc(N)nc(-c2ccccc2)c1-c1cc(C)nc(C(F)(F)F)c1] | (R)-2-((2-amino-5-(2- methyl-6- (trifluoromethyl)pyridin-4- yl)-6-phenylpyrimidin-4- yl)oxy)-N,N- diethylpropanamide | 474.0 | 303.5 | 160.6 | | |
| 14 | 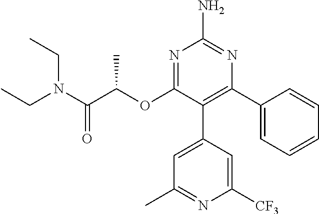 [SMILES: CCN(CC)C(=O)[C@H](C)Oc1nc(N)nc(-c2ccccc2)c1-c1cc(C)nc(C(F)(F)F)c1] | (S)-2-((2-amino-5-(2- methyl-6- (trifluoromethyl)pyridin-4- yl)-6-phenylpyrimidin-4- yl)oxy)-N,N- diethylpropanamide | 474.0 | 900.4 | 10000.0 | | |
| 15 | 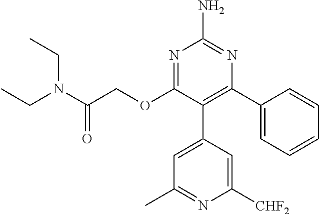 [SMILES: CCN(CC)C(=O)COc1nc(N)nc(-c2ccccc2)c1-c1cc(C)nc(C(F)F)c1] | 2-((2-amino-5-(2- (difluoromethyl)-6- methylpyridin-4-yl)-6- phenylpyrimidin-4-yl)oxy)- N,N-diethylacetamide | 442.1 | 3.2 | 0.5/2.1 | | |
| 16 | 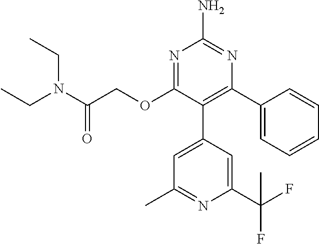 [SMILES: CCN(CC)C(=O)COc1nc(N)nc(-c2ccccc2)c1-c1cc(C)nc(C(C)(F)F)c1] | 2-((2-amino-5-(2-(1,1- difluoroethyl)-6- methylpyridin-4-yl)-6- phenylpyrimidin-4-yl)oxy)- N,N-diethylacetamide | 456.4 | 1.47 | 6.5 | | |
| 17 | 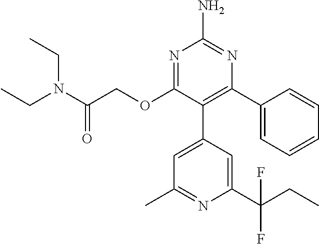 [SMILES: CCN(CC)C(=O)COc1nc(N)nc(-c2ccccc2)c1-c1cc(C)nc(C(F)(F)CC)c1] | 2-((2-amino-5-(2-(1,1- difluoropropyl)-6- methylpyridin-4-yl)-6- phenylpyrimidin-4-yl)oxy)- N,N-diethylacetamide | 470.0 | 9.01 | 7.5 | | |
| 18 | 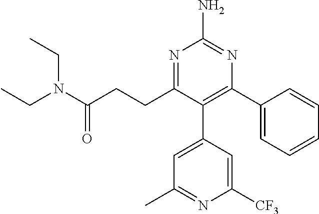 [SMILES: CCN(CC)C(=O)CCc1nc(N)nc(-c2ccccc2)c1-c1cc(C)nc(C(F)(F)F)c1] | 3-(2-amino-5-(2-methyl-6- (trifluoromethyl)pyridin-4- yl)-6-phenylpyrimidin-4- yl)-N,N- diethylpropanamide | 458.2 | 432.7 | | | |
| 19 | 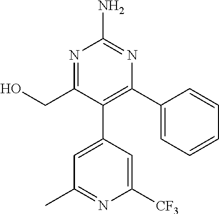 [SMILES: Cc1cc(-c2c(CO)nc(N)nc2-c2ccccc2)cc(C(F)(F)F)n1] | (2-amino-5-(2-methyl-6- (trifluoromethyl)pyridin-4- yl)-6-phenylpyrimidin-4- yl)methanol | 361.0 | 52.04 | | | |
| 20 | 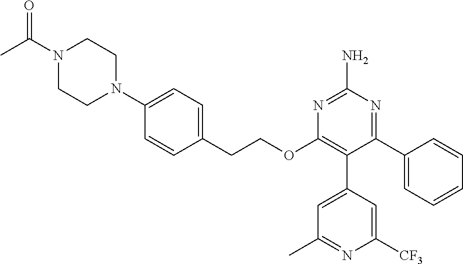 [SMILES: CC(=O)N1CCN(c2ccc(CCOc3nc(N)nc(-c4ccccc4)c3-c3cc(C)nc(C(F)(F)F)c3)cc2)CC1] | 1-(4-(4-(2-((2-amino-5-(2- methyl-6- (trifluoromethyl)pyridin-4- yl)-6-phenylpyrimidin-4- yl)oxy)ethyl)phenyl)piper- azin-1-yl)ethan-1-one | 577.3 | 26.58 | | | |
| 21 | 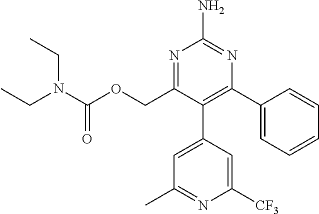 [SMILES: CCN(CC)C(=O)OCc1nc(N)nc(-c2ccccc2)c1-c1cc(C)nc(C(F)(F)F)c1] | (2-amino-5-(2-methyl-6- (trifluoromethyl)pyridin-4- yl)-6-phenylpyrimidin-4- yl)methyl diethylcarbamate | 460.0 | 110.6 | | | |
| 22 | 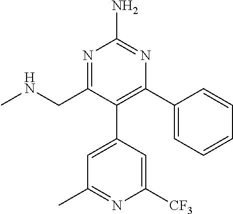 [SMILES: CNCc1nc(N)nc(-c2ccccc2)c1-c1cc(C)nc(C(F)(F)F)c1] | 5-(2-methyl-6- (trifluoromethyl)pyridin-4- yl)-4- ((methylamino)methyl)-6- phenylpyrimidin-2-amine | 374.2 | 1785 | | | |
| 23 | 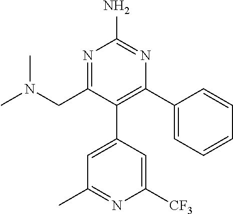 [SMILES: Cc1cc(-c2c(CN(C)C)nc(N)nc2-c2ccccc2)cc(C(F)(F)F)n1] | 4- ((dimethylamino)methyl)- 5-(2-methyl-6- (trifluoromethyl)pyridin-4- yl)-6-phenylpyrimidin-2- amine | 388.3 | 1779 | | | |
| 24 | 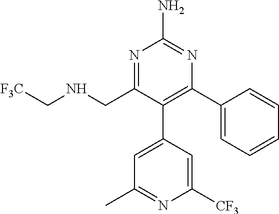 [SMILES: Cc1cc(-c2c(CNCC(F)(F)F)nc(N)nc2-c2ccccc2)cc(C(F)(F)F)n1] | 5-(2-methyl-6- (trifluoromethyl)pyridin-4- yl)-4-phenyl-6-(((2,2,2- trifluoroethyl)amino) methyl)pyrimidin-2-amine | 442.2 | 117.7 | | | |
| 25 | 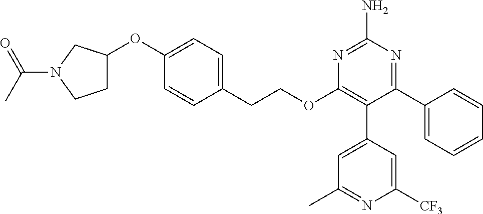 [SMILES: CC(=O)N1CCC(Oc2ccc(CCOc3nc(N)nc(-c4ccccc4)c3-c3cc(C)nc(C(F)(F)F)c3)cc2)C1] | 1-(3-(4-(2-((2-amino-5-(2- methyl-6- (trifluoromethyl)pyridin-4- yl)-6-phenylpyrimidin-4- yl)oxy)ethyl)phenoxy) pyrrolidin-1-yl)ethan-1-one | 578.3 | 32.05 | | | |
| 26 | 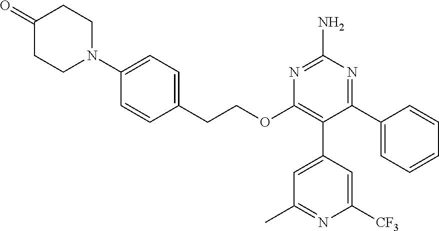 [SMILES: Cc1cc(-c2c(OCCc3ccc(N4CCC(=O)CC4)cc3)nc(N)nc2-c2ccccc2)cc(C(F)(F)F)n1] | 1-(4-(2-((2-amino-5-(2- methyl-6- (trifluoromethyl)pyridin-4- yl)-6-phenylpyrimidin-4- yl)oxy)ethyl)phenyl) piperidin-4-one | 548.2 | 42.99/ 26.78 | | | |
| 27 | 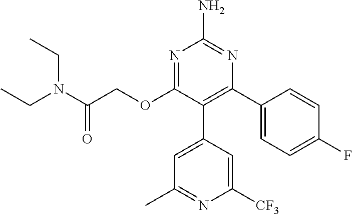 [SMILES: CCN(CC)C(=O)COc1nc(N)nc(-c2ccc(F)cc2)c1-c1cc(C)nc(C(F)(F)F)c1] | 2-((2-amino-6-(4- fluorophenyl)-5-(2-methyl- 6-(trifluoromethyl)pyridin- 4-yl)pyrimidin-4-yl)oxy)- N,N-diethylacetamide | 478.1 | 15.89 | | | |
| 28 | 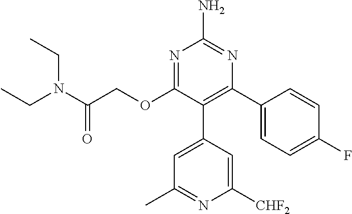 [SMILES: CCN(CC)C(=O)COc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)-N,N- diethylacetamide | 460.2 | 7.56 | 0.44/1.3 | | |
| 29 | 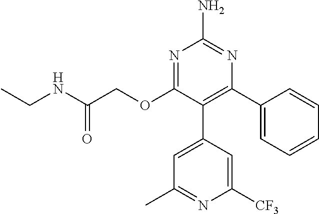 [SMILES: CCNC(=O)COc1nc(N)nc(-c2ccccc2)c1-c1cc(C)nc(C(F)(F)F)c1] | 2-((2-amino-5-(2-methyl- 6-(trifluoromethyl)pyridin- 4-yl)-6-phenylpyrimidin-4- yl)oxy)-N-ethylacetamide | 432.1 | 7.74 | 2.0/2.7 | | |
| 30 | 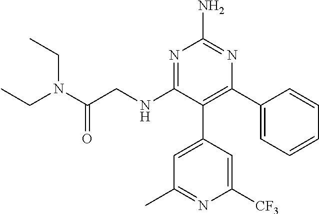 [SMILES: CCN(CC)C(=O)CNc1nc(N)nc(-c2ccccc2)c1-c1cc(C)nc(C(F)(F)F)c1] | 2-((2-amino-5-(2-methyl- 6-(trifiuoromethyl)pyridin- 4-yl)-6-phenylpyrimidin-4- yl)amino)-N,N- diethylacetamide | 459.1 | 9.95 | | | |
| 31 | 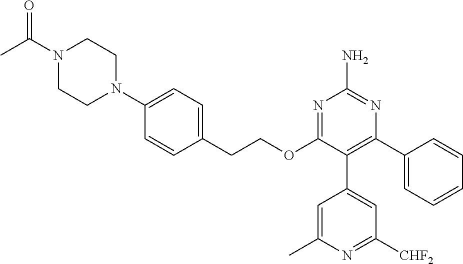 [SMILES: CC(=O)N1CCN(c2ccc(CCOc3nc(N)nc(-c4ccccc4)c3-c3cc(C)nc(C(F)F)c3)cc2)CC1] | 1-(4-(4-(2-((2-amino-5-(2- (difluoromethyl)-6- methylpyridin-4-yl)-6-(4- fluorophenyl)pyrimidin-4- yl)oxy)ethyl)phenyl) piperazin-1-yl)ethan-1-one | 577.3 | 8.35 | | | |
| 32 | 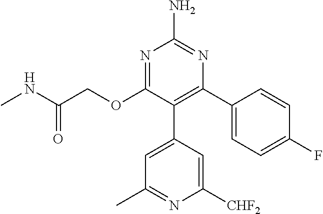 [SMILES: CNC(=O)COc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)-N- methylacetamide | 418.0 | 13.94 | 6.1/6.7 | | |
| 33 | 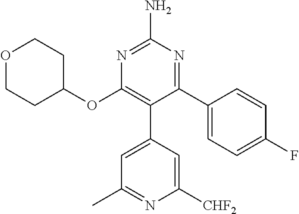 [SMILES: Cc1cc(-c2c(OC3CCOCC3)nc(N)nc2-c2ccc(F)cc2)cc(C(F)F)n1] | 5-((2-(difluoromethyl)-6- methylpyridin-4-yl)-4-(4- fluorophenyl)-6- ((tetrahydro-2H-pyran-4- yl)oxy)pyrimidin-2-amine | 431.1 | 66.55 | | | |
| 34 | 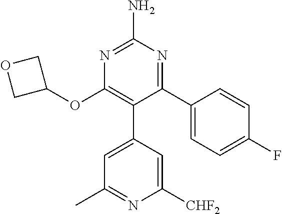 [SMILES: Cc1cc(-c2c(OC3COC3)nc(N)nc2-c2ccc(F)cc2)cc(C(F)F)n1] | 5-(2-(difluoromethyl)-6- methylpyridin-4-yl)-4-(4- fluorophenyl)-6-(oxetan-3- yloxy)pyrimidin-2-amine | 403.1 | 45.32 | | | |
| 35 | 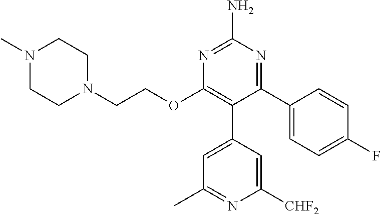 [SMILES: Cc1cc(-c2c(OCCN3CCN(C)CC3)nc(N)nc2-c2ccc(F)cc2)cc(C(F)F)n1] | 5-(2-(difluoromethyl)-6- methylpyridin-4-yl)-4-(4- fluorophenyl)-6-(2-(4- methylpiperazin-1- yl)ethoxy)pyrimidin-2- amine | 473.0 | 124.5 | | | |
| 36 | 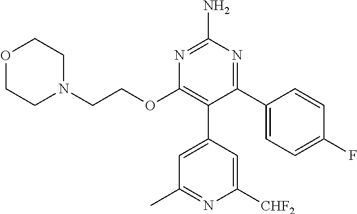 [SMILES: Cc1cc(-c2c(OCCN3CCOCC3)nc(N)nc2-c2ccc(F)cc2)cc(C(F)F)n1] | 5-(2-(difluoromethyl)-6- methylpyridin-4-yl)-4-(4- fluorophenyl)-6-(2- morpholinoethoxy) pyrimidin-2-amine | 460.2 | 11.45 | 77/2.3 | | |
| 37 | 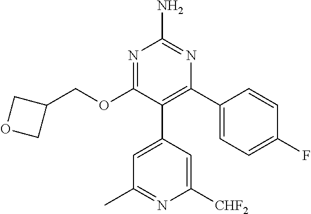 [SMILES: Cc1cc(-c2c(OCC3COC3)nc(N)nc2-c2ccc(F)cc2)cc(C(F)F)n1] | 5-(2-(difluoromethyl)-6- methylpyridin-4-yl)-4-(4- fluorophenyl)-6-(oxetan-3- ylmethoxy)pyrimidin-2- amine | 417.1 | 10.22 | | | |
| 38 | 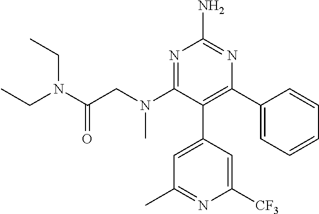 [SMILES: CCN(CC)C(=O)CN(C)c1nc(N)nc(-c2ccccc2)c1-c1cc(C)nc(C(F)(F)F)c1] | 2-((2-amino-5-(2-methyl- 6-(trifluoromethyl)pyridin- 4-yl)-6-phenylpyrimidin-4- yl)(methyl)amino)-N,N- diethylacetamide | 473.1 | 816.0 | | | |
| 39 | 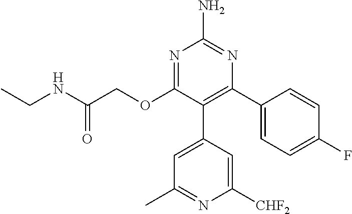 [SMILES: CCNC(=O)COc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)-N-ethylacetamide | 432.0 | 7.2/6.4 | 0.42/ 0.57/ 2.2 | | |
| 40 | 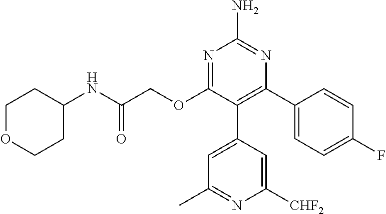 [SMILES: Cc1cc(-c2c(OCC(=O)NC3CCOCC3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(tetrahydro-2H- pyran-4-yl)acetamide | 488.3 | 295.0 | | | |
| 41 | 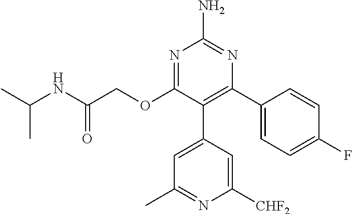 [SMILES: Cc1cc(-c2c(OCC(=O)NC(C)C)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N- isopropylacetamide | 446.1 | 6.7 | 4.1 | | |
| 42 | 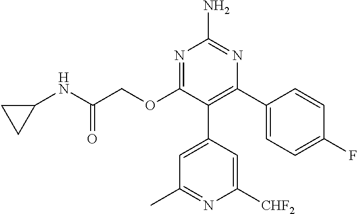 [SMILES: Cc1cc(-c2c(OCC(=O)NC3CC3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N- cyclopropylacetamide | 444.3 | 5.2 | 15/0.56 | | |
| 43 | 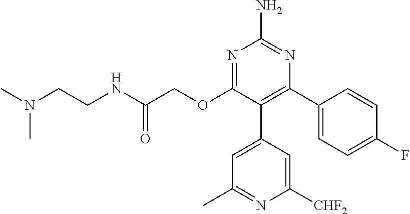 [SMILES: Cc1cc(-c2c(OCC(=O)NCCN(C)C)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(2- (dimethylamino)ethyl) acetamide | 475.1 | 266.0 | | | |
| 44 | 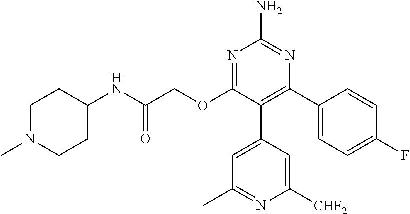 [SMILES: Cc1cc(-c2c(OCC(=O)NC3CCN(C)CC3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(1- methylpiperidin-4- yl)acetamide | 501.1 | 1210.0 | | | |
| 45 | 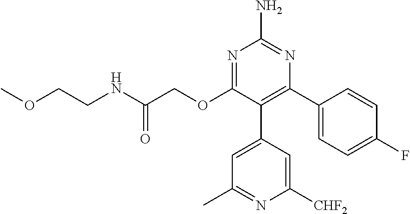 [SMILES: COCCNC(=O)COc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)-N-(2- methoxyethyl)acetamide | 462.1 | 25.7 | 66/8.9 | | |
| 46 | 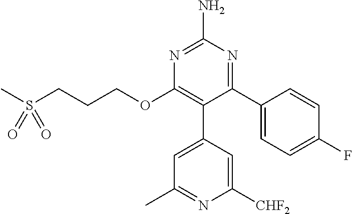 [SMILES: Cc1cc(-c2c(OCCCS(C)(=O)=O)nc(N)nc2-c2ccc(F)cc2)cc(C(F)F)n1] | 5-(2-(difluoromethyl)-6- methylpyridin-4-yl)-4-(4- fluorophenyl)-6-(3- (methylsulfonyl)propoxy) pyrimidin-2-amine | 467.1 | 5.7/5.9 | 0.45/ 0.84/ 2.1 | 1777/ 714 | |
| 47 | 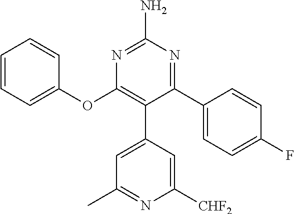 [SMILES: Cc1cc(-c2c(Oc3ccccc3)nc(N)nc2-c2ccc(F)cc2)cc(C(F)F)n1] | 5-(2-(difluoromethyl)-6- methylpyridin-4-yl)-4-(4- fluorophenyl)-6- phenoxypyrimidin-2- amine | 423.1 | 21.3 | | | |
| 48 | 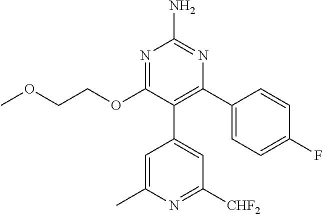 [SMILES: COCCOc1nc(N)nc(-c2ccc(F)cc2)c1-c1cc(C)nc(C(F)F)c1] | 5-(2-(difluoromethyl)-6- methylpyridin-4-yl)-4-(4- fluorophenyl)-6-(2- methoxyethoxy)pyrimidin- 2-amine | 405.1 | 3.7 | 0.72/ 0.18 | | |
| 49 | 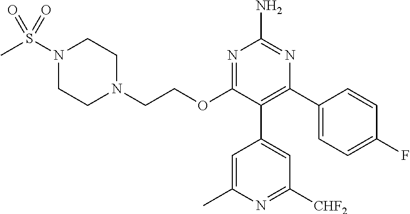 [SMILES: Cc1cc(-c2c(OCCN3CCN(S(C)(=O)=O)CC3)nc(N)nc2-c2ccc(F)cc2)cc(C(F)F)n1] | 5-(2-(difluoromethyl)-6- methylpyridin-4-yl)-4-(4- fluorophenyl)-6-(2-(4- (methylsulfonyl)piperazin- 1-yl)ethoxy)pyrimidin-2- amine | 537.2 | 13.9 | | | |
| 50 | 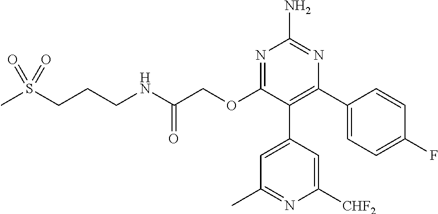 [SMILES: Cc1cc(-c2c(OCC(=O)NCCCS(C)(=O)=O)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(3- (methylsulfonyl)propyl) acetamide | 524.1 | 535.0 | | | |
| 51 | 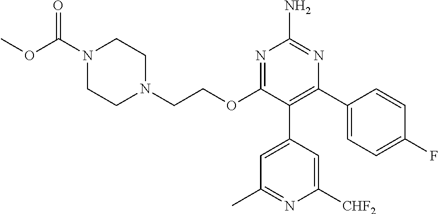 [SMILES: COC(=O)N1CCN(CCOc2nc(N)nc(-c3ccc(F)cc3)c2-c2cc(C)nc(C(F)F)c2)CC1] | methyl 4-(2-((2-amino-5- (2-(difluoromethyl)-6- methylpyridin-4-yl)-6-(4- fluorophenyl)pyrimidin-4- yl)oxy)ethyl)piperazine-1- carboxylate | 517.2 | 10 | 3.9 | | |
| 52 | 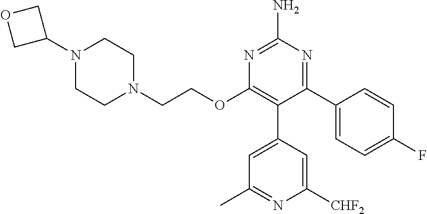 [SMILES: Cc1cc(-c2c(OCCN3CCN(C4COC4)CC3)nc(N)nc2-c2ccc(F)cc2)cc(C(F)F)n1] | 5-(2-(difluoromethyl)-6- methylpyridin-4-yl)-4-(4- fluorophenyl)-6-(2-(4- (oxetan-3-yl)piperazin-1- yl)ethoxy)pyrimidin-2- amine | 515.0 | 120 | 532 | | |
| 53 | 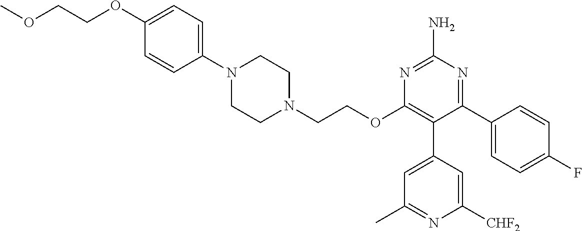 [SMILES: COCCOc1ccc(N2CCN(CCOc3nc(N)nc(-c4ccc(F)cc4)c3-c3cc(C)nc(C(F)F)c3)CC2)cc1] | 5-(2-(difluoromethyl)-6- methylpyridin-4-yl)-4-(4- fluorophenyl)-6-(2-(4-(4- (2- methoxyethoxy)phenyl) piperazin-1- yl)ethoxy)pyrimidin-2- amine | 609.3 | 13 | 0.67 | | |
| 54 | 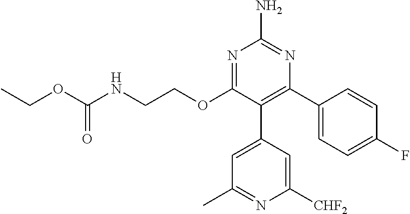 [SMILES: CCOC(=O)NCCOc1nc(N)nc(-c2ccc(F)cc2)c1-c1cc(C)nc(C(F)F)c1] | ethyl (2-((2-amino-5-(2- (difluoromethyl)-6- methylpyridin-4-yl)-6-(4- fluorophenyl)pyrimidin-4- yl)oxy)ethyl)carbamate | 462.1 | 2.1/10 | 0.22/ 1.45 | | |
| 55 | 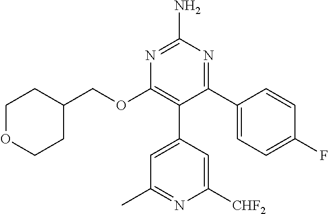 [SMILES: Cc1cc(-c2c(OCC3CCOCC3)nc(N)nc2-c2ccc(F)cc2)cc(C(F)F)n1] | 5-(2-(difluoromethyl)-6- methylpyridin-4-yl)-4-(4- fluorophenyl)-6- ((tetrahydro-2H-pyran-4- yl)methoxy)pyrimidin-2- amine | 445.0 | 0.7/5.4 | 0.54/ 0.49 | 96 | 93 |
| 56 | 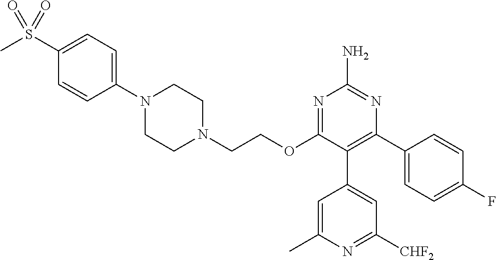 [SMILES: Cc1cc(-c2c(OCCN3CCN(c4ccc(S(C)(=O)=O)cc4)CC3)nc(N)nc2-c2ccc(F)cc2)cc(C(F)F)n1] | 5-(2-(difluoromethyl)-6- methylpyridin-4-yl)-4-(4- fluorophenyl)-6-(2-(4-(4- (methylsulfonyl)phenyl) piperazin-1- yl)ethoxy)pyrimidin-2- amine | 613.3 | 8.9 | 3.6 | | |
| 57 | 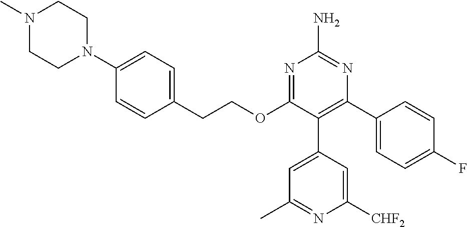 [SMILES: Cc1cc(-c2c(OCCc3ccc(N4CCN(C)CC4)cc3)nc(N)nc2-c2ccc(F)cc2)cc(C(F)F)n1] | 5-(2-(difluoromethyl)-6- methylpyridin-4-yl)-4-(4- fluorophenyl)-6-(4-(4- methylpiperazin-1- yl)phenethoxy)pyrimidin- 2-amine | 549.5 | 2.0/14 | 1.7 | | |
| 58 | 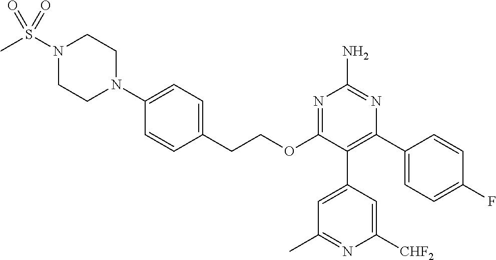 [SMILES: Cc1cc(-c2c(OCCc3ccc(N4CCN(S(C)(=O)=O)CC4)cc3)nc(N)nc2-c2ccc(F)cc2)cc(C(F)F)n1] | 5-(2-(diufluoromethyl)-6- methylpyridin-4-yl)-4-(4- fluorophenyl)-6-(4-(4- (methylsulfonyl)piperazin- 1- yl)phenethoxy)pyrimidin- 2-amine | 613.2 | 28 | | | |
| 59 | 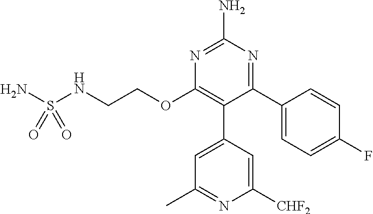 [SMILES: Cc1cc(-c2c(OCCNS(N)(=O)=O)nc(N)nc2-c2ccc(F)cc2)cc(C(F)F)n1] | (2-amino-5-(2- (difluoromethyl)-6- methylpyridin-4-yl)-6-(4- fluorophenyl)pyrimidin-4- yl)oxy)ethyl)sulfuric diamide | 469.1 | 1.8/7.8 | 2.2/1.3 | 663/ 394 | |
| 60 | 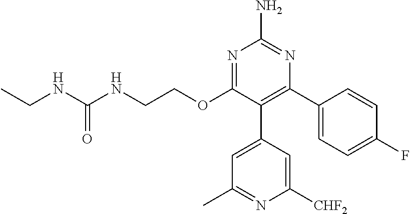 [SMILES: CCNC(=O)NCCOc1nc(N)nc(-c2ccc(F)cc2)c1-c1cc(C)nc(C(F)F)c1] | 1-(2-((2-amino-5-(2- (difluoromethyl)-6- methylpyridin-4-yl)-6-(4- fluorophenyl)pyrimidin-4- yl)oxy)ethyl)-3-ethylurea | 461.0 | 3.6/8.8 | 1.7/2.2 | | |
| 61 | 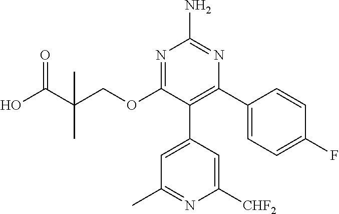 [SMILES: Cc1cc(-c2c(OCC(C)(C)C(=O)O)nc(N)nc2-c2ccc(F)cc2)cc(C(F)F)n1] | 3-((2-amino-5-(2- (difluoromethyl)-6- methylpyridin-4-yl)-6-(4- fluorophenyl)pyrimidin-4- yl)oxy)-2,2- dimethylpropanoic acid | 447.0 | 280 | 233 | | |
| 62 | 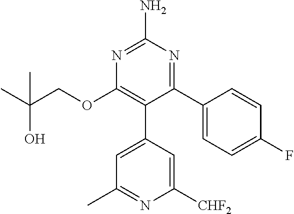 [SMILES: Cc1cc(-c2c(OCC(C)(C)O)nc(N)nc2-c2ccc(F)cc2)cc(C(F)F)n1] | 1-((2-amino-5-(2- (difluoromethyl)-6- methylpyridin-4-yl)-6-(4- fluorophenyl)pyrimidin-4- yl)oxy)-2-methylpropan-2- ol | 419.1 | 15 | 15 | | |
| 63 | 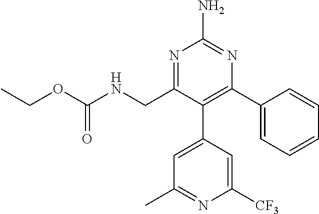 [SMILES: CCOC(=O)NCc1nc(N)nc(-c2ccccc2)c1-c1cc(C)nc(C(F)(F)F)c1] | ethyl ((2-amino-5-(2- methyl-6- (trifluoromethyl)pyridin-4- yl)-6-phenylpyrimidin-4- yl)methyl)carbamate | 432.0 | 37 | 8.3 | | |
| 64 | 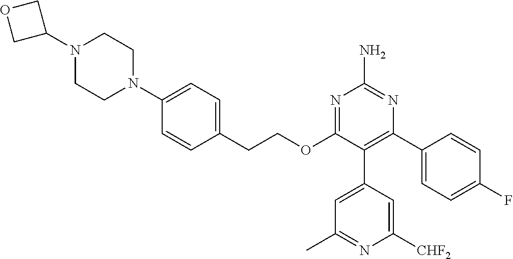 [SMILES: Cc1cc(-c2c(OCCc3ccc(N4CCN(C5COC5)CC4)cc3)nc(N)nc2-c2ccc(F)cc2)cc(C(F)F)n1] | 5-(2-(difluoromethyl)-6- methylpyridin-4-yl)-4-(4- fluorophenyl)-6-(4-(4- (oxetan-3-yl)piperazin-1- yl)phenethoxy)pyrimidin- 2-amine | 591.2 | 31 | | | |
| 65 | 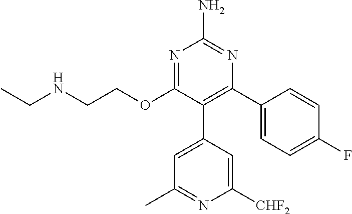 [SMILES: CCNCCOc1nc(N)nc(-c2ccc(F)cc2)c1-c1cc(C)nc(C(F)F)c1] | 5-(2-(difluoromethyl)-6- methylpyridin-4-yl)-4-(2- (ethylamino)ethoxy)-6-(4- fluorophenyl)pyrimidin-2- amine | 418.0 | 42 | 1.6 | 1010 | |
| 66 | 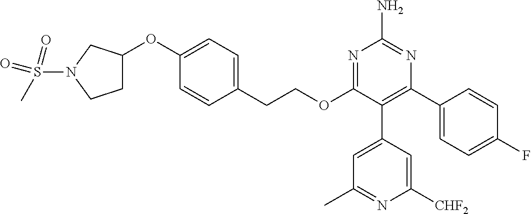 [SMILES: Cc1cc(-c2c(OCCc3ccc(OC4CCN(S(C)(=O)=O)C4)cc3)nc(N)nc2-c2ccc(F)cc2)cc(C(F)F)n1] | 5-(2-(difluoromethyl)-6- methylpyridin-4-yl)-4-(4- fluorophenyl)-6-(4-((1- (methylsulfonyl)pyrrolidin- 3- yl)oxy)phenethoxy) pyrimidin-2-amine | 614.2 | 95 | 337 | | |
| 67 | 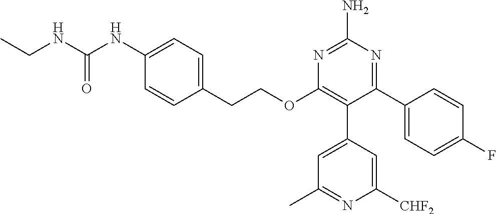 [SMILES: CCNC(=O)Nc1ccc(CCOc2nc(N)nc(-c3ccc(F)cc3)c2-c2cc(C)nc(C(F)F)c2)cc1] | 1-(4-(2-((2-amino-5-(2- (difluoromethyl)-6- methylpyridin-4-yl)-6-(4- fluorophenyl)pyrimidin-4- yl)oxy)ethyl)phenyl)-3- ethylurea | 537.2 | 27 | 4.7 | | |
| 68 | 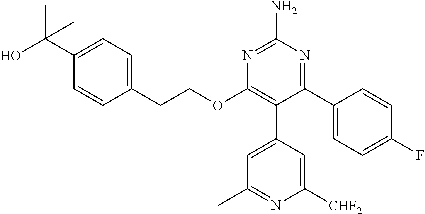 [SMILES: Cc1cc(-c2c(OCCc3ccc(C(C)(C)O)cc3)nc(N)nc2-c2ccc(F)cc2)cc(C(F)F)n1] | 2-(4-(2-((2-amino-5-(2- (difluoromethyl)-6- methylpyridin-4-yl)-6-(4- fluorophenyl)pyrimidin-4- yl)oxy)ethyl)phenyl) propan-2-ol | 509.2 | 22 | 2.7 | | |
| 69 | 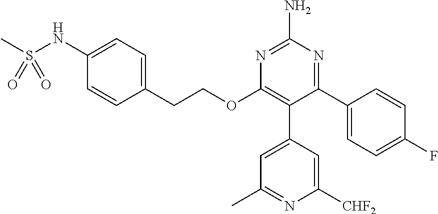 [SMILES: Cc1cc(-c2c(OCCc3ccc(NS(C)(=O)=O)cc3)nc(N)nc2-c2ccc(F)cc2)cc(C(F)F)n1] | N-(4-(2-((2-amino-5-(2- (difluoromethyl)-6- methylpyridin-4-yl)-6-(4- fluorophenyl)pyrimidin-4- yl)oxy)ethyl)phenyl) methanesulfonamide | 544.1 | 38 | 5.8 | | |
| 70 | 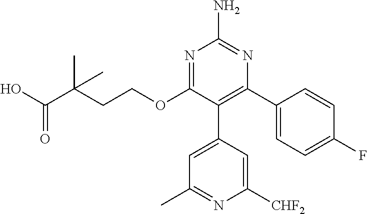 [SMILES: Cc1cc(-c2c(OCCC(C)(C)C(=O)O)nc(N)nc2-c2ccc(F)cc2)cc(C(F)F)n1] | 4-((2-amino-5-(2- (difluoromethyl)-6- methylpyridin-4-yl)-6-(4- fluorophenyl)pyrimidin-4- yl)oxy)-2,2- dimethylbutanoic acid | 461.1 | 3.6/1.0/ 4.0 | 0.2/1.2/ 1.1/1.6/ 3.3/1.9 | 513/ 315/ 231/ 229/ 143 | 673/ 604/ 1290 |
| 71 | 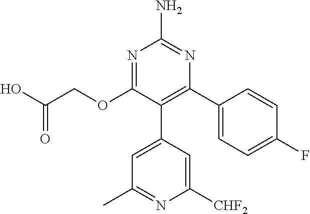 [SMILES: Cc1cc(-c2c(OCC(=O)O)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)acetic acid | 404.9 | >1000 | >1000 | | |
| 72 | 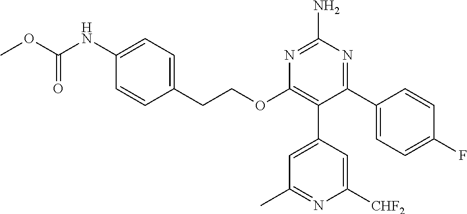 [SMILES: COC(=O)Nc1ccc(CCOc2nc(N)nc(-c3ccc(F)cc3)c2-c2cc(C)nc(C(F)F)c2)cc1] | methyl (4-(2-((2-amino-5- (2-(difluoromethyl)-6- methylpyridin-4-yl)-6-(4- fluorophenyl)pyrimidin-4- yl)oxy)ethyl)phenyl) carbamate | 524.1 | 41 | | | |
| 73 | 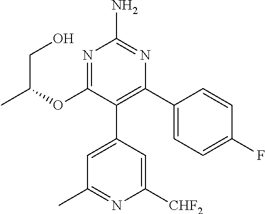 [SMILES: Cc1cc(-c2c(O[C@H](C)CO)nc(N)nc2-c2ccc(F)cc2)cc(C(F)F)n1] | (R)-2-((2-amino-5-(2- (difluoromethyl)-6- methylpyridin-4-yl)-6-(4- fluorophenyl)pyrimidin-4- yl)oxy)propan-1-ol | 405.1 | 25 | 3.6 | | |
| 74 | 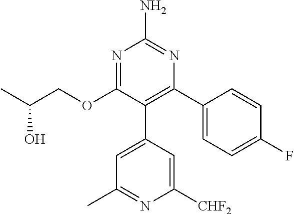 [SMILES: Cc1cc(-c2c(OC[C@@H](C)O)nc(N)nc2-c2ccc(F)cc2)cc(C(F)F)n1] | (R)-1-((2-amino-5-(2- (difluoromethyl)-6- methylpyridin-4-yl)-6-(4- fluorophenyl)pyrimidin-4- yl)oxy)propan-2-ol | 405.1 | 8.4/ 15.7 | 0.3/4.7/ 8.6/6.3 | 693 | |
| 75 | 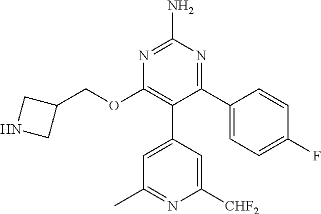 [SMILES: Cc1cc(-c2c(OCC3CNC3)nc(N)nc2-c2ccc(F)cc2)cc(C(F)F)n1] | 4-(azetidin-3-ylmethoxy)- 5-(2-(difluoromethyl)-6- methylpyridin-4-yl)-6-(4- fluorophenyl)pyrimidin-2- amine | 416.0 | 201 | 89 | | |
| 76 | 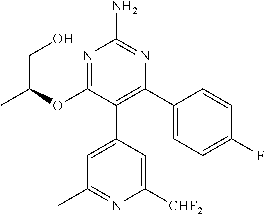 [SMILES: Cc1cc(-c2c(O[C@@H](C)CO)nc(N)nc2-c2ccc(F)cc2)cc(C(F)F)n1] | (S)-2-((2-amino-5-(2- (difluoromethyl)-6- methylpyridin-4-yl)-6-(4- fluorophenyl)pyrimidin-4- yl)oxy)propan-1-ol | 405.1 | 19 | 3.2 | | |
| 77 | 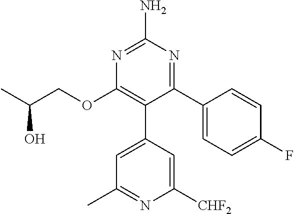 [SMILES: Cc1cc(-c2c(OC[C@H](C)O)nc(N)nc2-c2ccc(F)cc2)cc(C(F)F)n1] | (S)-1-((2-amino-5-(2- (difluoromethyl)-6- methylpyridin-4-yl)-6-(4- fluorophenyl)pyrimidin-4- yl)oxy)propan-2-ol | 405.1 | 3.9/4.2 | 0.19/ 2.0/ 6.9/2.3 | 547 | |
| 78 | 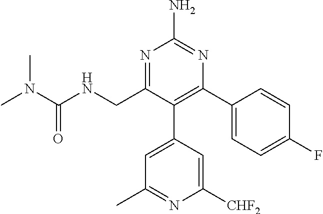 [SMILES: Cc1cc(-c2c(CNC(=O)N(C)C)nc(N)nc2-c2ccc(F)cc2)cc(C(F)F)n1] | 3-((2-amino-5-(2- (difluoromethyl)-6- methylpyridin-4-yl)-6-(4- fluorophenyl)pyrimidin-4- yl)methyl)-1,1- dimethylurea | 431.2 | 11 | 51 | | |
| 79 | 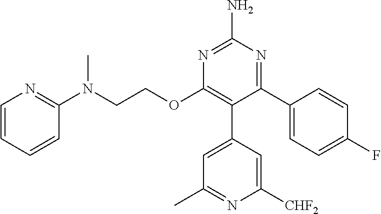 [SMILES: Cc1cc(-c2c(OCCN(C)c3ccccn3)nc(N)nc2-c2ccc(F)cc2)cc(C(F)F)n1] | 5-(2-(difluoromethyl)-6- methylpyridin-4-yl)-4-(4- fluorophenyl)-6-(2- (methyl(pyridin-2- yl)amino)ethoxy) pyrimidin-2-amine | 481.0 | 0.51 | 1.1/0.24/ 0.10 | 1.0 | 11.2 |
| 80 | 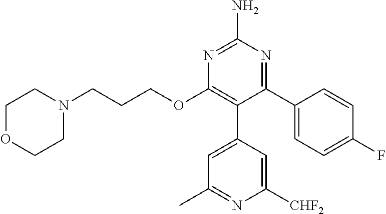 [SMILES: Cc1cc(-c2c(OCCCN3CCOCC3)nc(N)nc2-c2ccc(F)cc2)cc(C(F)F)n1] | 5-(2-(difluoromethyl)-6- methylpyridin-4-yl)-4-(4- fluorophenyl)-6-(3- morpholinopropoxy) pyrimidin-2-amine | 474.1 | 42 | 6.4 | | |
| 81 | 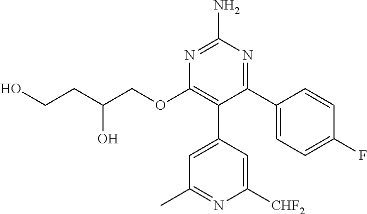 [SMILES: Cc1cc(-c2c(OCC(O)CCO)nc(N)nc2-c2ccc(F)cc2)cc(C(F)F)n1] | 4-((2-amino-5-(2- (difluoromethyl)-6- methylpyridin-4-yl)-6-(4- fluorophenyl)pyrimidin-4- yl)oxy)butane-1,3-diol | 435.1 | 5.6 | 21/5.9/ 8.4 | | |
| 82 | 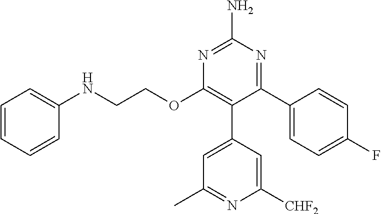 [SMILES: Cc1cc(-c2c(OCCNc3ccccc3)nc(N)nc2-c2ccc(F)cc2)cc(C(F)F)n1] | 5-(2-(difluoromethyl)-6- methylpyridin-4-yl)-4-(4- fluorophenyl)-6-(2- (phenylamino)ethoxy) pyrimidin-2-amine | 466.0 | 16 | 1.6 | 936 | |
| 83 | 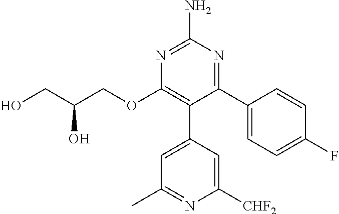 [SMILES: Cc1cc(-c2c(OC[C@@H](O)CO)nc(N)nc2-c2ccc(F)cc2)cc(C(F)F)n1] | (S)-3-((2-amino-5-(2- (difluoromethyl)-6- methylpyridin-4-yl)-6-(4- fluorophenyl)pyrimidin-4- yl)oxy)propane-1,2-diol | 421.0 | 13 | 2.9/7.3/ 22 | 1611 | |
| 84 | 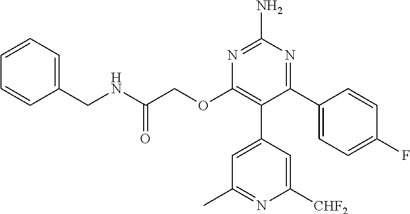 [SMILES: Cc1cc(-c2c(OCC(=O)NCc3ccccc3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N- benzylacetamide | 494.0 | 17 | 8.7 | | |
| 85 | 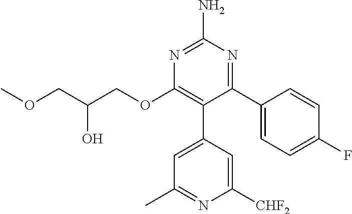 [SMILES: COCC(O)COc1nc(N)nc(-c2ccc(F)cc2)c1-c1cc(C)nc(C(F)F)c1] | 1-((2-amino-5-(2- (difluoromethyl)-6- methylpyridin-4-yl)-6-(4- fluorophenyl)pyrimidin-4- yl)oxy)-3-methoxypropan- 2-ol | 435.0 | 4.2 | 0.26/2.4/ 4.4 | | 469.3 |
| 86 | 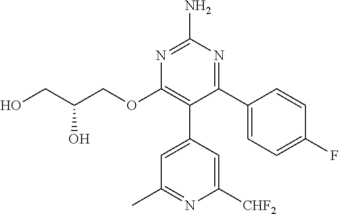 [SMILES: Cc1cc(-c2c(OC[C@H](O)CO)nc(N)nc2-c2ccc(F)cc2)cc(C(F)F)n1] | (R)-3-((2-amino-5-(2- methylpyridin-4-yl)-6-(4- fluorophenyl)pyrimidin-4- yl)oxy)propane-1,2-diol | 421.0 | 13 | 2/9.1/ 10.5 | 1069 | |
| 87 | 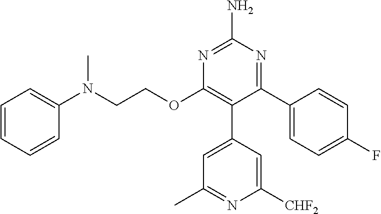 [SMILES: Cc1cc(-c2c(OCCN(C)c3ccccc3)nc(N)nc2-c2ccc(F)cc2)cc(C(F)F)n1] | 5-(2-(difluoromethyl)-6- methylpyridin-4-yl)-4-(4- fluorophenyl)-6-(2- (methyl(phenyl)amino) ethoxy)pyrimidin-2-amine | 480.0 | 18 | 2.2 | | |
| 88 | 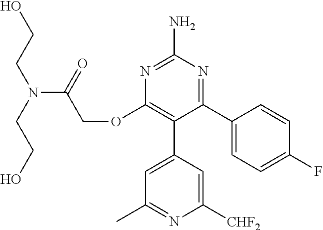 [SMILES: Cc1cc(-c2c(OCC(=O)N(CCO)CCO)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N,N-bis(2- hydroxyethyl)acetamide | 492.1 | 22 | 8.3 | 4261 | |
| 89 | 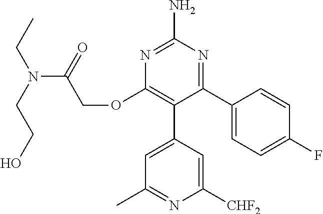 [SMILES: CCN(CCO)C(=O)COc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)-N-ethyl-N-(2- hydroxyethyl)acetamide | 476.0 | 6.1 | 2.2 | | |
| 90 | 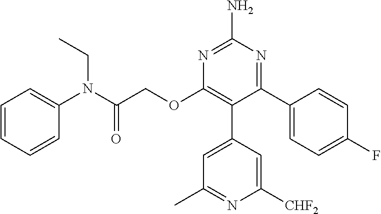 [SMILES: CCN(C(=O)COc1nc(N)nc(-c2ccc(F)cc2)c1-c1cc(C)nc(C(F)F)c1)c1ccccc1] | 2-((2-amino-5-(2- (difluoromethyl)-6- methylpyridin-4-yl)-6-(4- fluorophenyl)pyrimidin-4- yl)oxy)-N-ethyl-N- phenylacetamide | 508.1 | 23 | 6.8 | | |
| 91 | 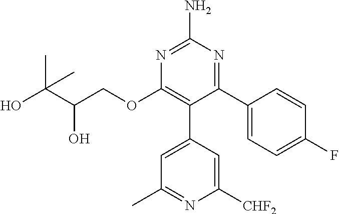 [SMILES: Cc1cc(-c2c(OCC(O)C(C)(C)O)nc(N)nc2-c2ccc(F)cc2)cc(C(F)F)n1] | 1-((2-amino-5-(2- (difluoromethyl)-6- methylpyridin-4-yl)-6-(4- fluorophenyl)pyrimidin-4- yl)oxy)-3-methylbutane- 2,3-diol | 449.1 | >1000 | | | |
| 92 | 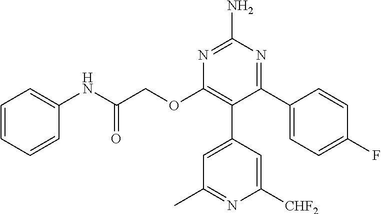 [SMILES: Cc1cc(-c2c(OCC(=O)Nc3ccccc3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N- phenylacetamide | 480.1 | 48 | 6.4 | | |
| 93 | 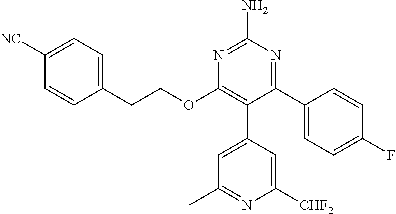 [SMILES: Cc1cc(-c2c(OCCc3ccc(C#N)cc3)nc(N)nc2-c2ccc(F)cc2)cc(C(F)F)n1] | 4-(2-((2-amino-5-(2- (difluoromethyl)-6- methylpyridin-4-yl)-6-(4- fluorophenyl)pyrimidin-4- yl)oxy)ethyl)benzonitrile | 476.1 | 133 | | | |
| 94 | 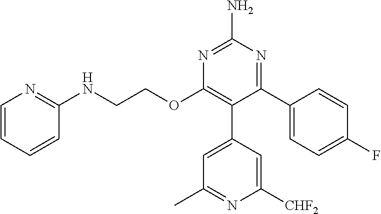 [SMILES: Cc1cc(-c2c(OCCNc3ccccn3)nc(N)nc2-c2ccc(F)cc2)cc(C(F)F)n1] | 5-(2-(difluoromethyl)-6- methylpyridin-4-yl)-4-(4- fluorophenyl)-6-(2- (pyridin-2- ylamino)ethoxy)pyrimidin- 2-amine | 467.1 | 2.4 | 0.39/2.9 | | |
| 95 | 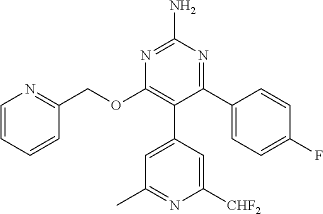 [SMILES: Cc1cc(-c2c(OCc3ccccn3)nc(N)nc2-c2ccc(F)cc2)cc(C(F)F)n1] | 5-(2-(difluoromethyl)-6- methylpyridin-4-yl)-4-(4- fluorophenyl)-6-(pyridin-2- ylmethoxy)pyrimidin-2- amine | 438.1 | 0.24 | 0.061/ 0.94 | 12.0 | 14.0 |
| 96 | 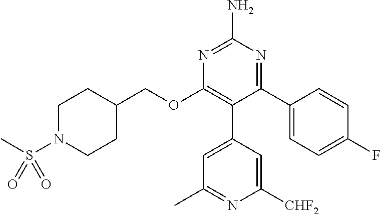 [SMILES: Cc1cc(-c2c(OCC3CCN(S(C)(=O)=O)CC3)nc(N)nc2-c2ccc(F)cc2)cc(C(F)F)n1] | 5-(2-(difluoromethyl)-6- methylpyridin-4-yl)-4-(4- fluorophenyl)-6-((1- (methylsulfonyl)piperidin- 4-yl)methoxy)pyrimidin-2- amine | 522.1 | 61 | | | |
| 97 | 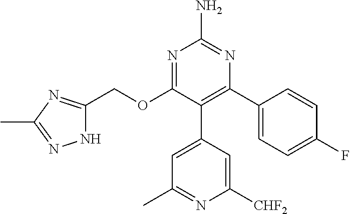 [SMILES: Cc1cc(-c2c(OCc3nc(C)n[nH]3)nc(N)nc2-c2ccc(F)cc2)cc(C(F)F)n1] | 5-(2-(difluoromethyl)-6- methylpyridin-4-yl)-4-(4- fluorophenyl)-6-((3- methyl-1H-1,2,4-triazol-5- yl)methoxy)pyrimidin-2- amine | 442.1 | 14 | 4.5 | 3308 | |
| 98 | 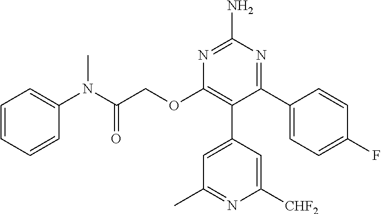 [SMILES: Cc1cc(-c2c(OCC(=O)N(C)c3ccccc3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-methyl-N- phenylacetamide | 494.1 | 5.9 | 1.2 | | |
| 99 | 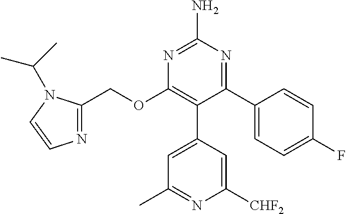 [SMILES: Cc1cc(-c2c(OCc3nccn3C(C)C)nc(N)nc2-c2ccc(F)cc2)cc(C(F)F)n1] | 5-(2-(difluoromethyl)-6- methylpyridin-4-yl)-4-(4- fluorophenyl)-6-((1- isopropyl-1H-imidazol-2- yl)methoxy)pyrimidin-2- amine | 469.1 | 0.06/ 1.8 | 0.064/ 0.19 | 32/22 | 47 |
| 100 | 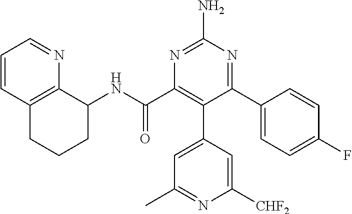 [SMILES: Cc1cc(-c2c(C(=O)NC3CCCc4cccnc43)nc(N)nc2-c2ccc(F)cc2)cc(C(F)F)n1] | 2-amino-5-(2- (difluoromethyl)-6- methylpyridin-4-yl)-6-(4- fluorophenyl)-N-(5,6,7,8- tetrahydroquinolin-8- yl)pyrimidine-4- carboxamide | 505.1 | 66 | | | |
| 101 | 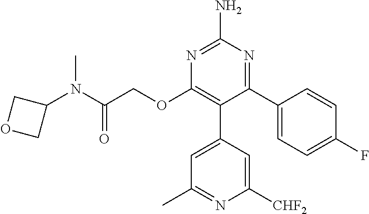 [SMILES: Cc1cc(-c2c(OCC(=O)N(C)C3COC3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-methyl-N- (oxetan-3-yl)acetamide | 474.0 | 70 | | | |
| 102 | 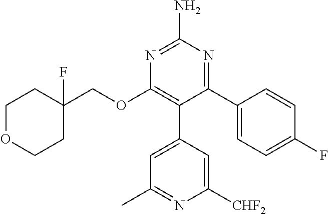 [SMILES: Cc1cc(-c2c(OCC3(F)CCOCC3)nc(N)nc2-c2ccc(F)cc2)cc(C(F)F)n1] | 5-(2-(difluoromethyl)-6- methylpyridin-4-yl)-4-(4- fluorophenyl)-6-((4- fluorotetrahydro-2H- pyran-4- yl)methoxy)pyrimidin-2- amine | 463.0 | 0.83 | 0.052/ 0.98 | 10.0 | |
| 103 | 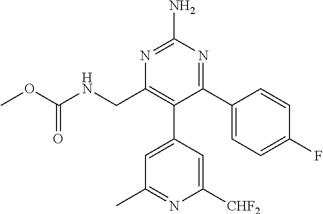 [SMILES: COC(=O)NCc1nc(N)nc(-c2ccc(F)cc2)c1-c1cc(C)nc(C(F)F)c1] | methyl ((2-amino-5-(2- (difluoromethyl)-6- methylpyridin-4-yl)-6-(4- fluorophenyl)pyrimidin-4- yl)methyl)carbamate | 418.0 | 47 | 11 | | |
| 104 | 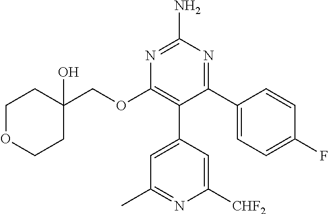 [SMILES: Cc1cc(-c2c(OCC3(O)CCOCC3)nc(N)nc2-c2ccc(F)cc2)cc(C(F)F)n1] | 4-(((2-amino-5-(2- (difluoromethyl)-6- methylpyridin-4-yl)-6-(4- fluorophenyl)pyrimidin-4- yl)oxy)methyl)tetrahydro- 2H-pyran-4-ol | 461.2 | >1000 | | | |
| 105 | 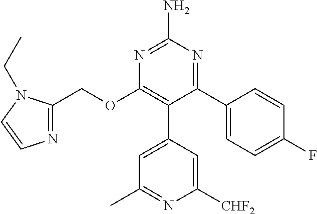 [SMILES: CCn1ccnc1COc1nc(N)nc(-c2ccc(F)cc2)c1-c1cc(C)nc(C(F)F)c1] | 5-(2-(difluoromethyl)-6- methylpyridin-4-yl)-4-((1- ethyl-1H-imidazol-2- yl)methoxy)-6-(4- fluorophenyl)pyrimidin-2- amine | 455.1 | 0.17/ 1.0 | 0.44/ 0.38 | 10 | 2.3/ 3.7/ 7.4 |
| 106 | 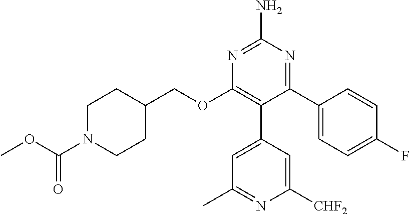 [SMILES: COC(=O)N1CCC(COc2nc(N)nc(-c3ccc(F)cc3)c2-c2cc(C)nc(C(F)F)c2)CC1] | methyl 4-((2-amino-5-(2- (difluoromethyl)-6- methylpyridin-4-yl)-6-(4- fluorophenyl)pyrimidin-4- yl)oxy)methyl)piperidine- 1-carboxylate | 502.0 | 38 | 43 | | |
| 107 | 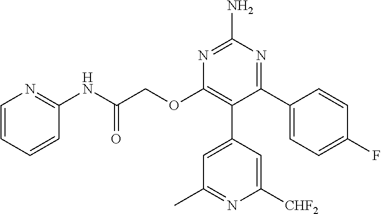 [SMILES: Cc1cc(-c2c(OCC(=O)Nc3ccccn3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(pyridin-2- yl)acetamide | 481.1 | 44 | | | |
| 108 | 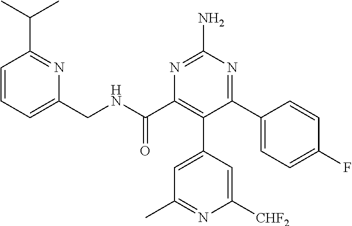 [SMILES: Cc1cc(-c2c(C(=O)NCc3cccc(C(C)C)n3)nc(N)nc2-c2ccc(F)cc2)cc(C(F)F)n1] | 2-amino-5-(2- (difluoromethyl)-6- methylpyridin-4-yl)-6-(4- fluorophenyl)-N-((6- isopropylpyridin-2- yl)methyl)pyrimidine-4- carboxamide | 507.1 | 222 | | | |
| 109 | 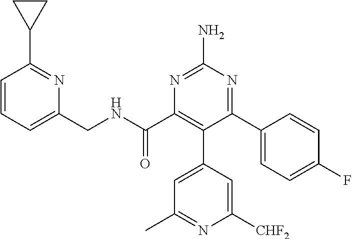 [SMILES: Cc1cc(-c2c(C(=O)NCc3cccc(C4CC4)n3)nc(N)nc2-c2ccc(F)cc2)cc(C(F)F)n1] | 2-amino-N-((6- cyclopropylpyridin-2- yl)methyl)-5-(2- (difluoromethyl)-6- methylpyridin-4-yl)-6-(4- fluorophenyl)pyrimidine-4- carboxamide | 505.1 | 205 | | | |
| 110 | 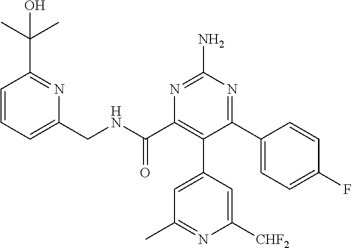 [SMILES: Cc1cc(-c2c(C(=O)NCc3cccc(C(C)(C)O)n3)nc(N)nc2-c2ccc(F)cc2)cc(C(F)F)n1] | 2-amino-5-(2- (difluoromethyl)-6- methylpyridin-4-yl)-6-(4- fluorophenyl)-N-((6-(2- hydroxypropan-2- yl)pyridin-2- yl)methyl)pyrimidine-4- carboxamide | 523.1 | 250 | | | |
| 111 | 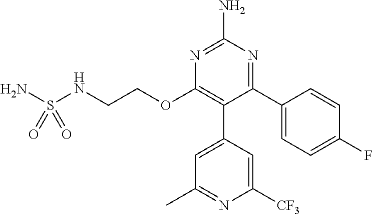 [SMILES: Cc1cc(-c2c(OCCNS(N)(=O)=O)nc(N)nc2-c2ccc(F)cc2)cc(C(F)(F)F)n1] | (2-amino-5-(2- (trifluoromethyl)-6- methylpyridin-4-yl)-6-(4- fluorophenyl)pyrimidin-4- yl)oxy)ethyl)sulfuric diamide | 487.0 | 4.1 | 2.2/1.1 | | 1775 |
| 112 | 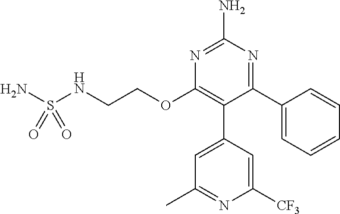 [SMILES: Cc1cc(-c2c(OCCNS(N)(=O)=O)nc(N)nc2-c2ccccc2)cc(C(F)(F)F)n1] | (2-amino-5-(2- (trmuoromethyl)-6- methylpyridin-4-yl)-6- phenyl)pyrimidin-4- yl)oxy)ethyl)sulfuric diamide | 469.0 | 6.7 | 3.1 | | |
| 113 | 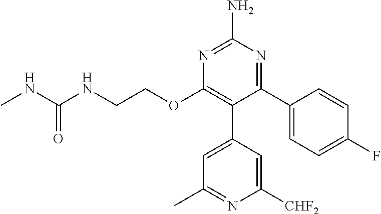 [SMILES: CNC(=O)NCCOc1nc(N)nc(-c2ccc(F)cc2)c1-c1cc(C)nc(C(F)F)c1] | 1-(2-((2-amino-5-(2- (difluoromethyl)-6- methylpyridin-4-yl)-6-(4- fluorophenyl)pyrimidin-4- yl)oxy)ethyl)-3-methylurea | 447.0 | 8.9 | 16/7.5 | | |
| 114 | 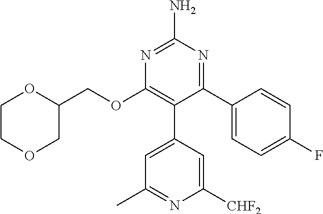 [SMILES: Cc1cc(-c2c(OCC3COCCO3)nc(N)nc2-c2ccc(F)cc2)cc(C(F)F)n1] | 4-((1,4-dioxan-2- yl)methoxy)-5-(2- (difluoromethyl)-6- methylpyridin-4-yl)-6-(4- fluorophenyl)pyrimidin-2- amine | 447.1 | 7.7 | 6.2/4.2 | | |
| 115 | 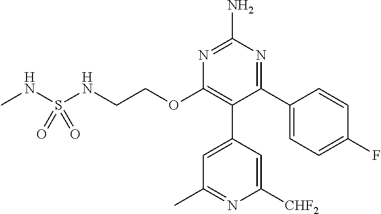 [SMILES: CNS(=O)(=O)NCCOc1nc(N)nc(-c2ccc(F)cc2)c1-c1cc(C)nc(C(F)F)c1] | 1-(2-amino-5-(2- (difluoromethyl)-6- methylpyridin-4-yl)-6-(4- fluorophenyl)pyrimidin-4- yl)oxy)ethyl)-3-methyl- sulfuric diamide | 482.9 | 19 | 9.9 | 820 | |
| 116 | 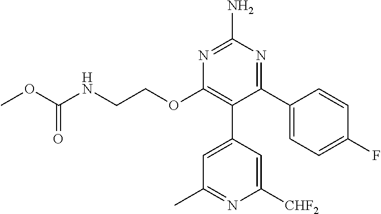 [SMILES: COC(=O)NCCOc1nc(N)nc(-c2ccc(F)cc2)c1-c1cc(C)nc(C(F)F)c1] | methyl (2-((2-amino-5-(2- (difluoromethyl)-6- methylpyridin-4-yl)-6-(4- fluorophenyl)pyrimidin-4- yl)oxy)ethyl)carbamate | 448.0 | 7.3 | 4.7/3.2 | | |
| 117 | 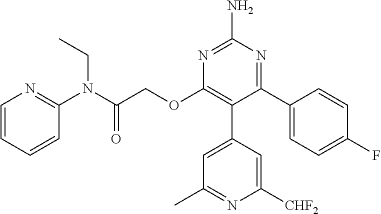 [SMILES: CCN(C(=O)COc1nc(N)nc(-c2ccc(F)cc2)c1-c1cc(C)nc(C(F)F)c1)c1ccccn1] | 2-((2-amino-5-(2- (difluoromethyl)-6- methylpyridin-4-yl)-6-(4- fluorophenyl)pyrimidin-4- yl)oxy)-N-ethyl-N-(pyridin- 2-yl)acetamide | 509.0 | 39.4 | 27 | | |
| 118 | 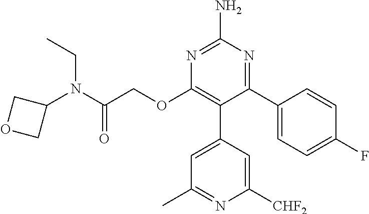 [SMILES: CCN(C(=O)COc1nc(N)nc(-c2ccc(F)cc2)c1-c1cc(C)nc(C(F)F)c1)C1COC1] | 2-((2-amino-5-(2- (difluoromethyl)-6- methylpyridin-4-yl)-6-(4- fluorophenyl)pyrimidin-4- yl)oxy)-N-ethyl-N-(oxetan- 3-yl)acetamide | 488.0 | 28.7 | 18 | | |
| 119 | 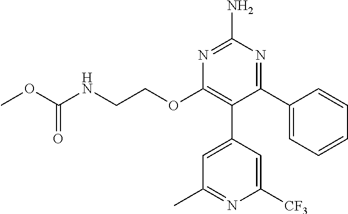 [SMILES: COC(=O)NCCOc1nc(N)nc(-c2ccccc2)c1-c1cc(C)nc(C(F)(F)F)c1] | methyl (2-((2-amino-5-(2- methyl-6- (trifluoromethyl)pyridin-4- yl)-6-phenylpyrimidin-4- yl)oxy)ethyl)carbamate | 448.0 | 16.8 | 5.8/5.4 | | |
| 120 | 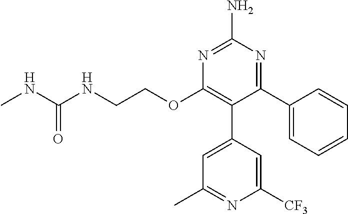 [SMILES: CNC(=O)NCCOc1nc(N)nc(-c2ccccc2)c1-c1cc(C)nc(C(F)(F)F)c1] | 1-(2-((2-amino-5-(2- methyl-6- (trifluoromethyl)pyridin-4- yl)-6-phenylpyrimidin-4- yl)oxy)ethyl)-3-methylurea | 447.0 | 8.8 | 13/8.2 | | |
| 121 | 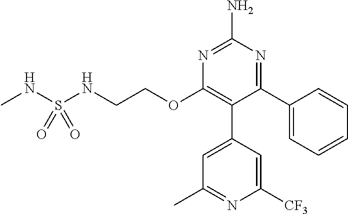 [SMILES: CNS(=O)(=O)NCCOc1nc(N)nc(-c2ccccc2)c1-c1cc(C)nc(C(F)(F)F)c1] | 1-(2-amino-5-(2- (trifluoromethyl)-6- methylpyridin-4-yl)-6- phenylpyrimidin-4- yl)oxy)ethyl)-3-methyl- sulfuric diamide | 482.9 | 3.9 | 1.5/1.4 | 55/139 | 478 |
| 122 | 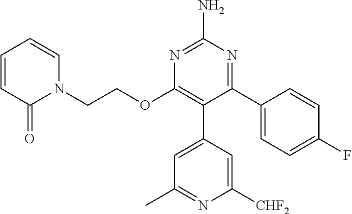 [SMILES: Cc1cc(-c2c(OCCn3ccccc3=O)nc(N)nc2-c2ccc(F)cc2)cc(C(F)F)n1] | 1-(2-((2-amino-5-(2- (difiuoromethyl)-6- methylpyridin-4-yl)-6-(4- fluorophenyl)pyrimidin-4- yl)oxy)ethyl)pyridin-2(1H)- one | 468.0 | 1.8 | 0.88/ 0.69 | 35/53 | 63 |
| 123 | 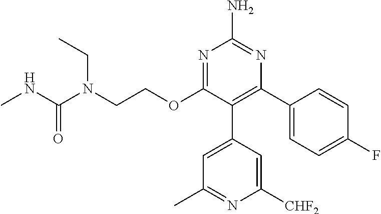 [SMILES: CCN(CCOc1nc(N)nc(-c2ccc(F)cc2)c1-c1cc(C)nc(C(F)F)c1)C(=O)NC] | 1-(2-((2-amino-5-(2- (difluoromethyl)-6- methylpyridin-4-yl)-6-(4- fluorophenyl)pyrimidin-4- yl)oxy)ethyl)-1,3- diethylurea | 489.1 | 1.2 | 0.87/ 0.67 | | |
| 124 | 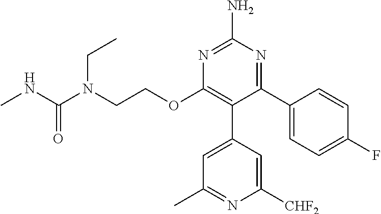 [SMILES: CCN(CCOc1nc(N)nc(-c2ccc(F)cc2)c1-c1cc(C)nc(C(F)F)c1)C(=O)NC] | 1-(2-((2-amino-5-(2- (difluoromethyl)-6- methylpyridin-4-yl)-6-(4- fluorophenyl)pyrimidin-4- yl)oxy)ethyl)-1-ethyl-3- methylurea | 475.1 | 1.7 | 1.1/0.5 | 535 | |
| 125 | 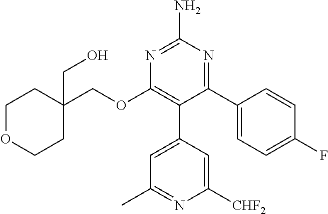 [SMILES: Cc1cc(-c2c(OCC3(CO)CCOCC3)nc(N)nc2-c2ccc(F)cc2)cc(C(F)F)n1] | (4-(((2-amino-5-(2- (difluoromethyl)-6- methylpyridin-4-yl)-6-(4- fluorophenyl)pyrimidin- 4- yl)oxy)methyl)tetrahydro- 2H-pyran-4- yl)methanol | 475.0 | 18.1 | 8.7 | 1886 | |
| 126 | 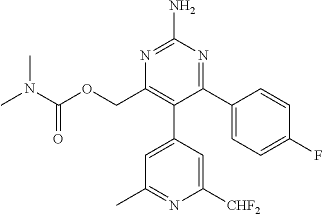 [SMILES: Cc1cc(-c2c(COC(=O)N(C)C)nc(N)nc2-c2ccc(F)cc2)cc(C(F)F)n1] | (2-amino-5-(2- (difluoromethyl)-6- methylpyridin-4-yl)-6-(4- fluorophenyl)pyrimidin-4- yl)methyl dimethylcarbamate | | | | | |
| 127 | 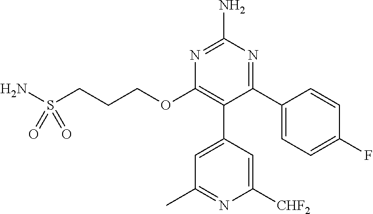 [SMILES: Cc1cc(-c2c(OCCCS(N)(=O)=O)nc(N)nc2-c2ccc(F)cc2)cc(C(F)F)n1] | 3-((2-amino-5-(2- (difluoromethyl)-6- methylpyridin-4-yl)-6-(4- fluorophenyl)pyrimidin-4- yl)oxy)propane-1- sulfonamide | | | | | |
| 128 | 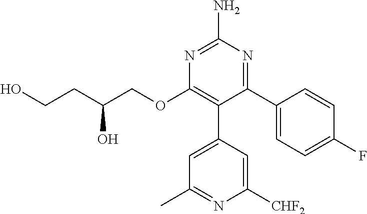 [SMILES: Cc1cc(-c2c(OC[C@@H](O)CCO)nc(N)nc2-c2ccc(F)cc2)cc(C(F)F)n1] | (S)-4-((2-amino-5-(2- (difluoromethyl)-6- methylpyridin-4-yl)-6-(4- fluorophenyl)pyrimidin-4- yl)oxy)butane-1,3-diol | | | | | |
| 129 | 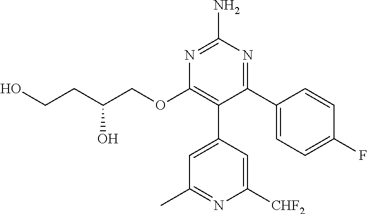 [SMILES: Cc1cc(-c2c(OC[C@H](O)CCO)nc(N)nc2-c2ccc(F)cc2)cc(C(F)F)n1] | (R)-4-((2-amino-5-(2- (difluoromethyl)-6- methylpyridin-4-yl)-6-(4- fluorophenyl)pyrimidin-4- yl)oxy)butane-1,3-diol | | | | | |
| 130 | 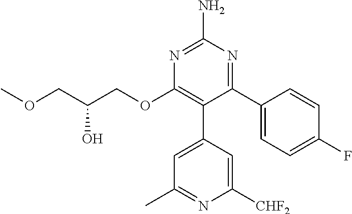 [SMILES: COC[C@@H](O)COc1nc(N)nc(-c2ccc(F)cc2)c1-c1cc(C)nc(C(F)F)c1] | (R)-1-((2-amino-5-(2- (difluoromethyl)-6- methylpyridin-4-yl)-6-(4- fluorophenyl)pyrimidin-4- yl)oxy)-3-methoxypropan- 2-ol | | | | | |
| 131 | 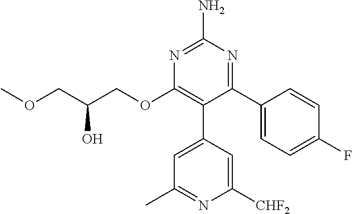 [SMILES: COC[C@H](O)COc1nc(N)nc(-c2ccc(F)cc2)c1-c1cc(C)nc(C(F)F)c1] | (S)-1-((2-amino-5-(2- (difluoromethyl)-6- methylpyridin-4-yl)-6-(4- fluorophenyl)pyrimidin-4- yl)oxy)-3-methoxypropan- 2-ol | | | | | |
| 132 | 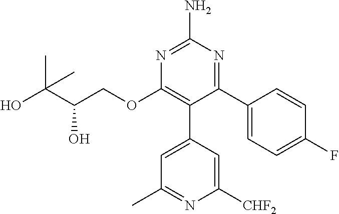 [SMILES: Cc1cc(-c2c(OC[C@H](O)C(C)(C)O)nc(N)nc2-c2ccc(F)cc2)cc(C(F)F)n1] | (S)-1-((2-amino-5-(2- (difluoromethyl)-6- methylpyridin-4-yl)-6-(4- fluorophenyl)pyrimidin-4- yl)oxy)-3-methylbutane- 2,3-diol | | | | | |
| 133 | 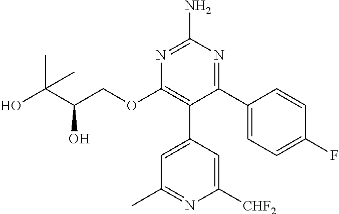 [SMILES: Cc1cc(-c2c(OC[C@@H](O)C(C)(C)O)nc(N)nc2-c2ccc(F)cc2)cc(C(F)F)n1] | (R)-1-((2-amino-5-(2- (difluoromethyl)-6- methylpyridin-4-yl)-6-(4- fluorophenyl)pyrimidin-4- yl)oxy)-3-methylbutane- 2,3-diol | | | | | |
| 134 | 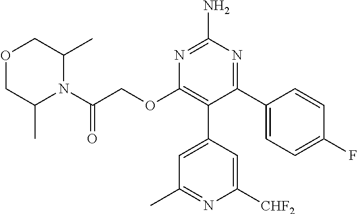 [SMILES: Cc1cc(-c2c(OCC(=O)N3C(C)COCC3C)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-1-(3,5- dimethylmorpholino)ethan- 1-one | | | | | |
| 135 | 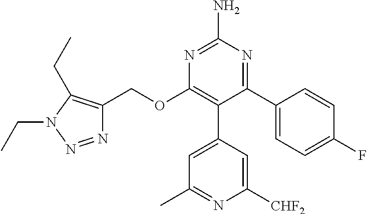 [SMILES: CCc1c(COc2nc(N)nc(-c3ccc(F)cc3)c2-c2cc(C)nc(C(F)F)c2)nnn1CC] | 4-((1,5-diethyl-1H-1,2,3- triazol-4-yl)methoxy)-5-(2- (difluoromethyl)-6- methylpyridin-4-yl)-6-(4- fluorophenyl)pyrimidin-2- amine | 484.2 | 1.2/4.3 | 3.1/2.6 | 77.0 | 93.2 |
| 136 | 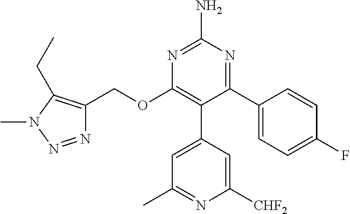 [SMILES: CCc1c(COc2nc(N)nc(-c3ccc(F)cc3)c2-c2cc(C)nc(C(F)F)c2)nnn1C] | 5-(2-(difluoromethyl)-6- methylpyridin-4-yl)-4-((5- ethyl-1-methyl-1H-1,2,3- triazol-4-yl)methoxy)-6-(4- fluorophenyl)pyrimidin-2- amine | 470.2 | 0.6/1.1 | 2.3 | 29/27 | 15.8 |
| 137 | 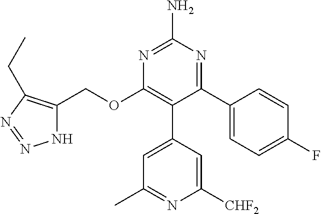 [SMILES: CCc1nn[nH]c1COc1nc(N)nc(-c2ccc(F)cc2)c1-c1cc(C)nc(C(F)F)c1] | 5-(2-(difluoromethyl)-6- methylpyridin-4-yl)-4-((4- ethyl-1H-1,2,3-triazol-5- yl)methoxy)-6-(4- fluorophenyl)pyrimidin-2- amine | 456.1 | 92 | 40 | | |
| 138 | 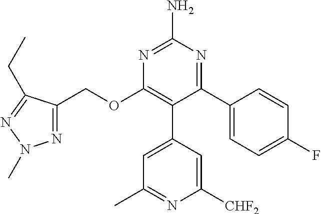 [SMILES: CCc1nn(C)nc1COc1nc(N)nc(-c2ccc(F)cc2)c1-c1cc(C)nc(C(F)F)c1] | 5-(2-(difluoromethyl)-6- methylpyridin-4-yl)-4-((5- ethyl-2-methyl-2H-1,2,3- triazol-4-yl)methoxy)-6-(4- fluorophenyl)pyrimidin-2- amine | | | | | |
| 139 | 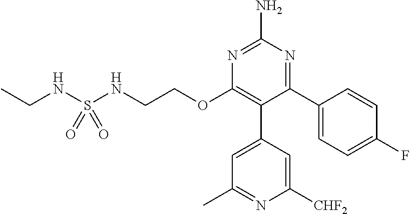 [SMILES: CCNS(=O)(=O)NCCOc1nc(N)nc(-c2ccc(F)cc2)c1-c1cc(C)nc(C(F)F)c1] | 1-(2-amino-5-(2- (difluoromethyl)-6- methylpyridin-4-yl)-6-(4- fluorophenyl)pyrimidin-4- yl)oxy)ethyl)-3-ethyl- sulfuric diamide | | | | | |
| 140 | 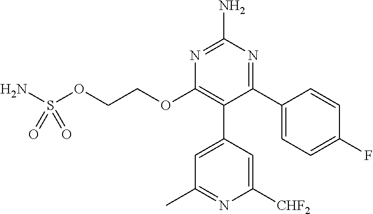 [SMILES: Cc1cc(-c2c(OCCOS(N)(=O)=O)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)ethyl sulfamate | | | | | |
| 141 | 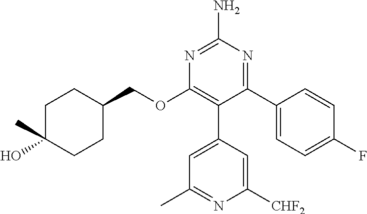 [SMILES: Cc1cc(-c2c(OC[C@H]3CC[C@](C)(O)CC3)nc(N)nc2-c2ccc(F)cc2)cc(C(F)F)n1] | (1r,4r)-4-(((2-amino-5-(2- (difluoromethyl)-6- methylpyridin-4-yl)-6-(4- fluorophenyl)pyrimidin-4- yl)oxy)methyl)-1- methylcyclohexan-1-ol | | | | | |
| 142 | 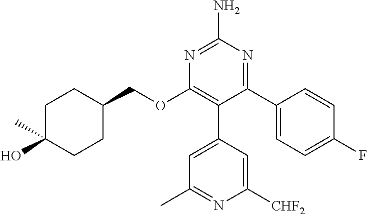 [SMILES: Cc1cc(-c2c(OC[C@H]3CC[C@@](C)(O)CC3)nc(N)nc2-c2ccc(F)cc2)cc(C(F)F)n1] | (1s,4s)-4-(((2-amino-5-(2- (difluoromethyl)-6- methylpyridin-4-yl)-6-(4- fluorophenyl)pyrimidin-4- yl)oxy)methyl)-1- methylcyclohexan-1-ol | | | | | |
| 143 | 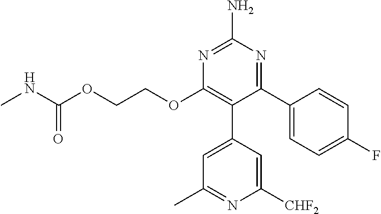 [SMILES: CNC(=O)OCCOc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)ethyl methylcarbamate | 448.1 | 11.7 | 8.1 | | |
| 144 | 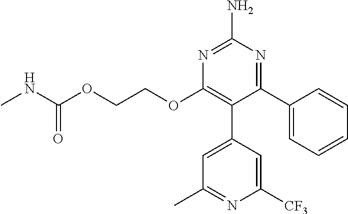 [SMILES: CNC(=O)OCCOc1nc(N)nc(-c2ccccc2)c1-c1cc(C)nc(C(F)(F)F)c1] | 2-((2-amino-5-(2-methyl- 6-(trifluoromethyl)pyridin- 4-yl)-6-phenylpyrimidin-4- yl)oxy)ethyl methylcarbamate | 447.9 | 4.5 | 4.2 | | |
| 145 | 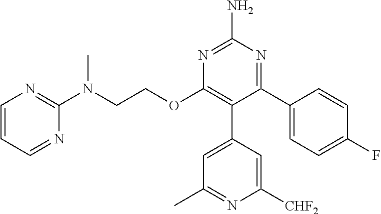 [SMILES: Cc1cc(-c2c(OCCN(C)c3ncccn3)nc(N)nc2-c2ccc(F)cc2)cc(C(F)F)n1] | 5-(2-(difluoromethyl)-6- methylpyridin-4-yl)-4-(4- fluorophenyl)-6-(2- (methyl(pyrimidin-2- yl)amino)ethoxy) pyrimidin-2-amine | 482.0 | 0.1/1.2 | 0.9/1.2 | 123 | |
| 146 | 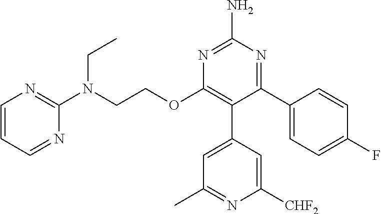 [SMILES: CCN(CCOc1nc(N)nc(-c2ccc(F)cc2)c1-c1cc(C)nc(C(F)F)c1)c1ncccn1] | 5-(2-(difluoromethyl)-6- methylpyridin-4-yl)-4-(2- (ethyl(pyrimidin-2- yl)amino)ethoxy)-6-(4- fluorophenyl)pyrimidin-2- amine | 496.0 | 4.0 | 5.6 | | |
| 147 | 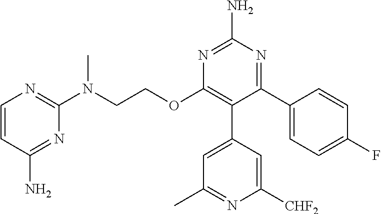 [SMILES: Cc1cc(-c2c(OCCN(C)c3nccc(N)n3)nc(N)nc2-c2ccc(F)cc2)cc(C(F)F)n1] | N²-(2-((2-amino-5-(2- (difluoromethyl)-6- methylpyridin-4-yl)-6-(4- fluorophenyl)pyrimidin-4- yl)oxy)ethyl)-N²- methylpyrimidine-2,4- diamine | 497.2 | 13.0 | | 5459 | |
| 148 | 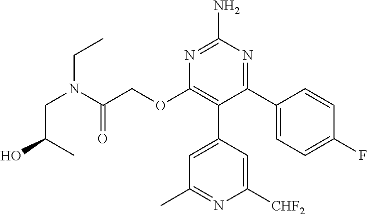 [SMILES: CCN(C[C@@H](C)O)C(=O)COc1nc(N)nc(-c2ccc(F)cc2)c1-c1cc(C)nc(C(F)F)c1] | (R)-2-((2-amino-5-(2- (difluoromethyl)-6- methylpyridin-4-yl)-6-(4- fluorophenyl)pyrimidin-4- yl)oxy)-N-ethyl-N-(2- hydroxypropyl)acetamide | | | | | |
| 149 | 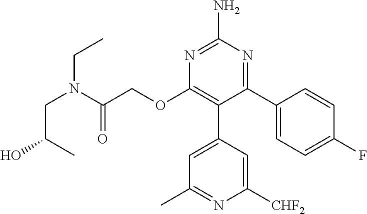 [SMILES: CCN(C[C@H](C)O)C(=O)COc1nc(N)nc(-c2ccc(F)cc2)c1-c1cc(C)nc(C(F)F)c1] | (S)-2-((2-amino-5-(2- (difluoromethyl)-6- methylpyridin-4-yl)-6-(4- fluorophenyl)pyrimidin-4- yl)oxy)-N-ethyl-N-(2- hydroxypropyl)acetamide | | | | | |
| 150 | 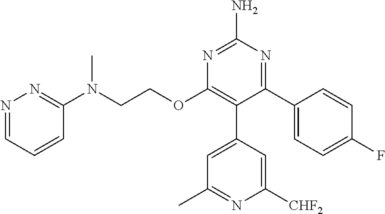 [SMILES: Cc1cc(-c2c(OCCN(C)c3cccnn3)nc(N)nc2-c2ccc(F)cc2)cc(C(F)F)n1] | N-(2-((2-amino-5-(2- (difluoromethyl)-6- methylpyridin-4-yl)-6-(4- fluorophenyl)pyrimidin-4- yl)oxy)ethyl)-N- methylpyridazin-3-amine | 482.0 | 2.7/3.2 | | 98 | |
| 151 | 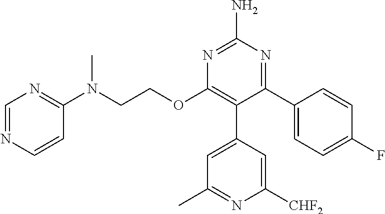 [SMILES: Cc1cc(-c2c(OCCN(C)c3ccncn3)nc(N)nc2-c2ccc(F)cc2)cc(C(F)F)n1] | 5-(2-(difluoromethyl)-6- methylpyridin-4-yl)-4-(4- fluorophenyl)-6-(2- (methyl(pyrimidin-4- yl)amino)ethoxy) pyrimidin-2-amine | 482.2 | | | >25000 | |
| 152 | 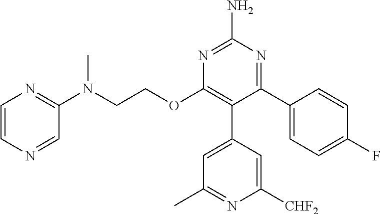 [SMILES: Cc1cc(-c2c(OCCN(C)c3cnccn3)nc(N)nc2-c2ccc(F)cc2)cc(C(F)F)n1] | 5-(2-(difluoromethyl)-6- methylpyridin-4-yl)-4-(4- fluorophenyl)-6-(2- (methyl(pyrazin-2- yl)amino)ethoxy) pyrimidin-2-amine | 482.2 | 5.1 | | 351 | |
| 153 | 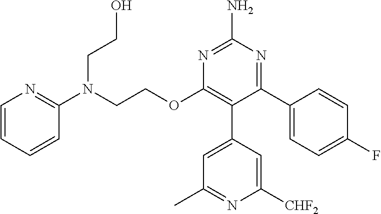 [SMILES: Cc1cc(-c2c(OCCN(CCO)c3ccccn3)nc(N)nc2-c2ccc(F)cc2)cc(C(F)F)n1] | 2-((2-((2-amino-5-(2- (difluoromethyl)-6- methylpyridin-4-yl)-6-(4- fluorophenyl)pyrimidin-4- yl)oxy)ethyl)(pyridin-2- yl)amino)ethan-1-ol | 511.0 | 8.9 | 4.1 | | |
| 154 | 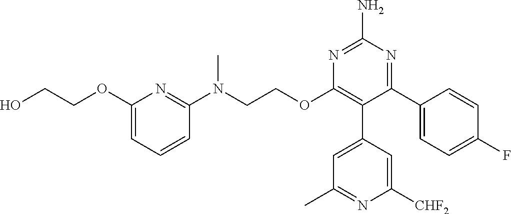 [SMILES: Cc1cc(-c2c(OCCN(C)c3cccc(OCCO)n3)nc(N)nc2-c2ccc(F)cc2)cc(C(F)F)n1] | 2-((6-((2-((2-amino-5-(2- (difluoromethyl)-6- methylpyridin-4-yl)-6-(4- fluorophenyl)pyrimidin-4- yl)oxy)ethyl)(methyl) amino)pyridin-2-yl)oxy) ethan-1-ol | 541.3 | 1.4/4.2 | 1.3 | 11.0 | 41.6 |
| 155 | 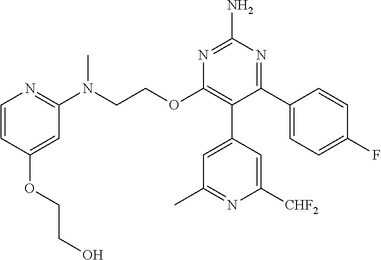 [SMILES: Cc1cc(-c2c(OCCN(C)c3cc(OCCO)ccn3)nc(N)nc2-c2ccc(F)cc2)cc(C(F)F)n1] | 2-((2-((2-((2-amino-5-(2- (difluoromethyl)-6- methylpyridin-4-yl)-6-(4- fluorophenyl)pyrimidin-4- yl)oxy)ethyl)(methyl) amino)pyridin-4-yl)oxy) ethan-1-ol | 541.2 | 24.0 | 5.4 | | |
| 156 | 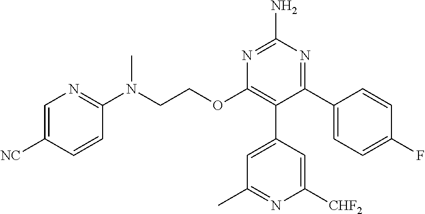 [SMILES: Cc1cc(-c2c(OCCN(C)c3ccc(C#N)cn3)nc(N)nc2-c2ccc(F)cc2)cc(C(F)F)n1] | 6-((2-((2-amino-5-(2- (difluoromethyl)-6- methylpyridin-4-yl)-6-(4- fluorophenyl)pyrimidin-4- yl)oxy)ethyl)(methyl) amino)nicotinonitrile | 506.0 | 84 | 57 | | |
| 157 | 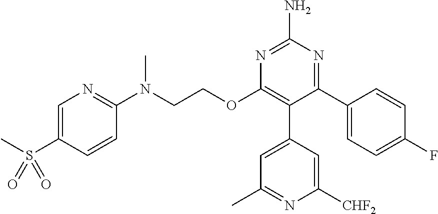 [SMILES: Cc1cc(-c2c(OCCN(C)c3ccc(S(C)(=O)=O)cn3)nc(N)nc2-c2ccc(F)cc2)cc(C(F)F)n1] | 5-(2-(difluoromethyl)-6- methylpyridin-4-yl)-4-(4- fluorophenyl)-6-(2- (methyl(5- (methylsulfonyl)pyridin-2- yl)amino)ethoxy) pyrimidin-2-amine | 558.9 | 79 | 52 | | |
| 158 | 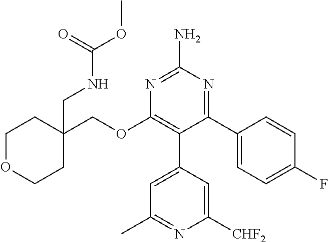 [SMILES: COC(=O)NCC1(COc2nc(N)nc(-c3ccc(F)cc3)c2-c2cc(C)nc(C(F)F)c2)CCOCC1] | methyl ((4-(((2-amino-5- (2-(difluoromethyl)-6- methylpyridin-4-yl)-6-(4- fluorophenyl)pyrimidin-4- yl)oxy)methyl)tetrahydro- 2H-pyran-4- yl)methyl)carbamate | 532.2 | 627 | 231 | | |
| 159 | 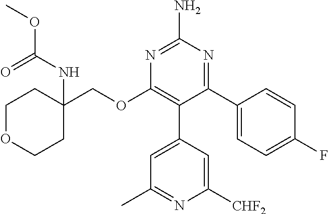 [SMILES: COC(=O)NC1(COc2nc(N)nc(-c3ccc(F)cc3)c2-c2cc(C)nc(C(F)F)c2)CCOCC1] | methyl (4-(((2-amino-5-(2- (difluoromethyl)-6- methylpyridin-4-yl)-6-(4- fluorophenyl)pyrimidin-4- yl)oxy)methyl)tetrahydro- 2H-pyran-4-yl)carbamate | | | | | |
| 160 | 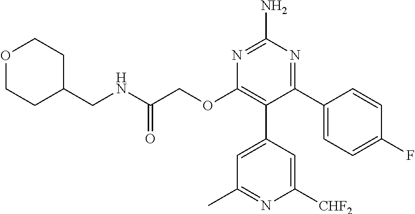 [SMILES: Cc1cc(-c2c(OCC(=O)NCC3CCOCC3)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-((tetrahydro-2H- pyran-4- yl)methyl)acetamide | 502.2 | 336 | 184 | | |
| 161 | 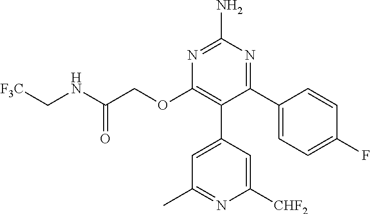 [SMILES: Cc1cc(-c2c(OCC(=O)NCC(F)(F)F)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(2,2,2- trifluoroethyl)acetamide | 486.2 | 44 | 14 | | |
| 162 | 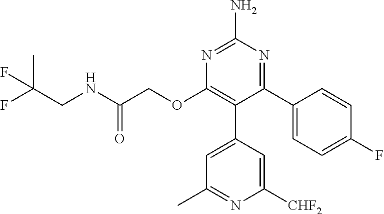 [SMILES: Cc1cc(-c2c(OCC(=O)NCC(C)(F)F)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)-N-(2,2- difluoropropyl)acetamide | 481.9 | 21.1 | 8.4 | | |
| 163 | 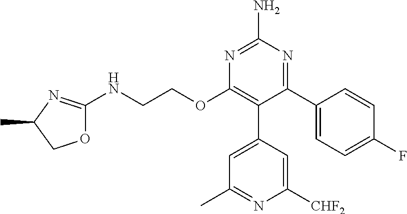 [SMILES: Cc1cc(-c2c(OCCNC3=N[C@H](C)CO3)nc(N)nc2-c2ccc(F)cc2)cc(C(F)F)n1] | (R)-N-(2-((2-amino-5-(2- (difluoromethyl)-6- methylpyridin-4-yl)-6-(4- fluorophenyl)pyrimidin-4- yl)oxy)ethyl)-4-methyl- 4,5-dihydrooxazol-2- amine | | | | | |
| 164 | 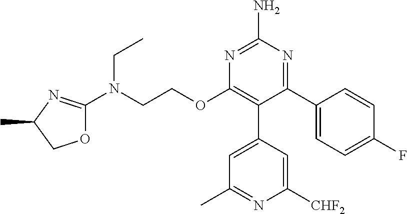 [SMILES: CCN(CCOc1nc(N)nc(-c2ccc(F)cc2)c1-c1cc(C)nc(C(F)F)c1)C1=N[C@H](C)CO1] | (R)-N-(2-((2-amino-5-(2- (difluoromethyl)-6- methylpyridin-4-yl)-6-(4- fluorophenyl)pyrimidin-4- yl)oxy)ethyl)-N-ethyl-4- methyl-4,5-dihydrooxazol- 2-amine | | | | | |
| 165 | 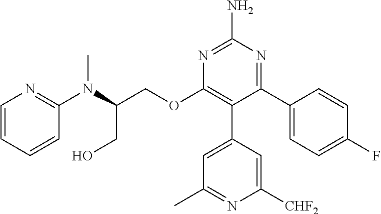 [SMILES: Cc1cc(-c2c(OC[C@@H](CO)N(C)c3ccccn3)nc(N)nc2-c2ccc(F)cc2)cc(C(F)F)n1] | (R)-3-((2-amino-5-(2- (difluoromethyl)-6- methylpyridin-4-yl)-6-(4- fluorophenyl)pyrimidin-4- yl)oxy)-2-(methyl(pyridin- 2-yl)amino)propan-1-ol | | | | | |
| 166 | 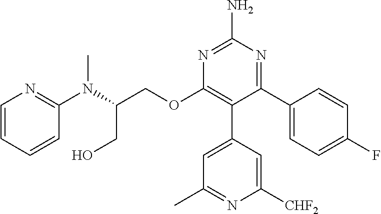 [SMILES: Cc1cc(-c2c(OC[C@H](CO)N(C)c3ccccn3)nc(N)nc2-c2ccc(F)cc2)cc(C(F)F)n1] | (S)-3-((2-amino-5-(2- (difluoromethyl)-6- methylpyridin-4-yl)-6-(4- fluorophenyl)pyrimidin-4- yl)oxy)-2-(methyl(pyridin- 2-yl)amino)propan-1-ol | | | | | |
| 167 | 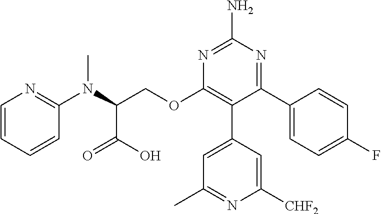 [SMILES: Cc1cc(-c2c(OC[C@@H](C(=O)O)N(C)c3ccccn3)nc(N)nc2-c2ccc(F)cc2)cc(C(F)F)n1] | O-(2-amino-5-(2- (difluoromethyl)-6- methylpyridin-4-yl)-6-(4- fluorophenyl)pyrimidin-4- yl)-N-methyl-N-(pyridin-2- yl)-L-serine | | | | | |
| 168 | 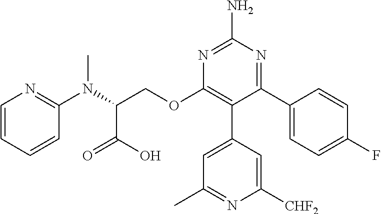 [SMILES: Cc1cc(-c2c(OC[C@H](C(=O)O)N(C)c3ccccn3)nc(N)nc2-c2ccc(F)cc2)cc(C(F)F)n1] | O-(2-amino-5-(2- (difluoromethyl)-6- methylpyridin-4-yl)-6-(4- fluorophenyl)pyrimidin-4- yl)-N-methyl-N-(pyridin-2- yl)-D-serine | | | | | |
| 169 | 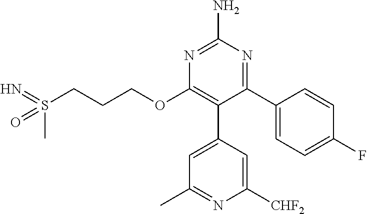 [SMILES: Cc1cc(-c2c(OCCCS(C)(=N)=O)nc(N)nc2-c2ccc(F)cc2)cc(C(F)F)n1] | (3-((2-amino-5-(2- (difluoromethyl)-6- methylpyridin-4-yl)-6-(4- fluorophenyl)pyrimidin-4- yl)oxy)propyl)(imino) (methyl)-λ⁶-sulfanone | 465.9 | 9.7 | 7.1 | | |
| 170 | 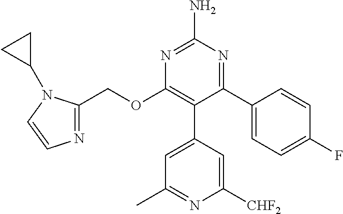 [SMILES: Cc1cc(-c2c(OCc3nccn3C3CC3)nc(N)nc2-c2ccc(F)cc2)cc(C(F)F)n1] | 4-((1-cyclopropyl-1H- imidazol-2-yl)methoxy)-5- (2-(difluoromethyl)-6- methylpyridin-4-yl)-6-(4- fluorophenyl)pyrimidin-2- amine | 467.2 | 1.1/1.1/ 2.9 | 1.1/1.6 | 33 | |
| 171 | 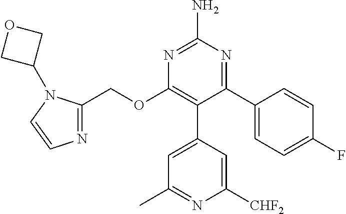 [SMILES: Cc1cc(-c2c(OCc3nccn3C3COC3)nc(N)nc2-c2ccc(F)cc2)cc(C(F)F)n1] | 5-(2-(difluoromethyl)-6- methylpyridin-4-yl)-4-(4- fluorophenyl)-6-((1- (oxetan-3-yl)-1H-imidazol- 2-yl)methoxy)pyrimidin-2- amine | 483.0 | 3.6 | 4.1 | | |
| 172 | 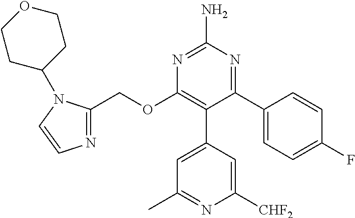 [SMILES: Cc1cc(-c2c(OCc3nccn3C3CCOCC3)nc(N)nc2-c2ccc(F)cc2)cc(C(F)F)n1] | 5-(2-(difluoromethyl)-6- methylpyridin-4-yl)-4-(4- fluorophenyl)-6-((1- (tetrahydro-2H-pyran-4- yl)-1H-imidazol-2- yl)methoxy)pyrimidin-2- amine | 511.0 | 165.0 | | | |
| 173 | 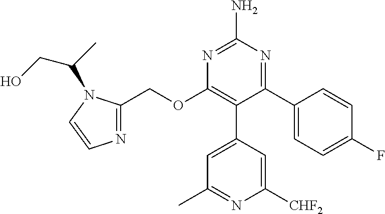 | (S)-2-(2-(((2-amino-5-(2- (difluoromethyl)-6- methylpyridin-4-yl)-6-(4- fluorophenyl)pyrimidin-4- yl)oxy)methyl)-1H- imidazol-1-yl)propan-1-ol | | | | | |
| 174 | 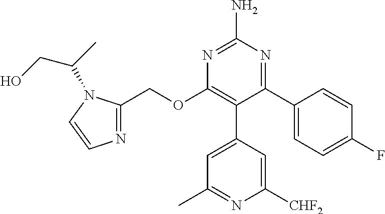 | (S)-2-(2-(((2-amino-5-(2- (difluoromethyl)-6- methylpyridin-4-yl)-6-(4- fluorophenyl)pyrimidin-4- yl)oxy)methyl)-1H- imidazol-1-yl)propan-1-ol | | | | | |
| 175 | 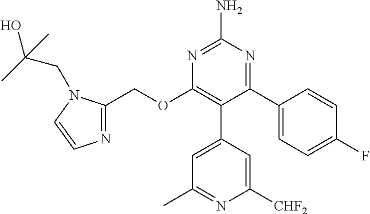 [SMILES: Cc1cc(-c2c(OCc3nccn3CC(C)(C)O)nc(N)nc2-c2ccc(F)cc2)cc(C(F)F)n1] | 1-(2-(((2-amino-5-(2- (difluoromethyl)-6- methylpyridin-4-yl)-6-(4- fluorophenyl)pyrimidin-4- yl)oxy)methyl)-1H- imidazol-1-yl)-2- methylpropan-2-ol | 499.0 | 15.0 | 19.0 | | |
| 176 | 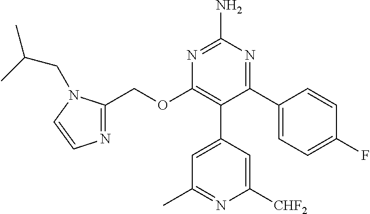 [SMILES: Cc1cc(-c2c(OCc3nccn3CC(C)C)nc(N)nc2-c2ccc(F)cc2)cc(C(F)F)n1] | 5-(2-(difluoromethyl)-6- methylpyridin-4-yl)-4-(4- fluorophenyl)-6-((1- isobutyl-1H-imidazol-2- yl)methoxy)pyrimidin-2- amine | 483.2 | 3.6 | 7.9 | | |
| 177 | 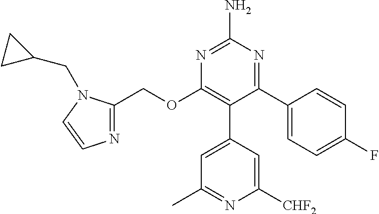 [SMILES: Cc1cc(-c2c(OCc3nccn3CC3CC3)nc(N)nc2-c2ccc(F)cc2)cc(C(F)F)n1] | 4-((1-(cyclopropylmethyl)- 1H-imidazol-2- yl)methoxy)-5-(2- (difluoromethyl)-6- methylpyridin-4-yl)-6-(4- fluorophenyl)pyrimidin-2- amine | 481.2 | 0.2 | 2.6/2.9 | 271 | |
| 178 | 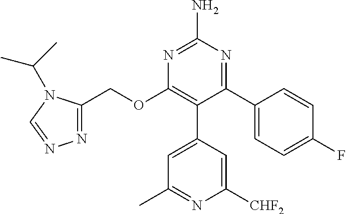 [SMILES: Cc1cc(-c2c(OCc3nncn3C(C)C)nc(N)nc2-c2ccc(F)cc2)cc(C(F)F)n1] | 5-(2-(difluoromethyl)-6- methylpyridin-4-yl)-4-(4- fluorophenyl)-6-((4- isopropyl-4H-1,2,4-triazol- 3-yl)methoxy)pyrimidin-2- amine | 470.2 | 1.4/3.2 | 1.5/1.5 | | |
| 179 | 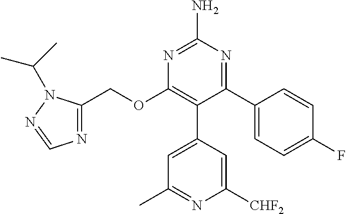 [SMILES: Cc1cc(-c2c(OCc3ncnn3C(C)C)nc(N)nc2-c2ccc(F)cc2)cc(C(F)F)n1] | 5-(2-(difluoromethyl)-6- methylpyridin-4-yl)-4-(4- fluorophenyl)-6-((1- isopropyl-1H-1,2,4-triazol- 5-yl)methoxy)pyrimidin-2- amine | 470.1 | 0.7/1.0 | 2.9 | 16/57 | |
| 180 | 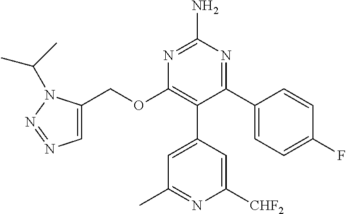 [SMILES: Cc1cc(-c2c(OCc3cnnn3C(C)C)nc(N)nc2-c2ccc(F)cc2)cc(C(F)F)n1] | 5-(2-(difluoromethyl)-6- methylpyridin-4-yl)-4-(4- fluorophenyl)-6-((1- isopropyl-1H-1,2,3-triazol- 5-yl)methoxy)pyrimidin-2- amine | 470.0 | 15.0 | 2.5 | | |
| 181 | 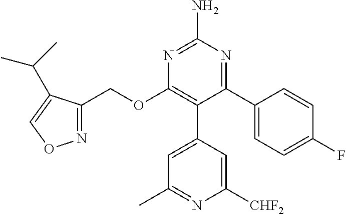 [SMILES: Cc1cc(-c2c(OCc3nocc3C(C)C)nc(N)nc2-c2ccc(F)cc2)cc(C(F)F)n1] | 5-(2-(difluoromethyl)-6- methylpyridin-4-yl)-4-(4- fluorophenyl)-6-((4- isopropylisoxazol-3- yl)methoxy)pyrimidin-2- amine | | | | | |
| 182 | 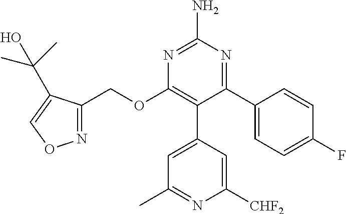 [SMILES: Cc1cc(-c2c(OCc3nocc3C(C)(C)O)nc(N)nc2-c2ccc(F)cc2)cc(C(F)F)n1] | 2-(3-(((2-amino-5-(2- (difluoromethyl)-6- methylpyridin-4-yl)-6-(4- fluorophenyl)pyrimidin-4- yl)oxy)methyl)isoxazol-4- yl)propan-2-ol | | | | | |
| 183 | 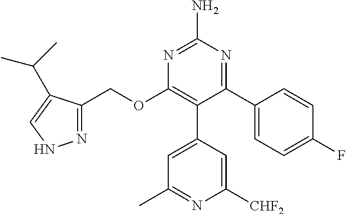 [SMILES: Cc1cc(-c2c(OCc3n[nH]cc3C(C)C)nc(N)nc2-c2ccc(F)cc2)cc(C(F)F)n1] | 5-(2-(difluoromethyl)-6- methylpyridin-4-yl)-4-(4- fluorophenyl)-6-((4- isopropyl-1H-pyrazol-3- yl)methoxy)pyrimidin-2- amine | 469.2 | 46 | 12.0 | | |
| 184 | 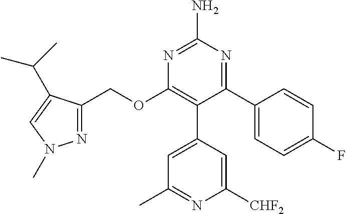 [SMILES: Cc1cc(-c2c(OCc3nn(C)cc3C(C)C)nc(N)nc2-c2ccc(F)cc2)cc(C(F)F)n1] | 5-(2-(difluoromethyl)-6- methylpyridin-4-yl)-4-(4- fluorophenyl)-6-((4- isopropyl-1-methyl-1H- pyrazol-3- yl)methoxy)pyrimidin-2- amine | 483.2 | 33 | 8.8 | | |
| 185 | 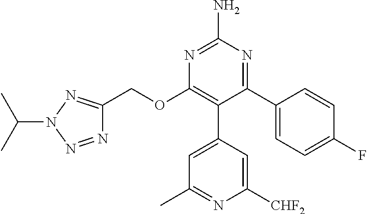 [SMILES: Cc1cc(-c2c(OCc3nnn(C(C)C)n3)nc(N)nc2-c2ccc(F)cc2)cc(C(F)F)n1] | 5-(2-(difluoromethyl)-6- methylpyridin-4-yl)-4-(4- fluorophenyl)-6-((2- isopropyl-2H-tetrazol-5- yl)methoxy)pyrimidin-2- amine | 471.2 | 7.1 | | 6.0/ 25.0 | |
| 186 | 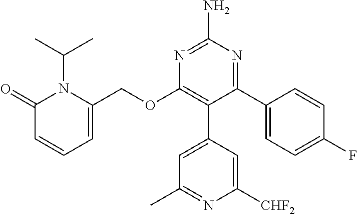 [SMILES: Cc1cc(-c2c(OCc3cccc(=O)n3C(C)C)nc(N)nc2-c2ccc(F)cc2)cc(C(F)F)n1] | 6-(((2-amino-5-(2- (difluoromethyl)-6- methylpyridin-4-yl)-6-(4- fluorophenyl)pyrimidin-4- yl)oxy)methyl)-1- isopropylpyridin-2(1H)- one | 496.0 | 2.6 | 4.1 | 10472 | |
| 187 | 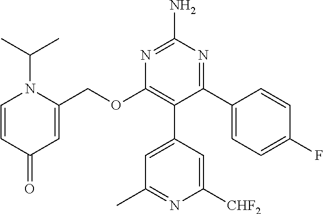 [SMILES: Cc1cc(-c2c(OCc3cc(=O)ccn3C(C)C)nc(N)nc2-c2ccc(F)cc2)cc(C(F)F)n1] | 2-(((2-amino-5-(2- (difluoromethyl)-6- methylpyridin-4-yl)-6-(4- fluorophenyl)pyrimidin-4- yl)oxy)methyl)-1- isopropylpyridin-4(1H)- one | | | | | |
| 188 | 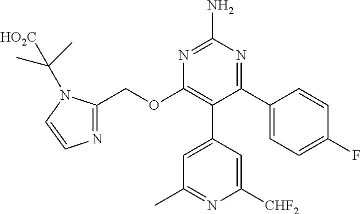 [SMILES: Cc1cc(-c2c(OCc3nccn3C(C)(C)C(=O)O)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1H- imidazol-1-yl)-2- methylpropanoic acid | | | | | |
| 189 | 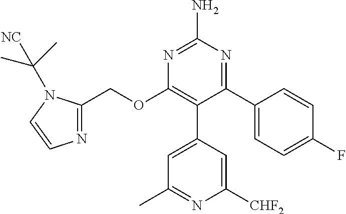 [SMILES: Cc1cc(-c2c(OCc3nccn3C(C)(C)C#N)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1H- imidazol-1-yl)-2- methylpropanenitrile | | | | | |
| 190 | 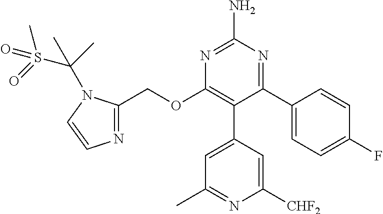 [SMILES: Cc1cc(-c2c(OCc3nccn3C(C)(C)S(C)(=O)=O)nc(N)nc2-c2ccc(F)cc2)cc(C(F)F)n1] | 5-(2-(difluoromethyl)-6- methylpyridin-4-yl)-4-(4- fluorophenyl)-6-((1-(2- (methylsulfonyl)propan-2- yl)-1H-imidazol-2- yl)methoxy)pyrimidin-2- amine | | | | | |
| 191 | 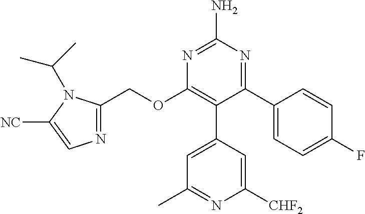 [SMILES: Cc1cc(-c2c(OCc3ncc(C#N)n3C(C)C)nc(N)nc2-c2ccc(F)cc2)cc(C(F)F)n1] | 2-(((2-amino-5-(2- (difluoromethyl)-6- methylpyridin-4-yl)-6-(4- fluorophenyl)pyrimidin-4- yl)oxy)methyl)-1- isopropyl-1H-imidazole-5- carbonitrile | 494.2 | 39 | 12 | | |
| 192 | 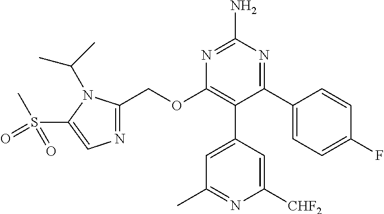 [SMILES: Cc1cc(-c2c(OCc3ncc(S(C)(=O)=O)n3C(C)C)nc(N)nc2-c2ccc(F)cc2)cc(C(F)F)n1] | 5-(2-(difluoromethyl)-6- methylpyridin-4-yl)-4-(4- fluorophenyl)-6-((1- isopropyl-5- (methylsulfonyl)-1H- imidazol-2- yl)methoxy)pyrimidin-2- amine | 547.2 | 26 | 9.7 | | |
| 193 | 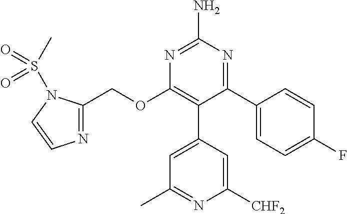 [SMILES: Cc1cc(-c2c(OCc3nccn3S(C)(=O)=O)nc(N)nc2-c2ccc(F)cc2)cc(C(F)F)n1] | 5-(2-(difluoromethyl)-6- methylpyridin-4-yl)-4-(4- fluorophenyl)-6-((1- (methylsulfonyl)-1H- imidazol-2- yl)methoxy)pyrimidin-2- amine | | | | | |
| 194 | 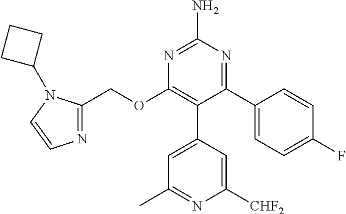 [SMILES: Cc1cc(-c2c(OCc3nccn3C3CCC3)nc(N)nc2-c2ccc(F)cc2)cc(C(F)F)n1] | 4-((1-cyclobutyl-1H- imidazol-2-yl)methoxy)-5- (2-(difluoromethyl)-6- methylpyridin-4-yl)-6-(4- fluorophenyl)pyrimidin-2- amine | 481.0 | 0.6/1.7 | 1.5 | 151 | |
| 195 | 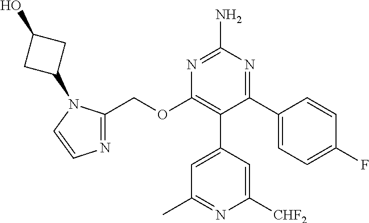 | (1r,3r)-3-(2-(((2-amino-5- (2-(difluoromethyl)-6- methylpyridin-4-yl)-6-(4- fluorophenyl)pyrimidin-4- yl)oxy)methyl)-1H- imidazol-1-yl)cyclobutan- 1-ol | | | | | |
| 196 | 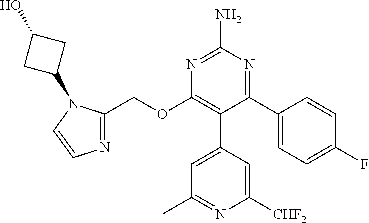 | (1s,3s)-3-(2-(((2-amino-5- (2-(difluoromethyl)-6- methylpyridin-4-yl)-6-(4- fluorophenyl)pyrimidin-4- yl)oxy)methyl)-1H- imidazol-1-yl)cyclobutan- 1-ol | | | | | |
| 197 | 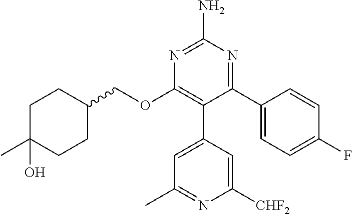 [SMILES: Cc1cc(-c2c(OCC3CCC(C)(O)CC3)nc(N)nc2-c2ccc(F)cc2)cc(C(F)F)n1] | 4-(((2-amino-5-(2- (difluoromethyl)-6- methylpyridin-4-yl)-6-(4- fluorophenyl)pyrimidin-4- yl)oxy)methyl)-1- methylcyclohexan-1-ol | 473.0 | 19.1 | 6.3 | | |
| 198 | 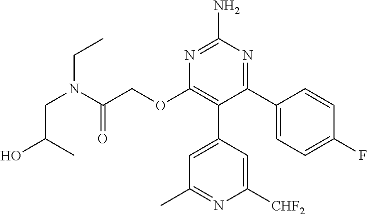 [SMILES: CCN(CC(C)O)C(=O)COc1nc(N)nc(-c2ccc(F)cc2)c1-c1cc(C)nc(C(F)F)c1] | 2-((2-amino-5-(2- (difluoromethyl)-6- methylpyridin-4-yl)-6-(4- fluorophenyl)pyrimidin-4- yl)oxy)-N-ethyl-N-(2- hydroxypropyl)acetamide | 490.1 | 14.9 | 10.0 | | |
| 199 | 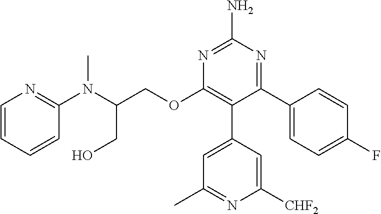 [SMILES: Cc1cc(-c2c(OCC(CO)N(C)c3ccccn3)nc(N)nc2-c2ccc(F)cc2)cc(C(F)F)n1] | 3-((2-amino-5-(2- (difluoromethyl)-6- methylpyridin-4-yl)-6-(4- fluorophenyl)pyrimidin-4- yl)oxy)-2-(methyl(pyridin- 2-yl)amino)propan-1-ol | 511.3 | 7.2 | | 148 | |
| 200 | 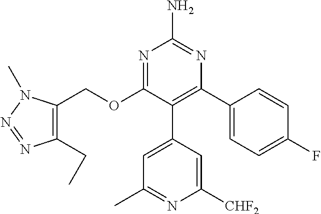 [SMILES: CCc1nnn(C)c1COc1nc(N)nc(-c2ccc(F)cc2)c1-c1cc(C)nc(C(F)F)c1] | 5-(2-(difluoromethyl)-6- methylpyridin-4-yl)-4-((4- ethyl-1-methyl-1H-1,2,3- triazol-5-yl)methoxy)-6-(4- fluorophenyl)pyrimidin-2- amine | 470.2 | 13.0 | | | |
| 201 | 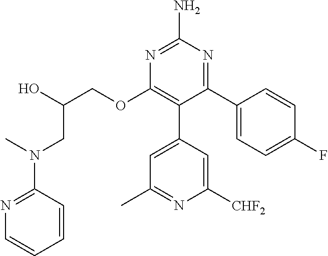 [SMILES: Cc1cc(-c2c(OCC(O)CN(C)c3ccccn3)nc(N)nc2-c2ccc(F)cc2)cc(C(F)F)n1] | 1-((2-amino-5-(2- (difluoromethyl)-6- methylpyridin-4-yl)-6-(4- fluorophenyl)pyrimidin-4- yl)oxy)-3-(methyl(pyridin- 2-yl)amino)propan-2-ol | 511.2 | 1.0/6.5 | 4.2/3.5 | | |
| 202 | 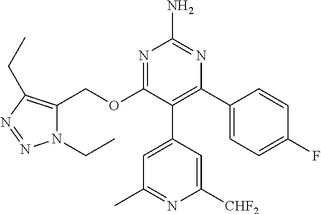 [SMILES: CCc1nnn(CC)c1COc1nc(N)nc(-c2ccc(F)cc2)c1-c1cc(C)nc(C(F)F)c1] | 4-((1,4-diethyl-1H-1,2,3- triazol-5-yl)methoxy)-5-(2- (difluoromethyl)-6- methylpyridin-4-yl)-6-(4- fluorophenyl)pyrimidin-2- amine | 484.2 | 3.5 | 16.0 | | |
| 203 | 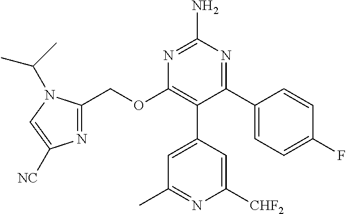 [SMILES: Cc1cc(-c2c(OCc3nc(C#N)cn3C(C)C)nc(N)nc2-c2ccc(F)cc2)cc(C(F)F)n1] | 2-((2-amino-5-(2- (difluoromethyl)-6- methylpyridin-4-yl)-6-(4- fluorophenyl)pyrimidin-4- yl)oxy)methyl)-1- isopropyl-1H-imidazole-4- carbonitrile | 494.2 | 3.0 | 9.6 | | |
| 204 | 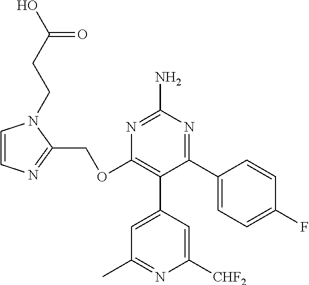 [SMILES: Cc1cc(-c2c(OCc3nccn3CCC(=O)O)nc(N)nc2-c2ccc(F)cc2)cc(C(F)F)n1] | 3-(2-(((2-amino-5-(2- (difluoromethyl)-6- methylpyridin-4-yl)-6-(4- fluorophenyl)pyrimidin-4- yl)oxy)methyl)-1H- imidazol-1-yl)propanoic acid | 498.9 | 13.0 | 24.0 | | 455.5 |
| 205 | 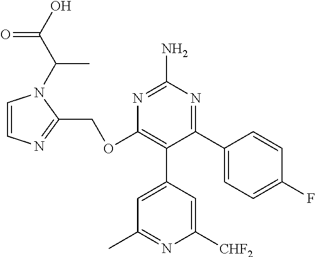 [SMILES: Cc1cc(-c2c(OCc3nccn3C(C)C(=O)O)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1H- imidazol-1-yl)propanoic acid | 499.2 | 12.0 | 29.0 | | |
| 206 | 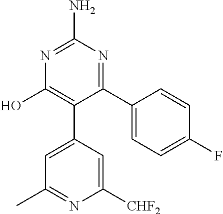 [SMILES: Cc1cc(-c2c(O)nc(N)nc2-c2ccc(F)cc2)cc(C(F)F)n1] | 2-amino-5-(2- (difluoromethyl)-6- methylpyridin-4-yl)-6-(4- fluorophenyl)pyrimidin-4- ol | 346.9 | 9.2 | 10.0 | 4664 | |
| 207 | 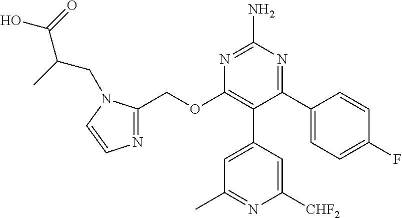 [SMILES: Cc1cc(-c2c(OCc3nccn3CC(C)C(=O)O)nc(N)nc2-c2ccc(F)cc2)cc(C(F)F)n1] | 3-(2-(((2-amino-5-(2- (difluoromethyl)-6- methylpyridin-4-yl)-6-(4- fluorophenyl)pyrimidin-4- yl)oxy)methyl)-1H- imidazol-1-yl)-2- methylpropanoic acid | 513.0 | 28 | 29 | | |
| 208 | 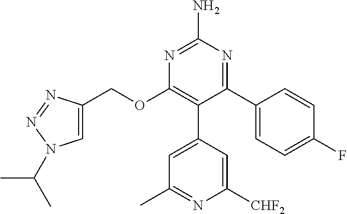 [SMILES: Cc1cc(-c2c(OCc3cn(C(C)C)nn3)nc(N)nc2-c2ccc(F)cc2)cc(C(F)F)n1] | 5-(2-(difluoromethyl)-6- methylpyridin-4-yl)-4-(4- fluorophenyl)-6-((1- isopropyl-1H-1,2,3-triazol- 4-yl)methoxy)pyrimidin-2- amine | 470.0 | 3.4 | 1.4/1.0 | 394 | |
| 209 | 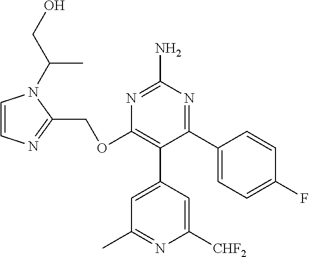 [SMILES: Cc1cc(-c2c(OCc3nccn3C(C)CO)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1H- imidazol-1-yl)propan-1-ol | 485.0 | 1.9/4.2 | 1.6/2.4 | 84/97 | 125 |
| 210 | 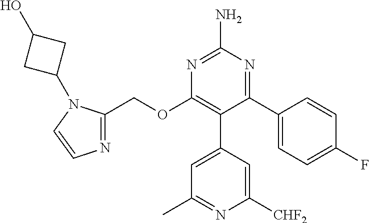 [SMILES: Cc1cc(-c2c(OCc3nccn3C3CC(O)C3)nc(N)nc2-c2ccc(F)cc2)cc(C(F)F)n1] | 3-(2-(((2-amino-5-(2- (difluoromethyl)-6- methylpyridin-4-yl)-6-(4- fluorophenyl)pyrimidin-4- yl)oxy)methyl)-1H- imidazol-1-yl)cyclobutan- 1-ol | 497.0 | 3.4/5.4 | 3.7/5.4 | 2250 | |
| 211 | 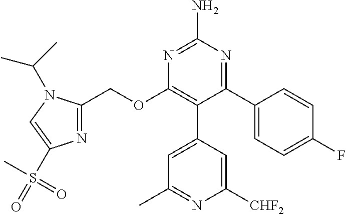 [SMILES: Cc1cc(-c2c(OCc3nc(S(C)(=O)=O)cn3C(C)C)nc(N)nc2-c2ccc(F)cc2)cc(C(F)F)n1] | 5-(2-(difluoromethyl)-6- methylpyridin-4-yl)-4-(4- fluorophenyl)-6-((1- isopropyl-4- (methylsulfonyl)-1H- imidazol-2- yl)methoxy)pyrimidin-2- amine | 546.9 | 47 | 7.3 | | |
| 212 | 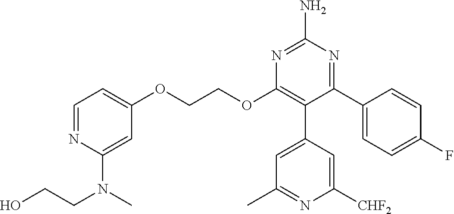 [SMILES: Cc1cc(-c2c(OCCOc3ccnc(N(C)CCO)c3)nc(N)nc2-c2ccc(F)cc2)cc(C(F)F)n1] | 2-((4-(2-((2-amino-5-(2- (difluoromethyl)-6- methylpyridin-4-yl)-6-(4- fluorophenyl)pyrimidin-4- yl)oxy)ethoxy)pyridin-2- yl)(methyl)amino)ethan-1- ol | 541.3 | 174 | 62 | | |
| 213 | 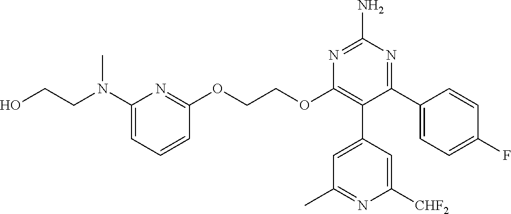 [SMILES: Cc1cc(-c2c(OCCOc3cccc(N(C)CCO)n3)nc(N)nc2-c2ccc(F)cc2)cc(C(F)F)n1] | 2-((6-(2-((2-amino-5-(2- (difluoromethyl)-6- methylpyridin-4-yl)-6-(4- fluorophenyl)pyrimidin-4- yl)oxy)ethoxy)pyridin-2- yl)(methyl)amino)ethan-1- ol | 541.3 | 9.3 | 3.4 | 7.16 | |
| 214 | 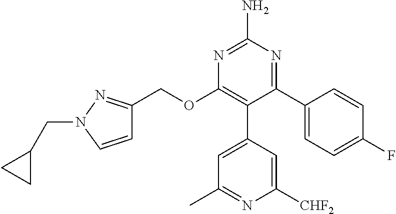 [SMILES: Cc1cc(-c2c(OCc3ccn(CC4CC4)n3)nc(N)nc2-c2ccc(F)cc2)cc(C(F)F)n1] | 4-(1-(cyclopropylmethyl)- 1H-pyrazol-3-yl)methoxy)- 5-(2-(difluoromethyl)-6- methylpyridin-4-yl)-6-(4- fluorophenyl)pyrimidin-2- amine | 481.1 | 4.5 | | 327 | |
| 215 | 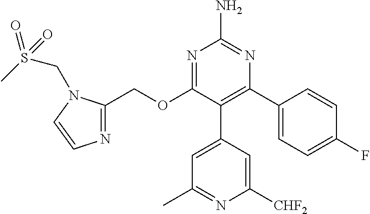 [SMILES: Cc1cc(-c2c(OCc3nccn3CS(C)(=O)=O)nc(N)nc2-c2ccc(F)cc2)cc(C(F)F)n1] | 5-(2-(difluoromethyl)-6- methylpyridin-4-yl)-4-(4- fluorophenyl)-6-((1- ((methylsulfonyl)methyl)- 1H-imidazol-2- yl)methoxy)pyrimidin-2- amine | 518.9 | 9.7 | | 965 | |
| 216 | 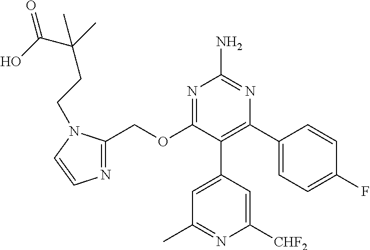 [SMILES: Cc1cc(-c2c(OCc3nccn3CCC(C)(C)C(=O)O)nc(N)nc2-c2ccc(F)cc2)cc(C(F)F)n1] | 4-(2-(((2-amino-5-(2- (dilfluoromethyl)-6- methylpyridin-4-yl)-6-(4- fluorophenyl)pyrimidin-4- yl)oxy)methyl)-1H- imidazol-1-yl)-2,2- dimethylbutanoic acid | 541.0 | 306 | | | |
| 217 | 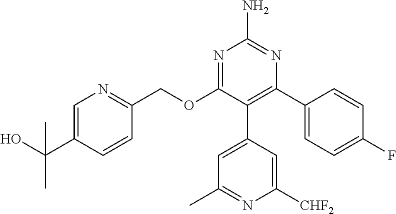 [SMILES: Cc1cc(-c2c(OCc3ccc(C(C)(C)O)cn3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)pyridin-3- yl)propan-2-ol | 496.2 | 21 | | 6495 | |
| 218 | 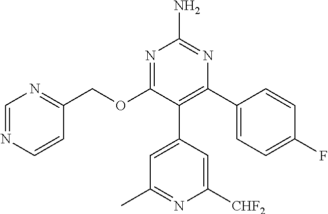 [SMILES: Cc1cc(-c2c(OCc3ccncn3)nc(N)nc2-c2ccc(F)cc2)cc(C(F)F)n1] | 5-(2-(difluoromethyl)-6- methylpyridin-4-yl)-4-(4- fluorophenyl)-6- (pyrimidin-4- ylmethoxy)pyrimidin-2- amine | 439.2 | 1.7 | | 76/48 | |
| 219 | 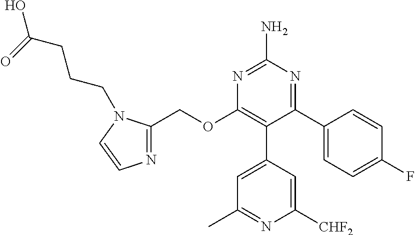 [SMILES: Cc1cc(-c2c(OCc3nccn3CCCC(=O)O)nc(N)nc2-c2ccc(F)cc2)cc(C(F)F)n1] | 4-(2-(((2-amino-5-(2- (difluoromethyl)-6- methylpyridin-4-yl)-6-(4- fluorophenyl)pyrimidin-4- yl)oxy)methyl)-1H- imidazol-1-yl)butanoic acid | 513.2 | 9.0 | | | 170.4 |
| 220 | 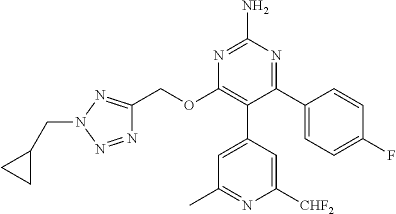 [SMILES: Cc1cc(-c2c(OCc3nnn(CC4CC4)n3)nc(N)nc2-c2ccc(F)cc2)cc(C(F)F)n1] | 4-((2-(cyclopropylmethyl)- 2H-tetrazol-5- yl)methoxy)-5-(2- (difluoromethyl)-6- methylpyridin-4-yl)-6-(4- fluorophenyl)pyrimidin-2- amine | 483.2 | 14.0 | | | |
| 221 | 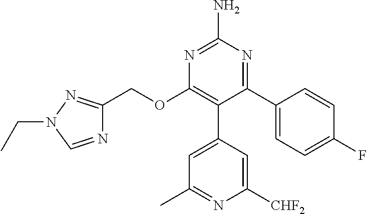 [SMILES: CCn1cnc(COc2nc(N)nc(-c3ccc(F)cc3)c2-c2cc(C)nc(C(F)F)c2)n1] | 5-(2-(difluoromethyl)-6- methyipyridin-4-yl)-4-((1- ethyl-1H-1,2,4-triazol-3- yl)methoxy)-6-(4- fluorophenyl)pyrimidin-2- amine | 456.2 | 24.0 | | 444 | |
| 222 | 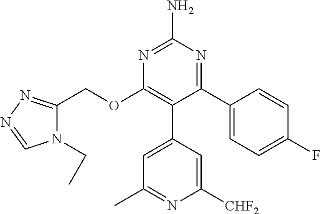 [SMILES: CCn1cnnc1COc1nc(N)nc(-c2ccc(F)cc2)c1-c1cc(C)nc(C(F)F)c1] | 5-(2-(difluoromethyl)-6- methylpyridin-4-yl)-4-((4- ethyl-4H-1,2,4-triazol-3- yl)methoxy)-6-(4- fluorophenyl)pyrimidin-2- amine | 456.2 | 15.0 | | | |
| 223 | 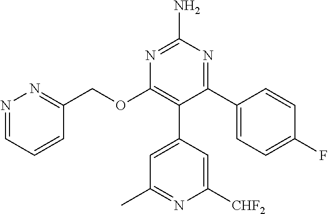 [SMILES: Cc1cc(-c2c(OCc3cccnn3)nc(N)nc2-c2ccc(F)cc2)cc(C(F)F)n1] | 5-(2-(difluoromethyl)-6- methylpyridin-4-yl)-4-(4- fluorophenyl)-6- (pyridazin-3- ylmethoxy)pyrimidin-2- amine | 439.1 | 4.1 | | 788 | |
| 224 | 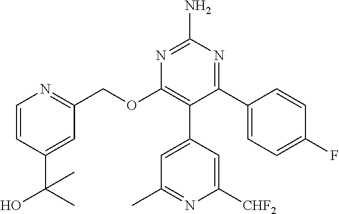 [SMILES: Cc1cc(-c2c(OCc3cc(C(C)(C)O)ccn3)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)pyridin-4- yl)propan-2-ol | 496.2 | 9.5 | | 1321 | |
| 225 | 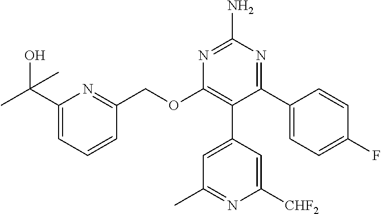 [SMILES: Cc1cc(-c2c(OCc3cccc(C(C)(C)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)propan-2-ol | 496.2 | 18.1 | | 136/ 200/ 176 | 15.1 |
| 226 | 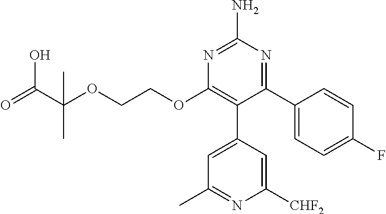 [SMILES: Cc1cc(-c2c(OCCOC(C)(C)C(=O)O)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)ethoxy)-2- methylpropanoic acid | 477.1 | | | >2500 | |
| 227 | 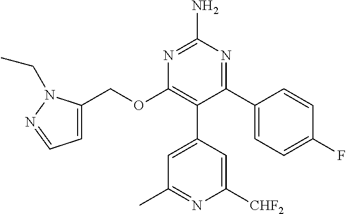 [SMILES: CCn1nccc1COc1nc(N)nc(-c2ccc(F)cc2)c1-c1cc(C)nc(C(F)F)c1] | 5-(2-(difluoromethyl)-6- methylpyridin-4-yl)-4-((1- ethyl-1H-pyrazol-5- yl)methoxy)-6-(4- fluorophenyl)pyrimidin-2- amine | 455.2 | | | 29/97/ 71 | 17.8 |
| 228 | 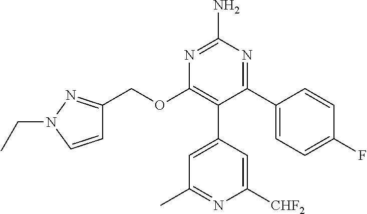 [SMILES: CCn1ccc(COc2nc(N)nc(-c3ccc(F)cc3)c2-c2cc(C)nc(C(F)F)c2)n1] | 5-(2-(difluoromethyl)-6- methylpyridin-4-yl)-4-((1- ethyl-1H-pyrazol-3- yl)methoxy)-6-(4- fluorophenyl)pyrimidin-2- amine | 455.0 | 2.5/3.6 | 0.9 | 162 | |
| 229 | 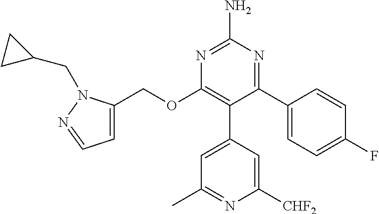 [SMILES: Cc1cc(-c2c(OCc3ccnn3CC3CC3)nc(N)nc2-c2ccc(F)cc2)cc(C(F)F)n1] | 4-((1-(cyclopropylmethyl)- 1H-pyrazol-5-yl)methoxy)- 5-(2-(difluoromethyl)-6- methylpyridin-4-yl)-6-(4- fluorophenyl)pyrimidin-2- amine | 481.2 | | | 107 | |
| 230 | 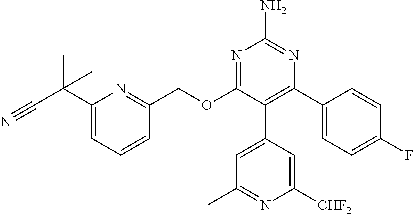 [SMILES: Cc1cc(-c2c(OCc3cccc(C(C)(C)C#N)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-2-methylpropanenitrile | 505.2 | | | 7.0/40/ 64 | 32.4/ 37.6 |
| 231 | 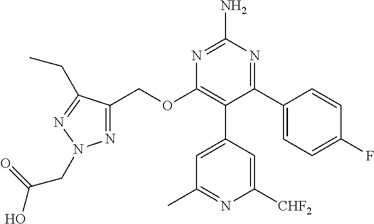 [SMILES: CCc1nn(CC(=O)O)nc1COc1nc(N)nc(-c2ccc(F)cc2)c1-c1cc(C)nc(C(F)F)c1] | 2-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)acetic acid | 514.2 | | | >25000 | |
| 232 | 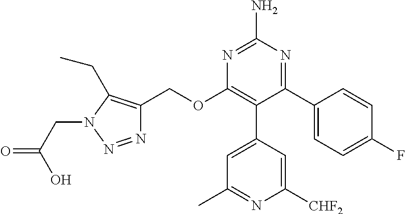 [SMILES: CCc1c(COc2nc(N)nc(-c3ccc(F)cc3)c2-c2cc(C)nc(C(F)F)c2)nnn1CC(=O)O] | 2-(4-(((2-amino-5-(2- (difluoromethyl)-6- methylpyridin-4-yl)-6-(4- fluorophenyl)pyrimidin-4- yl)oxy)methyl)-5-ethyl-1H- 1,2,3-triazol-1-yl)acetic acid | 514.2 | | | 4506 | |
| 233 | 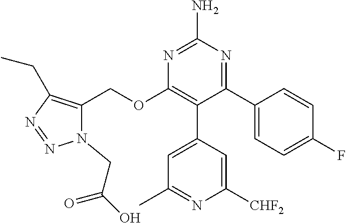 [SMILES: CCc1nnn(CC(=O)O)c1COc1nc(N)nc(-c2ccc(F)cc2)c1-c1cc(C)nc(C(F)F)c1] | 2-(5-(((2-amino-5-(2- (difluoromethyl)-6- methylpyridin-4-yl)-6-(4- fluorophenyl)pyrimidin-4- yl)oxy)methyl)-4-ethyl-1H- 1,2,3-triazol-1-yl)acetic acid | 514.2 | | | 16339 | |
| 234 | 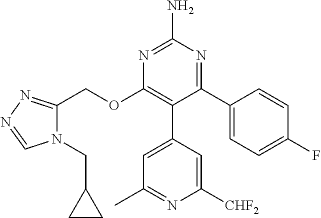 [SMILES: Cc1cc(-c2c(OCc3nncn3CC3CC3)nc(N)nc2-c2ccc(F)cc2)cc(C(F)F)n1] | 4-((4-cyclopropylmethyl)- 1,2,4-triazol-3- yl)methoxy)-5-(2- (difluoromethyl)-6- methylpyridin-4-yl)-6-(4- fluorophenyl)pyrimidin-2- amine | 482.2 | | | 647 | |
| 235 | 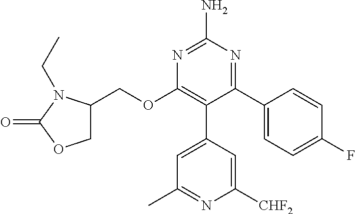 [SMILES: CCN1C(=O)OCC1COc1nc(N)nc(-c2ccc(F)cc2)c1-c1cc(C)nc(C(F)F)c1] | 4-(((2-amino-5-(2- (difluoromethyl)-6- methylpyridin-4-yl)-6-(4- fluorophenyl)pyrimidin-4- yl)oxy)methyl)-3- ethyloxazolidin-2-one | 474.2 | | | 3708 | |
| 236 | 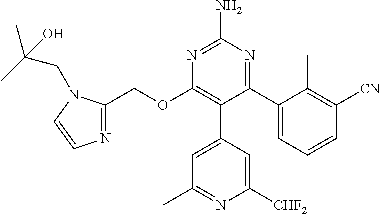 [SMILES: Cc1cc(-c2c(OCc3nccn3CC(C)(C)O)nc(N)nc2-c2cccc(C#N)c2C)cc(C(F)F)n1] | 3-(2-amino-5-(2- (difluoromethyl)-6- methylpyridin-4-yl)-6-((1- (2-hydroxy-2- methylpropyl)-1H- imidazol-2- yl)methoxy)pyrimidin-4- yl)-2-methylbenzonitrile | 520.2 | | | >25000 | |
| 237 | 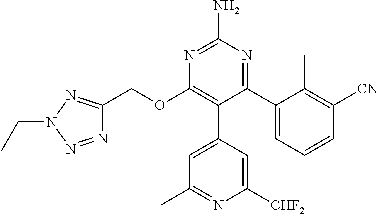 [SMILES: CCn1nnc(COc2nc(N)nc(-c3cccc(C#N)c3C)c2-c2cc(C)nc(C(F)F)c2)n1] | 3-(2-amino-5-(2- (difluoromethyl)-6- methylpyridin-4-yl)-6-((2- ethyl-2H-tetrazol-5- yl)methoxy)pyrimidin-4- yl)-2-methylbenzonitrile | 478.2 | | | 2119 | |
| 238 | 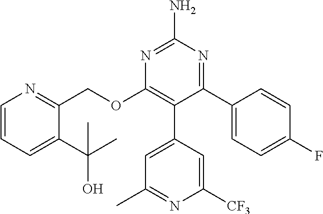 [SMILES: Cc1cc(-c2c(OCc3ncccc3C(C)(C)O)nc(N)nc2-c2ccc(F)cc2)cc(C(F)(F)F)n1] | 2-(2-(((2-amino-5-(2- (difluoromethyl)-6- methylpyridin-4-yl)-6-(4- fluorophenyl)pyrimidin-4- yl)oxy)methyl)pyridin-3- yl)propan-2-ol | 496.1 | | | 313 | 36.3 |
| 239 | 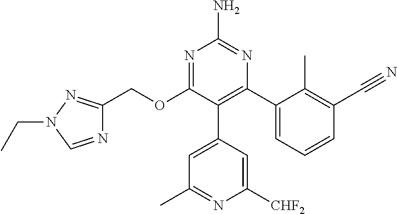 [SMILES: CCn1cnc(COc2nc(N)nc(-c3cccc(C#N)c3C)c2-c2cc(C)nc(C(F)F)c2)n1] | 3-(2-amino-5-(2- (difluoromethyl)-6- methylpyridin-4-yl)-6-((1- ethyl-1H-1,2,4-triazol-3- yl)methoxy)pyrimidin-4- yl)-2-methylbenzonitrile | 477.2 | | | 7919 | |
| 240 | 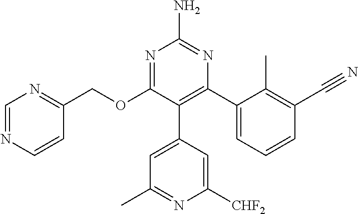 [SMILES: Cc1cc(-c2c(OCc3ccncn3)nc(N)nc2-c2cccc(C#N)c2C)cc(C(F)F)n1] | 3-(2-amino-5-(2- (difluoromethyl)-6- methylpyridin-4-yl)-6- (pyrimidin-4- ylmethoxy)pyrimidin-4-yl)- 2-methylbenzonitrile | 460.2 | | | 17037 | |
| 241 | 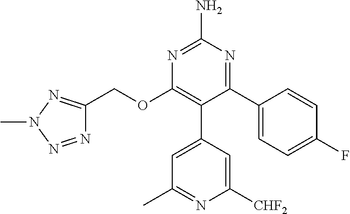 [SMILES: Cc1cc(-c2c(OCc3nnn(C)n3)nc(N)nc2-c2ccc(F)cc2)cc(C(F)F)n1] | 5-(2-(difluoromethyl)-6- methylpyridin-4-yl)-4-(4- fluorophenyl)-6-((2- methyl-2H-tetrazol-5- yl)methoxy)pyrimidin-2- amine | 443.1 | | | 213 | 221 |
| 242 | 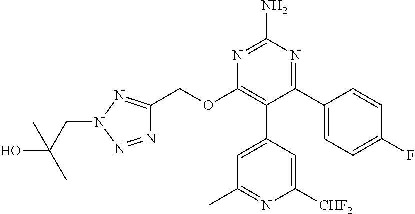 [SMILES: Cc1cc(-c2c(OCc3nnn(CC(C)(C)O)n3)nc(N)nc2-c2ccc(F)cc2)cc(C(F)F)n1] | 1-(5-(((2-amino-5-(2- (difluoromethyl)-6- methylpyridin-4-yl)-6-(4- fluorophenyl)pyrimidin-4- yl)oxy)methyl)-2H- tetrazol-2-yl)-2- methylpropan-2-ol | 501.2 | | | 2024 | |
| 243 | 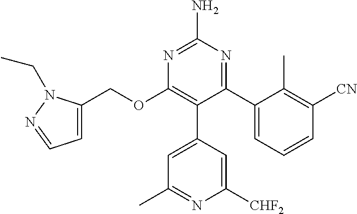 [SMILES: CCn1nccc1COc1nc(N)nc(-c2cccc(C#N)c2C)c1-c1cc(C)nc(C(F)F)c1] | 3-(2-amino-5-(2- (difluoromethyl)-6- methylpyridin-4-yl)-6-((1- ethyl-1H-pyrazol-5- yl)methoxy)pyrimidin-4- yl)-2-methylbenzonitrile | 476.1 | | | 2949 | |
| 244 | 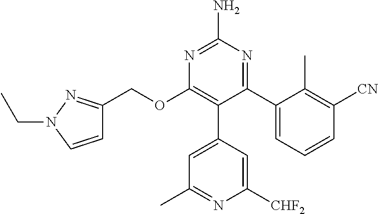 [SMILES: CCn1ccc(COc2nc(N)nc(-c3cccc(C#N)c3C)c2-c2cc(C)nc(C(F)F)c2)n1] | 3-(2-amino-5-(2- (difluoromethyl)-6- methylpyridin-4-yl)-6-((1- ethyl-1H-pyrazol-3- yl)methoxy)pyrimidin-4- yl)-2-methylbenzonitrile | 476.2 | | | 756 | |
| 245 | 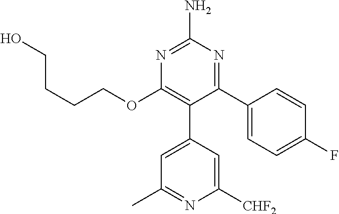 [SMILES: Cc1cc(-c2c(OCCCCO)nc(N)nc2-c2ccc(F)cc2)cc(C(F)F)n1] | 4-((2-amino-5-(2- (difluoromethyl)-6- methylpyridin-4-yl)-6-(4- fluorophenyl)pyrimidin-4- yl)oxy)butan-1-ol | 419.2 | | | 265 | |
| 246 | 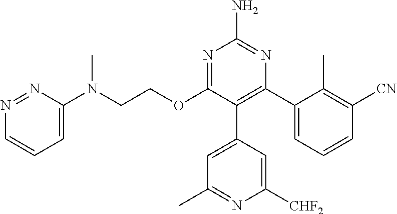 [SMILES: Cc1cc(-c2c(OCCN(C)c3cccnn3)nc(N)nc2-c2cccc(C#N)c2C)cc(C(F)F)n1] | 3-(2-amino-5-(2- (difluoromethyl)-6- methylpyridin-4-yl)-6-(2- (methyl(pyridazin-3- yl)amino)ethoxy)pyrimidin- 4-yl)-2-methylbenzonitrile | 503.2 | | | 981 | |
| 247 | 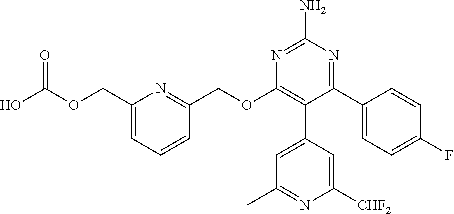 [SMILES: Cc1cc(-c2c(OCc3cccc(COC(=O)O)n3)nc(N)nc2-c2ccc(F)cc2)cc(C(F)F)n1] | 2-((6-(((2-amino-5-(2- (difluoromethyl)-6- methylpyridin-4-yl)-6-(4- fluorophenyl)pyrimidin-4- yl)oxy)methyl)pyridin-2- yl)oxy)acetic acid | 512.2 | | | 549/ 760/ 913 | 205 |
| 248 | 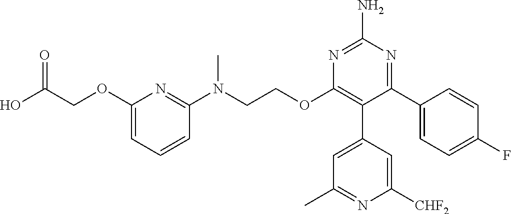 [SMILES: Cc1cc(-c2c(OCCN(C)c3cccc(OCC(=O)O)n3)nc(N)nc2-c2ccc(F)cc2)cc(C(F)F)n1] | 2-((6-((2-((2-amino-5-(2- (difluoromethyl)-6- methylpyridin-4-yl)-6-(4- fluorophenyl)pyrimidin-4- yl)oxy)ethyl)(methyl) amino)pyridin-2-yl)oxy) acetic | 555.3 | | | 552/ 742/ 1096 | 77/11 |
| 249 | 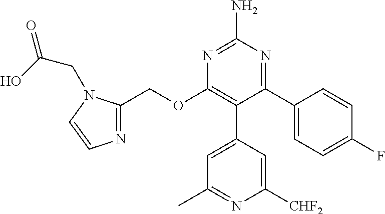 [SMILES: Cc1cc(-c2c(OCc3nccn3CC(=O)O)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1H- imidazol-1-yl)acetic acid | 485.1 | | | 7242 | |
| 250 | 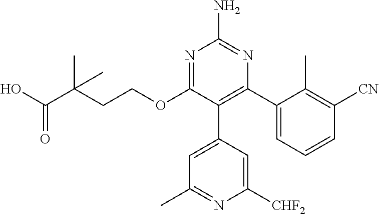 [SMILES: Cc1cc(-c2c(OCCC(C)(C)C(=O)O)nc(N)nc2-c2cccc(C#N)c2C)cc(C(F)F)n1] | 4-((2-amino-6-(3-cyano-2- methylphenyl)-5-(2- (difluoromethyl)-6- methylpyridin-4- yl)pyrimidin-4-yl)oxy)-2,2- dimethylbutanoic acid | 482.2 | | | 8573 | |
| 251 | 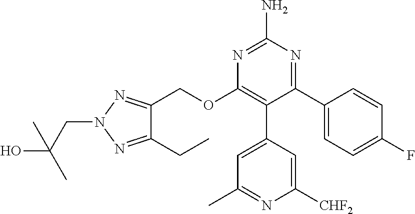 [SMILES: CCc1nn(CC(C)(C)O)nc1COc1nc(N)nc(-c2ccc(F)cc2)c1-c1cc(C)nc(C(F)F)c1] | 1-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)-2- methylpropan-2-ol | 528.3 | | | 1278 | |
| 252 | 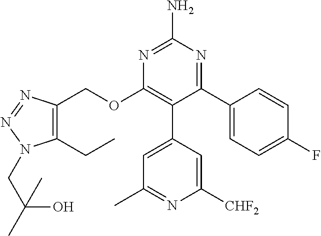 [SMILES: CCc1c(COc2nc(N)nc(-c3ccc(F)cc3)c2-c2cc(C)nc(C(F)F)c2)nnn1CC(C)(C)O] | 1-(4-(((2-ainino-5-(2- (difluoromethyl)-6- methylpyridin-4-yl)-6-(4- fluorophenyl)pyrimidin-4- yl)oxy)methyl)-5-ethyl-1H- 1,2,3-triazol-1-yl)-2- methylpropan-2-ol | 528.3 | | | 1264 | |
| 253 | 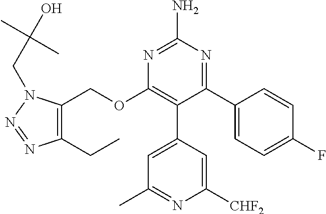 [SMILES: CCc1nnn(CC(C)(C)O)c1COc1nc(N)nc(-c2ccc(F)cc2)c1-c1cc(C)nc(C(F)F)c1] | 1-(5-(((2-amino-5-(2- (difluoromethyl)-6- methylpyridin-4-yl)-6-(4- fluorophenyl)pyrimidin-4- yl)oxy)methyl)-4-ethyl-1H- 1,2,3-triazol-1-yl)-2- methylpropan-2-ol | 528.3 | | | 2155 | |
| 254 | 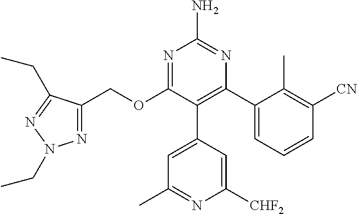 [SMILES: CCc1nn(CC)nc1COc1nc(N)nc(-c2cccc(C#N)c2C)c1-c1cc(C)nc(C(F)F)c1] | 3-(2-amino-6-((2,5- diethyl-2H-1,2,3-triazol-4- yl)methoxy)-5-(2- (difluoromethyl)-6- methylpyridin-4- yl)pyrimidin-4-yl)-2- methylbenzonitrile | 505.2 | | | 23546 | |
| 255 | 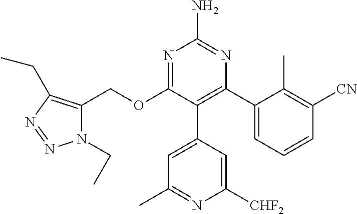 [SMILES: CCc1nnn(CC)c1COc1nc(N)nc(-c2cccc(C#N)c2C)c1-c1cc(C)nc(C(F)F)c1] | 3-(2-amino-6-((1,4- diethyl-1H-1,2,3-triazol-5- yl)methoxy)-5-(2- (difluoromethyl)-6- methylpyridin-4- yl)pyrimidin-4-yl)-2- methylbenzonitrile | 505.3 | | | >25000 | |
| 256 | 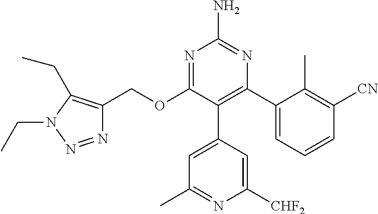 [SMILES: CCc1c(COc2nc(N)nc(-c3cccc(C#N)c3C)c2-c2cc(C)nc(C(F)F)c2)nnn1CC] | 3-(2-amino-6-((1,5- diethyl-1H-1,2,3-triazol-4- yl)methoxy)-5-(2- (difluoromethyl)-6- methylpyridin-4- yl)pyrimidin-4-yl)-2- methylbenzonitrile | 505.3 | | | 2915 | |
| 257 | 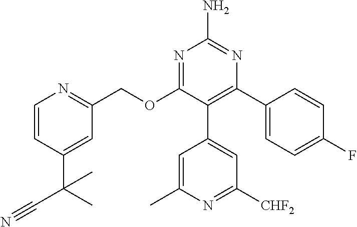 [SMILES: Cc1cc(-c2c(OCc3cc(C(C)(C)C#N)ccn3)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)pyridin-4- yl)-2-methylpropanenitrile | 505.2 | | | 250/ 171 | 19 |
| 258 | 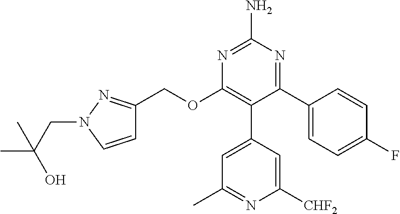 [SMILES: Cc1cc(-c2c(OCc3ccn(CC(C)(C)O)n3)nc(N)nc2-c2ccc(F)cc2)cc(C(F)F)n1] | 1-(3-(((2-amino-5-(2- (difluoromethyl)-6- methylpyridin-4-yl)-6-(4- fluorophenyl)pyrimidin-4- yl)oxy)methyl)-1H- pyrazol-1-yl)-2- methylpropan-2-ol | 499.2 | | | 2395 | |
| 259 | 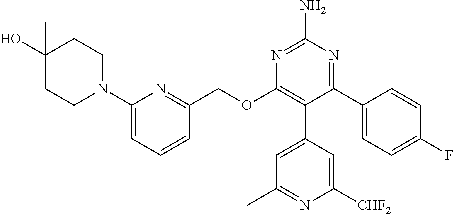 [SMILES: Cc1cc(-c2c(OCc3cccc(N4CCC(C)(O)CC4)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)-4-methylpiperidin-4-ol | 551.2 | | | >25000 | |
| 260 | 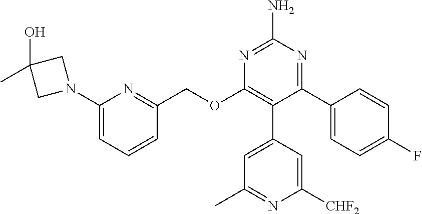 [SMILES: Cc1cc(-c2c(OCc3cccc(N4CC(C)(O)C4)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)-3-methylazetidin-3-ol | 523.2 | | | 1365 | |
| 261 | 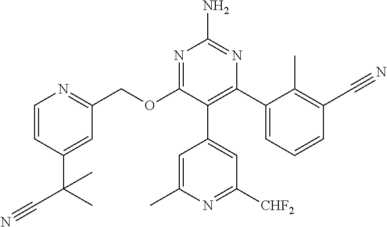 [SMILES: Cc1cc(-c2c(OCc3cc(C(C)(C)C#N)ccn3)nc(N)nc2-c2cccc(C#N)c2C)cc(C(F)F)n1] | 3-(2-amino-6-((4-(2- cyanopropan-2-yl)pyridin- 2-yl)methoxy)-5-(2- (difluoromethyl)-6- methylpyridin-4- yl)pyrimidin-4-yl)-2- methylbenzonitrile | 526.2 | | | 2190 | |
| 262 | 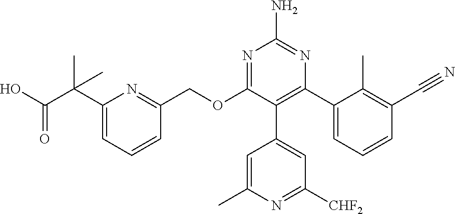 [SMILES: Cc1cc(-c2c(OCc3cccc(C(C)(C)C(=O)O)n3)nc(N)nc2-c2cccc(C#N)c2C)cc(C(F)F)n1] | 2-(6-(((2-amino-6-(3- cyano-2-methylphenyl)-5- (2-(difluoromethyl)-6- methylpyridin-4- yl)pyrimidin-4- yl)oxy)methyl)pyridin-2- yl)-2-methylpropanoic acid | 545.2 | | | | |
| 263 | 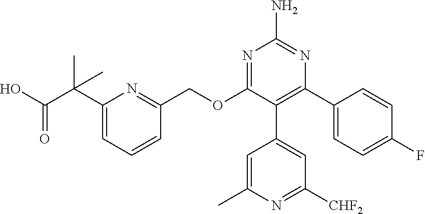 [SMILES: Cc1cc(-c2c(OCc3cccc(C(C)(C)C(=O)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-2-methylpropanoic acid | 524.2 | | | 33/168/ 95/126 | 82/114 |
| 264 | 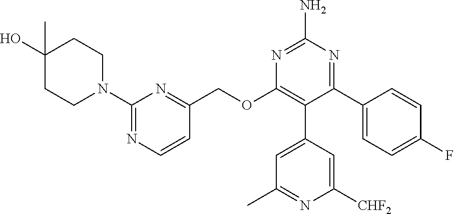 [SMILES: Cc1cc(-c2c(OCc3ccnc(N4CCC(C)(O)CC4)n3)nc(N)nc2-c2ccc(F)cc2)cc(C(F)F)n1] | 1-(4-(((2-amino-5-(2- (difluoromethyl)-6- methylpyridin-4-yl)-6-(4- fluorophenyl)pyrimidin-4- yl)oxy)methyl)pyrimidin-2- yl)-4-methylpiperidin-4-ol | 552.2 | | | | |
| 265 | 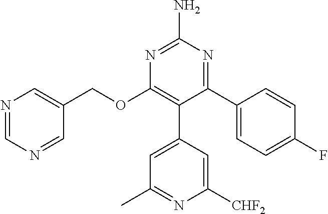 [SMILES: Cc1cc(-c2c(OCc3cncnc3)nc(N)nc2-c2ccc(F)cc2)cc(C(F)F)n1] | 5-(2-(difluoromethyl)-6- methylpyridin-4-yl)-4-(4- fluorophenyl)-6- (pyrimidin-5- ylmethoxy)pyrimidin-2- amine | 439.1 | | | | |
| 266 | 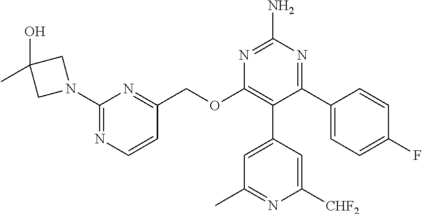 [SMILES: Cc1cc(-c2c(OCc3ccnc(N4CC(C)(O)C4)n3)nc(N)nc2-c2ccc(F)cc2)cc(C(F)F)n1] | 1-(4-(((2-amino-5-(2- (difluoromethyl)-6- methylpyridin-4-yl)-6-(4- fluorophenyl)pyrimidin-4- yl)oxy)methyl)pyrimidin-2- yl)-3-methylazetidin-3-ol | 524.2 | | | | 4909 |
| 267 | 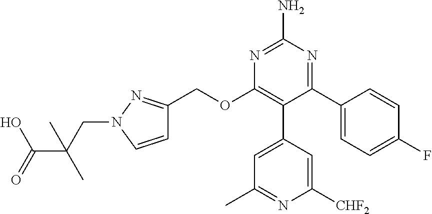 [SMILES: Cc1cc(-c2c(OCc3ccn(CC(C)(C)C(=O)O)n3)nc(N)nc2-c2ccc(F)cc2)cc(C(F)F)n1] | 3-(3-(((2-amino-5-(2- (difluoromethyl)-6- methylpyridin-4-yl)-6-(4- fluorophenyl)pyrimidin-4- yl)oxy)methyl)-1H- pyrazol-1-yl)-2,2- dimethylpropanoic acid | 527.2 | | | | 5583 |
| 268 | 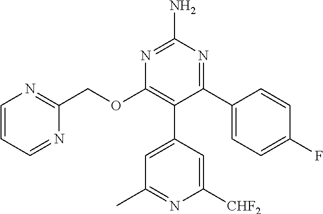 [SMILES: Cc1cc(-c2c(OCc3ncccn3)nc(N)nc2-c2ccc(F)cc2)cc(C(F)F)n1] | 5-(2-(difluoromethyl)-6- methylpyridin-4-yl)-4-(4- fluorophenyl)-6- (pyrimidin-2- ylmethoxy)pyrimidin-2- amine | 439.1 | | | | |
| 269 | 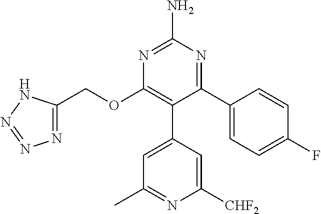 [SMILES: Cc1cc(-c2c(OCc3nnn[nH]3)nc(N)nc2-c2ccc(F)cc2)cc(C(F)F)n1] | 4-((1H-tetrazol-5- yl)methoxy)-5-(2- (difluoromethyl)-6- methylpyridin-4-yl)-6-(4- fluorophenyl)pyrimidin-2- amine | 429.1 | | | >25000 | |
| 270 | 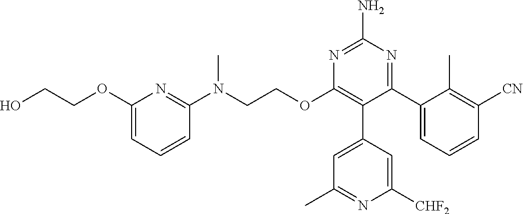 [SMILES: Cc1cc(-c2c(OCCN(C)c3cccc(OCCO)n3)nc(N)nc2-c2cccc(C#N)c2C)cc(C(F)F)n1] | 3-(2-amino-5-(2- (difluoromethyl)-6- methylpyridin-4-yl)-6-(2- ((6-(2- hydroxyethoxy)pyridin-2- yl)(methyl)amino)ethoxy) pyrimidin-4-yl)-2- methylbenzonitrile | 562.3 | | | | |
| 271 | 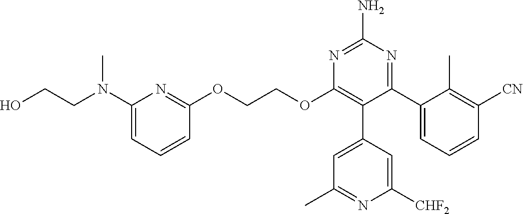 [SMILES: Cc1cc(-c2c(OCCOc3cccc(N(C)CCO)n3)nc(N)nc2-c2cccc(C#N)c2C)cc(C(F)F)n1] | 3-(2-amino-5-(2- (difluoromethyl)-6- methylpyridin-4-yl)-6-(2- ((6-((2- hydroxyethyl)(methyl) amino)pyridin-2- yl)oxy)ethoxy)pyrimidin-4- yl)-2-methylbenzonitrile | 562.3 | | | | |
| 272 | 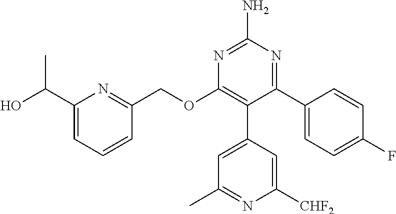 [SMILES: Cc1cc(-c2c(OCc3cccc(C(C)O)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)ethan-1-ol | 482.2 | | | 88/161 | |
| 273 | 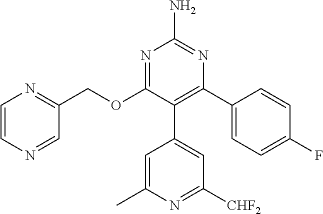 [SMILES: Cc1cc(-c2c(OCc3cnccn3)nc(N)nc2-c2ccc(F)cc2)cc(C(F)F)n1] | 5-(2-(difluoromethyl)-6- methylpyridin-4-yl)-4-(4- fluorophenyl)-6-(pyrazin- 2-ylmethoxy)pyrimidin-2- amine | 439.2 | | | 1661 | |
| 274 | 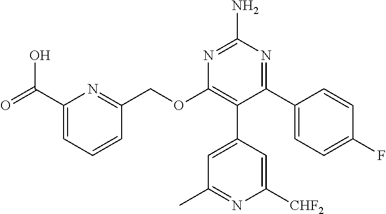 [SMILES: Cc1cc(-c2c(OCc3cccc(C(=O)O)n3)nc(N)nc2-c2ccc(F)cc2)cc(C(F)F)n1] | 6-(((2-amino-5-(2- (difluoromethyl)-6- methylpyridin-4-yl)-6-(4- fluorophenyl)pyrimidin-4- yl)oxy)methyl)picolinic acid | 482.2 | | | 3212 | |
| 275 | 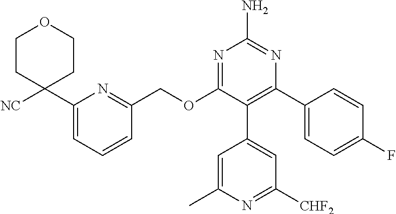 [SMILES: Cc1cc(-c2c(OCc3cccc(C4(C#N)CCOCC4)n3)nc(N)nc2-c2ccc(F)cc2)cc(C(F)F)n1] | 4-(6-(((2-amino-5-(2- (difluoromethyl)-6- methylpyridin-4-yl)-6-(4- fluorophenyl)pyrimidin-4- yl)oxy)methyl)pyridin-2- yl)tetrahydro-2H-pyran-4- carbonitrile | 547.2 | | | 321 | |
| 276 | 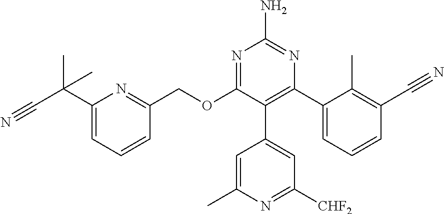 [SMILES: Cc1cc(-c2c(OCc3cccc(C(C)(C)C#N)n3)nc(N)nc2-c2cccc(C#N)c2C)cc(C(F)F)n1] | 3-(2-amino-6-((6-(2- cyanopropan-2-yl)pyridin- 2-yl)methoxy)-5-(2- (difluoromethyl)-6- methylpyridin-4- yl)pyrimidin-4-yl)-2- methylbenzonitrile | 526.3 | | | | |
| 277 | 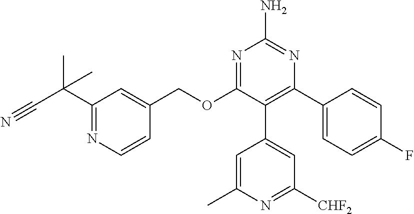 [SMILES: Cc1cc(-c2c(OCc3ccnc(C(C)(C)C#N)c3)nc(N)nc2-c2ccc(F)cc2)cc(C(F)F)n1] | 2-(4-(((2-amino-5-(2- (difluoromethyl)-6- methylpyridin-4-yl)-6-(4- fluorophenyl)pyrimidin-4- yl)oxy)methyl)pyridin-2- yl)-2-methylpropanenitrile | 505.3 | | | 455 | |
| 278 | 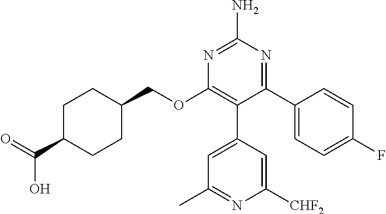 [SMILES: Cc1cc(-c2c(OC[C@H]3CC[C@@H](C(=O)O)CC3)nc(N)nc2-c2ccc(F)cc2)cc(C(F)F)n1] | (1,4-cis)-4-(((2-amino-5- (2-(difluoromethyl)-6- methylpyridin-4-yl)-6-(4- fluorophenyl)pyrimidin-4- yl)oxy)methyl) cyclohexane- 1-carboxylic acid | 487.2 | | | 19949 | |
| 279 | 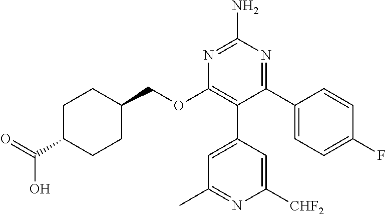 [SMILES: Cc1cc(-c2c(OC[C@H]3CC[C@H](C(=O)O)CC3)nc(N)nc2-c2ccc(F)cc2)cc(C(F)F)n1] | (1,4-trans)-4-(((2-amino- 5-(2-(difluoromethyl)-6- methylpyridin-4-yl)-6-(4- fluorophenyl)pyrimidin-4- yl)oxy)methyl) cyclohexane- 1-carboxylic acid | 487.1 | | | >25000 | |
| 280 | 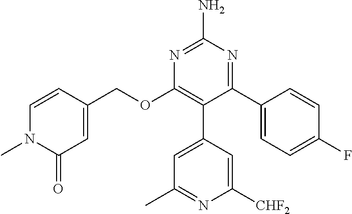 [SMILES: Cc1cc(-c2c(OCc3ccn(C)c(=O)c3)nc(N)nc2-c2ccc(F)cc2)cc(C(F)F)n1] | 4-(((2-amino-5-(2- (difluoromethyl)-6- methylpyridin-4-yl)-6-(4- fluorophenyl)pyrimidin-4- yl)oxy)methyl)-1- methylpyridin-2(1H)-one | 468.1 | | | 5796 | |
| 281 | 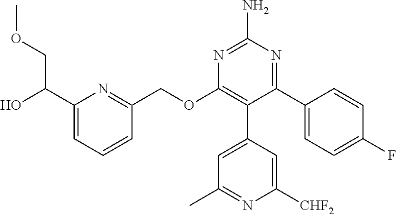 [SMILES: COCC(O)c1cccc(COc2nc(N)nc(-c3ccc(F)cc3)c2-c2cc(C)nc(C(F)F)c2)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)-2-methoxyethan-1-ol | 512.2 | | | 609 | |
| 282 | 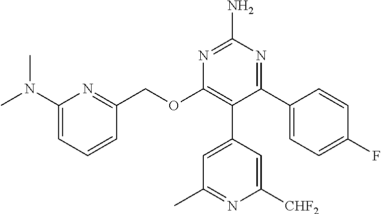 [SMILES: Cc1cc(-c2c(OCc3cccc(N(C)C)n3)nc(N)nc2-c2ccc(F)cc2)cc(C(F)F)n1] | 5-(2-(difluoromethyl)-6- methylpyridin-4-yl)-4-((6- (dimethylamino)pyridin-2- yl)methoxy)-6-(4- fluorophenyl)pyrimidin-2- amine | 481.2 | | | 90/21/ 100 | 50 |
| 283 | 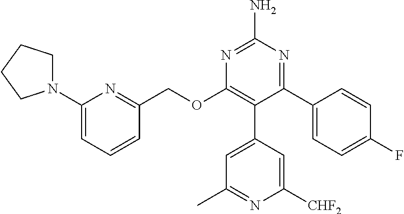 [SMILES: Cc1cc(-c2c(OCc3cccc(N4CCCC4)n3)nc(N)nc2-c2ccc(F)cc2)cc(C(F)F)n1] | 5-(2-(difluoromethyl)-6- methylpyridin-4-yl)-4-(4- fluorophenyl)-6-((6- (pyrrolidin-1-yl)pyridin-2- yl)methoxy)pyrimidin-2- amine | 507.2 | | | 1171 | |
| 284 | 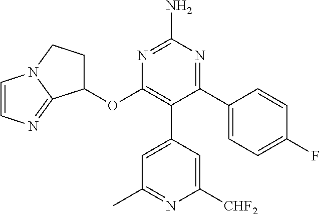 [SMILES: Cc1cc(-c2c(OC3CCn4ccnc43)nc(N)nc2-c2ccc(F)cc2)cc(C(F)F)n1] | 5-(2-(difluoromethyl)-6- methylpyridin-4-yl)-4- ((6,7-dihydro-5H- pyrrolo[1,2-a]imidazol-7- yl)oxy)-6-(4- fluorophenyl)pyrimidin-2- amine | 453.1 | | | 382/ 289 | 392 |
| 285 | 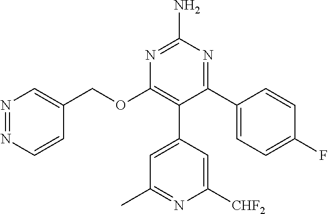 [SMILES: Cc1cc(-c2c(OCc3ccnnc3)nc(N)nc2-c2ccc(F)cc2)cc(C(F)F)n1] | 5-(2-(difluoromethyl)-6- methylpyridin-4-yl)-4-(4- fluorophenyl)-6- (pyridazin-4- ylmethoxy)pyrimidin-2- amine | 439.1 | | | 378/ 272 | 179 |
| 286 | 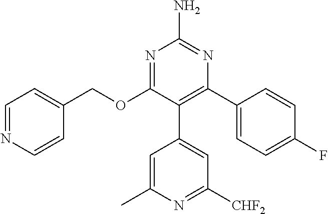 [SMILES: Cc1cc(-c2c(OCc3ccncc3)nc(N)nc2-c2ccc(F)cc2)cc(C(F)F)n1] | 5-(2-(difluoromethyl)-6- methylpyridin-4-yl)-4-(4- fluorophenyl)-6-(pyridin-4- ylmethoxy)pyrimidin-2- amine | 438.2 | | | 1803 | |
| 287 | 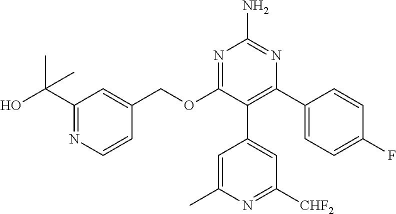 [SMILES: Cc1cc(-c2c(OCc3ccnc(C(C)(C)O)c3)nc(N)nc2-c2ccc(F)cc2)cc(C(F)F)n1] | 2-(4-(((2-amino-5-(2- (difluoromethyl)-6- methylpyridin-4-yl)-6-(4- fluorophenyl)pyrimidin-4- yl)oxy)methyl)pyridin-2- yl)propan-2-ol | 496.2 | | | 835 | |
| 288 | 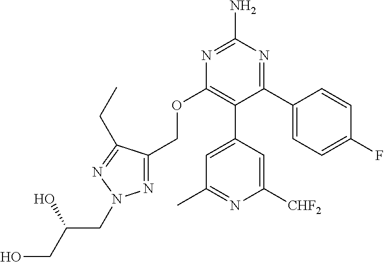 [SMILES: CCc1nn(C[C@@H](O)CO)nc1COc1nc(N)nc(-c2ccc(F)cc2)c1-c1cc(C)nc(C(F)F)c1] | (R)-3-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)propane- 1,2-diol | 530.3 | | | 2390 | |
| 289 | 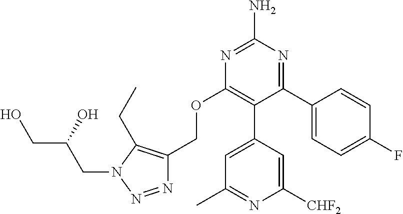 [SMILES: CCc1c(COc2nc(N)nc(-c3ccc(F)cc3)c2-c2cc(C)nc(C(F)F)c2)nnn1C[C@@H](O)CO] | (R)-3-(4-(((2-amino-5-(2- (difluoromethyl)-6- methylpyridin-4-yl)-6-(4- fluorophenyl)pyrimidin-4- yl)oxy)methyl)-5-ethyl-1H- 1,2,3-triazol-1-yl)propane- 1,2-diol | 530.2 | | | 2088 | |
| 290 | 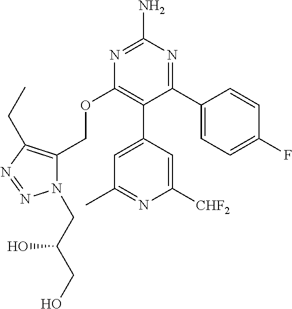 [SMILES: CCc1nnn(C[C@@H](O)CO)c1COc1nc(N)nc(-c2ccc(F)cc2)c1-c1cc(C)nc(C(F)F)c1] | (R)-3-(5-(((2-amino-5-(2- (trifluoromethyl)-6- methylpyridin-4-yl)-6-(4- fluorophenyl)pyrimidin-4- yl)oxy)methyl)-4-ethyl-1H- 1,2,3-triazol-1-yl)propane- 1,2-diol | 530.3 | | | 1313 | |
| 291 | 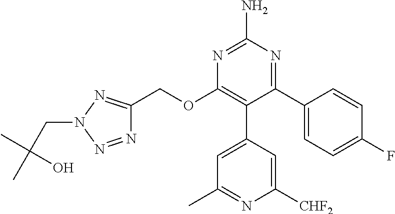 [SMILES: Cc1cc(-c2c(OCc3nnn(CC(C)(C)O)n3)nc(N)nc2-c2ccc(F)cc2)cc(C(F)F)n1] | 1-(5-(((2-amino-5-(2- (difluoromethyl)-6- methylpyridin-4-yl)-6-(4- fluorophenyl)pyrimidin-4- yl)oxy)methyl)-2H- tetrazol-2-yl)-2- methylpropan-2-ol | 501.2 | | | 5492 | |
| 292 | 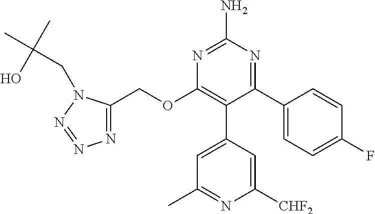 [SMILES: Cc1cc(-c2c(OCc3nnnn3CC(C)(C)O)nc(N)nc2-c2ccc(F)cc2)cc(C(F)F)n1] | 1-(5-(((2-amino-5-(2- (difluoromethyl)-6- methylpyridin-4-yl)-6-(4- fluorophenyl)pyrimidin-4- yl)oxy)methyl)-1H- tetrazol-1-yl)-2- methylpropan-2-ol | 501.2 | | | 708 | |
| 293 | 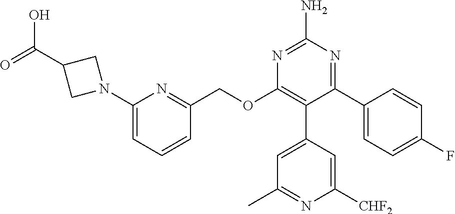 [SMILES: Cc1cc(-c2c(OCc3cccc(N4CC(C(=O)O)C4)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)azetidine-3-carboxylic acid | 537.2 | | | 4351 | |
| 294 | 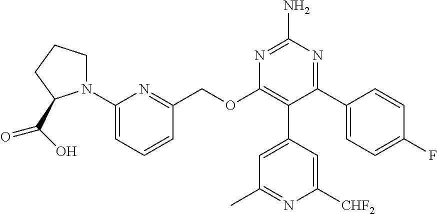 [SMILES: Cc1cc(-c2c(OCc3cccc(N4CCC[C@@H]4C(=O)O)n3)nc(N)nc2-c2ccc(F)cc2)cc(C(F)F)n1] | (6-(((2-amino-5-(2- (difluoromethyl)-6- methylpyridin-4-yl)-6-(4- fluorophenyl)pyrimidin-4- yl)oxy)methyl)pyridin-2- yl)-D-proline | 551.2 | | | 19654 | |
| 295 | 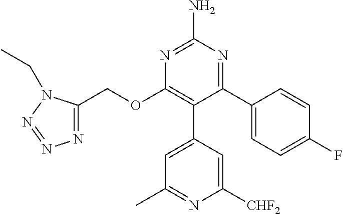 [SMILES: CCn1nnnc1COc1nc(N)nc(-c2ccc(F)cc2)c1-c1cc(C)nc(C(F)F)c1] | 5-(2-(difluoromethyl)-6- methylpyridin-4-yl)-4-((1- ethyl-1H-tetrazol-5- yl)methoxy)-6-(4- fluorophenyl)pyrimidin-2- amine | 457.0 | 7.4 | | 175/69/ 201 | |
| 296 | 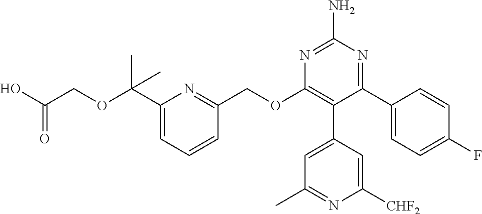 [SMILES: Cc1cc(-c2c(OCc3cccc(C(C)(C)OCC(=O)O)n3)nc(N)nc2-c2ccc(F)cc2)cc(C(F)F)n1] | 2-((2-(6-(((2-amino-5-(2- (difluoromethyl)-6- methylpyridin-4-yl)-6-(4- fluorophenyl)pyrimidin-4- yl)oxy)methyl)pyridin-2- yl)propan-2-yl)oxy)acetic acid | 554.2 | | | 3628 | |
| 297 | 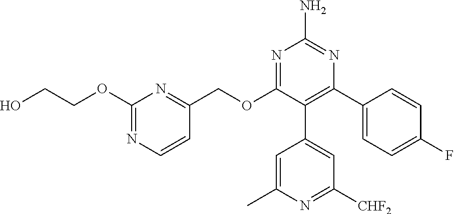 [SMILES: Cc1cc(-c2c(OCc3ccnc(OCCO)n3)nc(N)nc2-c2ccc(F)cc2)cc(C(F)F)n1] | 2-((4-(((2-amino-5-(2- (difluoromethyl)-6- methylpyridin-4-yl)-6-(4- fluorophenyl)pyrimidin-4- yl)oxy)methyl)pyrimidin-2- yl)oxy)ethan-1-ol | 499.2 | | | 278/ 228 | 521 |
| 298 | 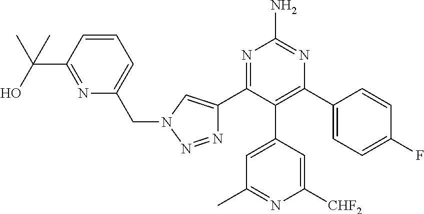 [SMILES: Cc1cc(-c2c(-c3ccc(F)cc3)nc(N)nc2-c2cn(Cc3cccc(C(C)(C)O)n3)nn2)cc(C(F)F)n1] | 2-(6-((4-(2-amino-5-(2- (difluoromethyl)-6- methylpyridin-4-yl)-6-(4- fluorophenyl)pyrimidin-4- yl)-1H-1,2,3-triazol-1- yl)methyl)pyridin-2- yl)propan-2-ol | 547.2 | | | >10000 | |
| 299 | 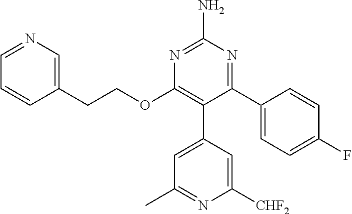 [SMILES: Cc1cc(-c2c(OCCc3cccnc3)nc(N)nc2-c2ccc(F)cc2)cc(C(F)F)n1] | 5-(2-(difluoromethyl)-6- methylpyridin-4-yl)-4-(4- fluorophenyl)-6-(2- (pyridin-3- yl)ethoxy)pyrimidin-2- amine | 452.2 | | | 1112 | |
| 300 | 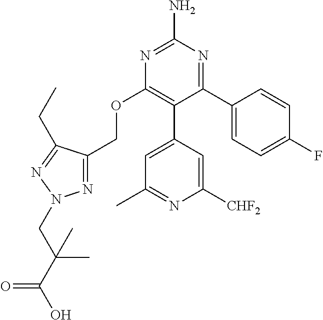 [SMILES: CCc1nn(CC(C)(C)C(=O)O)nc1COc1nc(N)nc(-c2ccc(F)cc2)c1-c1cc(C)nc(C(F)F)c1] | 3-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)-2,2- dimethylpropanoic acid | 556.3 | | | >10000 | |
| 301 | 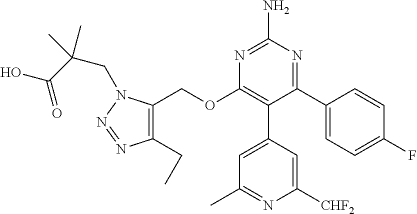 [SMILES: CCc1nnn(CC(C)(C)C(=O)O)c1COc1nc(N)nc(-c2ccc(F)cc2)c1-c1cc(C)nc(C(F)F)c1] | 3-(5-(((2-amino-5-(2- (difluoromethyl)-6- methylpyridin-4-yl)-6-(4- fluorophenyl)pyrimidin-4- yl)oxy)methyl)-4-ethyl-1H- 1,2,3-triazol-1-yl)-2,2- dimethylpropanoic acid | 556.2 | | | >10000 | |
| 302 | 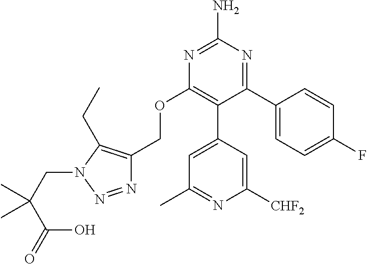 [SMILES: CCc1c(COc2nc(N)nc(-c3ccc(F)cc3)c2-c2cc(C)nc(C(F)F)c2)nnn1CC(C)(C)C(=O)O] | 3-(4-(((2-amino-5-(2- (difluoromethyl)-6- methylpyridin-4-yl)-6-(4- fluorophenyl)pyrimidin-4- yl)oxy)methyl)-5-ethyl-1H- 1,2,3-triazol-1-yl)-2,2- dimethylpropanoic acid | 556.2 | | | >10000 | |
| 303 | 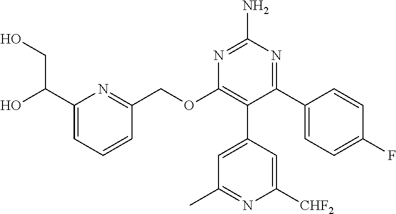 [SMILES: Cc1cc(-c2c(OCc3cccc(C(O)CO)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)ethane-1,2-diol | 498.2 | | | 965 | |
| 304 | 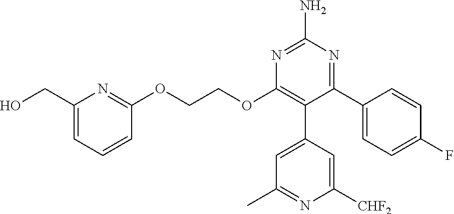 [SMILES: Cc1cc(-c2c(OCCOc3cccc(CO)n3)nc(N)nc2-c2ccc(F)cc2)cc(C(F)F)n1] | (6-(2-((2-amino-5-(2- (difluoromethyl)-6- methylpyridin-4-yl)-6-(4- fluorophenyl)pyrimidin-4- yl)oxy)ethoxy)pyridin-2- yl)methanol | 498.1 | | | 681 | |
| 305 | 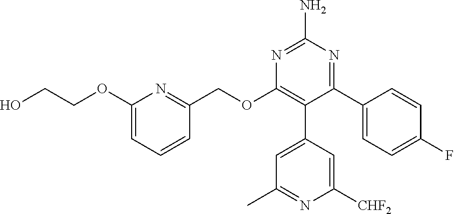 [SMILES: Cc1cc(-c2c(OCc3cccc(OCCO)n3)nc(N)nc2-c2ccc(F)cc2)cc(C(F)F)n1] | 2-((6-(((2-amino-5-(2- (difluoromethyl)-6- methylpyridin-4-yl)-6-(4- fluorophenyl)pyrimidin-4- yl)oxy)methyl)pyridin-2- yl)oxy)ethan-1-ol | 498.2 | | | 196 | 61.7 |
| 306 | 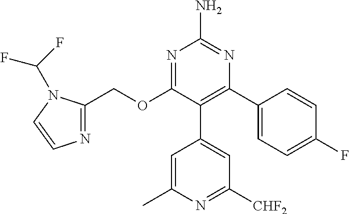 [SMILES: Cc1cc(-c2c(OCc3nccn3C(F)F)nc(N)nc2-c2ccc(F)cc2)cc(C(F)F)n1] | 4-((1-(difluoromethyl)-1H- imidazol-2-yl)methoxy)-5- (2-(difluoromethyl)-6- methylpyridin-4-yl)-6-(4- fluorophenyl)pyrimidin-2- amine | 477.2 | | | 26/23 | 6.9 |
| 307 | 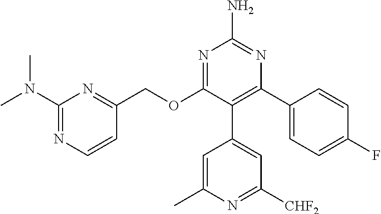 [SMILES: Cc1cc(-c2c(OCc3ccnc(N(C)C)n3)nc(N)nc2-c2ccc(F)cc2)cc(C(F)F)n1] | 4-(((2-amino-5-(2- (difluoromethyl)-6- methylpyridin-4-yl)-6-(4- fluorophenyl)pyrimidin-4- yl)oxy)methyl)-N,N- dimethylpyrimidin-2- amine | 482.2 | | | 211/ 246 | |
| 308 | 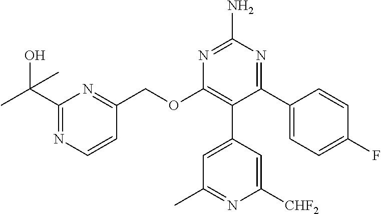 [SMILES: Cc1cc(-c2c(OCc3ccnc(C(C)(C)O)n3)nc(N)nc2-c2ccc(F)cc2)cc(C(F)F)n1] | 2-(4-(((2-amino-5-(2- (difluoromethyl)-6- methylpyridin-4-yl)-6-(4- fluorophenyl)pyrimidin-4- yl)oxy)methyl)pyrimidin-2- yl)propan-2-ol | 497.2 | | | 164/ 197/ 159/ 113 | 28.0/ 7.0 |
| 309 | 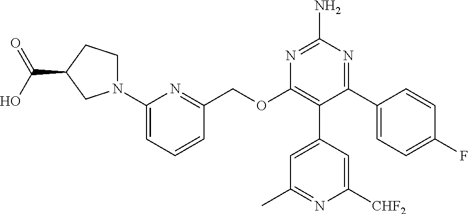 [SMILES: Cc1cc(-c2c(OCc3cccc(N4CC[C@H](C(=O)O)C4)n3)nc(N)nc2-c2ccc(F)cc2)cc(C(F)F)n1] | (S)-1-(6-(((2-amino-5-(2- (difluoromethyl)-6- methylpyridin-4-yl)-6-(4- fluorophenyl)pyrimidin-4- yl)oxy)methyl)pyridin-2- yl)pyrrolidine-3-carboxylic acid | 551.2 | | | 1373 | |
| 310 | 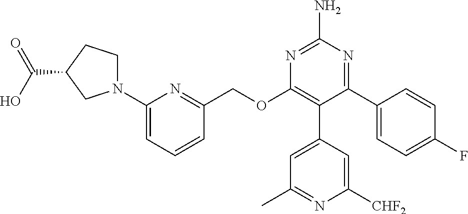 [SMILES: Cc1cc(-c2c(OCc3cccc(N4CC[C@@H](C(=O)O)C4)n3)nc(N)nc2-c2ccc(F)cc2)cc(C(F)F)n1] | (R)-1-(6-(((2-amino-5-(2- (difluoromethyl)-6- methylpyridin-4-yl)-6-(4- fluorophenyl)pyrimidin-4- yl)oxy)methyl)pyridin-2- yl)pyrrolidine-3-carboxylic acid | 551.2 | | | 2488 | |
| 311 | 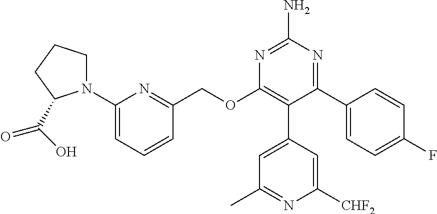 [SMILES: Cc1cc(-c2c(OCc3cccc(N4CCC[C@H]4C(=O)O)n3)nc(N)nc2-c2ccc(F)cc2)cc(C(F)F)n1] | (6-(((2-amino-5-(2- (difluoromethyl)-6- methylpyridin-4-yl)-6-(4- fluorophenyl)pyrimidin-4- yl)oxy)methyl)pyridin-2- yl)-L-proline | 551.2 | | | >10000 | |
| 312 | 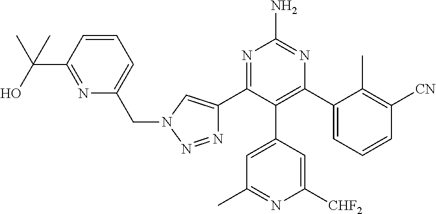 [SMILES: Cc1cc(-c2c(-c3cn(Cc4cccc(C(C)(C)O)n4)nn3)nc(N)nc2-c2cccc(C#N)c2C)cc(C(F)F)n1] | 3-(2-amino-5-(2- (difluoromethyl)-6- methylpyridin-4-yl)-6-(1- ((6-(2-hydroxypropan-2- yl)pyridin-2-yl)methyl)-1H- 1,2,3-triazol-4- yl)pyrimidin-4-yl)-2- methylbenzonitrile | 568.3 | | | 16624 | |
| 313 | 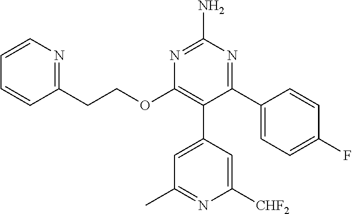 [SMILES: Cc1cc(-c2c(OCCc3ccccn3)nc(N)nc2-c2ccc(F)cc2)cc(C(F)F)n1] | 5-(2-(difluoromethyl)-6- methylpyridin-4-yl)-4-(4- fluorophenyl)-6-(2- (pyridin-2- yl)ethoxy)pyrimidin-2- amine | 452.1 | | | 20/64 | 71.5 |
| 314 | 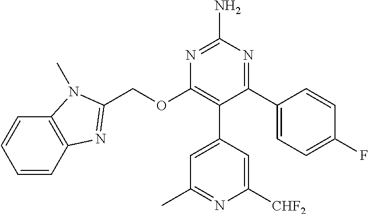 [SMILES: Cc1cc(-c2c(OCc3nc4ccccc4n3C)nc(N)nc2-c2ccc(F)cc2)cc(C(F)F)n1] | 5-(2-(difluoromethyl)-6- methylpyridin-4-yl)-4-(4- fluorophenyl)-6-((1- methyl-1H- benzo[d]imidazol-2- yl)methoxy)pyrimidin-2- amine | 491.2 | | | 0.4/1 | 9.8 |
| 315 | 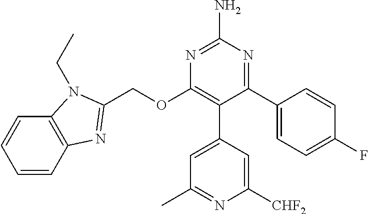 [SMILES: CCn1c(COc2nc(N)nc(-c3ccc(F)cc3)c2-c2cc(C)nc(C(F)F)c2)nc2ccccc21] | 5-(2-(difluoromethyl)-6- methylpyridin-4-yl)-4-((1- ethyl-1H- benzo[d]imidazol-2- yl)methoxy)-6-(4- fluorophenyl)pyrimidin-2- amine | 505.2 | | | 1.8/15 | 24.8 |
| 316 | 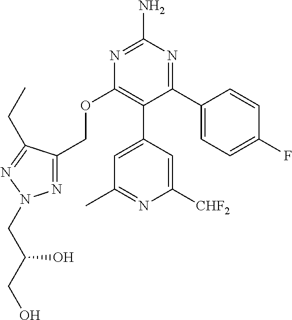 [SMILES: CCc1nn(C[C@H](O)CO)nc1COc1nc(N)nc(-c2ccc(F)cc2)c1-c1cc(C)nc(C(F)F)c1] | (S)-3-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)propane- 1,2-diol | 530.2 | | | 1081 | |
| 317 | 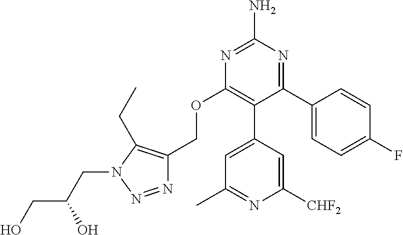 [SMILES: CCc1c(COc2nc(N)nc(-c3ccc(F)cc3)c2-c2cc(C)nc(C(F)F)c2)nnn1C[C@H](O)CO] | (S)-3-(4-(((2-amino-5-(2- (difluoromethyl)-6- methylpyridin-4-yl)-6-(4- fluorophenyl)pyrimidin-4- yl)oxy)methyl)-5-ethyl-1H- 1,2,3-triazol-1-yl)propane- 1,2-diol | 530.2 | | | 1434 | |
| 318 | 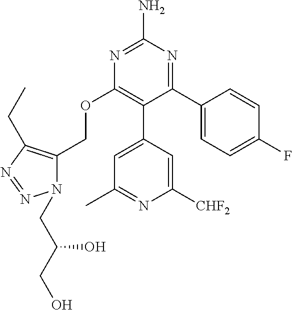 [SMILES: CCc1nnn(C[C@H](O)CO)c1COc1nc(N)nc(-c2ccc(F)cc2)c1-c1cc(C)nc(C(F)F)c1] | (S)-3-(5-(((2-amino-5-(2- (difluoromethyl)-6- methylpyridin-4-yl)-6-(4- fluorophenyl)pyrimidin-4- yl)oxy)methyl)-4-ethyl-1H- 1,2,3-triazol-1-yl)propane- 1,2-diol | 530.2 | | | 1353 | |
| 319 | 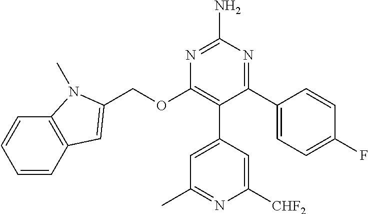 [SMILES: Cc1cc(-c2c(OCc3cc4ccccc4n3C)nc(N)nc2-c2ccc(F)cc2)cc(C(F)F)n1] | 5-(2-(difluoromethyl)-6- methylpyridin-4-yl)-4-(4- fluorophenyl)-6-((1- methyl-1H-indol-2- yl)methoxy)pyrimidin-2- amine | 490.2 | | | 209/ 256 | |
| 320 | 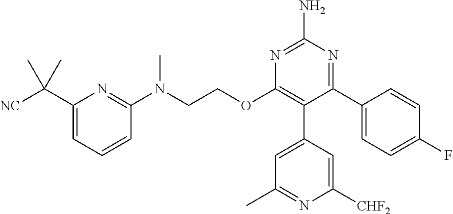 [SMILES: Cc1cc(-c2c(OCCN(C)c3cccc(C(C)(C)C#N)n3)nc(N)nc2-c2ccc(F)cc2)cc(C(F)F)n1] | 2-(6-((2-((2-amino-5-(2- (difluoromethyl)-6- methylpyridin-4-yl)-6-(4- fluorophenyl)pyrimidin-4- yl)oxy)ethyl)(methyl) amino)pyridin-2-yl)-2- methylpropanenitrile | 548.2 | | | 13/26/ 8.7 | 141.4 |
| 321 | 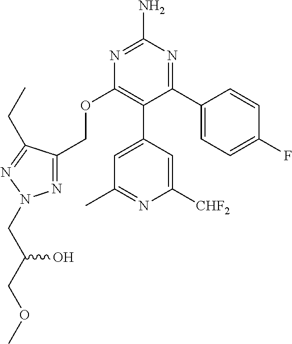 [SMILES: CCc1nn(CC(O)COC)nc1COc1nc(N)nc(-c2ccc(F)cc2)c1-c1cc(C)nc(C(F)F)c1] | 1-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)-3- methoxypropan-2-ol | 544.2 | | | 2288 | |
| 322 | 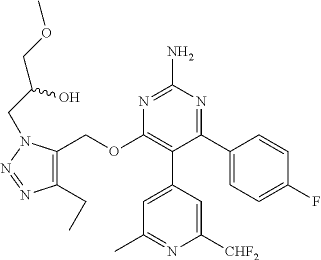 [SMILES: CCc1nnn(CC(O)COC)c1COc1nc(N)nc(-c2ccc(F)cc2)c1-c1cc(C)nc(C(F)F)c1] | 1-(5-(((2-amino-5-(2- (difluoromethyl)-6- methylpyridin-4-yl)-6-(4- fluorophenyl)pyrimidin-4- yl)oxy)methyl)-4-ethyl-1H- 1,2,3-triazol-1-yl)-3- methoxypropan-2-ol | 544.2 | | | 2471 | |
| 323 | 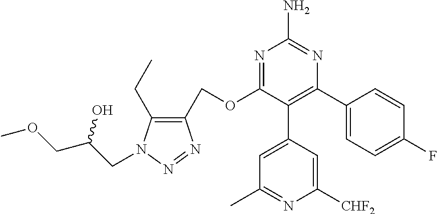 [SMILES: CCc1c(COc2nc(N)nc(-c3ccc(F)cc3)c2-c2cc(C)nc(C(F)F)c2)nnn1CC(O)COC] | 1-(4-(((2-amino-5-(2- (difluoromethyl)-6- methylpyridin-4-yl)-6-(4- fluorophenyl)pyrimidin-4- yl)oxy)methyl)-5-ethyl-1H- 1,2,3-triazol-1-yl)-3- methoxypropan-2-ol | 544.2 | | | 1678 | |
| 324 | 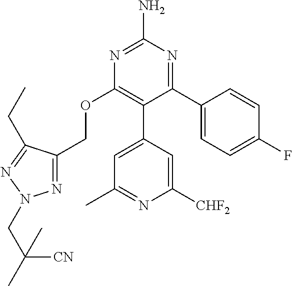 [SMILES: CCc1nn(CC(C)(C)C#N)nc1COc1nc(N)nc(-c2ccc(F)cc2)c1-c1cc(C)nc(C(F)F)c1] | 3-(4-(((2-amino-5-(2- (difluoromethyl)-6- methylpyridin-4-yl)-6-(4- fluorophenyl)pyrimidin-4- yl)oxy)methyl)-5-ethyl-2H- 1,2,3-triazol-2-yl)-2,2- dimethylpropanenitrile | 537.3 | | | 2083 | |
| 325 | 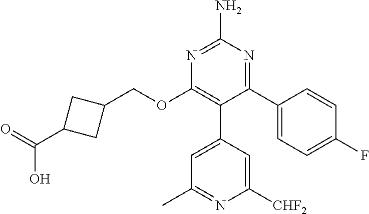 [SMILES: Cc1cc(-c2c(OCC3CC(C(=O)O)C3)nc(N)nc2-c2ccc(F)cc2)cc(C(F)F)n1] | 3-(((2-amino-5-(2- (difluoromethyl)-6- methylpyridin-4-yl)-6-(4- fluorophenyl)pyrimidin-4- yl)oxy)methyl)cyclobutane- 1-carboxylic acid | 459.1 | | | >25000 | |
| 326 | 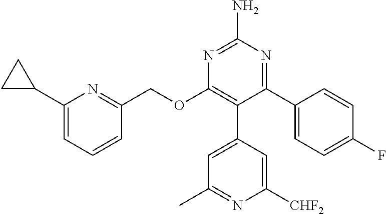 [SMILES: Cc1cc(-c2c(OCc3cccc(C4CC4)n3)nc(N)nc2-c2ccc(F)cc2)cc(C(F)F)n1] | 4-((6-cyclopropylpyridin- 2-yl)methoxy)-5-(2- (difluoromethyl)-6- methylpyridin-4-yl)-6-(4- fluorophenyl)pyrimidin-2- amine | 478.2 | | | 90/144/ 84 | 57.1 |
| 327 | 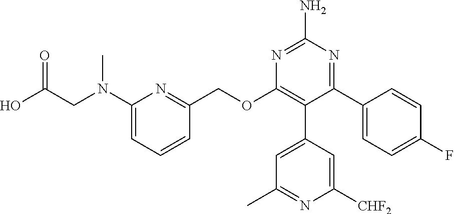 [SMILES: Cc1cc(-c2c(OCc3cccc(N(C)CC(=O)O)n3)nc(N)nc2-c2ccc(F)cc2)cc(C(F)F)n1] | N-(6-(((2-amino-5-(2- (difluoromethyl)-6- methylpyridin-4-yl)-6-(4- fluorophenyl)pyrimidin-4- yl)oxy)methyl)pyridin-2- yl)-N-methylglycine | 525.2 | | | 4391 | |
| 328 | 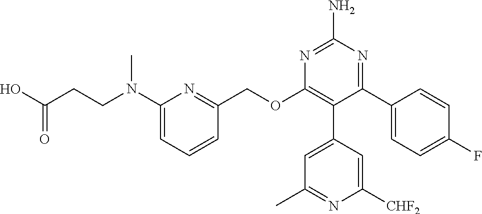 [SMILES: Cc1cc(-c2c(OCc3cccc(N(C)CCC(=O)O)n3)nc(N)nc2-c2ccc(F)cc2)cc(C(F)F)n1] | 3-((6-(((2-amino-5-(2- (difluoromethyl)-6- methylpyridin-4-yl)-6-(4- fluorophenyl)pyrimidin-4- yl)oxy)methyl)pyridin-2- yl)(methyl)amino) propanoic acid | 539.2 | | | 4513 | |
| 329 | 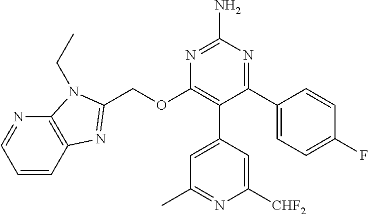 [SMILES: CCn1c(COc2nc(N)nc(-c3ccc(F)cc3)c2-c2cc(C)nc(C(F)F)c2)nc2cccnc21] | 5-(2-(difluoromethyl)-6- methylpyridin-4-yl)-4-((3- ethyl-3H-imidazo[4,5- b]pyridin-2-yl)methoxy)-6- (4-fluorophenyl)pyrimidin- 2-amine | 506.2 | | | 87/249/ 153 | 127.8 |
| 330 | 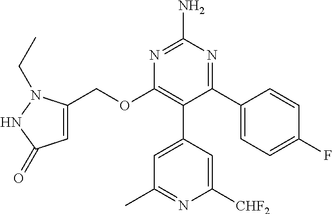 [SMILES: CCn1[nH]c(=O)cc1COc1nc(N)nc(-c2ccc(F)cc2)c1-c1cc(C)nc(C(F)F)c1] | 5-(((2-amino-5-(2- (difluoromethyl)-6- methylpyridin-4-yl)-6-(4- fluorophenyl)pyrimidin-4- yl)oxy)methyl)-1-ethyl- 1,2-dihydro-3H-pyrazol-3- one | 471.2 | | | 2551 | |
| 331 | 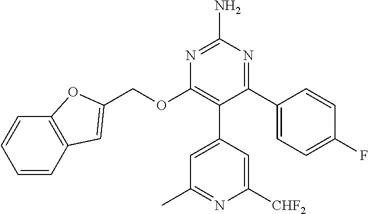 [SMILES: Cc1cc(-c2c(OCc3cc4ccccc4o3)nc(N)nc2-c2ccc(F)cc2)cc(C(F)F)n1] | 4-(benzofuran-2- ylmethoxy)-5-(2- (difluoromethyl)-6- methylpyridin-4-yl)-6-(4- fluorophenyl)pyrimidin-2- amine | 477.2 | | | 648 | |
| 332 | 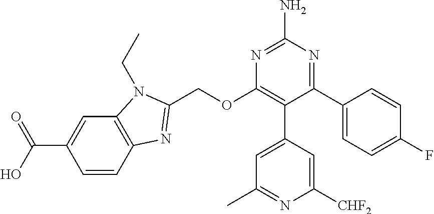 [SMILES: CCn1c(COc2nc(N)nc(-c3ccc(F)cc3)c2-c2cc(C)nc(C(F)F)c2)nc2ccc(C(=O)O)cc21] | 2-(((2-amino-5-(2- (difluoromethyl)-6- methylpyridin-4-yl)-6-(4- fluorophenyl)pyrimidin-4- yl)oxy)methyl)-1-ethyl-1H- benzo[d]imidazole-6- carboxylic acid | 549.2 | | | >5000 | |
| 333 | 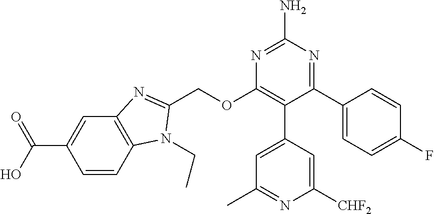 [SMILES: CCn1c(COc2nc(N)nc(-c3ccc(F)cc3)c2-c2cc(C)nc(C(F)F)c2)nc2cc(C(=O)O)ccc21] | 2-(((2-amino-5-(2- (difluoromethyl)-6- methylpyridin-4-yl)-6-(4- fluorophenyl)pyrimidin-4- yl)oxy)methyl)-1-ethyl-1H- benzo[d]imidazole-5- carboxylic acid | 549.2 | | | >5000 | |
| 334 | 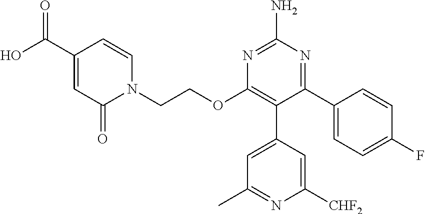 [SMILES: Cc1cc(-c2c(OCCn3ccc(C(=O)O)cc3=O)nc(N)nc2-c2ccc(F)cc2)cc(C(F)F)n1] | 1-(2-((2-amino-5-(2- (difluoromethyl)-6- methylpyridin-4-yl)-6-(4- fluorophenyl)pyrimidin-4- yl)oxy)ethyl)-2-oxo-1,2- dihydropyridine-4- carboxylic acid | 512.2 | | | 3166 | |
| 335 | 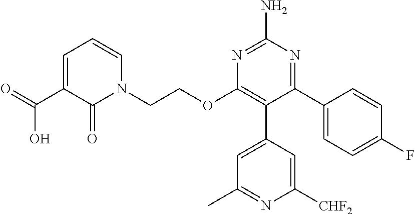 [SMILES: Cc1cc(-c2c(OCCn3cccc(C(=O)O)c3=O)nc(N)nc2-c2ccc(F)cc2)cc(C(F)F)n1] | 1-(2-((2-amino-5-(2- (difluoromethyl)-6- methylpyridin-4-yl)-6-(4- fluorophenyl)pyrimidin-4- yl)oxy)ethyl)-2-oxo-1,2- dihydropyridine-3- carboxylic acid | 512.2 | | | >25000 | |
| 336 | 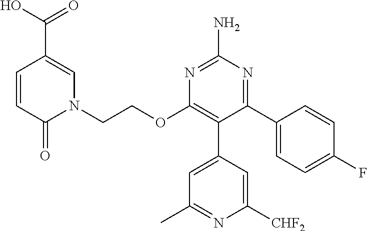 [SMILES: Cc1cc(-c2c(OCCn3cc(C(=O)O)ccc3=O)nc(N)nc2-c2ccc(F)cc2)cc(C(F)F)n1] | 1-(2-((2-amino-5-(2- (difluoromethyl)-6- methylpyridin-4-yl)-6-(4- fluorophenyl)pyrimidin-4- yl)oxy)ethyl)-6-oxo-1,6- dihydropyridine-3- carboxylic acid | 512.2 | | | >25000 | |
| 337 | 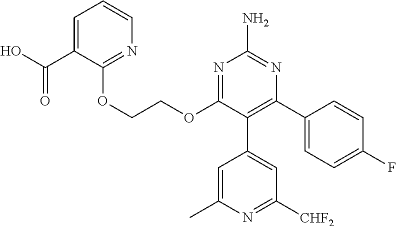 [SMILES: Cc1cc(-c2c(OCCOc3ncccc3C(=O)O)nc(N)nc2-c2ccc(F)cc2)cc(C(F)F)n1] | 2-(2-((2-amino-5-(2- (difluoromethyl)-6- methylpyridin-4-yl)-6-(4- fluorophenyl)pyrimidin-4- yl)oxy)ethoxy)nicotinic acid | 512.2 | | | >25000 | |
| 338 | 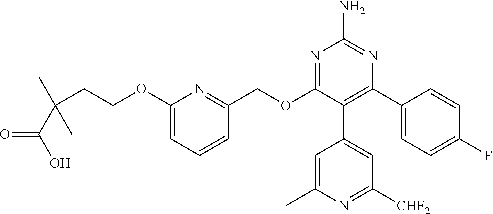 [SMILES: Cc1cc(-c2c(OCc3cccc(OCCC(C)(C)C(=O)O)n3)nc(N)nc2-c2ccc(F)cc2)cc(C(F)F)n1] | 4-((6-(((2-amino-5-(2- (difluoromethyl)-6- methylpyridin-4-yl)-6-(4- fluorophenyl)pyrimidin-4- yl)oxy)methyl)pyridin-2- yl)oxy)-2,2- dimethylbutanoic acid | 568.2 | | | 2889 | |
| 339 | 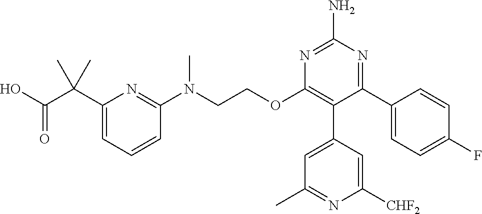 [SMILES: Cc1cc(-c2c(OCCN(C)c3cccc(C(C)(C)C(=O)O)n3)nc(N)nc2-c2ccc(F)cc2)cc(C(F)F)n1] | 2-(6-((2-((2-amino-5-(2- (difluoromethyl)-6- methylpyridin-4-yl)-6-(4- fluorophenyl)pyrimidin-4- yl)oxy)ethyl)(methyl) amino)pyridin-2-yl)-2- methylpropanoic acid | 567.2 | | | 561/ 795/ 230 | 166 |
| 340 | 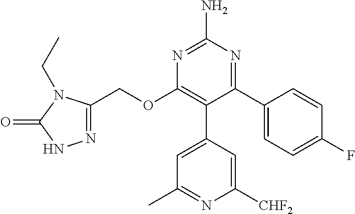 [SMILES: CCn1c(COc2nc(N)nc(-c3ccc(F)cc3)c2-c2cc(C)nc(C(F)F)c2)n[nH]c1=O] | 5-(((2-amino-5-(2- (difluoromethyl)-6- methylpyridin-4-yl)-6-(4- fluorophenyl)pyrimidin-4- yl)oxy)methyl)-4-ethyl- 2,4-dihydro-3H-1,2,4- triazol-3-one | 472.2 | | | 4078 | |
| 341 | 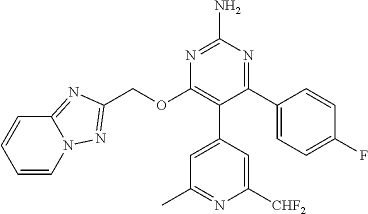 [SMILES: Cc1cc(-c2c(OCc3nc4ccccn4n3)nc(N)nc2-c2ccc(F)cc2)cc(C(F)F)n1] | 4-([1,2,4]triazolo[1,5- a]pyridin-2-ylmethoxy)-5- (2-(difluoromethyl)-6- methylpyridin-4-yl)-6-(4- fluorophenyl)pyrimidin-2- amine | 478.1 | | | 1401 | |
| 342 | 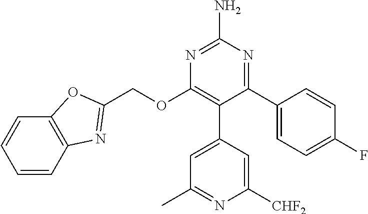 [SMILES: Cc1cc(-c2c(OCc3nc4ccccc4o3)nc(N)nc2-c2ccc(F)cc2)cc(C(F)F)n1] | 4-(benzo[d]oxazol-2- ylmethoxy)-5-(2- (difluoromethyl)-6- methylpyridin-4-yl)-6-(4- fluorophenyl)pyrimidin-2- amine | 478.1 | | | 419 | |
| 343 | 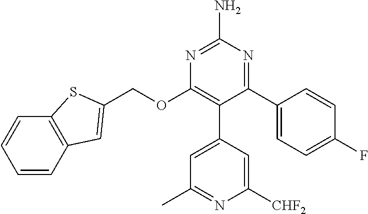 [SMILES: Cc1cc(-c2c(OCc3cc4ccccc4s3)nc(N)nc2-c2ccc(F)cc2)cc(C(F)F)n1] | 4-(benzo[b]thiophen-2- ylmethoxy)-5-(2- (difluoromethyl)-6- methylpyridin-4-yl)-6-(4- fluorophenyl)pyrimidin-2- amine | 493.1 | | | 1337 | |
| 344 | 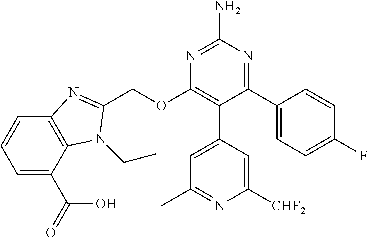 [SMILES: CCn1c(COc2nc(N)nc(-c3ccc(F)cc3)c2-c2cc(C)nc(C(F)F)c2)nc2cccc(C(=O)O)c21] | 2-(((2-amino-5-(2- (difluoromethyl)-6- methylpyridin-4-yl)-6-(4- fluorophenyl)pyrimidin-4- yl)oxy)methyl)-1-ethyl-1H- benzo[d]imidazole-7- carboxylic acid | 549.2 | | | >25000 | |
| 345 | 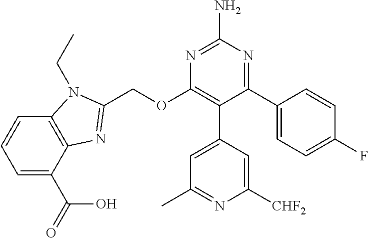 [SMILES: CCn1c(COc2nc(N)nc(-c3ccc(F)cc3)c2-c2cc(C)nc(C(F)F)c2)nc2c(C(=O)O)cccc21] | 2-(((2-amino-5-(2- (difluoromethyl)-6- methylpyridin-4-yl)-6-(4- fluorophenyl)pyrimidin-4- yl)oxy)methyl)-1-ethyl-1H- benzo[d]imidazole-4- carboxylic acid | 549.2 | | | 10321 | |
| 346 | 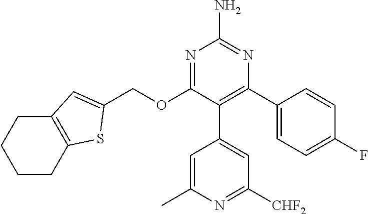 [SMILES: Cc1cc(-c2c(OCc3cc4c(s3)CCCC4)nc(N)nc2-c2ccc(F)cc2)cc(C(F)F)n1] | 5-(2-(difluoromethyl)-6- methylpyridin-4-yl)-4-(4- fluorophenyl)-6-((4,5,6,7- tetrahydrobenzo[b] thiophen-2- yl)methoxy)pyrimidin-2- amine | 497.1 | | | 3993 | |
| 347 | 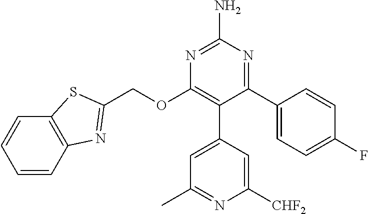 [SMILES: Cc1cc(-c2c(OCc3nc4ccccc4s3)nc(N)nc2-c2ccc(F)cc2)cc(C(F)F)n1] | 4-(benzo[d]thiazol-2- ylmethoxy)-5-(2- (difluoromethyl)-6- methylpyridin-4-yl)-6-(4- fluorophenyl)pyrimidin-2- amine | 494.1 | | | 634 | |
| 348 | 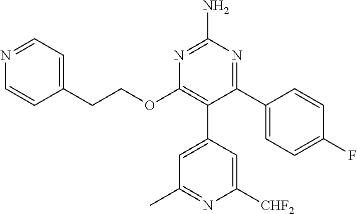 [SMILES: Cc1cc(-c2c(OCCc3ccncc3)nc(N)nc2-c2ccc(F)cc2)cc(C(F)F)n1] | 5-(2-(difluoromethyl)-6- methylpyridin-4-yl)-4-(4- fluorophenyl)-6-(2- (pyridin-4- yl)ethoxy)pyrimidin-2- amine | 452.3 | | | 2618 | |
| 349 | 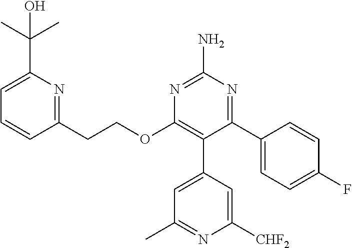 [SMILES: Cc1cc(-c2c(OCCc3cccc(C(C)(C)O)n3)nc(N)nc2-c2ccc(F)cc2)cc(C(F)F)n1] | 2-(6-(2-((2-amino-5-(2- (difluoromethyl)-6- methylpyridin-4-yl)-6-(4- fluorophenyl)pyrimidin-4- yl)oxy)ethyl)pyridin-2- yl)propan-2-ol | 510.2 | | | 261/ 120 | 55 |
| 350 | 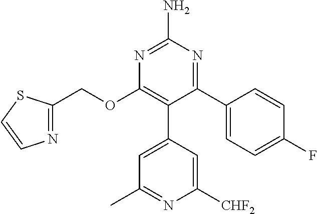 [SMILES: Cc1cc(-c2c(OCc3nccs3)nc(N)nc2-c2ccc(F)cc2)cc(C(F)F)n1] | 5-(2-(difluoromethyl)-6- methylpyridin-4-yl)-4-(4- fluorophenyl)-6-(thiazol-2- ylmethoxy)pyrimidin-2- amine | 444.1 | | | 26.1/ 5.9/ 30 | 4 |
| 351 | 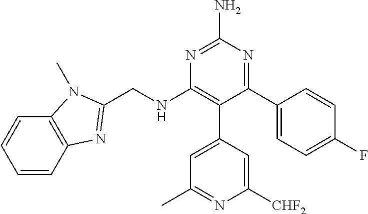 [SMILES: Cc1cc(-c2c(NCc3nc4ccccc4n3C)nc(N)nc2-c2ccc(F)cc2)cc(C(F)F)n1] | 5-(2-(difluoromethyl)-6- methylpyridin-4-yl)-6-(4- fluorophenyl)-N⁴-((1- methyl-1H- benzo[d]imidazol-2- yl)methyl)pyrimidine-2,4- diamine | 490.2 | | | 13100 | |
| 352 | 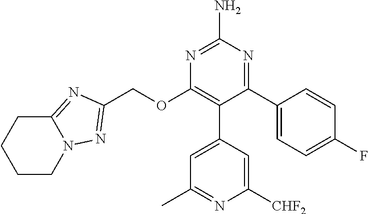 [SMILES: Cc1cc(-c2c(OCc3nc4n(n3)CCCC4)nc(N)nc2-c2ccc(F)cc2)cc(C(F)F)n1] | 5-(2-(difluoromethyl)-6- methylpyridin-4-yl)-4-(4- fluorophenyl)-6-((5,6,7,8- tetrahydro- [1,2,4]triazolo[1,5- a]pyridin-2- yl)methoxy)pyrimidin-2- amine | 482.2 | | | 1792 | |
| 353 | 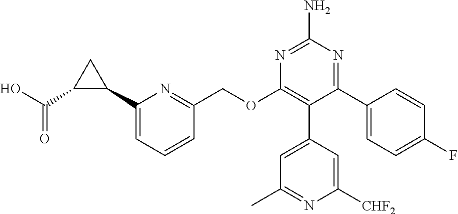 [SMILES: Cc1cc(-c2c(OCc3cccc([C@@H]4C[C@H]4C(=O)O)n3)nc(N)nc2-c2ccc(F)cc2)cc(C(F)F)n1] | (1,2-trans)-2-(6-(((2- amino-5-(2- (difluoromethyl)-6- methylpyridin-4-yl)-6-(4- fluorophenyl)pyrimidin-4- yl)oxy)methyl)pyridin-2- yl)cyclopropane-1- carboxylic acid | 522.2 | | | 1235 | 374 |
| 354 | 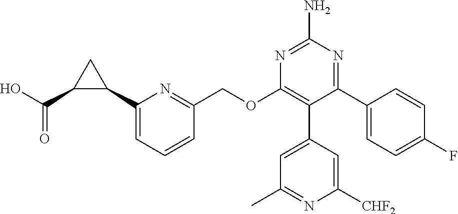 [SMILES: Cc1cc(-c2c(OCc3cccc([C@@H]4C[C@@H]4C(=O)O)n3)nc(N)nc2-c2ccc(F)cc2)cc(C(F)F)n1] | (1,2-cis)-2-(6-(((2-amino- 5-(2-(difluoromethyl)-6- methylpyridin-4-yl)-6-(4- fluorophenyl)pyrimidin-4- yl)oxy)methyl)pyridin-2- yl)cyclopropane-1- carboxylic acid | 522.2 | | | 563/ 977 | 549 |
| 355 | 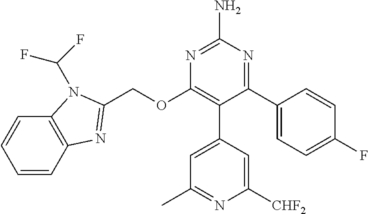 [SMILES: Cc1cc(-c2c(OCc3nc4ccccc4n3C(F)F)nc(N)nc2-c2ccc(F)cc2)cc(C(F)F)n1] | 4-((1-(difluoromethyl)-1H- benzo[d]imidazol-2- yl)methoxy)-5-(2- (difluoromethyl)-6- methylpyridin-4-yl)-6-(4- fluorophenyl)pyrimidin-2- amine | 527.1 | | | 10.2/ 11/13 | 5.7 |
| 356 | 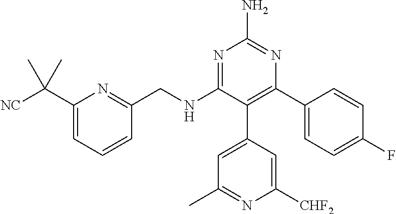 [SMILES: Cc1cc(-c2c(NCc3cccc(C(C)(C)C#N)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)amino)methyl)pyridin-2- yl)-2-methylpropanenitrile | 504.2 | | | 1421 | |
| 357 | 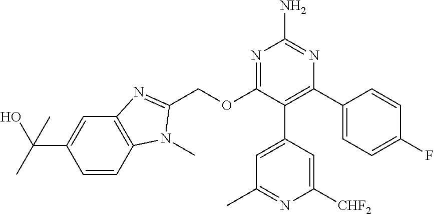 [SMILES: Cc1cc(-c2c(OCc3nc4cc(C(C)(C)O)ccc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-5- yl)propan-2-ol | 549.2 | | | 3910/ 3926 | |
| 358 | 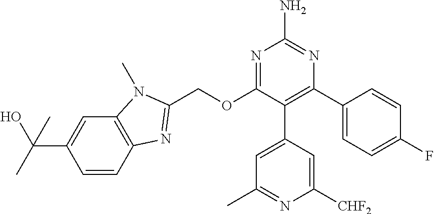 [SMILES: Cc1cc(-c2c(OCc3nc4ccc(C(C)(C)O)cc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-6- yl)propan-2-ol | 549.2 | | | 940/ 1061 | |
| 359 | 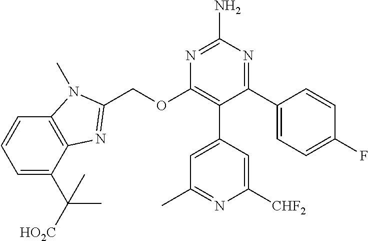 [SMILES: Cc1cc(-c2c(OCc3nc4c(C(C)(C)C(=O)O)cccc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-4- yl)-2-methylpropanoic acid | 577.2 | | | 9466/ 8889 | |
| 360 | 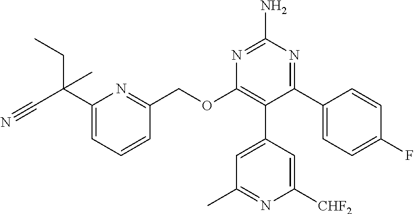 [SMILES: CCC(C)(C#N)c1cccc(COc2nc(N)nc(-c3ccc(F)cc3)c2-c2cc(C)nc(C(F)F)c2)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-2-methylbutanenitrile | 519.3 | | | 50/51/ 115 | |
| 361 | 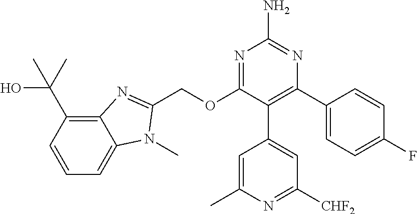 [SMILES: Cc1cc(-c2c(OCc3nc4c(C(C)(C)O)cccc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-4- yl)propan-2-ol | 549.2 | | | 854/ 1110 | |
| 362 | 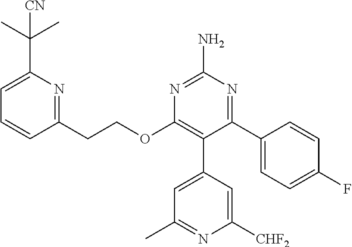 [SMILES: Cc1cc(-c2c(OCCc3cccc(C(C)(C)C#N)n3)nc(N)nc2-c2ccc(F)cc2)cc(C(F)F)n1] | 2-(6-(2-((2-amino-5-(2- (difluoromethyl)-6- methylpyridin-4-yl)-6-(4- fluorophenyl)pyrimidin-4- yl)oxy)ethyl)pyridin-2-yl)- 2-methylpropanenitrile | 519.3 | | | 85/ 158/45 | |
| 363 | 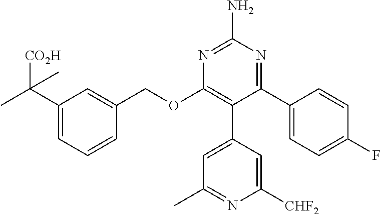 [SMILES: Cc1cc(-c2c(OCc3cccc(C(C)(C)C(=O)O)c3)nc(N)nc2-c2ccc(F)cc2)cc(C(F)F)n1] | 2-(3-(((2-amino-5-(2- (difluoromethyl)-6- methylpyridin-4-yl)-6-(4- fluorophenyl)pyrimidin-4- yl)oxy)methyl)phenyl)-2- methylpropanoic acid | 523.2 | | | 4803 | |
| 364 | 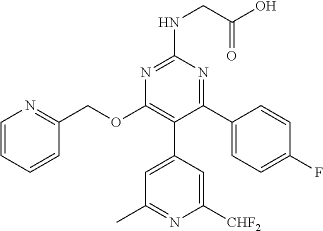 [SMILES: Cc1cc(-c2c(OCc3ccccn3)nc(NCC(=O)O)nc2-c2ccc(F)cc2)cc(C(F)F)n1] | (5-(2-(difluoromethyl)-6- methylpyridin-4-yl)-4-(4- fluorophenyl)-6-(pyridin-2- ylmethoxy)pyrimidin-2- yl)glycine | 496.2 | | | >25000 | |
| 365 | 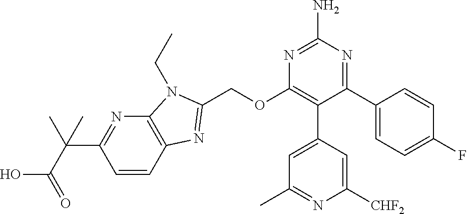 [SMILES: CCn1c(COc2nc(N)nc(-c3ccc(F)cc3)c2-c2cc(C)nc(C(F)F)c2)nc2ccc(C(C)(C)C(=O)O)nc21] | 2-(2-(((2-amino-5-(2- (difluoromethyl)-6- methylpyridin-4-yl)-6-(4- fluorophenyl)pyrimidin-4- yl)oxy)methyl)-3-ethyl-3H- imidazo[4,5-b]pyridin-5- yl)-2-methylpropanoic acid | 592.3 | | | >25000 | |
| 366 | 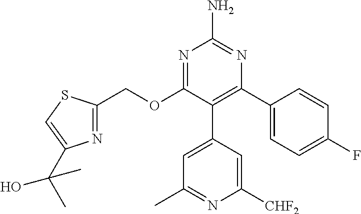 [SMILES: Cc1cc(-c2c(OCc3nc(C(C)(C)O)cs3)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)thiazol-4- yl)propan-2-ol | 502.1 | | | 1692 | |
| 367 | 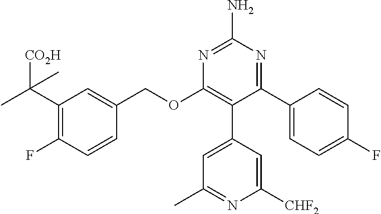 [SMILES: Cc1cc(-c2c(OCc3ccc(F)c(C(C)(C)C(=O)O)c3)nc(N)nc2-c2ccc(F)cc2)cc(C(F)F)n1] | 2-(5-(((2-amino-5-(2- (difluoromethyl)-6- methylpyridin-4-yl)-6-(4- fluorophenyl)pyrimidin-4- yl)oxy)methyl)-2- fluorophenyl)-2- methylpropanoic acid | 541.2 | | | 11782 | |
| 368 | 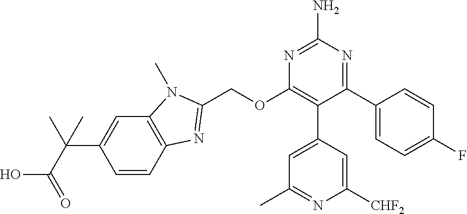 [SMILES: Cc1cc(-c2c(OCc3nc4ccc(C(C)(C)C(=O)O)cc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-6- yl)-2-methylpropanoic acid | 577.2 | | | >25000 | |
| 369 | 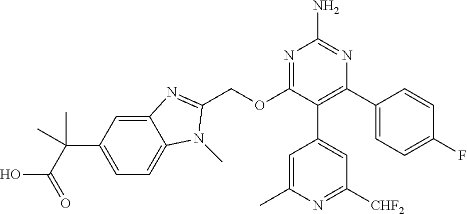 [SMILES: Cc1cc(-c2c(OCc3nc4cc(C(C)(C)C(=O)O)ccc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-5- yl)-2-methylpropanoic acid | 577.2 | | | >25000 | |
| 370 | 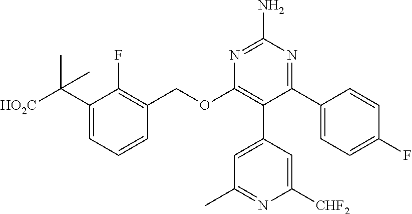 [SMILES: Cc1cc(-c2c(OCc3cccc(C(C)(C)C(=O)O)c3F)nc(N)nc2-c2ccc(F)cc2)cc(C(F)F)n1] | 2-(3-(((2-amino-5-(2- (difluoromethyl)-6- methylpyridin-4-yl)-6-(4- fluorophenyl)pyrimidin-4- yl)oxy)methyl)-2- fluorophenyl)-2- methylpropanoic acid | 541.2 | | | 5434 | |
| 371 | 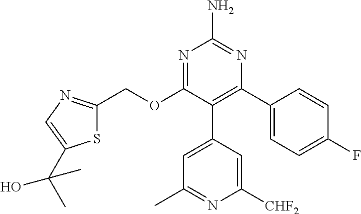 [SMILES: Cc1cc(-c2c(OCc3ncc(C(C)(C)O)s3)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)thiazol-5- yl)propan-2-ol | 502.2 | | | 2358 | |
| 372 | 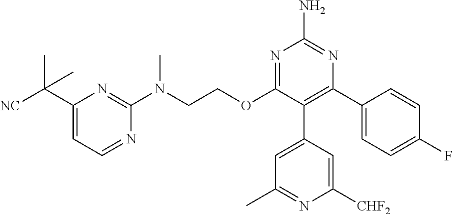 [SMILES: Cc1cc(-c2c(OCCN(C)c3nccc(C(C)(C)C#N)n3)nc(N)nc2-c2ccc(F)cc2)cc(C(F)F)n1] | 2-(2-((2-((2-amino-5-(2- (difluoromethyl)-6- methylpyridin-4-yl)-6-(4- fluorophenyl)pyrimidin-4- yl)oxy)ethyl)(methyl) amino)pyrimidin-4-yl)-2- methylpropanenitrile | 549.2 | | | 128/ 130/ 160 | |
| 373 | 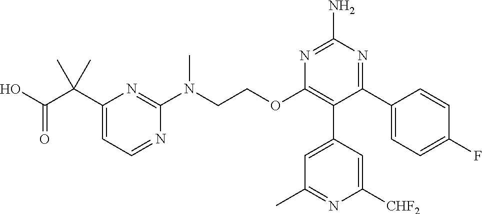 [SMILES: Cc1cc(-c2c(OCCN(C)c3nccc(C(C)(C)C(=O)O)n3)nc(N)nc2-c2ccc(F)cc2)cc(C(F)F)n1] | 2-(2-((2-((2-amino-5-(2- (difluoromethyl)-6- methylpyridin-4-yl)-6-(4- fluorophenyl)pyrimidin-4- yl)oxy)ethyl)(methyl) amino)pyrimidin-4-yl)-2- methylpropanoic acid | 568.3 | | | 240/ 248/ 387/ 191 | 421 |
| 374 | 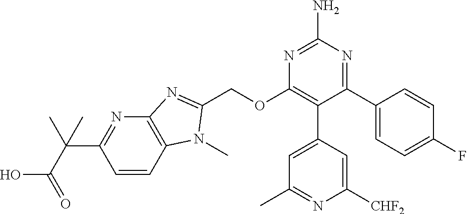 [SMILES: Cc1cc(-c2c(OCc3nc4nc(C(C)(C)C(=O)O)ccc4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-imidazo[4,5-b]pyridin- 5-yl)-2-methylpropanoic acid | 578.2 | | | >25000 | |
| 375 | 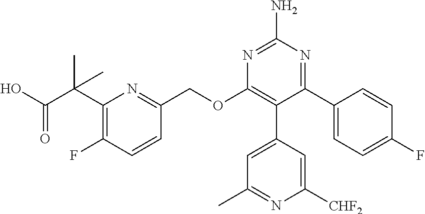 [SMILES: Cc1cc(-c2c(OCc3ccc(F)c(C(C)(C)C(=O)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)-3- fluoropyridin-2-yl)-2- methylpropanoic acid | 542.2 | | | 17602/ 23255 | |
| 376 | 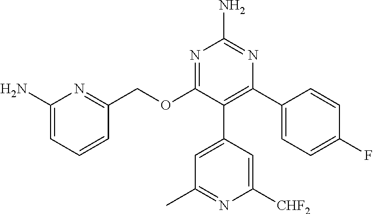 [SMILES: Cc1cc(-c2c(OCc3cccc(N)n3)nc(N)nc2-c2ccc(F)cc2)cc(C(F)F)n1] | 4-((6-aminopyridin-2- yl)methoxy)-5-(2- (difluoromethyl)-6- methylpyridin-4-yl)-6-(4- fluorophenyl)pyrimidin-2- amine | 453.2 | | | 133/ 209/ 375/ 142/48 | 7.2/ 31.4 |
| 377 | 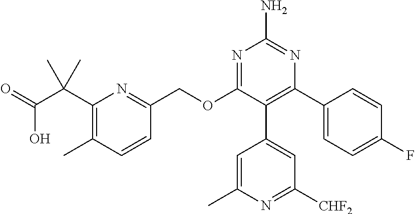 [SMILES: Cc1cc(-c2c(OCc3ccc(C)c(C(C)(C)C(=O)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)-3- methylpyridin-2-yl)-2- methylpropanoic acid | 538.3 | | | 21083 | |
| 378 | 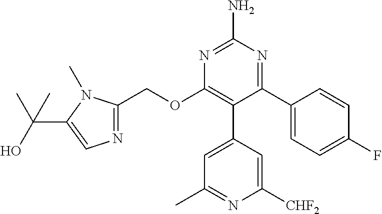 [SMILES: Cc1cc(-c2c(OCc3ncc(C(C)(C)O)n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-imidazol-5-yl)propan- 2-ol | 499.2 | | | 430/ 173/ 247 | |
| 379 | 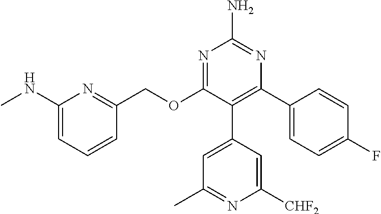 [SMILES: CNc1cccc(COc2nc(N)nc(-c3ccc(F)cc3)c2-c2cc(C)nc(C(F)F)c2)n1] | 5-(2-(difluoromethyl)-6- methylpyridin-4-yl)-4-(4- fluorophenyl)-6-((6- (methylamino)pyridin-2- yl)methoxy)pyrimidin-2- amine | 467.2 | | | 230/ 119/72 | 13.1/ 10.7 |
| 380 | 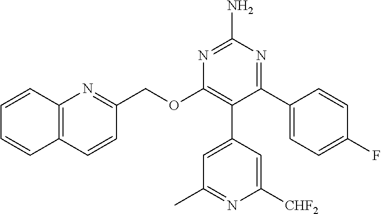 [SMILES: Cc1cc(-c2c(OCc3ccc4ccccc4n3)nc(N)nc2-c2ccc(F)cc2)cc(C(F)F)n1] | 5-(2-(difluoromethyl)-6- methylpyridin-4-yl)-4-(4- fluorophenyl)-6-(quinolin- 2-ylmethoxy)pyrimidin-2- amine | 488.2 | | | 548/ 469/ 502 | |
| 381 | 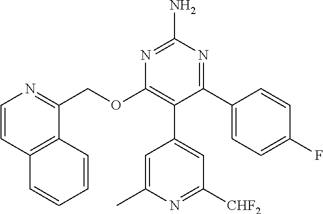 [SMILES: Cc1cc(-c2c(OCc3nccc4ccccc34)nc(N)nc2-c2ccc(F)cc2)cc(C(F)F)n1] | 5-(2-(difluoromethyl)-6- methylpyridin-4-yl)-4-(4- fluorophenyl)-6- (isoquinolin-1- ylmethoxy)pyrimidin-2- amine | 488.2 | | | 16/3.2 | 36 |
| 382 | 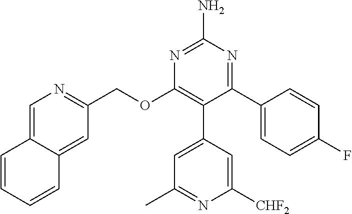 [SMILES: Cc1cc(-c2c(OCc3cc4ccccc4cn3)nc(N)nc2-c2ccc(F)cc2)cc(C(F)F)n1] | 5-(2-(difluoromethyl)-6- methylpyridin-4-yl)-4-(4- fluorophenyl)-6- (isoquinolin-3- ylmethoxy)pyrimidin-2- amine | 488.1 | | | 955 | |
| 383 | 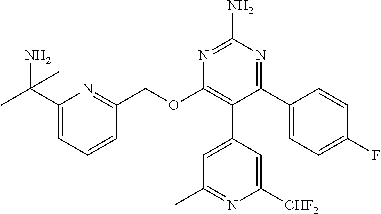 [SMILES: Cc1cc(-c2c(OCc3cccc(C(C)(C)N)n3)nc(N)nc2-c2ccc(F)cc2)cc(C(F)F)n1] | 4-((6-(2-aminopropan-2- yl)pyridin-2-yl)methoxy)- 5-(2-(difluoromethyl)-6- methylpyridin-4-yl)-6-(4- fluorophenyl)pyrimidin-2- amine | 495.2 | | | 1010 | |
| 384 | 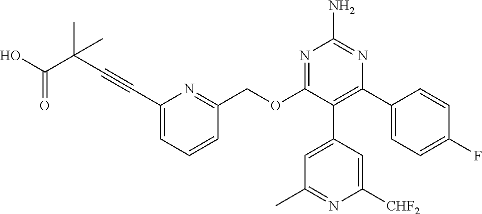 [SMILES: Cc1cc(-c2c(OCc3cccc(C#CC(C)(C)C(=O)O)n3)nc(N)nc2-c2ccc(F)cc2)cc(C(F)F)n1] | 4-(6-(((2-amino-5-(2- (difluoromethyl)-6- methylpyridin-4-yl)-6-(4- fluorophenyl)pyrimidin-4- yl)oxy)methyl)pyridin-2- yl)-2,2-dimethylbut-3- ynoic acid | 548.2 | | | 15418 | |
| 385 | 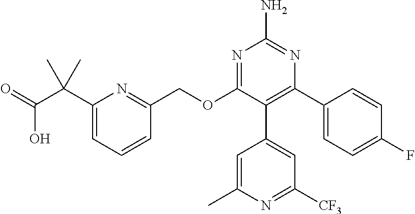 [SMILES: Cc1cc(-c2c(OCc3cccc(C(C)(C)C(=O)O)n3)nc(N)nc2-c2ccc(F)cc2)cc(C(F)(F)F)n1] | 2-(6-(((2-amino-6-(4- fluorophenyl)-5-(2-methyl- 6-(trifluoromethyl)pyridin- 4-yl)pyrimidin-4- yl)oxy)methyl)pyridin-2- yl)-2-methylpropanoic acid | 542.2 | | | 2332/ 1015 | 1461 |
| 386 | 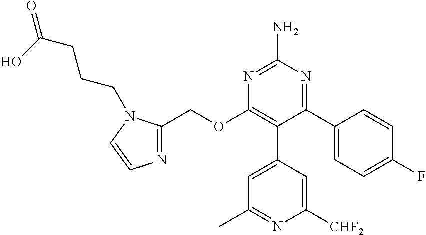 [SMILES: Cc1cc(-c2c(OCc3nccn3CCCC(=O)O)nc(N)nc2-c2ccc(F)cc2)cc(C(F)F)n1] | 4-(2-(((2-amino-5-(2- (difluoromethyl)-6- methylpyridin-4-yl)-6-(4- fluorophenyl)pyrimidin-4- yl)oxy)methyl)-1H- imidazol-1-yl)butanoic acid | 513.2 | | | 3819 | |
| 387 | 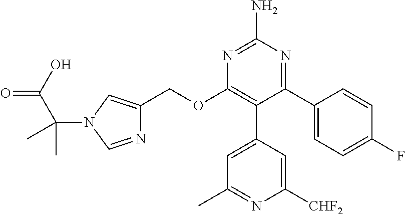 [SMILES: Cc1cc(-c2c(OCc3cn(C(C)(C)C(=O)O)cn3)nc(N)nc2-c2ccc(F)cc2)cc(C(F)F)n1] | 2-(4-(((2-amino-5-(2- (difluoromethyl)-6- methylpyridin-4-yl)-6-(4- fluorophenyl)pyrimidin-4- yl)oxy)methyl)-1H- imidazol-1-yl)-2- methylpropanoic acid | 513.2 | | | 16322 | |
| 388 | 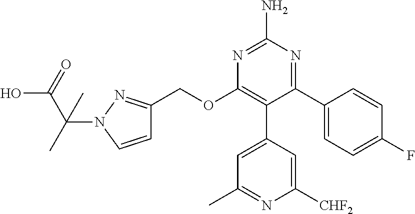 [SMILES: Cc1cc(-c2c(OCc3ccn(C(C)(C)C(=O)O)n3)nc(N)nc2-c2ccc(F)cc2)cc(C(F)F)n1] | 2-(3-(((2-amino-5-(2- (difluoromethyl)-6- methylpyridin-4-yl)-6-(4- fluorophenyl)pyrimidin-4- yl)oxy)methyl)-1H- pyrazol-1-yl)-2- methylpropanoic acid | 513.2 | | | 7662 | |
| 389 | 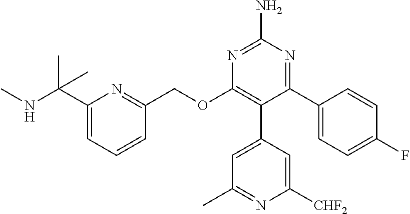 [SMILES: CNC(C)(C)c1cccc(COc2nc(N)nc(-c3ccc(F)cc3)c2-c2cc(C)nc(C(F)F)c2)n1] | 5-(2-(difluoromethyl)-6- methylpyridin-4-yl)-4-(4- fluorophenyl)-6-((6-(2- (methylamino)propan-2- yl)pyridin-2- yl)methoxy)pyrimidin-2- amine | 509.3 | | | 231/ 114/ 100 | 60 |
| 390 | 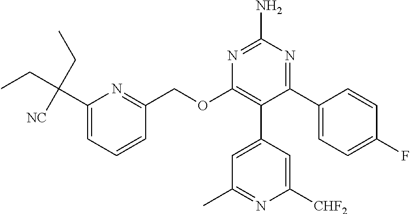 [SMILES: CCC(C#N)(CC)c1cccc(COc2nc(N)nc(-c3ccc(F)cc3)c2-c2cc(C)nc(C(F)F)c2)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-2-ethylbutanenitrile | 533.3 | | | 85/98/ 51 | |
| 391 | 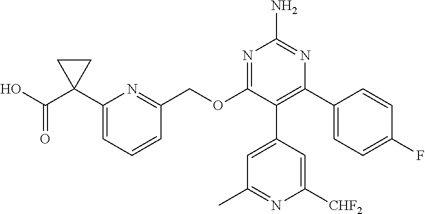 [SMILES: Cc1cc(-c2c(OCc3cccc(C4(C(=O)O)CC4)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)cyclopropane-1- carboxylic acid | 522.2 | | | 1129/ 979 | 602 |
| 392 | 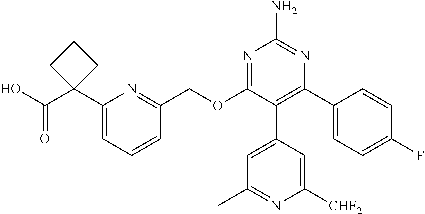 [SMILES: Cc1cc(-c2c(OCc3cccc(C4(C(=O)O)CCC4)n3)nc(N)nc2-c2ccc(F)cc2)cc(C(F)F)n1] | 1-(6-(((2-amino-5-(2- (difluoromethyl)-6- methylpyridin-4-yl)-6-(4- fluorophenyl)pyrimidin-4- yl)oxy)methyl)pyridin-2- yl)cyclobutane-1- carboxylic acid | 536.2 | | | 186/ 139/ 180/ 209 | 353 |
| 393 | 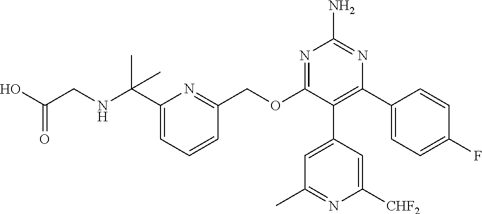 [SMILES: Cc1cc(-c2c(OCc3cccc(C(C)(C)NCC(=O)O)n3)nc(N)nc2-c2ccc(F)cc2)cc(C(F)F)n1] | (2-(6-(((2-amino-5-(2- (difluoromethyl)-6- methylpyridin-4-yl)-6-(4- fluorophenyl)pyrimidin-4- yl)oxy)methyl)pyridin-2- yl)propan-2-yl)glycine | 553.3 | | | 628/ 597/ 892/ 826/ 566/ 686 | 6.9/ 8.4/ 42.0 |
| 394 | 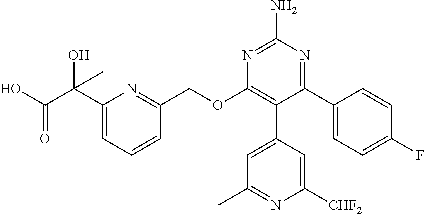 [SMILES: Cc1cc(-c2c(OCc3cccc(C(C)(O)C(=O)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-2-hydroxypropanoic acid | 526.1 | | | 865/ 496 | |
| 395 | 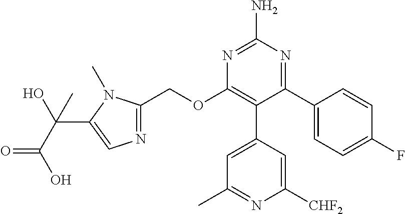 [SMILES: Cc1cc(-c2c(OCc3ncc(C(C)(O)C(=O)O)n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-imidazol-5-yl)-2- hydroxypropanoic acid | 529.2 | | | >50000 | |
| 396 | 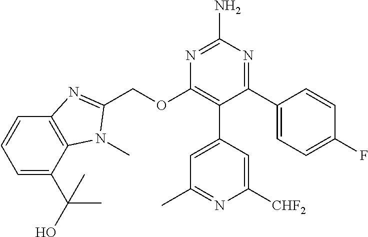 [SMILES: Cc1cc(-c2c(OCc3nc4cccc(C(C)(C)O)c4n3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-benzo[d]imidazol-7- yl)propan-2-ol | 549.2 | | | 869/ 634 | |
| 397 | 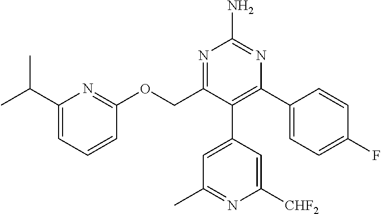 [SMILES: Cc1cc(-c2c(COc3cccc(C(C)C)n3)nc(N)nc2-c2ccc(F)cc2)cc(C(F)F)n1] | 5-(2-(difluoromethyl)-6- methylpyridin-4-yl)-4-(4- fluorophenyl)-6-(((6- isopropylpyridin-2- yl)oxy)methyl)pyrimidin-2- amine | 480.2 | | | 9.4/32/ 26/17 | |
| 398 | 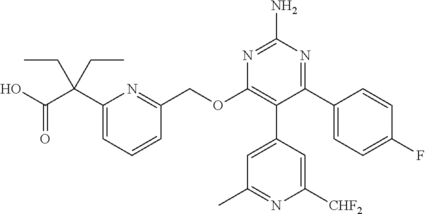 [SMILES: CCC(CC)(C(=O)O)c1cccc(COc2nc(N)nc(-c3ccc(F)cc3)c2-c2cc(C)nc(C(F)F)c2)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-2-ethylbutanoic acid | 551.3 | | | 20/70/ 68/141 | 43.6/ 26.2 |
| 399 | 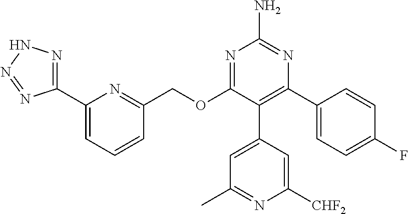 [SMILES: Cc1cc(-c2c(OCc3cccc(-c4nn[nH]n4)n3)nc(N)nc2-c2ccc(F)cc2)cc(C(F)F)n1] | 4-((6-(2H-tetrazol-5- yl)pyridin-2-yl)methoxy)- 5-(2-(difluoromethyl)-6- methylpyridin-4-yl)-6-(4- fluorophenyl)pyrimidin-2- amine | 506.2 | | | 273/ 362/ 482/ 440 | 201 |
| 400 | 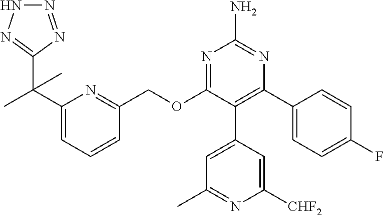 [SMILES: Cc1cc(-c2c(OCc3cccc(C(C)(C)c4nn[nH]n4)n3)nc(N)nc2-c2ccc(F)cc2)cc(C(F)F)n1] | 4-((6-(2-(2H-tetrazol-5- yl)propan-2-yl)pyridin-2- yl)methoxy)-5-(2- (difluoromethyl)-6- methylpyridin-4-yl)-6-(4- fluorophenyl)pyrimidin-2- amine | 548.2 | | | 1003/ 1470/ 1435 | |
| 401 | 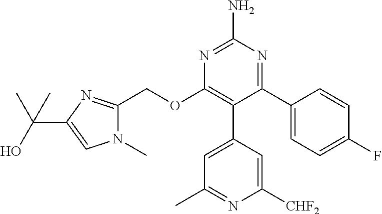 [SMILES: Cc1cc(-c2c(OCc3nc(C(C)(C)O)cn3C)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-imidazol-4-yl)propan- 2-ol | 499.2 | | | 318/ 246/ 259 | |
| 402 | 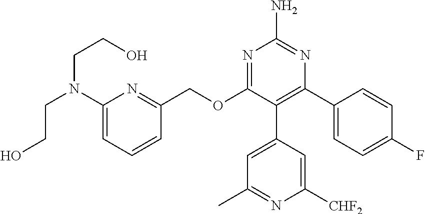 [SMILES: Cc1cc(-c2c(OCc3cccc(N(CCO)CCO)n3)nc(N)nc2-c2ccc(F)cc2)cc(C(F)F)n1] | 2,2′-((6-(((2-amino-5-(2- (difluoromethyl)-6- methylpyridin-4-yl)-6-(4- fluorophenyl)pyrimidin-4- yl)oxy)methyl)pyridin-2- yl)azanediyl)bis(ethan-1- ol) | 541.2 | | | 515/ 579/ 560 | 692 |
| 403 | 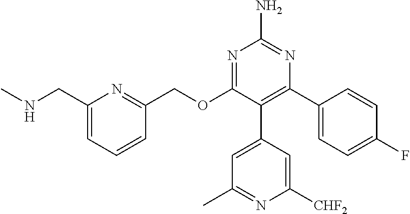 [SMILES: CNCc1cccc(COc2nc(N)nc(-c3ccc(F)cc3)c2-c2cc(C)nc(C(F)F)c2)n1] | 5-(2-(difluoromethyl)-6- methylpyridin-4-yl)-4-(4- fluorophenyl)-6-((6- ((methylamino)methyl) pyridin-2- yl)methoxy)pyrimidin-2- amine | 481.2 | | | 249/ 322/ 311 | 108 |
| 404 | 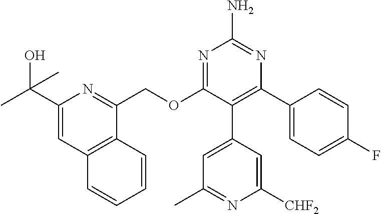 [SMILES: Cc1cc(-c2c(OCc3nc(C(C)(C)O)cc4ccccc34)nc(N)nc2-c2ccc(F)cc2)cc(C(F)F)n1] | 2-(1-(((2-amino-5-(2- (difluoromethyl)-6- methylpyridin-4-yl)-6-(4- fluorophenyl)pyrimidin-4- yl)oxy)methyl)isoquinolin- 3-yl)propan-2-ol | 546.2 | | | 1367/ 1804 | |
| 405 | 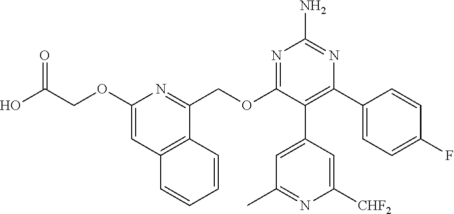 [SMILES: Cc1cc(-c2c(OCc3nc(OCC(=O)O)cc4ccccc34)nc(N)nc2-c2ccc(F)cc2)cc(C(F)F)n1] | 2-((1-(((2-amino-5-(2- (difluoromethyl)-6- methylpyridin-4-yl)-6-(4- fluorophenyl)pyrimidin-4- yl)oxy)methyl)isoquinolin- 3-yl)oxy)acetic acid | 562.2 | | | 619 | |
| 406 | 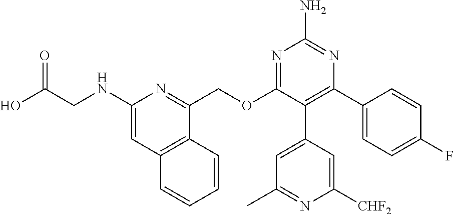 [SMILES: Cc1cc(-c2c(OCc3nc(NCC(=O)O)cc4ccccc34)nc(N)nc2-c2ccc(F)cc2)cc(C(F)F)n1] | (1-(((2-amino-5-(2- (difluoromethyl)-6- methylpyridin-4-yl)-6-(4- fluorophenyl)pyrimidin-4- yl)oxy)methyl)isoquinolin- 3-yl)glycine | 561.2 | | | 719 | |
| 407 | 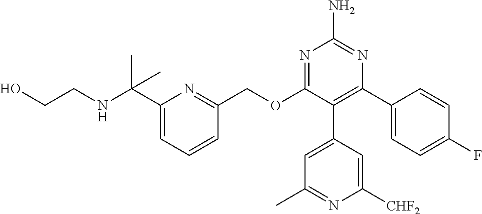 [SMILES: Cc1cc(-c2c(OCc3cccc(C(C)(C)NCCO)n3)nc(N)nc2-c2ccc(F)cc2)cc(C(F)F)n1] | 2-((2-(6-(((2-amino-5-(2- (difluoromethyl)-6- methylpyridin-4-yl)-6-(4- fluorophenyl)pyrimidin-4- yl)oxy)methyl)pyridin-2- yl)propan-2- yl)amino)ethan-1-ol | 539.2 | | | 107/88 | 91.5 |
| 408 | 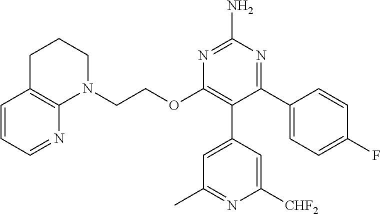 [SMILES: Cc1cc(-c2c(OCCN3CCCc4cccnc43)nc(N)nc2-c2ccc(F)cc2)cc(C(F)F)n1] | 5-(2-(difluoromethyl)-6- methylpyridin-4-yl)-4-(2- (3,4-dihydro-1,8- naphthyridin-1(2H)- yl)ethoxy)-6-(4- fluorophenyl)pyrimidin-2- amine | 507.2 | | | 68/41/ 19/74 | 37.9 |
| 409 | 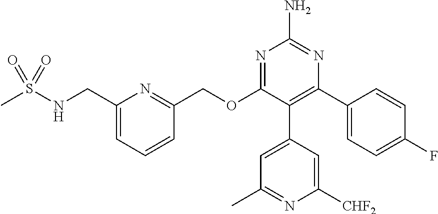 [SMILES: Cc1cc(-c2c(OCc3cccc(CNS(C)(=O)=O)n3)nc(N)nc2-c2ccc(F)cc2)cc(C(F)F)n1] | N-((6-(((2-amino-5-(2- (difluoromethyl)-6- methylpyridin-4-yl)-6-(4- fluorophenyl)pyrimidin-4- yl)oxy)methyl)pyridin-2- yl)methyl) methanesulfonamide | 545.2 | | | 977/ 822 | |
| 410 | 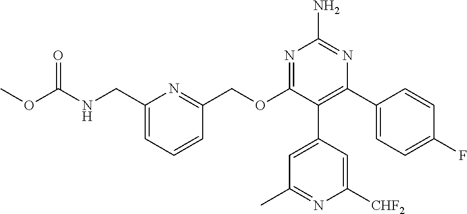 [SMILES: COC(=O)NCc1cccc(COc2nc(N)nc(-c3ccc(F)cc3)c2-c2cc(C)nc(C(F)F)c2)n1] | methyl ((6-(((2-amino-5- (2-(difluoromethyl)-6- methylpyridin-4-yl)-6-(4- fluorophenyl)pyrimidin-4- yl)oxy)methyl)pyridin-2- yl)methyl)carbamate | 525.2 | | | 97/ 142/ 124/ 178 | 20 |
| 411 | 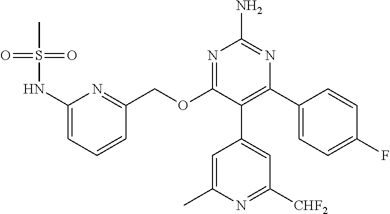 [SMILES: Cc1cc(-c2c(OCc3cccc(NS(C)(=O)=O)n3)nc(N)nc2-c2ccc(F)cc2)cc(C(F)F)n1] | N-(6-(((2-amino-5-(2- (difluoromethyl)-6- methylpyridin-4-yl)-6-(4- fluorophenyl)pyrimidin-4- yl)oxy)methyl)pyridin-2- yl)methanesulfonamide | 531.2 | | | 3194 | |
| 412 | 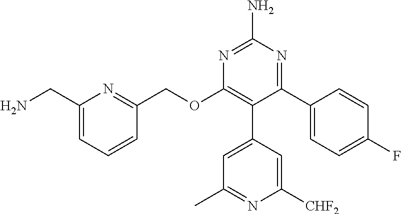 [SMILES: Cc1cc(-c2c(OCc3cccc(CN)n3)nc(N)nc2-c2ccc(F)cc2)cc(C(F)F)n1] | 4-((6- (aminomethyl)pyridin-2- yl)methoxy)-5-(2- (difluoromethyl)-6- methylpyridin-4-yl)-6-(4- fluorophenyl)pyrimidin-2- amine | 467.1 | | | 503/ 747 | |
| 413 | 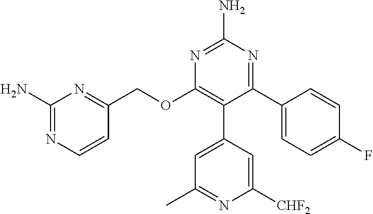 [SMILES: Cc1cc(-c2c(OCc3ccnc(N)n3)nc(N)nc2-c2ccc(F)cc2)cc(C(F)F)n1] | 4-((2-aminopyrimidin-4- yl)methoxy)-5-(2- (difluoromethyl)-6- methylpyridin-4-yl)-6-(4- fluorophenyl)pyrimidin-2- amine | 454.1 | | | 4.5/ 19/14 | 50.2/ 40.8 |
| 414 | 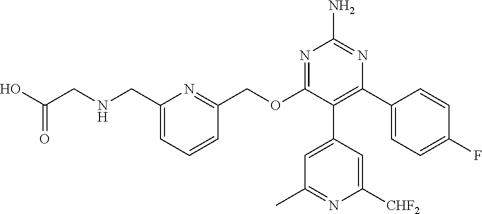 [SMILES: Cc1cc(-c2c(OCc3cccc(CNCC(=O)O)n3)nc(N)nc2-c2ccc(F)cc2)cc(C(F)F)n1] | ((6-(((2-amino-5-(2- (difluoromethyl)-6- methylpyridin-4-yl)-6-(4- fluorophenyl)pyrimidin-4- yl)oxy)methyl)pyridin-2- yl)methyl)glycine | N/A | | | 2053 | |
| 415 | 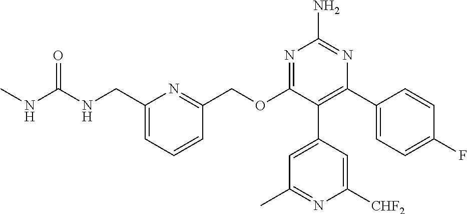 [SMILES: CNC(=O)NCc1cccc(COc2nc(N)nc(-c3ccc(F)cc3)c2-c2cc(C)nc(C(F)F)c2)n1] | 1-((6-(((2-amino-5-(2- (difluoromethyl)-6- methylpyridin-4-yl)-6-(4- fluorophenyl)pyrimidin-4- yl)oxy)methyl)pyridin-2- yl)methyl)-3-methylurea | 524.2 | | | 97/ 152/ 129 | 16 |
| 416 | 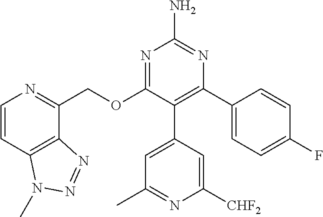 [SMILES: Cc1cc(-c2c(OCc3nccc4c3nnn4C)nc(N)nc2-c2ccc(F)cc2)cc(C(F)F)n1] | 5-(2-(difluoromethyl)-6- methylpyridin-4-yl)-4-(4- fluorophenyl)-6-((1- methyl-1H- [1,2,3]triazolo[4,5- c]pyridin-4- yl)methoxy)pyrimidin-2- amine | 493.1 | | | 498/ 615 | |
| 417 | 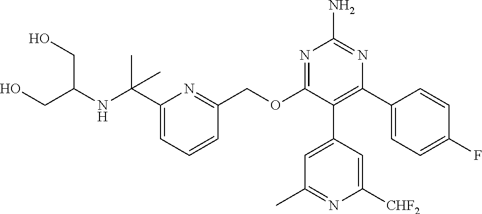 [SMILES: Cc1cc(-c2c(OCc3cccc(C(C)(C)NC(CO)CO)n3)nc(N)nc2-c2ccc(F)cc2)cc(C(F)F)n1] | 2-((2-(6-(((2-amino-5-(2- (difluoromethyl)-6- methylpyridin-4-yl)-6-(4- fluorophenyl)pyrimidin-4- yl)oxy)methyl)pyridin-2- yl)propan-2- yl)amino)propane-1,3-diol | 569.4 | | | 60/44 | 38.7 |
| 418 | 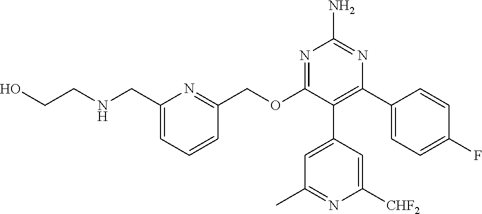 [SMILES: Cc1cc(-c2c(OCc3cccc(CNCCO)n3)nc(N)nc2-c2ccc(F)cc2)cc(C(F)F)n1] | 2-(((6-(((2-amino-5-(2- (difluoromethyl)-6- methylpyridin-4-yl)-6-(4- fluorophenyl)pyrimidin-4- yl)oxy)methyl)pyridin-2- yl)methyl)amino)ethan-1- ol | 511.2 | | | 191/ 177 | 89.3 |
| 419 | 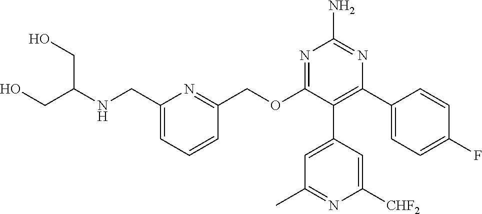 [SMILES: Cc1cc(-c2c(OCc3cccc(CNC(CO)CO)n3)nc(N)nc2-c2ccc(F)cc2)cc(C(F)F)n1] | 2-(((6-(((2-amino-5-(2- (difluoromethyl)-6- methylpyridin-4-yl)-6-(4- fluorophenyl)pyrimidin-4- yl)oxy)methyl)pyridin-2- yl)methyl)amino)propane- 1,3-diol | 541.3 | | | 470/ 311 | 87.8 |
| 420 | 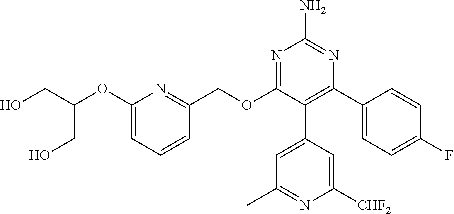 [SMILES: Cc1cc(-c2c(OCc3cccc(OC(CO)CO)n3)nc(N)nc2-c2ccc(F)cc2)cc(C(F)F)n1] | 2-(((6-(((2-amino-5-(2- (difluoromethyl)-6- methylpyridin-4-yl)-6-(4- fluorophenyl)pyrimidin-4- yl)oxy)methyl)pyridin-2- yl)oxy)propane-1,3-diol | 528.2 | | | 122/ 112 | 55.7 |
| 421 | 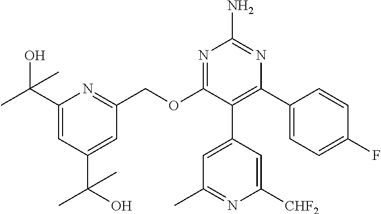 [SMILES: Cc1cc(-c2c(OCc3cc(C(C)(C)O)cc(C(C)(C)O)n3)nc(N)nc2-c2ccc(F)cc2)cc(C(F)F)n1] | 2,2′-(6-(((2-amino-5-(2- (difluoromethyl)-6- methylpyridin-4-yl)-6-(4- fluorophenyl)pyrimidin-4- yl)oxy)methyl)pyridine- 2,4-diyl)bis(propan-2-ol) | 554.2 | | | 3425 | |
| 422 | 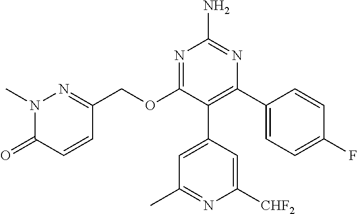 [SMILES: Cc1cc(-c2c(OCc3ccc(=O)n(C)n3)nc(N)nc2-c2ccc(F)cc2)cc(C(F)F)n1] | 6-(((2-amino-5-(2- (difluoromethyl)-6- methylpyridin-4-yl)-6-(4- fluorophenyl)pyrimidin-4- yl)oxy)methyl)-2- methylpyridazin-3(2H)- one | 469.2 | | | 432/ 338 | 423.5 |
| 423 | 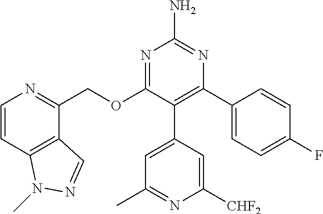 [SMILES: Cc1cc(-c2c(OCc3nccc4c3cnn4C)nc(N)nc2-c2ccc(F)cc2)cc(C(F)F)n1] | 5-(2-(difluoromethyl)-6- methylpyridin-4-yl)-4-(4- fluorophenyl)-6-((1- methyl-1H-pyrazolo[4,3- c]pyridin-4- yl)methoxy)pyrimidin-2- amine | 492.2 | | | 435/ 732 | |
| 424 | 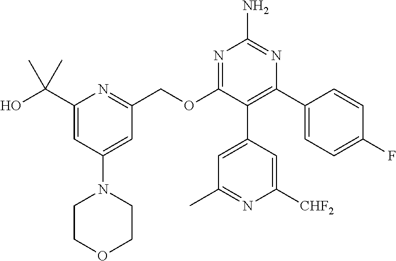 [SMILES: Cc1cc(-c2c(OCc3cc(N4CCOCC4)cc(C(C)(C)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)-4- morpholinopyridin-2- yl)propan-2-ol | 581.3 | | | 5931/ 9834 | |
| 425 | 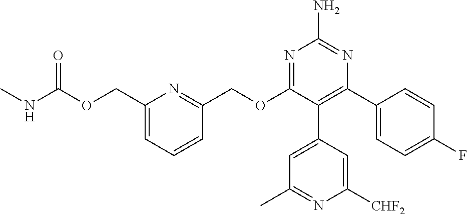 [SMILES: CNC(=O)OCc1cccc(COc2nc(N)nc(-c3ccc(F)cc3)c2-c2cc(C)nc(C(F)F)c2)n1] | (6-(((2-amino-5-(2- (difluoromethyl)-6- methylpyridin-4-yl)-6-(4- fluorophenyl)pyrimidin-4- yl)oxy)methyl)pyridin-2- yl)methyl methylcarbamate | 525.2 | | | 35/74/ 23 | 33.3/ 29 |
| 426 | 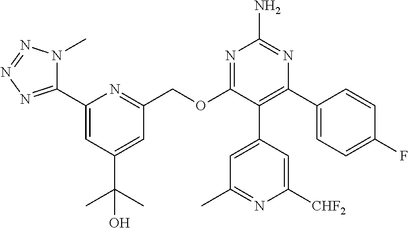 [SMILES: Cc1cc(-c2c(OCc3cc(C(C)(C)O)cc(-c4nnnn4C)n3)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-6-(1- methyl-1H-tetrazol-5- yl)pyridin-4-yl)propan-2-ol | 578.2 | | | 7978/ 6194 | |
| 427 | 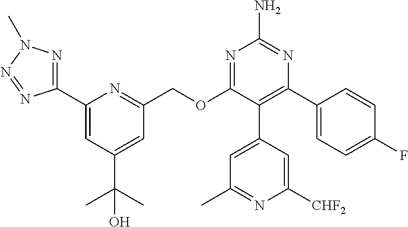 [SMILES: Cc1cc(-c2c(OCc3cc(C(C)(C)O)cc(-c4nnn(C)n4)n3)nc(N)nc2-c2ccc(F)cc2)cc(C(F)F)n1] | 2-(2-(((2-amino-5-(2- (difluoromethyl)-6- methylpyridin-4-yl)-6-(4- fluorophenyl)pyrimidin-4- yl)oxy)methyl)-6-(2- methyl-2H-tetrazol-5- yl)pyridin-4-yl)propan-2-ol | 578.3 | | | 5148/ 6010 | |
| 428 | 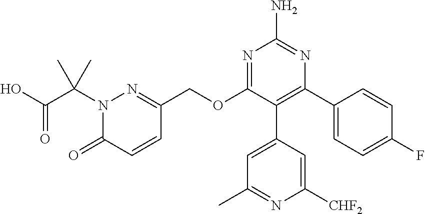 [SMILES: Cc1cc(-c2c(OCc3ccc(=O)n(C(C)(C)C(=O)O)n3)nc(N)nc2-c2ccc(F)cc2)cc(C(F)F)n1] | 2-(3-(((2-amino-5-(2- (difluoromethyl)-6- methylpyridin-4-yl)-6-(4- fluorophenyl)pyrimidin-4- yl)oxy)methyl)-6- oxopyridazin-1(6H)-yl)-2- methylpropanoic acid | 541.3 | | | >30000 | |
| 429 | 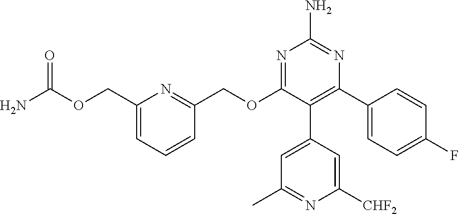 [SMILES: Cc1cc(-c2c(OCc3cccc(COC(N)=O)n3)nc(N)nc2-c2ccc(F)cc2)cc(C(F)F)n1] | (6-(((2-amino-5-(2- (difluoromethyl)-6- methylpyridin-4-yl)-6-(4- fluorophenyl)pyrimidin-4- yl)oxy)methyl)pyridin-2- yl)methyl carbamate | 511.1 | | | 59/43 | 33 |
| 430 | 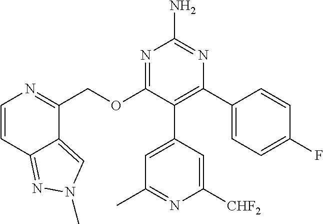 [SMILES: Cc1cc(-c2c(OCc3nccc4nn(C)cc34)nc(N)nc2-c2ccc(F)cc2)cc(C(F)F)n1] | 5-(2-(difluoromethyl)-6- methylpyridin-4-yl)-4-(4- fluorophenyl)-6-((2- methyl-2H-pyrazolo[4,3- c]pyridin-4- yl)methoxy)pyrimidin-2- amine | 492.2 | | | 2459 | |
| 431 | 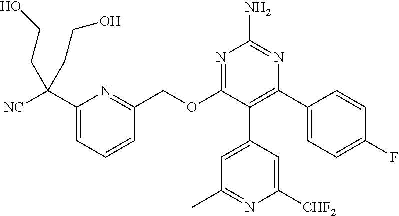 [SMILES: Cc1cc(-c2c(OCc3cccc(C(C#N)(CCO)CCO)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-4-hydroxy-2-(2- hydroxyethyl)butanenitrile | 565.3 | | | 412/ 315/ 310 | 1998 |
| 432 | 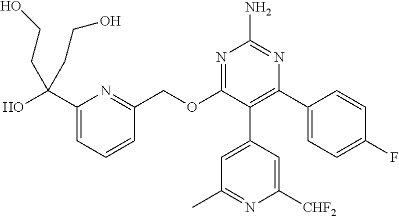 [SMILES: Cc1cc(-c2c(OCc3cccc(C(O)(CCO)CCO)n3)nc(N)nc2-c2ccc(F)cc2)cc(C(F)F)n1] | 3-(6-(((2-amino-5-(2- (difluoromethyl)-6- methylpyridin-4-yl)-6-(4- fluorophenyl)pyrimidin-4- yl)oxy)methyl)pyridin-2- yl)pentane-1,3,5-triol | 556.3 | | | 1256 | |
| 433 | 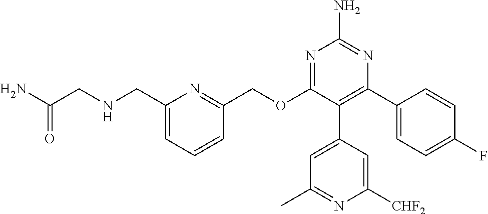 [SMILES: Cc1cc(-c2c(OCc3cccc(CNCC(N)=O)n3)nc(N)nc2-c2ccc(F)cc2)cc(C(F)F)n1] | 2-(((6-(((2-amino-5-(2- (difluoromethyl)-6- methylpyridin-4-yl)-6-(4- fluorophenyl)pyrimidin-4- yl)oxy)methyl)pyridin-2- yl)methyl)amino)acetamide | 524.2 | | | 342/ 210 | 59 |
| 434 | 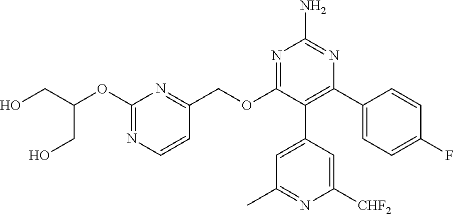 [SMILES: Cc1cc(-c2c(OCc3ccnc(OC(CO)CO)n3)nc(N)nc2-c2ccc(F)cc2)cc(C(F)F)n1] | 2-((4-(((2-amino-5-(2- (difluoromethyl)-6- methylpyridin-4-yl)-6-(4- fluorophenyl)pyrimidin-4- yl)oxy)methyl)pyrimidin-2- yl)oxy)propane-1,3-diol | 529.3 | | | 346/ 290 | 411 |
| 435 | 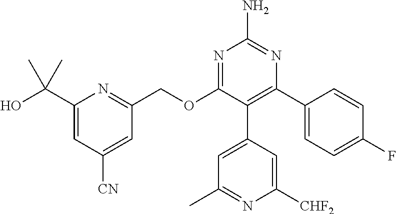 [SMILES: Cc1cc(-c2c(OCc3cc(C#N)cc(C(C)(C)O)n3)nc(N)nc2-c2ccc(F)cc2)cc(C(F)F)n1] | 2-(((2-amino-5-(2- (difluoromethyl)-6- methylpyridin-4-yl)-6-(4- fluorophenyl)pyrimidin-4- yl)oxy)methyl)-6-(2- hydroxypropan-2- yl)isonicotinonitrile | 521.3 | | | 506 | |
| 436 | 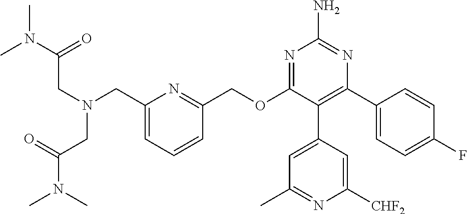 [SMILES: Cc1cc(-c2c(OCc3cccc(CN(CC(=O)N(C)C)CC(=O)N(C)C)n3)nc(N)nc2-c2ccc(F)cc2)cc(C(F)F)n1] | 2,2′-(((6-(((2-amino-5-(2- (difluoromethyl)-6- methylpyridin-4-yl)-6-(4- fluorophenyl)pyrimidin-4- yl)oxy)methyl)pyridin-2- yl)methyl)azanediyl)bis(N, N-dimethylacetamide) | 637.3 | | | 894/ 530 | |
| 437 | 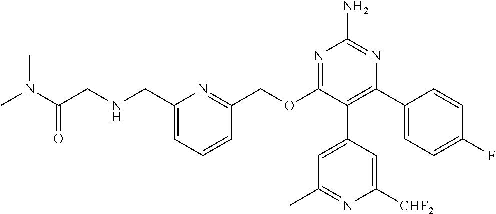 [SMILES: Cc1cc(-c2c(OCc3cccc(CNCC(=O)N(C)C)n3)nc(N)nc2-c2ccc(F)cc2)cc(C(F)F)n1] | 2-(((6-(((2-amino-5-(2- (difluoromethyl)-6- methylpyridin-4-yl)-6-(4- fluorophenyl)pyrimidin-4- yl)oxy)methyl)pyridin-2- yl)methyl)amino)-N,N- dimethylacetamide | 552.3 | | | 282 | 232 |
| 438 | 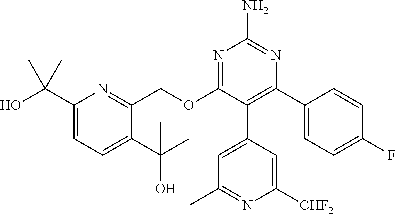 [SMILES: Cc1cc(-c2c(OCc3nc(C(C)(C)O)ccc3C(C)(C)O)nc(N)nc2-c2ccc(F)cc2)cc(C(F)F)n1] | 2,2-(6-(((2-amino-5-(2- (difluoromethyl)-6- methylpyridin-4-yl)-6-(4- fluorophenyl)pyrimidin-4- yl)oxy)methyl)pyridine- 2,5-diyl)bis(propan-2-ol) | 554.3 | | | 3183 | |
| 439 | 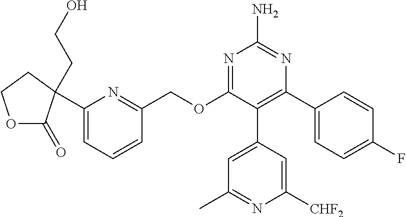 [SMILES: Cc1cc(-c2c(OCc3cccc(C4(CCO)CCOC4=O)n3)nc(N)nc2-c2ccc(F)cc2)cc(C(F)F)n1] | 3-(6-(((2-amino-5-(2- (difluoromethyl)-6- methylpyridin-4-yl)-6-(4- fluorophenyl)pyrimidin-4- yl)oxy)methyl)pyridin-2- yl)-3-(2- hydroxyethyl)dihydrofuran- 2(3H)-one | 566.2 | | | 103 | 252 |
| 440 | 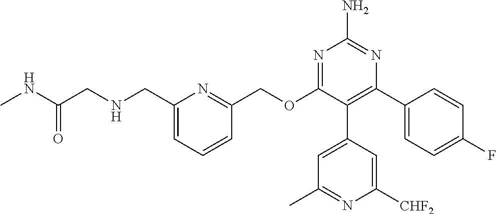 [SMILES: CNC(=O)CNCc1cccc(COc2nc(N)nc(-c3ccc(F)cc3)c2-c2cc(C)nc(C(F)F)c2)n1] | 2-(((6-(((2-amino-5-(2- (difluoromethyl)-6- methylpyridin-4-yl)-6-(4- fluorophenyl)pyrimidin-4- yl)oxy)methyl)pyridin-2- yl)methyl)amino)-N- methylacetamide | 538.3 | | | 215 | 95 |
| 441 | 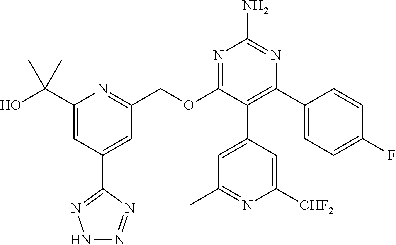 [SMILES: Cc1cc(-c2c(OCc3cc(-c4nn[nH]n4)cc(C(C)(C)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)-4-(2H- tetrazol-5-yl)pyridin-2- yl)propan-2-ol | 564.3 | | | >3000 | |
| 442 | 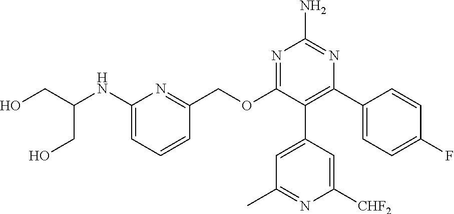 [SMILES: Cc1cc(-c2c(OCc3cccc(NC(CO)CO)n3)nc(N)nc2-c2ccc(F)cc2)cc(C(F)F)n1] | 2-((6-(((2-amino-5-(2- (difluoromethyl)-6- methylpyridin-4-yl)-6-(4- fluorophenyl)pyrimidin-4- yl)oxy)methyl)pyridin-2- yl)amino)propane-1,3-diol | 527.3 | | | 177 | 208 |
| 443 | 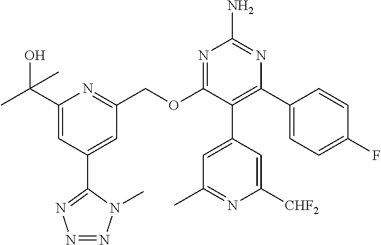 [SMILES: Cc1cc(-c2c(OCc3cc(-c4nnnn4C)cc(C(C)(C)O)n3)nc(N)nc2-c2ccc(F)cc2)cc(C(F)F)n1] | 2-(6-(((2-amino-5-(2- (difluoromethyl)-6- methylpyridin-4-yl)-6-(4- fluorophenyl)pyrimidin-4- yl)oxy)methyl)-4-(1- methyl-1H-tetrazol-5- yl)pyridin-2-yl)propan-2-ol | 578.2 | | | 23152 | |
| 444 | 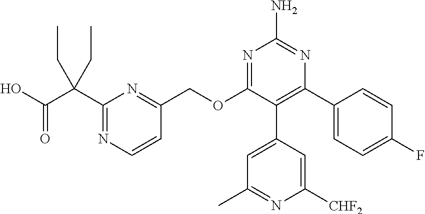 [SMILES: CCC(CC)(C(=O)O)c1nccc(COc2nc(N)nc(-c3ccc(F)cc3)c2-c2cc(C)nc(C(F)F)c2)n1] | 2-(4-(((2-amino-5-(2- (difluoromethyl)-6- methylpyridin-4-yl)-6-(4- fluorophenyl)pyrimidin-4- yl)oxy)methyl)pyrimidin-2- yl)-2-ethylbutanoic acid | | | | | |
| 445 | 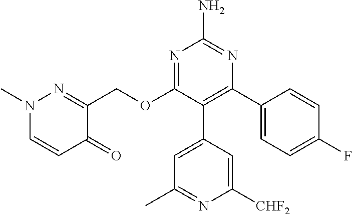 [SMILES: Cc1cc(-c2c(OCc3nn(C)ccc3=O)nc(N)nc2-c2ccc(F)cc2)cc(C(F)F)n1] | 3-(((2-amino-5-(2- (difluoromethyl)-6- methylpyridin-4-yl)-6-(4- fluorophenyl)pyrimidin-4- yl)oxy)methyl)-1- methylpyridazin-4(1H)- one | | | | | |
| 446 | 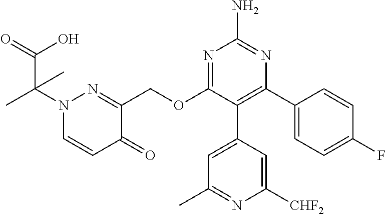 [SMILES: Cc1cc(-c2c(OCc3nn(C(C)(C)C(=O)O)ccc3=O)nc(N)nc2-c2ccc(F)cc2)cc(C(F)F)n1] | 2-(3-(((2-amino-5-(2- (difluoromethyl)-6- methylpyridin-4-yl)-6-(4- fluorophenyl)pyrimidin-4- yl)oxy)methyl)-4- oxopyridazin-1(4H)-yl)-2- methylpropanoic acid | | | | | |
| 447 | 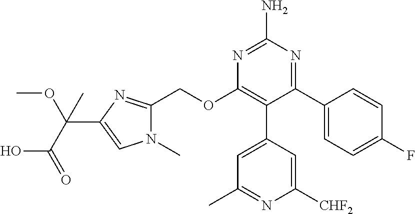 [SMILES: COC(C)(C(=O)O)c1cn(C)c(COc2nc(N)nc(-c3ccc(F)cc3)c2-c2cc(C)nc(C(F)F)c2)n1] | 2-(2-(((2-amino-5-(2- (difluoromethyl)-6- methylpyridin-4-yl)-6-(4- fluorophenyl)pyrimidin-4- yl)oxy)methyl)-1-methyl- 1H-imidazol-4-yl)-2- methoxypropanoic acid | | | | | |
| 448 | 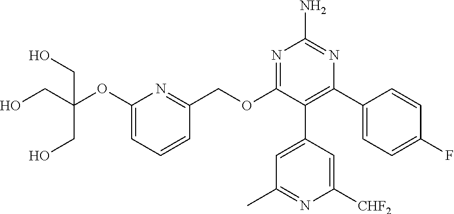 [SMILES: Cc1cc(-c2c(OCc3cccc(OC(CO)(CO)CO)n3)nc(N)nc2-c2ccc(F)cc2)cc(C(F)F)n1] | 2-((6-(((2-amino-5-(2- (difluoromethyl)-6- methylpyridin-4-yl)-6-(4- fluorophenyl)pyrimidin-4- yl)oxy)methyl)pyridin-2- yl)oxy)-2- (hydroxymethyl)propane- 1,3-diol | | | | | |
| 449 | 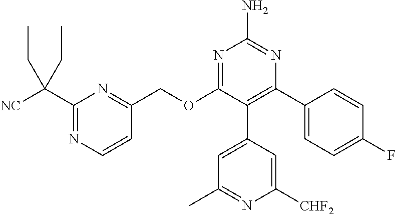 [SMILES: CCC(C#N)(CC)c1nccc(COc2nc(N)nc(-c3ccc(F)cc3)c2-c2cc(C)nc(C(F)F)c2)n1] | 2-(4-(((2-amino-5-(2- (difluoromethyl)-6- methylpyridin-4-yl)-6-(4- fluorophenyl)pyrimidin-4- yl)oxy)methyl)pyrimidin-2- yl)-2-ethylbutanenitrile | | | | | |
| 450 | 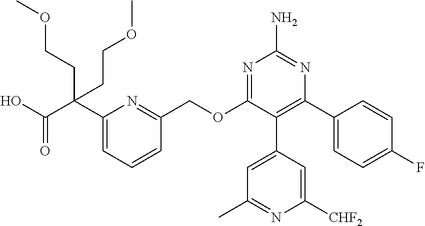 [SMILES: COCCC(CCOC)(C(=O)O)c1cccc(COc2nc(N)nc(-c3ccc(F)cc3)c2-c2cc(C)nc(C(F)F)c2)n1] | 2-(6-(((2-amino-5-(2- (difluoromethyl)-6- methylpyridin-4-yl)-6-(4- fluorophenyl)pyrimidin-4- yl)oxy)methyl)pyridin-2- yl)-4-methoxy-2-(2- methoxyethyl)butanoic acid | | | | | |

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A compound of Formula (I): 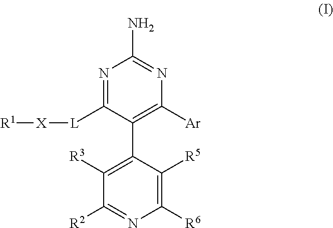
wherein:
L is [X]—(C(R^{a})₂)ₙ—O—,
where [X] indicates which end of L is attached to X in Formula (I);
and
X is absent (i.e., it represents a bond between L and R¹), (CR^{a}₂)ₙ, C(═O), [R¹]—(CR^{a}₂)ₙ—NR^{b}—, [R¹]—(CR^{a}₂)ₙ—O—, [R¹]—O—(CR^{a}₂)ₙ—, [R¹]—NR^{b}—(CR^{a}₂)ₙ—, [R¹]—(CR^{a}₂)ₙ—S(O)ₘ—, [R¹]—S(O)ₘ—(CR^{a}₂)ₙ—, [R¹]—C(O)—O—, [R¹]—C(O)—NR^{b}—, [R¹]—(CR^{a}₂)ₙ—NR^{b}—C(O)—, [R¹]—NR^{b}—C(O)—NR^{b}—, [R¹]—NR^{b}—C(O)—O—, C₁₋₄ alkyl, C₃-C₈ cycloalkyl, a 3-8 membered heterocyclic ring, phenyl, or a 5-12 membered heteroaryl ring; where [R¹] indicates which end of X is attached to R¹;
wherein the C₁₋₄ alkyl, C₃-C₈ cycloalkyl, 3-8 membered heterocyclic ring, phenyl, or 5-12 membered heteroaryl ring is optionally substituted with one to three groups selected from C₁-C₃ alkyl, —OH, oxo, COOR¹⁰, —NR⁸R⁹, C(O)NR⁸R⁹, SO₂R¹¹, SO₂NR⁸R⁹, —S(═O)(═NR^{b})R¹¹, NR^{b}C(O)OR¹¹, NR^{b}C(O)NR⁸R⁹, C₁₋₃ alkyl optionally substituted with OH, OMe, Cx or —O—Cx, and C₁₋₃ alkoxy optionally substituted with OH, OMe, Cx, or —O—Cx;
wherein each Cx is independently selected from C3-Cs cycloalkyl, 4-6 membered heterocyclyl having one or two heteroatoms selected from N, O and S as ring members, phenyl, and 5-12 membered heteroaryl having up to four heteroatoms selected from N, O and S as ring members,
where each Cx is optionally substituted with one or two groups selected from halo, oxo, CN, C₁₋₃ alkyl, C₁₋₃ haloalkyl, C₁₋₃ alkoxy, and OH;
each R^{a} and R^{b} is independently H, —OR^{c}, —COOR^{c}, or C₁-C₃ alkyl optionally substituted with one or two groups selected from halo, oxo, —COOR^{c}, —OR^{c}, and —N(R^{c})₂;
where each R^{c} is independently H or C₁-C₃ alkyl optionally substituted with one to three groups independently selected from halo, OH, oxo, and methoxy;
R¹ is selected from the group consisting of H, OH, R⁷, OR⁷, —NR⁷R⁸, —NR⁸R⁹, —S(O)ₘR⁷, —(CR^{a}₂)₀₋₂—Cy, (CR^{a}₂)₀₋₂—O—Cy, —O—(CR^{a}₂)₁₋₂—Cy, —C(O)R¹⁰, —C(O)OR¹⁰, —C(O)NR⁸R⁹, —NR^{b}C(O)R¹⁰, —NR^{b}COOR¹¹, —NR^{b}C(O)NR⁸R⁹, —NR^{b}SO₂R¹¹, —NR^{b}SO₂NR⁸R⁹, —SO₂R¹¹, —SO₂NR⁸R⁹, OSO₂R¹¹, —OSO₂NR⁸R⁹, —S(═O)(═NR^{b})R¹¹, —OC(O)NR⁸R⁹, —OC(O)R¹¹), —P(O)(R¹¹)₂, —P(O)(OR¹⁰)₂, —P(O)(OR¹⁰)—R¹¹, —P(O)(NR⁸R⁹)₂, —O—P(O)(OR¹⁰)₂, —O—P(O)(OR¹⁰)—R¹¹, and —P(O)(NR⁸R⁹)—R¹¹;
Cy is a cyclic group selected from phenyl, C₃-C₈ cycloalkyl, a 5-12 membered monocyclic heteroaryl group having up to four heteroatoms selected from N, O and S as ring members, and a 3-8 membered heterocyclic ring comprising one or two heteroateoms selected from N, O and S as ring members, and is optionally fused to a phenyl or a 5-12 membered heteroaryl or a heterocyclic ring having one or two heteroatoms selected from N, O and S as ring members or a C₃-C₈ cycloalkyl ring to form a bicyclic group
wherein the cyclic or bicyclic group Cy is optionally substituted with up to three groups independently selected from R⁷, —OR⁷, oxo, halo, —OH, C₁-C₃ alkyl, C₁-C₃ haloalkyl, C₃-C₈ cycloalkyl, C₁-C₃ alkoxy, C₃-C₈ cycloalkyl, COOR¹⁰, CN, SO₂R¹¹, C(O)R¹⁰, —NR⁸R⁹, —NR⁷R⁸, —C(O)NR⁸R⁹, NR^{b}COOR¹¹, NR^{b}SO₂R¹¹, and C₁-C₃ alkyl that is substituted with one or two groups selected from OH, OMe, COOR¹⁰, CN, SO₂R¹¹, C(O)R¹⁰, and C(O)NR⁸R⁹;
R⁷ is C₁-C₆ alkyl, C₂-C₆ alkenyl, C₂-C₆ alkynyl, C₁-C₃ haloalkyl, C₃-C₈ cycloalkyl, or 3-8 membered heterocyclic group having one or two heteroatoms selected from N, O and S as ring members,
wherein the C₁-C₆ alkyl, C₂-C₆ alkenyl, C₂-C₆ alkynyl, C₁-C₃ haloalkyl, C₃-C₈ cycloalkyl, or 3-8 membered heterocyclic group is optionally substituted with one to three groups selected from —OH, OR¹⁰, CN, oxo, COOR¹⁰, C(O)R¹⁰, —NR⁸R⁹, C(O)NR⁸R⁹, SO₂R″, SO₂NR⁸R⁹, —S(═O)(═NR^{b})R¹¹, NR⁸SO₂R¹¹, NR^{b}C(O)OR¹¹, NR^{b}C(O)NR⁸R⁹, OC(O)NR⁸R⁹, Cz, C₁₋₃ alkyl optionally substituted with OH, OMe, Cz, SO₂R¹¹, COOR¹⁰, or —O—Cz, and C₁₋₃ alkoxy optionally substituted with OH, OMe, SO₂R¹¹, COOR¹⁰, Cz, or —O—Cz;
wherein each Cz is independently selected from C₃-C₈ cycloalkyl, 4-6 membered heterocyclyl having one or two heteroatoms selected from N, O and S as ring members, phenyl, and 5-12 membered heteroaryl having up to four heteroatoms selected from N, O and S as ring members,
where each Cz is optionally substituted with one or two groups selected from halo, C₁₋₃ alkyl, C₁₋₃ haloalkyl, C₁₋₃ alkoxy, and OH;
R⁸ and R⁹ are independently at each occurrence selected from H, C(O)R¹⁰, C(O)OR¹⁰, C₁-C₄ haloalkyl, and C₁-C₄ alkyl, C₃-C₈ cycloalkyl or 4-8 membered heterocyclyl having one or two heteroatoms selected from N, O and S as ring members, wherein the C₁-C₄ alkyl, C₃-C₈ cycloalkyl or 4-6 membered heterocyclyl are each optionally substituted with one or two groups independently selected from —OH, Me, —OR¹¹, —NR¹²R¹³, —SO₂R¹¹, COOR¹⁰, C(O)NR¹²R¹³, SO₂NR¹²R¹³, NR^{b}C(O)OR¹¹, and NR^{b}C(O)NR¹²R¹³,
or R⁸ and R⁹ taken together with N to which both are attached form a 4 to 8 membered heterocyclic ring optionally containing an additional N, O, or S as a ring member and optionally substituted with one or two groups selected from OH, OR¹⁰, oxo, halo, CN, C₁-C₃ alkyl, C₁-C₃ haloalkyl, C₁-C₃ alkoxy, —C(O)R¹⁰, —COOR¹⁰, NR¹²R¹³, C(O)NR¹²R¹³, and —SO₂R¹¹;
R¹⁰ is independently at each occurrence H, C₁-C₄ alkyl optionally substituted with one to three groups selected from halo, —OH, and C₁-C₃ alkoxy;
R¹¹ is independently at each occurrence C₁-C₄ alkyl optionally substituted with one to three groups selected from halo, —OH, and C₁-C₃ alkoxy;
R¹² and R¹³ are independently at each occurrence selected from H, C(O)R¹⁴, C(O)OR¹⁴, C₁-C₄ haloalkyl, and C₁-C₄ alkyl optionally substituted with —OH or —OR¹⁴;
where R¹⁴ is independently at each occurrence C₁-C₄ alkyl optionally substituted with one to three groups selected from halo, —OH, and C₁-C₃ alkoxy;
or R¹² and R¹³ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, and C₁-C₃ alkoxy, and C₁-C₄ alkyl substituted with one or two groups selected from —OH, C₁-C₃ alkoxy, CN, SO₂R¹¹, —COOR¹⁰, —NR¹⁵R¹⁶, —NR^{b}C(O)R¹¹, and —CONR¹⁵R¹⁶;
R² and R⁶ are independently selected from H, halo, C₁₋₄ alkoxy, C₁₋₄ haloalkyl, C₁₋₄ haloalkoxy, CN and C₁₋₄ alkyl optionally substituted with one or two groups selected from the group consisting of halo, CN, hydroxy and C₁-C₃ alkoxy;
R³ and R⁵ are independently selected from H, halo, C₁₋₄ alkyl, C₁₋₄ alkoxy, C₁₋₄ haloalkyl, C₁₋₄ haloalkoxy, and CN;
Ar is phenyl or a 5-12 membered heteroaryl ring, and is optionally substituted by 1-3 groups independently selected from halo, hydroxy, C₁₋₄ alkyl, C₁₋₄ alkoxy, C₁₋₄ haloalkyl, C₁₋₄ haloalkoxy, CN, —SO₂R¹¹, —COOR¹⁰, —NR¹⁵R¹⁶, —NR^{b}C(O)R¹⁰, —CONR¹⁵R^{16,} and C₁-C₄ alkyl substituted with one or two groups selected from —OH, C₁-C₃ alkoxy, CN, SO₂R¹¹, —COOR¹⁰, —NR¹R¹⁶, —NR^{b}C(O)R¹¹—CONR¹⁵R¹⁶;
wherein R¹⁵ and R¹⁶ are independently H or C₁₋₄ alkyl;
or R¹⁵ and R¹⁶ taken together with N to which both are attached form a 4 to 8 membered heterocyclic ring optionally containing an additional N, O, or S as a ring member and optionally substituted with one or two groups selected from OH, oxo, halo, C₁-C₃ alkyl, C₁-C₃ haloalkyl, C₁-C₃ alkoxy, —C(O)R¹⁰, —COOR¹⁰, and —SO₂R¹¹;
each n is independently an integer selected from 0, 1, 2 and 3; and
each m is independently an integer selected from 0, 1 and 2;
or a pharmaceutically acceptable salt thereof.

2. The compound of claim 1, wherein R² is H, halo, C₁₋₄ alkyl, C₁₋₄ alkoxy, C₁₋₄ haloalkyl, or C₁₋₄ haloalkoxy.

3. The compound of claim 1, wherein R⁶ is halo, C₁₋₄ alkyl, C₁₋₄ alkoxy, C₁₋₄ haloalkyl, or C₁₋₄ haloalkoxy.

4. The compound of claim 1, wherein R³ is H, halo, C₁₋₄ alkyl, or C₁₋₄ haloalkyl.

5. The compound of claim 1, wherein R⁵ is is H, halo, C₁₋₄ alkyl, or C₁₋₄ haloalkyl.

6. (canceled)

7. The compound of claim 1, wherein Ar is phenyl or furanyl and is optionally substituted with one or two groups selected from halo, hydroxy, C₁₋₄ alkyl, C₁₋₄ alkoxy, C₁₋₄ haloalkyl, C₁₋₄ haloalkoxy, and CN.

8. The compound of claim 7, wherein Ar is phenyl optionally substituted with one or two groups selected from halo, C₁₋-C₂ alkyl, CN, and C₁-C₂ haloalkyl.

9. The compound of any one of the preceding claims claim 1, wherein L is O, [X]—CH₂—O—, or [X]—CH₂CH₂—O—.

10. The compound of claim 1, wherein R³ and R⁵ each represent H.

11. The compound of claim 1, wherein R² is C1-C2 alkyl.

12. The compound of claim 1, wherein R⁶ is C₁-C₂ haloalkyl.

13. The compound of claim 1, wherein X is (CH₂)₁₋₃ or pyridinyl or phenyl.

14. The compound claim 1, wherein X is —CHR^{a}— or —C(Me)₂—

15. The compound of claim 1, which is a compound of Formula (IA): 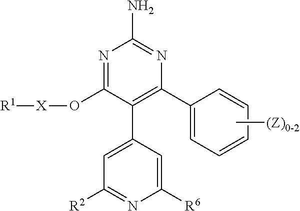
wherein each Z is independently selected from halo, C₁-C₄ alkyl, C₁-C₄ alkoxy, CN, C₁-C₂ haloalkyl, and C₁-C₂ haloalkoxy;
R² and R⁶ are independently selected from C₁₋₄ haloalkyl, C₁₋₄ haloalkoxy, and C₁₋₄ alkyl optionally substituted with one or two groups selected from the group consisting of halo, CN, hydroxy and C₁-C₃ alkoxy; and
X and R¹ are as set forth in claim 1;
or a pharmaceutically acceptable salt thereof

16-18. (canceled)

19. The compound of claim 15, wherein X is (CH₂)₁₋₃ or pyridinyl or phenyl.

20. The compound of claim 15, wherein X is —C(R^{a})₂— or —C(R^{a})₂—C(R^{a})₂—.

21. (canceled)

22. The compound of claim 1, wherein R¹ is —(CR^{a}₂)₀₋₂—Cy, (CR^{a}₂)₀₋₂—O—Cy, or —O—(CR^{a}₂)₁₋₂—Cy, wherein
Cy is a cyclic group selected from phenyl, C₃-C₈ cycloalkyl, a 5-6 membered monocyclic heteroaryl group having up to four heteroatoms selected from N, O and S as ring members, and a 3-8 membered heterocyclic ring comprising one or two heteroateoms selected from N, O and S as ring members, and is optionally fused to a phenyl or a 5-6 membered heteroaryl or a heterocyclic ring having one or two heteroatoms selected from N, O and S as ring members or a C₃-C₈ cycloalkyl ring, to form a bicyclic group; wherein
the cyclic or bicyclic group Cy is optionally substituted as described in claim 1.

23-24. (canceled)

24. (canceled)

25. The compound of claim 1, which is selected from the compounds in Table 1, or a pharmaceutically acceptable salt thereof.

26. A pharmaceutical composition comprising the compound of claim 1 or a pharmaceutically acceptable salt thereof, admixed with at least one pharmaceutically acceptable excipient.

27. A method to treat a proliferative disorder, cancer, inflammatory disease, renal disease, diabetes, vascular disease, lung disease, or an autoimmune disease, which comprises administering to a subject in need of such treatment the compound according to claim 1 or a pharmaceutically acceptable salt thereof, or the pharmaceutical composition of claim 26.

28. (canceled)
